(19) United States
(12) Patent Application Publication
(10) Pub. No.: US 2007/0156179 A1
(43) Pub. Date: Jul. 5, 2007

(54) Multi-channel and multi dimensional system and method

(76) Inventor: Karashurov S.E., St. Pervomayskiya Prospect (RU)

Correspondence Address: Angenehm Law Firm, Ltd., P.O. Box 48755, Coon Rapids, MN, 55448-0755, US

(21) Appl. No.: 11/220,729

(22) Filed: Sep. 6, 2005

(30) Foreign Application Priority Data
Mar. 6, 2003 (IL) 154801

(51) Int. Cl. A61N 1/18

(52) U.S. Cl. 607/2

(57) ABSTRACT

An implanted system for treatment of human diseases by electric stimulation and/or electric blocking of the body tissues, comprising sensor and/or biosensor means for measuring variables in the body, processor means connected to the sensors and biosensors for processing the measured variables and for deciding in real time whether to apply an electric signal to the body tissues, and electrode means implanted at predefined locations and connected to the processor means, for applying the stimulation and/or electric blocking signals to the body tissues. A method for treatment of human diseases using an implanted system by electric stimulation and/or electric blocking of the body tissues, comprising: A. measuring variables in the body using implanted sensor and/or biosensor means; B. processing the measured variables for deciding in real time whether to apply an electric signal to the body tissues; C. applying the stimulation and/or electric blocking signals to the body tissues.

### TECHNICAL FIELD

The present invention relates to a system and method for treatment of human diseases by electric stimulation and electric blocking of the body tissues using implanted, modular, multichannel, multisensor, multidimensional, adaptive and programmable structure with multidimensional sensitized electric stimulant microchips (visceral processors).

### BACKGROUND OF THE INVENTION

Treatment of human diseases by electric stimulation has been reported by researchers, and related patents have been issued.

Prior art analog systems have various disadvantages, for example:

1. Blocking of conductivity of the nervous impulses through the vagus nerve is carried out by means of electric stimulation, which is of low efficacy because the procedure is enabled by direct current only.

2. Electric stimulation of the vagus and other nerves (hypoglossus and glossopharyngius) can cause serious side effects, such as paresis of the above with salivation disorders, lingual deviations, pains; cardiac arrest, loss of voice.

Note: In the analog patents, the chip is called “Neurocybernetic prosthesis” (NCP). In the present disclosure, the following name is used:

“Multifunctional sensitized implanted microchip”.

3. The sensors control the chip's activation/deactivation mode only.

In the new system, however, this applies to adjustment of the chip to the body's needs. The algorithm can be selected automatically. Moreover, the new chips are multifunctional.

Examples of prior art, the following patents are included herein by reference:

Pat. No. Title

1 U.S. Pat. No. 6,473,644 Method to enhance cardiac capillary growth in heart failure patients
2 U.S. Pat. No. 5,928,272 Automatic activation of a neurostimulator device using a detection algorithm based on cardiac activity
3 U.S. Pat. No. 5,707,400 Treating refractory hypertension by nerve stimulation
4 U.S. Pat. No. 5,571,150 Treatment of patients in coma by nerve stimulation
5 U.S. Pat. No. 5,540,730 Treatment of motility disorders by nerve stimulation
6 U.S. Pat. No. 5,531,778 Circumneural electrode assembly
7 U.S. Pat. No. 5,351,394 Method of making a nerve electrode array
8 U.S. Pat. No. 5,335,657 Therapeutic treatment of sleep disorder by nerve stimulation
9 U.S. Pat. No. 5,330,515 Treatment of pain by vagal afferent stimulation
10 U.S. Pat. No. 5,304,206 Activation techniques for implantable medical device
11 U.S. Pat. No. 5,299,569 Treatment of neuropsychiatric disorders by nerve stimulation
12 U.S. Pat. No. 5,269,303 Treatment of dementia by nerve stimulation
13 U.S. Pat. No. 5,263,480 Treatment of eating disorders by nerve stimulation
14 U.S. Pat. No. 5,251,634 Helical nerve electrode
15 U.S. Pat. No. 5,237,991 Implantable medical device with dummy load for pre-implant testing in sterile package and facilitating electrical lead connection
16 U.S. Pat. No. 5,235,980 Implanted apparatus disabling switching regulator operation to allow radio frequency signal reception
17 U.S. Pat. No. 5,231,988 Treatment of endocrine disorders by nerve stimulation
18 U.S. Pat. No. 5,222,494 Implantable tissue stimulator output stabilization system
19 U.S. Pat. No. 5,215,089 Electrode assembly for nerve stimulation
20 U.S. Pat. No. 5,215,086 Therapeutic treatment of migraine symptoms by stimulation
21 U.S. Pat. No. 5,205,285 Voice suppression of vagal stimulation
22 U.S. Pat. No. 5,188,104 Treatment of eating disorders by nerve stimulation
23 U.S. Pat. No. 5,186,170 Simultaneous radio frequency and magnetic field microprocessor reset circuit
24 U.S. Pat. No. 5,179,950 Implanted apparatus having micro processor controlled current and voltage sources with reduced voltage levels when not providing stimulation
25 U.S. Pat. No. 5,154,172 Constant current sources with programmable voltage source
26 U.S. Pat. No. 4,979,511 Strain relief tether for implantable electrode

Other prior art patents include:
U.S. Pat. No. 5,700,282, Zabara: Heart rhythm stabilization using a neurocybernetic prosthesis
U.S. Pat. No. 5,540,734, Zabara: Cranial nerve stimulation treatment using neurocybernetic prosthesis.

The present inventor has been granted patents in the Russian Federation:
RU (11)2102090
RU (11) 2108121
RU (11) 2108817

A PCT application has been filed by the present inventor, International Application No.: PCT/RU 01/00126 filed on 27 Mar. 2001 and claiming priority from a prior application filed on 29 Mar. 2000.

### SUMMARY OF THE INVENTION

The present invention relates to an implanted system for the treatment of human diseases by electric stimulation and electric blocking of the body tissues (optional), and a method of operation of the system. The system and method use implanted modular, multichannel, multisensor, multidimensional, adaptive and programmable structure with multidimensional sensitized electric stimulant microchips (visceral processors).

A summary of the main inventive issues:

1 Hardware, structure of system and its component parts

1.1 System: modular, multichannel, multisensor, multidimensional, adaptive, programmable
1.2 Microchips—processor means
1.3 Sensors
1.4 Biosensors
1.5 Universal sensors
1.6 Electrodes
1.7 Wireless electrodes—Golden needle™
1.8 Autonomous power source
1.9 Contacts, or indirect body measurements using adaptive techniques
   2 Method of operation of the system, software, algorithms
   3 Method of treatment using the new system:
3.1 Treatment matrix. For each disease a matrix of: system structure
   method of operation of the system
   locations in the body for sensors, electrodes
3.2 Inverse treatment matrix. For a specific structure and implantation:
   List of all the diseases that are concurrently being treated (or can be treated, if diagnosed in the patient)
      4 Clinical results, practical experience using the new system and method are presented. The method has been kept secret, and the system is hidden inside the patient's body.

According to one aspect of the invention, it allows affecting [controlling] the functional activity of the systems and/or specific organs of the body by electric stimulation and/or blocking (with alternate and direct current respectively) thereof.

The affected systems and organs of the body include, for example: the nervous structure of the sympathetic nervous system or the parasympathetic system or the sympathetic nervous system and parasympathetic system and hypoglossal (sinocarotid collector of the Vegetative Nervous System-SCVNS), the central nervous system, as well as neurons of the organ and/or cutaneous nerves and/or depressor nerves.

In a preferred embodiment, a nervous band or group is formed, comprising all, or the majority of, the nerve branches innervating the carotid glome (glomus caroticum). The carotid glome is found in the area where the common carotid artery splits into the internal and external carotid arteries.

The above nervous band or group is formed using surgical tools.

According to another aspect of the invention, the system includes automatic adjustment of the microchip's channels to optimal algorithms of the software to electrically impact organs and tissues.

This becomes possible due to sensitization via multidimensional biosensors and sensors, to adapt the system to different conditions of various media of the body, fluids, gases dissolved in them, hormones versus electric and mechanical activity (including the murmurs) of the organs and structures of different systems of the body.

Thus the microchip can adapt, in real time, to changing conditions of the human body.

Furthermore, the system enables to directly and simultaneously control functions of several body organs or systems per different algorithms and to concurrently treat several diseases in one patient.

Moreover, the system allows for multi-purpose, modular use: chips of each generation can be used for treatment of various diseases. The only requirement to achieve that is to adequately select sensors, biosensors, electrodes and software's algorithms.

A multidimensional sensitization of the chips' coating (artificial skin-type with artificial multi-function sensor and biosensor receptors located on all the surfaces of the chip's shell and its electrodes). Both sensors, biosensors intended to measure one parameter of the body's homeostasis and those to monitor various parameters (for example: contents of substances, gases, analysis of electric and mechanical activity of the organs, etc.) can be located on each of the above-mentioned chip's parts.

This allows the chip simultaneously monitor functional activity of several systems of the body (nervous, cardiovascular, digestive, endocrine, urinary systems, etc.) and to finely adapt to the body's needs.

Multi-channel feature (for Chip 4 and more advanced versions), enabling to separately program all the parameters of outgoing pulses of the stimulation current, simulation modes (electric stimulation, blocking) (turning the chip on and off) in each of the channels. Linear, synchronized non-calibrated adjustment of each of the chip's channels to the optimal working algorithm while selecting the latter.

Multi-purpose use and multi-function feature of each channel (electric blocking, electric stimulation, etc.). A combination of channels can form an analog of a natural neuron net.

Novel cordless/wireless devices, such as electrodes connected to the mother chip via electromagnetic waves (for the Golden Needle™ type and others) in the multi-channel chips (Chip 6).

Up to 100 channels or more can be used with one chip, each channel capable of independent operation, each channel can treat a specific disease. Microscopic size of several chips (less than 1 mm) involving state-of-the art microelectronic technologies.

Microchips' sensitization: this feature is enabled due to a large number of microscopic biosensors and sensors located on all the surfaces of the microchips (similarly to the receptors on the human skin).

Microchips' implantation methods and their outlines: the chips are implanted using low-trauma surgeries (endoscopic procedures, etc.), as well as stereoscopic surgeries (“Golden Needle” chip and other similar versions). The microchip's shell is implanted into the subcutaneous fat in the patient's body, usually in the thorax. The electrodes are connected to various structures of the nervous and other systems of the body. The biosensors and sensors are connected to the relevant organs and systems.

The chip's adjustment to the optimal therapeutic plan is performed after the surgery, using the conventional diagnostic examination methods and a special external device to obtain a maximal therapeutic effect on an individual basis.

Additional Properties of the Microchips and Technologies:

1. Rules of connecting the electrodes to nervous structures: to the nerves, spinal cord, vegetative collectors (sinocarotid area), to neurons and nervous ganglia of the organs. The chip can be connected both to all of the above-mentioned structures together and separately to each other of them, depending on therapeutic tasks and objectives; from the right or left side only, or from both right and left side.

2. Biosensors—sensors

Each electrode and chip's shell can carry sensors and biosensors of various purposes, as well as these of one same purpose (for example, to measure blood oxygen level).

The number of sensors and biosensors may vary, for example between one and 16, according to the specific application.

Differences from the PCT application for Chip 3, PCT/RU 01/00126 include, among others, biosensors intended only to measure blood oxygen level, respiration rate and heart rate, but also other biosensors, as well as sensors of all types.

3. Mechanism of impact on the nervous structures: electric blocking with direct current (as in the patent—stimulation of the vagus in asthmatic patients), or electric stimulation with alternate current, or electric stimulation and blocking in different combinations, or all the above-mentioned used either together or separately.

4. Autonomous programming, controlling, power-supplying via a radio channel of each of the microchip's channels: setting activation and deactivation time, algorithms to amend the current's outgoing pulses in a dependence on signals from the biosensors, sensors of different types, separate setting of sensitivity thresholds for each of the biosensors.

Novel means and method enable to amend the chips' algorithms both by means of external reprogramming and by the microchip itself (Chip 5, Chip 6)

5. A possibility to impact all the nervous structures simultaneously, as well as separately: using both electric blocking and stimulation in different combinations and sequences.

6. Modes and software algorithms for each of the microchip's channels can be set as follows:

a) using an external computer-assisted unit.

b) using biosensors depending on their working algorithms.

c) manually by the physician or the patient themselves.

7. Object-oriented technique of imputing the algorithms: (for example, “to enhance the intestine's peristalsis”) with their automatic performance by the microchip.

8. A possibility to power the chip directly from kinetic energy of the patient's body: (see for example, FIG. 25B and the related disclosure), this allowing to increase the chips' useful life and reliability.

9. An option to locate the microchip's CPU both in an implanted device and an external one: on the patient's chest (attached to the patient's clothes in a form of a pin).

10. A possibility to telemetrically control and monitor the chip's operation and its properties.

11. Radio frequency performance devices without electrodes: implanted electrodes connected to the mother microchip (Chip 6) or only one of them, as well as sensors, biosensors (several or one in number).

See detailed description, for example with reference to FIGS. 12, 13 and relating to the Wireless electrodes—Golden needle™ below.

12. Multidimensional sensors enabling monitoring of various media of the body: contents of fluids, gases, hormones, electric, mechanical activity of organs and systems. Chip's activation indicator for the patient. Chip-assisted direct monitoring of the human nervous system's condition is carried out by analyzing electric activity of the nerves and brain.

13. Concepts of impacting organs and systems by the microchips: enhancing or suppressing the function of a specific organ or system, or its adjustment according to a priori set reference (adjustment to the patient's individual activity and needs).

14. Basic multifunctional algorithm with specific types of its implementation for each disease.

15. Each embodiment may include pairs of antipode diseases: for example: hypertension, hypotension.

16. The chip can be connected, for example, to any part of the sympathetic trunks, vagus nerves, spinal cord. Other locations are detailed in the present disclosure, see for example the disclosure below with reference to FIGS. 27 to 48.

17. The chip can be coated with Shungite, a mineral offering improved performance for implanted devices.

18. Using a learn mode, the system can initially use both sensors/biosensors and indirect sensors, whereas at a future stage it converts to using only the indirect sensors. This achieves reliable operation for prolonged time periods.

### BRIEF DESCRIPTION OF THE DRAWINGS

The invention is herein described, by way of example, with reference to the accompanying drawings:

FIG. 1—System structure

FIG. 2—Block diagram of microchip No. 1

FIG. 3—Structure of microchip No. 1

FIG. 4—Block diagram of microchip No. 2

FIG. 5—Structure of microchip No. 2

FIG. 6—Block diagram of microchip No. 3

FIG. 7—Structure of microchip No. 3

FIG. 8—Block diagram of microchip No. 4

FIG. 9—Structure of microchip No. 4

FIG. 10—Block diagram of microchip No. 5

FIG. 11—Structure of microchip No. 5

FIG. 12—Block diagram of microchip No. 6

FIG. 13—Structure of microchip No. 6

FIG. 14—Structure of sensors No. 8,9,10,16

FIG. 15—Structure of sensors No. 11,13

FIG. 16—Structure of sensors No. 12,15

FIG. 17—Structure of sensor No. 14

FIG. 18—Typical signals from sensors (codes)

FIG. 19—Structure of biosensor (Type A)

FIG. 20—Structure of biosensor (Type B)

FIG. 21—Typical signals from biosensors (codes)

FIG. 21B—Results of 24 hour sensors monitoring in a patient

FIG. 22—Structure of universal sensor

FIG. 23—Structure of electrode (Type A)

FIG. 24—Structure of electrode (Type B)

FIG. 25—Structure of electrode (Type C)

FIG. 25B—Structure and location of implanted power source

FIG. 25C—Structure of an external monitoring device

FIG. 26—Method of operation—flow chart

FIG. 27—Preferred implantation location in the SCVNS

FIG. 27B—Detail of Preferred implantation location in the SCVNS

FIGS. 28 to 48—Preferred implantation locations

FIGS. 49 to 52—Illustration of research methodology and results

FIGS. 53 to 57—Illustration of surgical procedure

FIGS. 58 to 67—Roentgen of patient with implanted system

FIG. 68—Structure of universal biosensor

FIG. 69—Structure of book-type bipolar electrode

FIG. 70—Structure of book-type multi-channel electrode

FIG. 71—Structure of book-type 2-8 polar or more electrode

FIG. 72—Structure of spiral electrode

FIG. 73—Structure of plate-like electrode

FIG. 74—Structure of wire gauze electrode

FIG. 75—Structure of coaxial electrode

FIG. 76—Structure of Golden Needle™ electrode

### DETAILED DESCRIPTION OF THE INVENTION

The principles and operation of the new system and method for treatment of human diseases by electric stimulation and electric blocking of the body tissues using an implanted programmable system may be better understood by way of example, with reference to the drawings and the accompanying description.

The description further includes the following tables.

### LIST OF TABLES

Table 1—System structure and therapeutic applications
Table 2—Sensors data
Table 3—Biosensors data
Table 4—Method of operation/algorithm for epilepsy
Table 5—Method of operation/algorithm for asthma
Table 6—Method of sensors/biosensors activation
Table 7—Treatment strategy. System structure and implant locations
Table 8—Electric stimulation parameters (A)
Table 9—Electric stimulation parameters (B)
Table 10—Experiments performed for each disease, in animals and humans
Table 11—Results of obesity treatment
Table 12—Statistics for obesity treatment
Table 13—Results of asthma treatment
Table 14—Asthma surgery data
Table 15—Epilepsy clinical examples
Table 16—Epilepsy surgery data
Table 17—Examples of gastric and duodenal ulcer treatment
Table 18—Clinical examples of dementia treatment
Table 19—Clinical examples of treatment of obliterating vascular diseases
Table 20—Conditions in a healthy patient that may be treated.

Novel features in the new system and method include, for example, a new approach to treatment of diseases using microchips from the existing conventional methods:

1. Enabling to directly and simultaneously control functions of several body organs or systems per different algorithms and to treat at a time several diseases in one patient.

2. Automatic adjustment of the microchip's channels to optimal algorithms of the software to electrically impact organs and tissues. This becomes possible due to sensitization via multidimensional biosensors and sensors guaranteeing adaptation to different conditions of various media of the body: fluids, gases dissolved in them, hormones versus electric and mechanical activity (including the murmurs) of the organs and structures of different systems of the body. Thus the microchip can adapt very precisely to a changing condition of the human body in a real-time mode.

3. Controlling functional activity of the systems and specific organs of the body not only by electric stimulation of nervous structures of the parasympathetic nervous system, but also by means of electric stimulation and blocking (with alternate and direct current) of structures of the sympathetic and parasympathetic nervous systems, as well as of the central nervous system (spinal cord), in different combinations, depending on a specific disease and the patient's condition.

Conceptual Differences of the New Chips from the Existing Similar Devices

1. Multi-purpose use: chips of each generation can be used for treatment of various diseases. The only requirement to achieve that is to adequately select sensors, biosensors, electrodes and software's algorithms.

2. Multidimensional sensitization of the chips' coating (artificial skin-type with artificial multi-function sensor and biosensor receptors located on all the surfaces of the chip's shell and its electrodes). Both sensors, biosensors intended to measure one parameter of the body's homeostasis and those to monitor various parameters (for example: contents of substances, gases, analysis of electric and mechanical activity of the organs, etc.) can be located on each of the above-mentioned chip's parts. This allows the chip simultaneously monitor functional activity of several systems of the body (nervous, cardiovascular, digestive, endocrine, urinary systems, etc.) and to finely adapt to the body's needs (absolute novelty).

3. Multi-channel feature (for Chip 4 and more advanced versions), enabling to separately program all the parameters of outgoing pulses of the stimulation current, simulation modes (electric stimulation, blocking) (turning the chip on and off) in each of the channels. Linear, synchronized non-calibrated adjustment of each of the chip's channels to the optimal working algorithm while selecting the latter.

4. Multi-purpose use and multi-function feature of each channel (electric blocking, electric stimulation, etc.). A combination of channels can form an analog of a natural neuron net.

5. Availability of cordless performance devices, such as electrodes connected to the mother chip via electromagnetic waves (“Golden Needle”-type and others) in the multi-channel chips (Chip 6) (absolute novelty).

6. Microscopic size of several chips (less than 1 mm) involving state-of-the art microelectronic technologies.

7. An option to power the microchips directly from the human body's kinetic energy allowing to increase the chips' useful life and reliability.

General Data on Multi-Organ Microchip Processors

1. Main components of the microchip: a CPU (microcomputer or micro-controller), multi-purpose electrodes with biosensors, sensors, an external programming and power-supplying unit connected to the computer.

2. Microchips' sensitization: this feature is enabled due to a large number of microscopic biosensors and sensors located on all the surfaces of the microchips (similarly to the receptors on the human skin).

3. Microchips' implantation methods and their outlines: the chips are implanted using low-trauma surgeries (endoscopic procedures, etc.), as well as stereoscopic surgeries (“Golden Needle” chip and other similar versions). The microchip's shell is implanted into the subcutaneous fat in the patient's body, usually in the thorax. The electrodes are connected to various structures of the nervous and other systems of the body. The biosensors and sensors are connected to the relevant organs and systems.

The chip's adjustment to the optimal therapeutic plan is performed after the surgery, using the conventional diagnostic examination methods and a special external device to obtain a maximal therapeutic effect on an individual basis.

1. Rules of connecting the electrodes to nervous structures: to the nerves, spinal cord, vegetative collectors (sinocarotid area), to neurons and nervous ganglia of the organs. The chip can be connected both to all of the above-mentioned structures together and separately to each other of them, depending on therapeutic tasks and objectives; from the right or left side only, or from both right and left side.

2. Biosensors—sensors

Each electrode and the chip's shell, can carry sensors and biosensors of various purposes, as well as these of one same purpose (for example, to measure blood oxygen level).

Differences from prior art and International Application PCT/RU 01/00126 include, among others, biosensors intended only to measure blood oxygen level, respiration rate and heart rate, but also other biosensors, as well as sensors of all types.

3. Mechanism of impact on the nervous structures: electric blocking with direct current (as in the patent stimulation of the vagus in asthmatic patients), or electric stimulation with alternate current, or electric stimulation and blocking in different combinations, or all the above-mentioned used either together or separately.

4. Autonomous programming, controlling, power-supplying via a radio channel of each of the microchip's channels: setting activation and deactivation time, algorithms to amend the current's outgoing pulses in a dependence on signals from the biosensors, sensors of different types, separate setting of sensitivity thresholds for each of he biosensors.

The novel structure and operation of the system allows to amend the chips' algorithms, both by means of external reprogramming and by the microchip itself (Chip 5, Chip 6).

5. A possibility to simultaneously affect a plurality of the nervous structures, as well as each one separately, using both electric blocking and/or stimulation, in various combinations and sequences.

6. Modes of operation and software algorithms for each of the microchip's channels can be set as follows: a) using an external computer-assisted unit; b) using biosensors depending on their working algorithms; c) manually by the physician or the patient themselves.

7. Object-oriented technique of imputing the algorithms (for example, “to enhance the intestine's peristalsis”) with their automatic performance by the microchip.

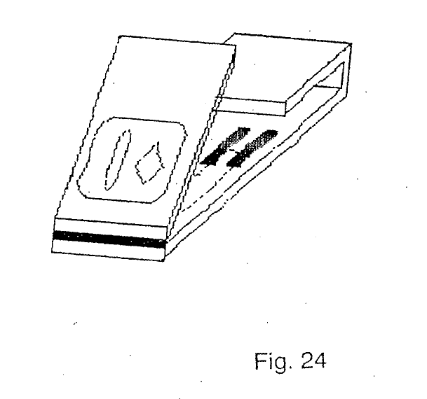

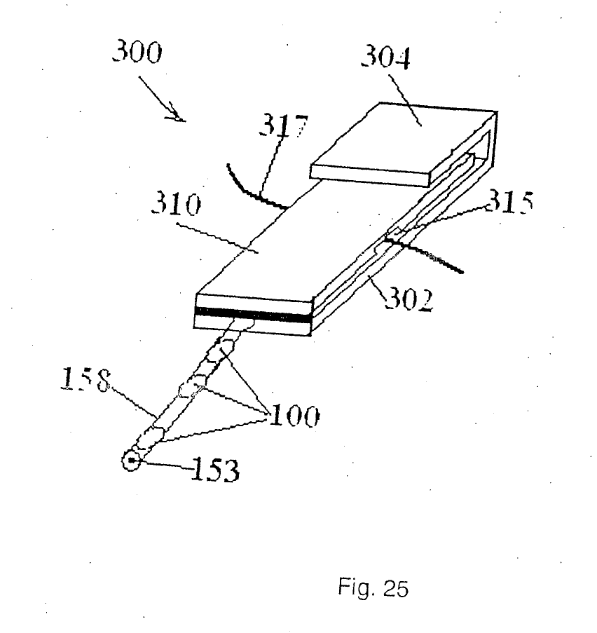

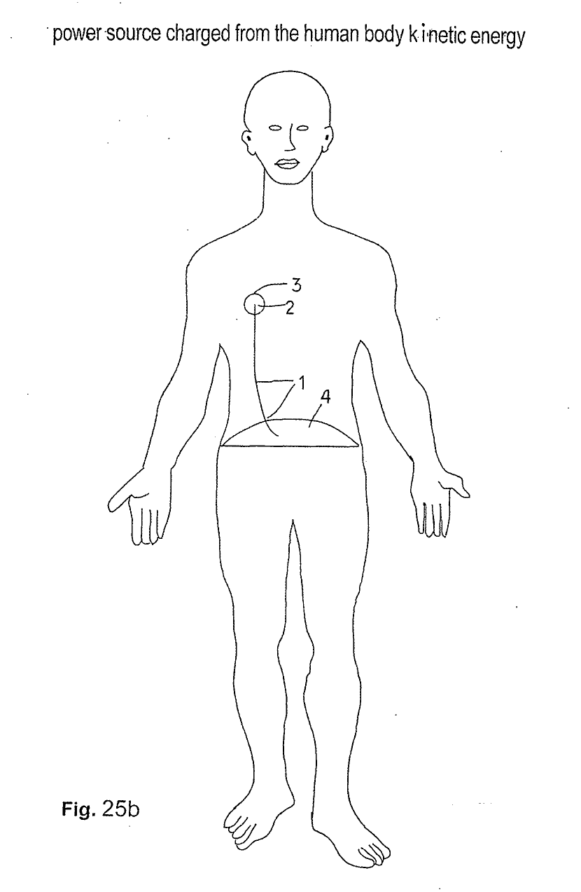

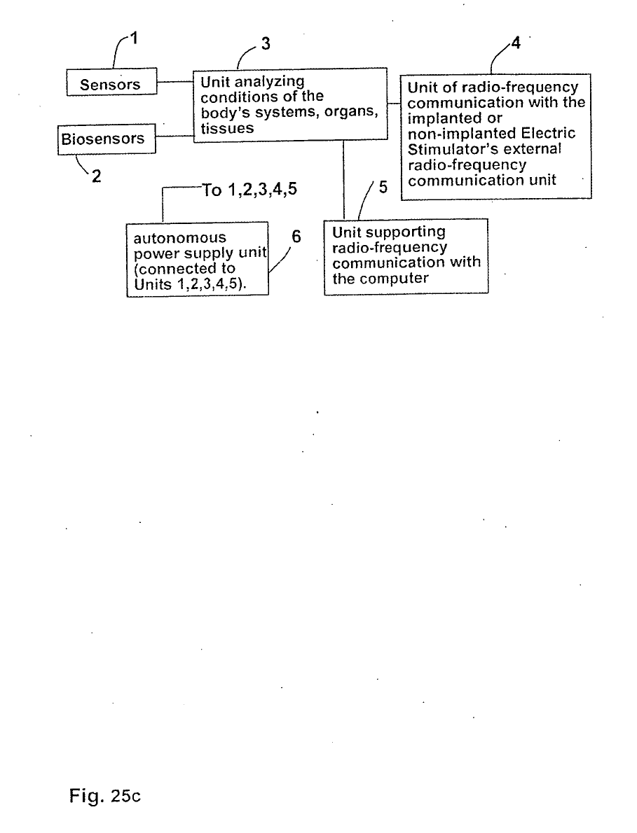

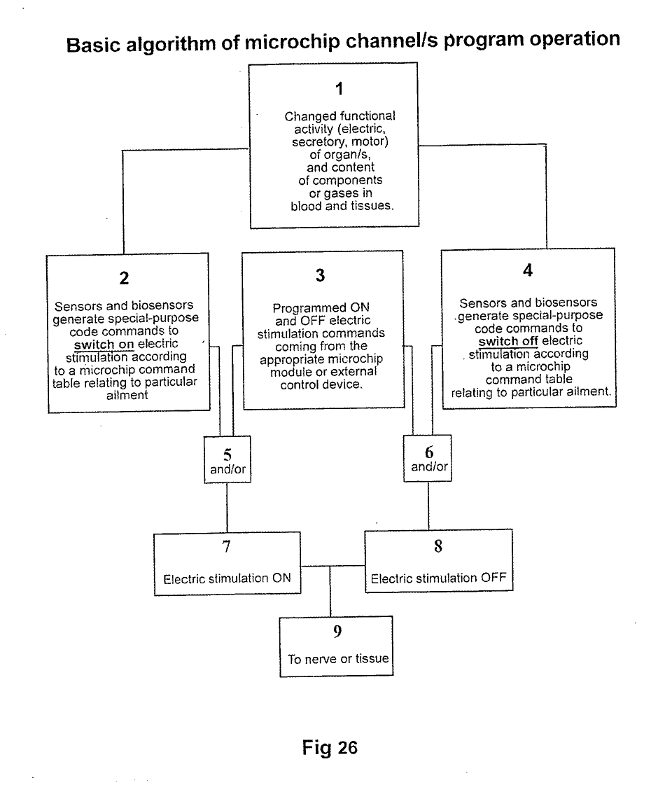

8. Means for powering the chip directly from kinetic energy of the patient's body (for example, see FIG. 25B—Structure and location of implanted power source).

9. An option to locate the microchip's CPU in an implanted device or an external one, on the patient's chest (attached to the patient's clothes in the form of a pin).

10. A possibility to control and monitor the chip's operation and its properties by remote control (telemetry), see details below.

11. Radio frequency performance devices without electrodes—implanted electrodes connected to the mother microchip (Chip 6) or only one of them, as well as sensors, biosensors (one or several at once).

12. Multidimensional sensors enabling monitoring of various media of the to body: contents of fluids, gases, hormones, electric, mechanical activity of organs and systems. Chip's activation indicator for the patient.

Chip-assisted direct monitoring of the human nervous system's condition carried out by analyzing electric activity of the nerves and brain.

13. Means for affecting organs and body systems by the microchips: enhancing or suppressing the function of a specific organ or system, or its normalization per a preset reference (adjustment to the patient's individual activity and needs).

14. Basic multifunctional algorithm with specific types of its implementation for each disease.

15. Each embodiment includes pairs of antipode diseases, for example: hypertension, hypotension.

16. The chip can be connected to any part of the sympathetic trunks, vagus nerves, spinal cord.

1 Hardware, Structure of System and its Component Parts

1.1 System: Modular, Multichannel, Multisensor, Multidimensional, Adaptive, Programmable

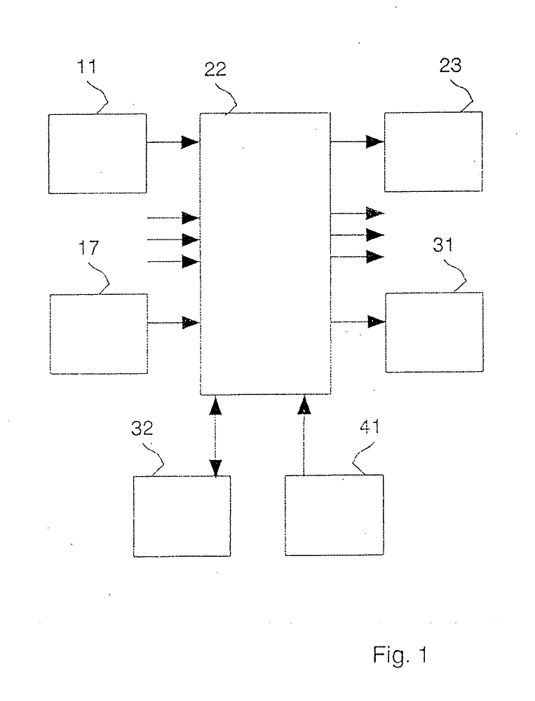

The system is detailed with reference to FIG. 1, and includes: a plurality of sensors 11, used to measure variables in the patient's body, and connected to a microchip 22 to transfer the results of the above measurements for processing. It may also include a plurality of biosensors 17 are used to measure in the patient's body, and also are connected to electrodes 23, 31 which are used to activate and/or block nerves in the body.

A manual activation and control means 32 is used to manually activate the device and/or read measurements values inside the patient's body.

The system further includes power supply means 41.

See Table 1—System structure and therapeutic applications.

1.2 Microchips

The chips' parts are preferably made of silicone, titanium, gold (999 purity 10 degree), platinum, stainless steel. Throughout the present disclosure, the term “Chip” or “Microchip” is used to designate a digital processor which may include a central processing unit CPU, memory and input/output channels.

Working Principle and Algorithm—Chip 1

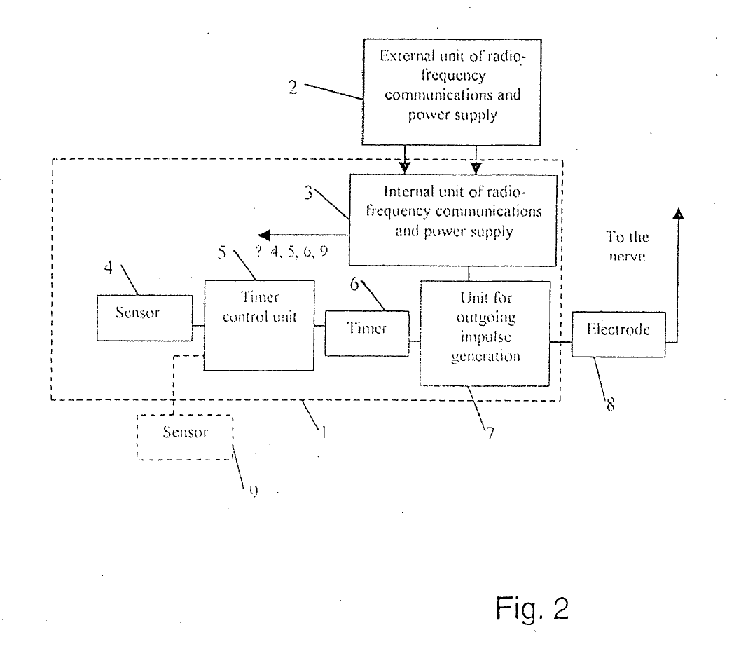

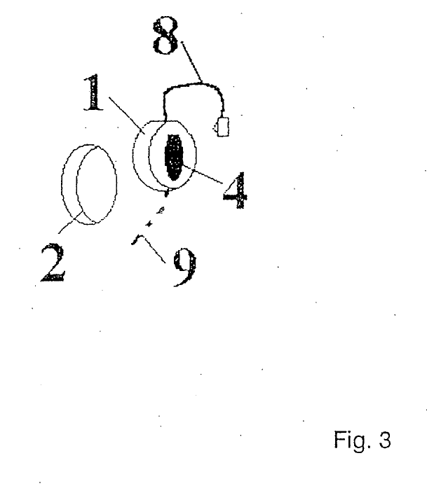

See FIG. 2—Block diagram of microchip No. 1 and FIG. 3—Structure of microchip No. 1

Unit 2 has two communication channels with Unit 3 enabled by electromagnetic waves. The first directly connects Unit 2, via Units 3,7 with the Electrode 8 thus making it possible to stimulate the nerve with impulses from Unit 2 when a breakdown of the chip occurs. The second channel transmits power to Units 3,4,5,6,7,9. Unit 7 is an impulse generator having preset, unchangeable through Units 2,3, parameters of outgoing impulses.

Unit 7 operates periodically this function being supported by the Timer 5 and by the Sensor located on the Chip's Shell 4 or the Sensor 9 from outside the shell. The Timer 6 sets the duration of stimulation sessions or duration of pauses between sessions. The Sensor controls only one parameter: duration of intervals between sessions or session duration.

Working Algorithm of Chip 1:

A change of the stimulation mode (stimulation session frequency or session duration) depends on the sensor's signal value (that, in its turn, depends on a particular time period of the day and the patient's activity); the signal is received by Unit 5 which either increases or reduces the algorithm (of increase or reduction), depending on the algorithm input in the chip's design at the manufacturing stage; its purpose is to solve any specific problem when different ailments are treated.

Examples of the Chip's Operation

Example 1

A heart rate meter (HRM) or a respiration rate meter (RRM) or an Arterial Pressure Meter (APM) or a muscular electric activity gage (MEAG) are used as a sensor. At day time the values of HRM, RRM, APM, MEAG are higher than at night.

These values also increase when the patient's physical activity becomes more intensive. This automatically, via the sensor, changes stimulation sessions frequency or session duration depending on the algorithm (increasing or reducing the frequency or the duration) input into Chip 1 and individually adapted for treatment of a specific ailment.

Example 2

A gage of the brain's paroxysmal activity (“peak-wave”-type complex) is used as a sensor in the epileptic patient. During or before an epileptic seizure, these complexes grow in number, which increases the signal transmitted from the Sensor 9 to Unit 5. This makes sessions of the sinocarotid nerve's stimulation more frequent thus preventing the seizure or quickly stopping it.

Novelty, Invention Standard

Adaptation of the stimulation mode to individual physical activity of the patient's body or to a symptoms severity degree of any specific ailment.

Working Principle and Algorithm—Chip 2

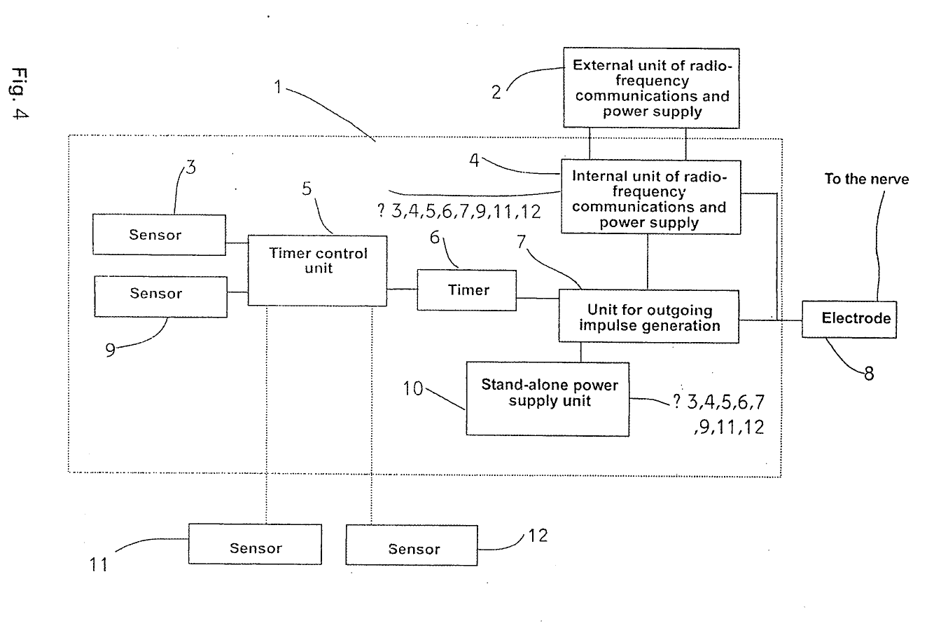

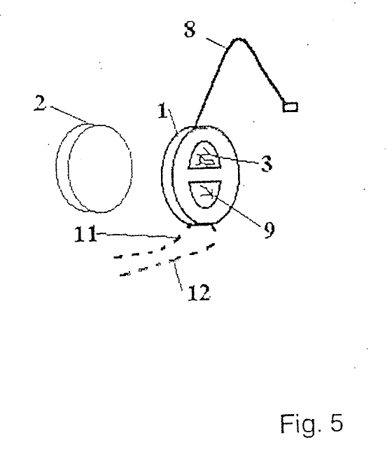

See FIG. 4—block diagram of microchip No. 2, and FIG. 5—Structure of microchip No. 2

Unit 2 has two communication channels with Unit 4 enabled by electromagnetic waves. The first directly connects Unit 2, via Units 4, 7 with the Electrode 8 thus making it possible to stimulate the nerve with impulses from Unit 2 when a breakdown of the chip occurs. The second channel transmits power to Units 3, 5, 6, 7, 9, 11, 12 via Unit 4 and charges power Unit 10 via Unit 4 and Unit 7.

Unit 7 is an impulse generator having preset, unchangeable through Units 2, 4, parameters of outgoing impulses. Unit 7 operates periodically this function being supported by the Timer 6, Timer control unit 5 and the Sensors located on the Chip's Shell 3, 9 from outside the shell 11, 12.

The Timer 6 sets the duration of stimulation sessions and duration of pauses between sessions. One Sensor controls the session duration, while the other is in charge of frequency of the sessions (i.e., duration of intervals between sessions).

Working Algorithm of Chip 2:

A change of the stimulation mode (stimulation session frequency and session duration) depends on the sensor's signal value (that, in its turn, depends on a particular time period of the day, the patient's activity, and severity of symptoms of the disease);

Frequency of the simulation sessions depends on the value of the first sensor's signal delivered to Unit 5 (which either increases or reduces the required algorithm depending on the built-in chip structure—increase or reduction designed to solve a particular problem), while the simulation sessions duration depending on the value of the first sensor's signal (which either increases or reduces the required algorithm depending on the built-in chip structure—increase or reduction designed to solve a particular treatment problem).

2 Examples of the Chip'S 2 Operation

Example 1

A heart rate meter (HRM) is used as the first sensor, and a respiration rate meter (RRM) is used as the second sensor. At day time the values of HRM, and RRM are higher than at night. These values also increase as the patient's physical activity becomes more intensive and the disease symptoms deteriorate. This automatically, via the appropriate sensor, changes stimulation sessions frequency and duration depending on the algorithm (increasing or reducing the frequency and the duration) input into Chip 2 and individually adapted for treatment of a specific ailment.

Example 2

A heart rate meter (HRM) is used as the first sensor, and a respiration rate meter (RRM) is used as the second sensor in the asthmatic patient. At the onset of dyspnea seizure, the patient develops a higher HRM and RRM, which increases the signal transmitted from the Sensors to Unit 5. This results in higher frequency of the sinocarotid nerve's stimulation sessions and a reflex dilatation of bronchi thus stopping the seizure.

Differences Between Chip 1 and Chip 2

The differences include, for example:

1. Chip 2 is equipped with a stand-alone power supply unit (which is inavailable in Chip 1)

2. Chip 2 has two sensors (Chip 1 has only one sensor)

3. In Chip 2, each sensor is in charge of one of the two stimulation mode parameters using the timer—sessions frequency or duration (the sensor in Chip 1 controls only one of these parameters), which allows to increase the accuracy of chip adaptation to specific needs of the patient.

Novelty, Invention Standard

Accurate adaptation of the stimulation mode to individual physical activity of the patient's body or to a symptoms severity degree of any specific ailment.

Working Principle and Algorithm—Chip 3

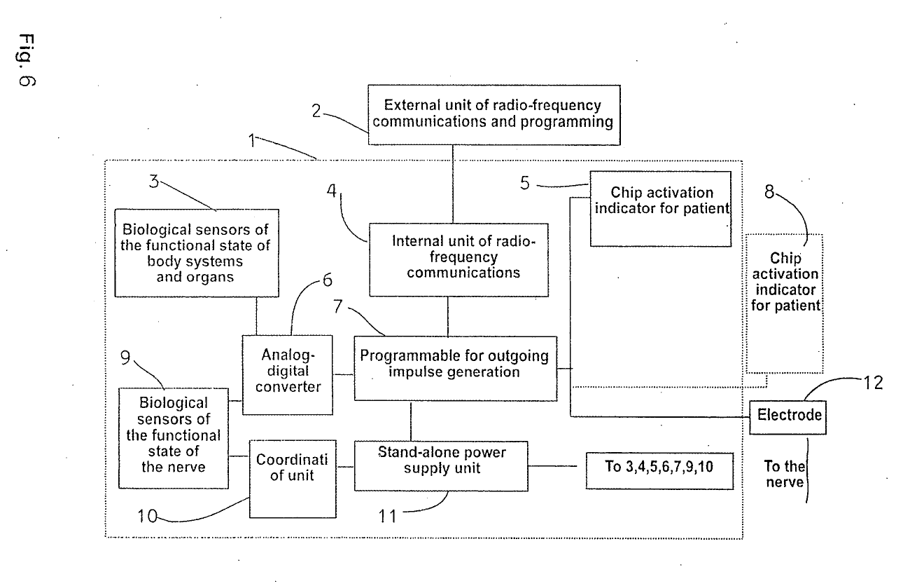

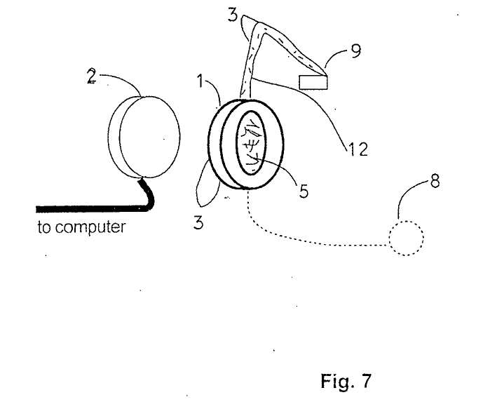

See FIG. 6—Block diagram of microchip No. 3, and FIG. 7—Structure of microchip No. 3

Function and Communications of Unit 2

1. Unit 2 records the chip operation algorithms described below to Unit 7 by means of electromagnetic waves and Unit 4.

2. Unit 2 enables programming of the following parameters of Unit 7 using Unit 4:

a) All outgoing pulses parameters, chip ON and OFF time.

b) ON and OFF time of internal singaling unit 5 or external singaling unit 8 to inform the patient on the start or end of the simulation session.

c) parameters of analog-digital converter 6, and—via Unit 6 parameters of biological sensors 3, 9 in the chip housing, in electrode 12, and in the contacts of electrode 9.

d) Unit 11 parameters—using Units 4 and 7.

3. Unit 2 via Unit 4, Unit 7 charges Unit 11 and allows to control its state.

4. Unit 2 via Unit 4, Unit 7 is directly connected with Electrode 12 via one of the two channels available in Units 2, 4, thus making it possible to transmit nerve simulation pulses when a breakdown of chip or discharge of Unit 11 power supply unit occur.

Function and Communications of Unit 3:

1. Unit 3 i.e., biological sensors 3 on the chip housing and on the housing of electrode 12,—transmits pulses to Unit 7 from Unit 6 (whose parameters depend on the functional state of the body systems controlled) by affecting the chip outgoing pulses parameters depending its working algorithm which was input from Unit 2 via Unit 4.

Function and Communications of Unit 9—Biological Sensors of the Functional State of the Nerve

1. The signal depending on the intensity of nerve electric activity is fed to Unit 7 from the Unit 9 biological sensors via Unit 6, and changes the chip outgoing pulses parameters according to algorithms algorithm which was input into Unit 7 from Unit 2.

2. Signal generated by Unit 9 biological sensors is fed to Power Supply Unit 11 via Unit 10 to charge Unit 11.

Function and Communications of Unit 11—the Stand-Alone Chip Power Supply Unit:

1. Unit 11 provides power supply to Units 3,4, 5, 6, 7, 9, 10.

2. Unit 11 is charged by Unit 9 via Unit 10 and by Unit 2 via Units 4 and 7.

Function and Communications of Unit 5 and Unit 8

Unit 5 built-in ON/OFF indicator for patient, Unit 8—similar internal indicator,—both Units are connected via the Unit 7 output with electrode 12

Working Algorithm of Chip 3

1. Increase or reduction of current, voltage, output pulses duration, and stimulation sessions frequency and duration by increasing or reducing signals of biological sensors (Units 3, 9).

Novelty, Invention Standard

1) Biological sensors are located directly on the chip and electrode housing.

2) The majority of chip units are connected with its radiofrequency communication component, which enables a direct control and adaptability thereof by means of the external programming unit.

3) Stimulation sessions and intervals of determining the nerve electric activity proceed in succession and not concurrently.

4) The chip operation modes are programmed by external programming unit (setup of simulation thresholds, working algorithms, etc.).

5) Power battery of the stand-alone power unit may be charged by the following sources: external programming unit, bioelectric activity of nerve, Means for powering the chip directly from kinetic energy of the patient's body (for example, see FIG. 25B—Structure and location of implanted power source).

Furthermore, power supply to the chip can be provided by electromagnetic waves transmitted from the external programming unit.

6) To add reliability, onset of seizure or deterioration of other ailment symptoms are identified according to a set of parameters.

7) Indicator informs the patients, when the stimulation device starts and finishes working.

Examples of the Chip'S 3 Operation

Example 1

Chip 3 was implanted into subcutaneous fat in the infraclavicular region of the astmatic patient, with the electrode connected to sinocarotid nerve (SCN). Chip ON indicator was implanted beside the chip (oscillator). The onset of seizure was associated with a reduced content of oxygen and hormones in the patient blood, and a higher electric activity of SCN. These changes were registered by the biological sensors. Afterwards, the sensors activated the chip for 10 minutes, according to its working algorithm, and the patient was prompted accordingly by the indicator.

Output pulses parameters were changed depending on the chip working algorithm, and the biosensors signals value.

Sinocarotid nerve's stimulation results in reflex dilatation of bronchi thus stopping the seizure. Oxygen content in the patient blood was increased with the SCN electric activity reduced. In response to these changes, the biological sensors disengaged the chip via Unit 7. The patient felt it, because the indicating oscillator stopped working. While stopping the seizure, the power supply unit was charged from the nerve.

Working Principle and Algorithm—Chip 4

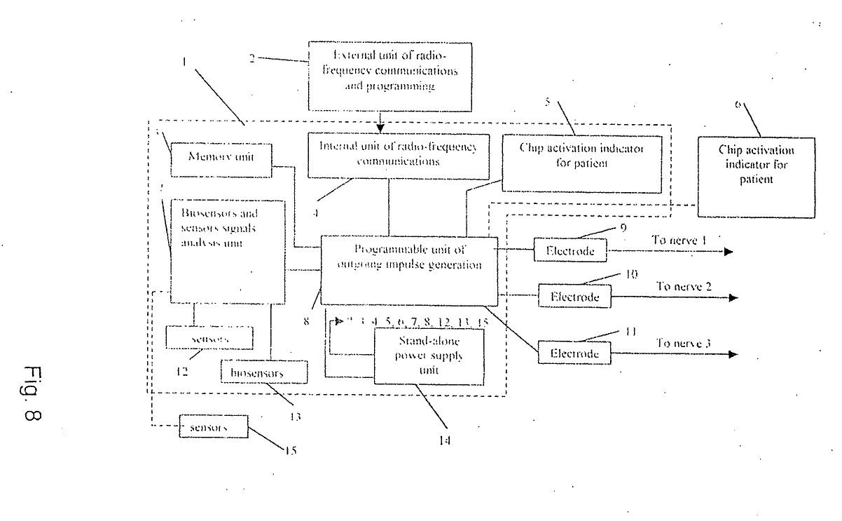

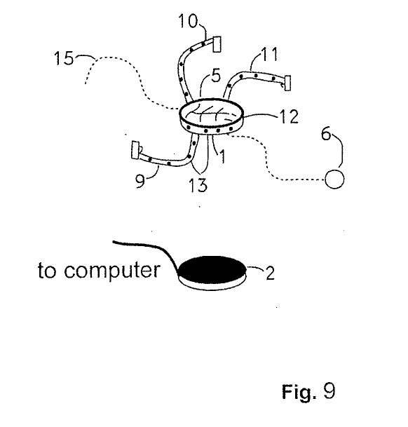

See FIG. 8—Block Diagram of Microchip No. 4, and FIG. 9—Structure of Microchip No. 4

Function and Communications of Unit 2

1. Unit 2 records the chip and chip channels operation algorithms, as described below, to Unit 8 by means of electromagnetic waves and Unit 4.

2. Unit 2 enables programming of the following parameters of Unit 8 using Unit 4:

a) All outgoing pulses parameters of all channels.

b) ON and OFF time of each channel, ON and OFF time of internal singaling unit 5 or external singaling unit 6 to inform the patient on the start or end of the simulation session via a certain channel.

c) Parameters of biosensors 13 and sensors 12, 15 in the housing of the chip and electrodes—via Unit 7.

3. Unit 2 charges Unit 14 via Unit 4 and Unit 8 and allows to control its state.

4. Unit 2 is directly connected with all electrodes in the chip channels via Unit 4, and Unit 8, thus making it possible to transmit nerve simulation pulses when a breakdown of chip or discharge of Unit 14 power supply unit occur.

Function and Communications of Unit 8

1. Unit 8 is in charge of creating non-connected channels to transmit output pulses to the electrodes connected to various organs and of controlling these channels according to the algorithms which were input to the memory unit 3 connected thereto.

2. Unit 8 is connected to sensors and biosensors via their signals analysis unit 7. Unit 7 chooses those signals of sensors and biosensors which are capable of changing the operation of Unit 8 and the channels controlled thereby according to the algorithm.

Sensors and biosensors are positioned both in the chip and internal electrodes housing and in special electrodes.

Function and Communications of Unit 14

1. Unit 14 is connected to all chip units, sensors, and biosensors, and supplies power thereto.

2. Unit 14 is connected to Unit 2 via Unit 4, and Unit 8, and can be chargeable via these Units.

Function and Communications of Unit 5 and Unit 6

Unit 5 and Unit 6—i.e., chip ON/OFF indicators, are connected to Unit 8, which, in its turn, further connects them to all chip channels.

Working Algorithm of Chip 4

1. Increase or reduction of current, voltage, output pulses duration, and stimulation sessions frequency and duration via each channel depending on an increase or reduction of signals of the Units 12, 13, 15 sensors and biological sensors.

2. Changing of the sequence and combination of activated chip channels depending on the current state of stimulated organs and tissues, as determined by sensors, biosensors, and algorithm stored in Memory Unit

3. Novelty, Invention Standard

1. Multichannel design

2. Biosensors design

3. An optimal automatic selection of organs and tissues stimulation parameters using biological sensors, sensors and chip memory algorithms.

4. A parallel control of severals organs.

Example of the Chip'S 4 Operation

A patient suffers from digestive and biliary disorders caused by gastric ulcer and dyskinesia of bile duct. Chip 3 was implanted into subcutaneous fat in the infraclavicular region. Gastric juice pH sensor is videolaparoscopically stitched to the stomach with the first channel electrode connected to sympathetic nerves of the stomach.

Gallbladder bile sensor is stitched to the gallbladder wall with the chip second channel electrode connected to the gallbladder muscular wall. Chip is programmed so that sympathetic nerves of the stomach be stimulated every three hours to reduce the higher gastric juice pH, which is one of the reasons of the gastric ulcer. Gastric juice pH sensor is programmed so that nerve stimulation be stopped as soon as gastric juice pH is reduced to 6. The channel connected to the gallbladder is programmed so that gallbladder contractions be induced during breakfast, lunch and dinner, which results in bile inflow to the duodenum and improves digestion.

Sensor stitched to the gallbladder is programmed so that the second channel responsible for stimulating gallbladder contractions be disconnected as soon as gallbladder is emptied. This creates conditions to facilitate healing of gastric ulcer and better digestion by means of programmed emptying of malfunctioning bile ducts.

Working Principle and Algorithm—Chip 5

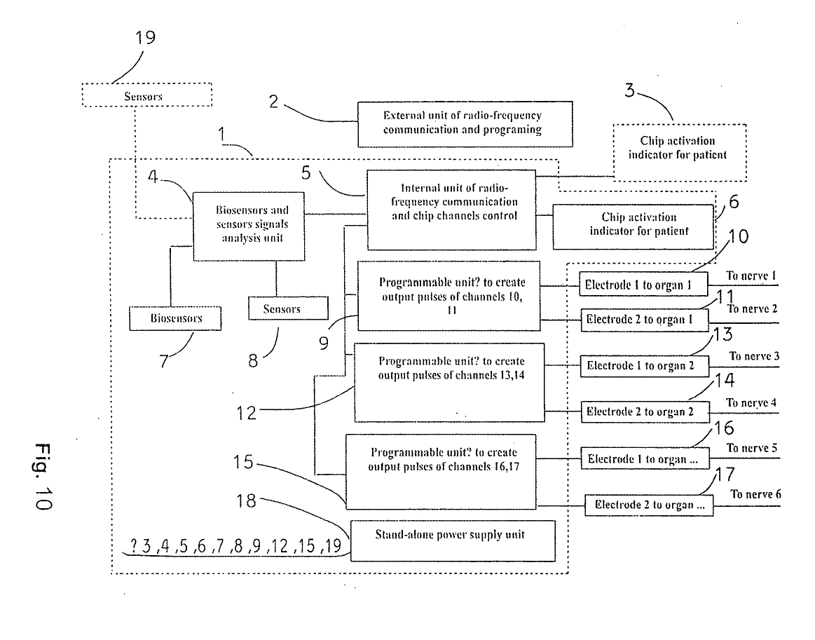

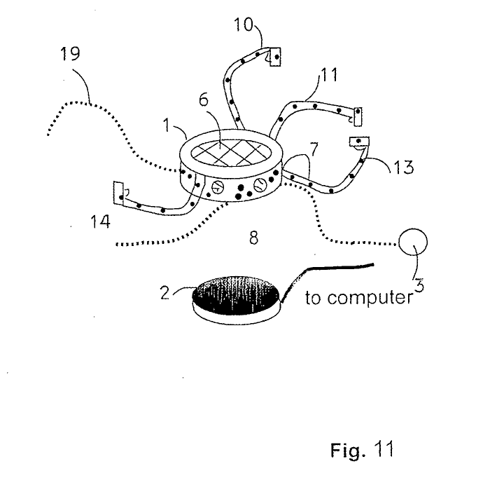

See FIG. 10—block diagram of microchip No. 5, and FIG. 11—Structure of microchip No. 5

The function and communications of Units 2, 4, 19, 7, 9, and 18 are similar to those of Chip 4.

The function and communications of Units 5, 9, 12, 15 and Units (electrodes) 10, 11, 12, 13, 14, 15, 16, 17 include, for example:

5 controls the order of connecting to channels ?, ?, ? of the electrodes depending on the algorithm and biosensors' signal values. Each channel is equipped with two additional channels (Units 10-17), whose output pulses can have the same or opposite sign. Paired electrodes are designed to stimulate similar structures on the right and left sides (such as vagus nerves).

Channels stimulation parameters are programmed individually per each channel using Unit 2.

Working Algorithm of Chip 5

1. Increase or reduction of current, voltage, output pulses duration, and stimulation sessions frequency and duration via each channel depending on an increase or reduction of signals of sensors and biological sensors.

2. Changing of sequence and combination of activated chip channels depending on the biosensors' signals values.

Novelty, Invention Standard (Chip 5)

1. A parallel control of several organs using various stimulation programs to resume their functions.

2. Biosensors design, concurrent monitoring of the state of several body organs and systems.

3. Use of at least two isolated channels to control each organ or system in order to provide a concurrent effect on several nerve centers and optimize the results of treatment.

Example of the Chip'S 5 Operation (Visceral Brain)

A patient suffers from a number of severe ailments:

1. Frequent attacks of angina pectoris

2. Hormone-caused diabetes mellitus of II degree.

3. Obliterating atherosclerosis of lower extremities of II-III degree.

Chip 5 was implanted to treat angina pectoris and other ailments. The first electrode of channel “A” was connected to the right sinocarotid nerve, the second electrode being connected to the left one. Sinocarotid nerve's stimulation results in reflex dilatation of coronaria and stopping attacks of angina pectoris.

A sensor of oxygen content in tissues and a heart rate meter were used to select optimal programs of nerve stimulation.

The first electrode of the second channel “B” was connected to the right vagus nerve to treat diabetes mellitus. A biological sensor of sugar content in blood was implanted to provide an automatic chip adjustment to the optimal stimulation program.

A special-purpose electrode was implanted in peridural space of the thorasic part of the spinal cord and connected to the second electrode of the channel “B” to treat obliterating atherosclerosis of lower extremities and angina pectoris.

Blood flow meter was implanted to femur.

The chip was programmed so that the said structures stimulation sessions result in a pronounced clinical effect, such as lower incidence of angina pectoris attacks, normal sugar level in blood, and better blood circulation in lower extremities.

Working Principle and Algorithm—Chip 6

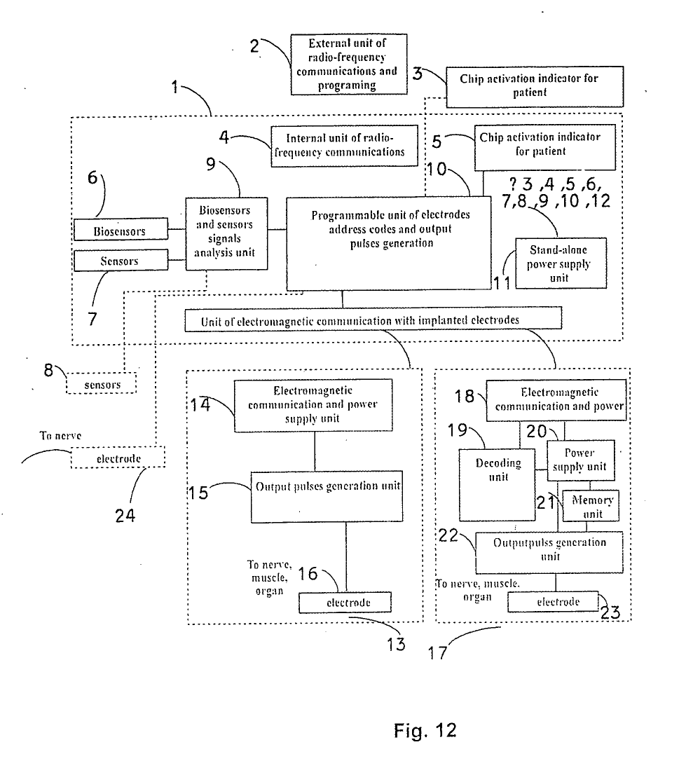

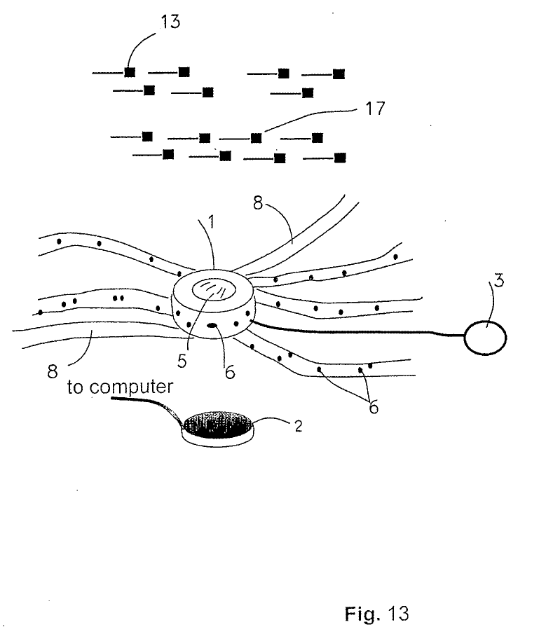

See FIG. 12-Block diagram of microchip No. 6, and FIG. 13-Structure of microchip No. 6

Chip 6 comprises three basic components:

The chip itself (Unit 1), which contains a sophisticated system of sensors and biosensors; a programming and communication unit (Unit 2) connected to the chip by means of electromagnetic waves; and various electrodes with the most important ones connected to the chip by means of electromagnetic waves (Gold Needle 1 (Unit 13), Gold Needle 2 (Unit 17).

The Chip can also be connected to conventional electrodes (Unit 24).

Numerous isolated channels (up to 100 or more), whose current parameters and activation modes can be programmed by Unit 10, allow to solve most complicated body functions control problems. Electrodes Gold Needle have address codes thus enabling an independent operation of channels. This is provided by encoding Unit 10 in the chip, and decoding Unit in Gold Needle No isolated connections with either channel are provided in Gold Needle 1. Radio frequency performance devices without electrodes: implanted electrodes connected to the mother microchip (Chip 6) or only one of them, as well as sensors, biosensors (one or more units).

Memory unit of Electrode 17 may contain a bank of address codes. Function and communications of Units 2, 3, 5, 4, 11, 6, 7, 8, 24, 15, 22, 16, 23, 20, 21 are basically similar to those of Chip 4. These are described in the summary table.

Working Algorithms of Chip 6

1. Increase or reduction of current, voltage, output pulses duration, and stimulation sessions frequency and duration via each channel depending on an increase or reduction of signals of sensors and biological sensors.

2. Changing of sequence and combination of activated chip channels depending on the current state of stimulated organs and tissues, as determined by biosensors.

Novelty, Invention Standard (Chip 6)

1. Wireless implantable families of secondary microelectrodes with individual programming of operation modes and parent chip connection by means of electromagnetic waves.

2. Biosensors design.

3. Monitoring of body systems and functions by means of various biological sensors.

4. Concurrent stimulation of over 100 nerves, organs, and muscles using various programs with a parallel monitoring of the results.

Example of the Chip'S 6 Operation, Using a Gold Needle™

A patient suffers from the following ailments:

1. paraphlegia caused by spine fracture; and

2. atony of urinary bladder and enuresis;

3. left crus ulcer caused by

4. angiotrophoneurosis as a complication of the above fracture. Chip 6 was implanted into subcutaneous fat in the infraclavicular region under local anesthesia.

To prevent muscular atrophy, a microelectrode such as the Golden Needle™ (Unit 17 in the chip diagram) was implanted by micropuncturing tissues of a motor point of each muscle with stereoscopic device using a nuclear magnetic resonance method. Joint motion sensors were applied on knee joints via subcutaneous fat to control stimulation-induced motions of lower extremities.

Chip was programmed to perform a 15 minutes long stimulation session of the above muscles three times a day in a certain sequence (each muscle, via a separate chip channel, according to a special program), which would induce flexing and straightening of the knee joint.

To treat atony of urinary bladder, golden needles were implanted into its muscular walls. Chip was programmed to maintain a moderate tension of urinary bladder, and to induce its emptying as soon as it is filled, and the patient so desires, by stimulating its muscles.

To treat angiotrophoneurosis and crus ulcer, a special-purpose electrode was implanted into peridural space of the spinal cord. Chip was programmed to perform three 20 minutes long spinal cord stimulation sessions per day, which would improve blood flow in the lower extremities resulting in the healing of ulcer.

A local blood flow meter was implanted in the crus area to enable an automatic selection of the optimal spinal cord stimulation program by the chip, which would improve blood circulation in lower extremities.

Novel Features of the Chips

Differences of the 1-6 Generation Implant Chips from Prior Art Implanted Neurostimulants and Microchips Include, Among Others:

1. Multi-Purpose Applicability: Chips of each generation can be used for treatment of different ailments (for each specific case the parameters are individually set: type of sensor, electrode, stimulation program).

2. A complete system approach including state-of-the-art biosensors and sensors, supporting an automatic adaptation of the chips to individual characteristics of the patient's body.

3. A multi-channel feature (for chips of the 4th and other advanced generations only) enabling to directly and simultaneously control functions of various organs and systems of the body per different programs thus creating unique possibilities to develop unprecedented novel technologies of treatment of human diseases.

4. Small and extra-small sizes of certain chips and their electrodes.

5. New solutions for chip power supply from the human body's kinetic energy guaranteeing the chips' durability.

6. Increased contents of the precious metals in the electrodes.

7. Using the algorithms developed on a basis of new theoretical knowledge to operate the chips.

Shungite Rock

The chip may be coated with Shungite, to achieve improved performance. Shungite rock—new type of carbon raw material

Joint stock company “NPK Carbon-Shungite” is presently excavating a deposit of shungite rock—the one and only in the world—Zazhoginskoye deposit. Zazhoginskoye deposit is situated in Zaonezhski peninsular (Medvezhjegorski region, Karelia, Russia). Scheme showing localities Shungite rock in its composition, structure and properties presents a unique formation. By its structure it is an original natural composite material: a homogeneous distribution of highly dispersed crystalline silicate particles in amorphous carbon matrix Carbon in shungite is highly active in oxidation-reduction reactions. Thanks to exceptionally well-developed contact between the active carbon and silicates heating of shungite rock triggers fast reduction of silica to metal silicon and silicon carbide.

Composite Shungite radio shielding materials can reduce electromagnetic energy in the range over 100 MHz and up to 100 dB or more.

They have certain ecological advantages over metal materials because they do not distort the Earth magnetic field. Shungite conductive materials may be used as heaters of low specific power, ecologically, fire- and scolding-safe, can be used for making of heated floors and other elements in houses. Shungite rock possesses sorption, catalytic and bactericidal properties. In the present invention, Shungite can be used in sensors, biosensors and electrodes, as detailed elsewhere in the present disclosure.

1.3 Sensors

Oxygen content in blood can be determined by an optoelectric method (similar to pulse oximetry), or using a new magnetoelectric or potentiometric technology.

The following drawings detail the structure of sensors usable with the new system:

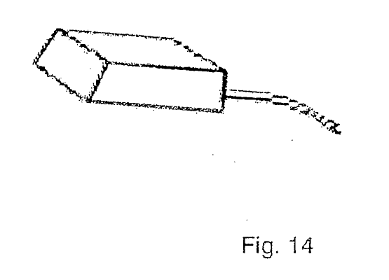

FIG. 14—Structure of sensors No. 8,9,10,16

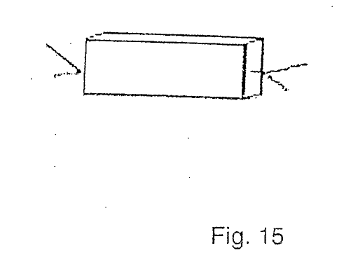

FIG. 15—Structure of sensors No. 11,13

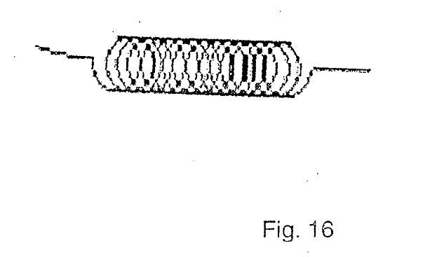

FIG. 16—Structure of sensors No. 12,15

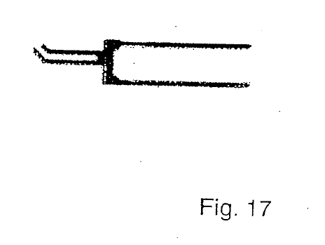

FIG. 17—Structure of sensor No. 14

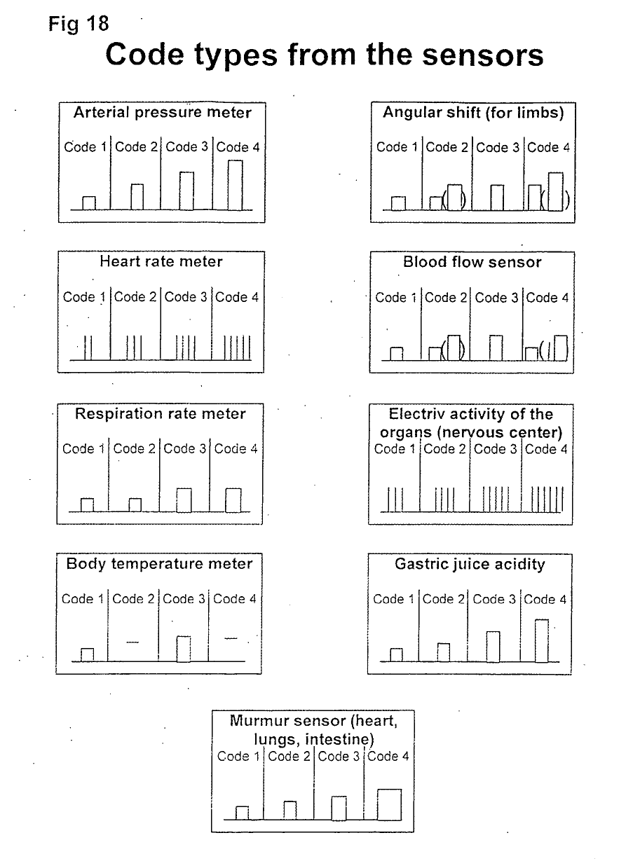

FIG. 18—Typical signals from sensors (codes)

Table 2 illustrates sensors data.

The number of sensors may vary between one and 16 for example, or as required by the specific application.

Shungite can be used in sensors, for improved performance.

1.4 Biosensors

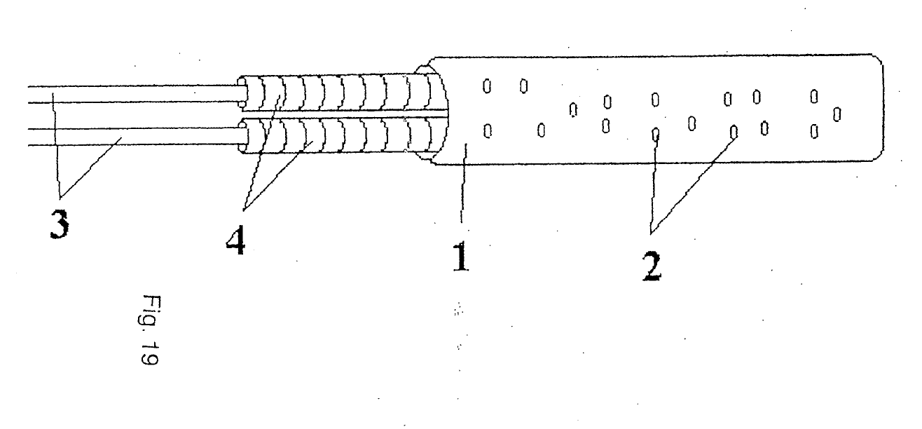

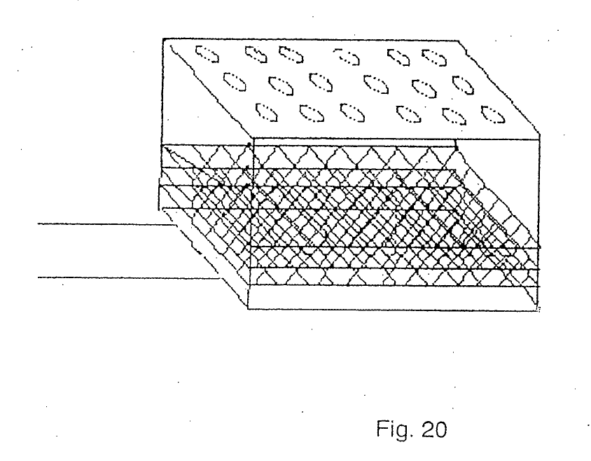

FIGS. 19 and 20 detail the Structure of a biosensor (Type A and B, respectively).

Design of one type of the biosensors in chip's electrode wire.

Index of the conventional symbols in FIGS. 19 20.

1—silicon shell acting as a membrane

2—silicon micro-pores

3—metal cores of the wires

4—shell coated with indicator substances of the biosensors' receptors.

The number of biosensors may vary between one and 16 for example, or as required by the specific application.

Shungite can be used in biosensors, for improved performance.

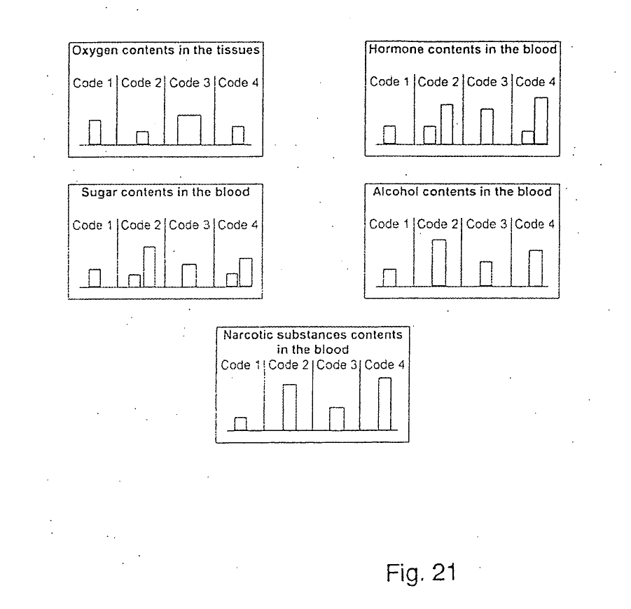

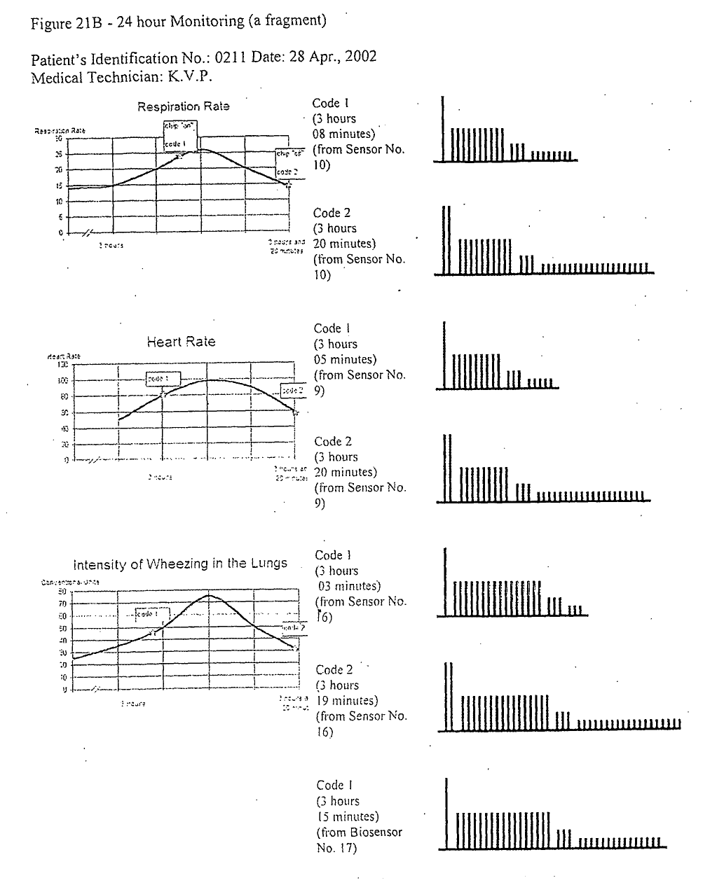

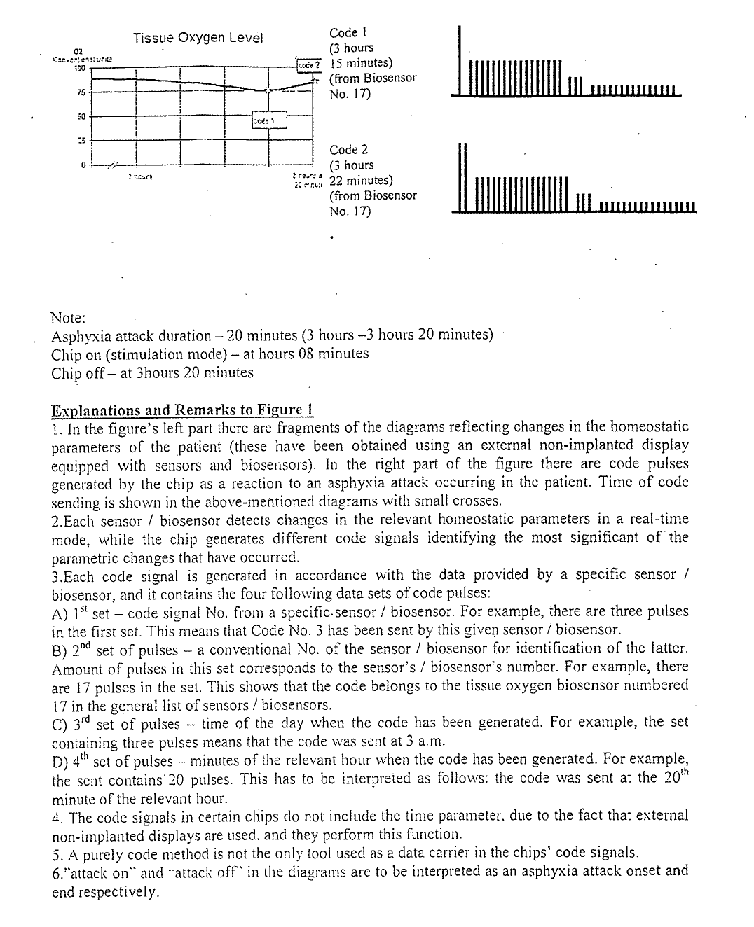

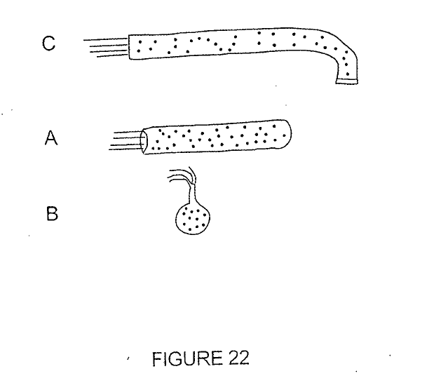

FIG. 21 details typical signals from biosensors (codes).

Table 3 illustrates Biosensors data

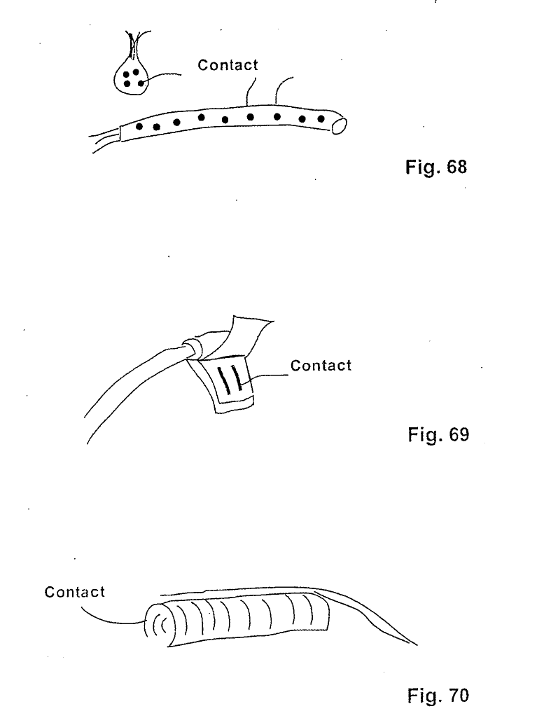

FIG. 68 details the structure of an universal biosensor.

Connection: To any structures of the body.

Design: Any shape, such as for the other electrodes, and size.

The Location of contacts, sensors and/or biosensors may be devised with a method implemented in a computer software.

FIG. 21B illustrates, by way of example, results of 24 hour monitoring in a patient (a fragment). In this example:

Code 1—(3 hours 08 minutes) from Sensor No. 10

Code 2—(3 hours 20 minutes) from Sensor No. 10

Code 1—(3 hours 05 minutes) from Sensor No. 9

Code 2—(3 hours 20 minutes) from Sensor No. 9

Code 1—(3 hours 03 minutes) from Sensor No. 16

Code 2—(3 hours 19 minutes) from Sensor No. 16

Code 1—(3 hours 15 minutes) from Biosensor No. 17

Code 2—(3 hours 22 minutes) from Biosensor No. 17

Note:

Asphyxia attack duration—20 minutes (3 hours to 3 hours 20 minutes)

Chip on (stimulation mode)—at hours 08 minutes

Chip off—at 3 hours 20 minutes

Explanations and Remarks:

1. In the figure's left part there are fragments of the diagrams reflecting changes in the homeostatic parameters of the patient (these have been obtained using an external non-implanted display equipped with sensors and biosensors). In the right part of the figure there are code pulses generated by the chip as a reaction to an asphyxia attack occurring in the patient. Time of code sending is shown in the above-mentioned diagrams with small crosses.

2. Each sensor/biosensor detects changes in the relevant homeostatic parameters in a real-time mode, while the chip generates different code signals identifying the most significant of the parametric changes that have occurred.

3. Each code signal is generated in accordance with the data provided by a specific sensor/biosensor, and it contains the four following data sets of code pulses:

A) 1st set—code signal No. from a specific sensor/biosensor. For example, there are three pulses in the first set. This means that Code No. 3 has been sent by this given sensor/biosensor.

B) 2nd set of pulses—a conventional No. of the sensor/biosensor for identification of the latter. Amount of pulses in this set corresponds to the sensor's/biosensor's number. For example, there are 17 pulses in the set. This shows that the code belongs to the tissue oxygen biosensor numbered 17 in the general list of sensors/biosensors.

C) 3rd set of pulses—time of the day when the code has been generated. For example, the set containing three pulses means that the code was sent at 3 a.m.

D) 4th set of pulses—minutes of the relevant hour when the code has been generated. For example, the sent contains 20 pulses. This has to be interpreted as follows: the code was sent at the 20th minute of the relevant hour.

4. The code signals in certain chips do not include the time parameter, due to the fact that external non-implanted displays are used, and they perform this function.

5. A purely code method is not the only tool that may be used as a data carrier in the chip's code signals.

6. Attack on and attack off in the diagrams are to be interpreted as an asphyxia attack onset and end respectively.

1.5 Universal Sensors

Universal Biosensor

Design and Working Principle

Using a special program of the chip, the sensors (all together or separately), biosensors (all together or separately), dot-shape electrodes (also located throughout the entire surface of the electrode—universal biosensor) can be activated on a selected zone of the electrode's surface (the entire surface or a specific part of it).

Method of Operation

For Example, Program No. 1:

1. The arterial pressure sensors are activated only the electrode's end.

2. The biosensors of all types (except for the oxygen biosensors), are activated in the electrode's middle part, while the oxygen biosensors are activated in the electrode's ¾ part.

3. The electrode's contacts are set into operation only at the beginning of the electrode on its anterior-superior surface.

To enable computer-aided control of the location of activated sensors, biosensors, contacts of the electrode, the latter is designed to have from 2 up to 100 cores supporting its function control.

4. such cores can be seen in FIG. 1.

The electrode-universal biosensor can vary in shape: it can be cylindrical (A), spherical (B), flat (C).

Thus, using the specially developed software, it is possible to change the location of activated sensors, biosensors, contacts, according to the needs, and, therefore, this design of the universal biosensor can replace an innumerable variety of usual electrodes of a fixed, unchangeable design and location of the above-listed elements. The universal biosensor-electrode is in fact an electrode with computer-aided control of localization of the sensors, biosensors, contacts.

FIG. 1

Index of Symbols:

●—Sensor

—Biosensor

?—Electrode

Novelty:

1. Sensors and biosensors of all types+electric contacts are located are spread on the entire surface.

2. Selective programmed activation of the components listed above by means of a computer command.

3. Three different forms, namely: cylindrical, spherical, flat

1.6 Electrodes

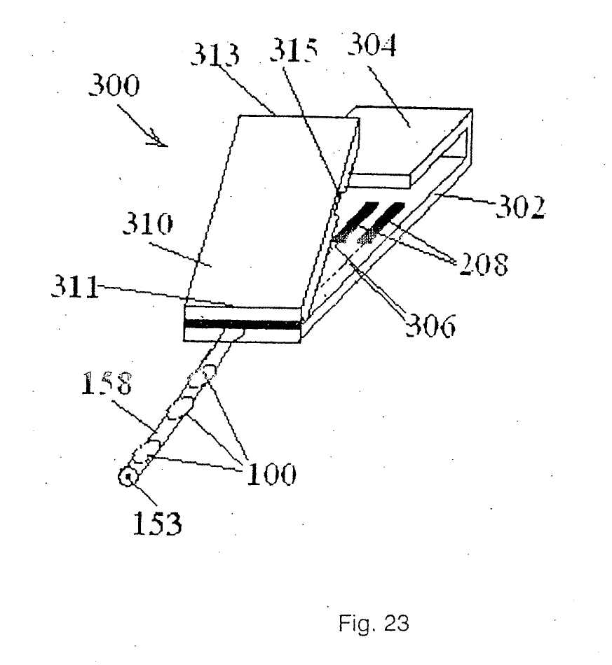

FIGS. 23, 24 and 25 illustrate structure of an electrode (Type A, B, C respectively), compatible with the chips in the present system.

Further electrodes compatible with the chips are detailed in FIGS. 69-77:

FIG. 69—Structure of book-type bipolar electrode.

To nerves and muscles. Silicone coating, metal contacts.

FIG. 70—Structure of book-type multi-channel electrode®Zebra ).

To nerves, blood vessels, various organs.

Silicone coating, “petals” of any size, contacts from inside (metal/silicone).

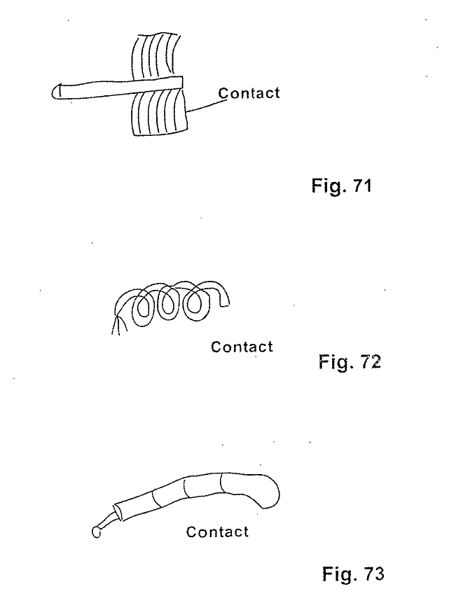

FIG. 71—Structure of book-type 2-8 polar or more electrode.

To nerves, blood vessels, various organs.

Silicone coating, length of the “book's” leafs is unlimited.

Contacts from inside or outside.

FIG. 72—Structure of Spiral Electrode.

To nerves, blood vessels, various organs.

Contacts can be located in any selected spot.

FIG. 73—Structure of Plate-Like Electrode.

To nerves, blood vessels, various organs.

An elastic band used as the electrode's base allows to fit the latter to various uneven surfaces. The contacts are from the inside.

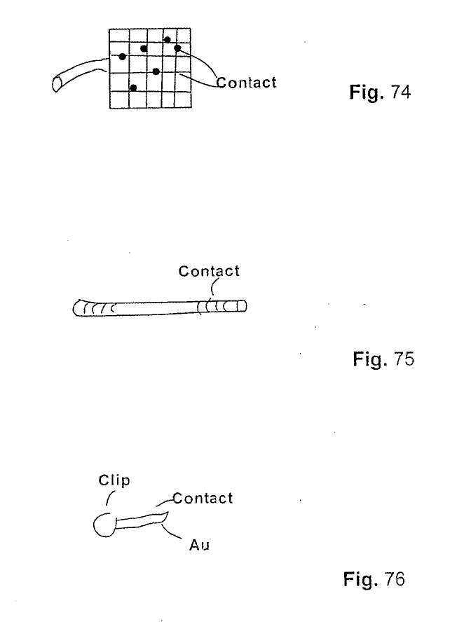

FIG. 74—Structure of Wire Gauze Electrode.

To various organs.

Any size, the contacts are made of metal, Schungite or other materials.

FIG. 75—Structure of Coaxial Electrode.

To the spinal cord, to various organs, to nerves.

Two or more contacts (of gold, platinum, etc.)

Diameter, length are unlimited.

1.7 Wireless Electrodes—Golden Needle™

Golden Needle™ and Chip No. 6

See FIG. 76, Structure of Golden Needle™ electrode. This electrode may connect to nerves, blood vessels and/or various organs.

The electrode is connected with the chip by means of electromagnetic waves. Chip No. 6 has an advanced structure, which allows to connect to it all types of electrodes, including the Golden needle.

There are at present two basic types of Golden needle (GN) electrodes:

1. GN type 1—this is a tiny electrode, shaped as a needle of a length of about 6 mm. It may be made of gold or is gold plated.

It is activated by wireless, for example using radio signals transmitted from the processor means which controls it, such as Chip No. 5 or 6.

The stimulation type is controlled through the radio signals. The electrode may not include its own power source, in which case it may be powered through RF from the processor means.

In a minimal configuration, the GN does not include autonomous facilities or capabilities.

GN can be used for the treatment of various diseases. There is no need for wires to connect it to the processor.

GN may be implanted using endoscopic surgery. For use in the Carotid collector there is no need for artery peeling and for the artery to grasp the tissue there—the GN pricks the artery wall. The artery wall contains plenty of thin nerve fibres, thus the GN touches them.

To prevent puncture of the artery itself, after the initial penetration of the artery wall, the GN tip bifurcates or contains means for its splitting and opening like a safety pin. Then the GN tip is not sharp anymore, and the danger of puncturing the artery is eliminated.

2. GN type 2—this is a small chip or device, that may be shaped as a coin or a flat cylinder, having a diameter of about 1 to 2 cm. It may have certain processing capabilities and also includes a tiny electrode, about 0.5-3 cm long.

Preferably the electrode is needle-shaped and made of gold or gold coated. This type of GN may contain the electrode itself, sensor means, a wireless receiver, a digital memory and an encoder. It may also include a wireless transmitter. The wireless link may be implemented in RF.

The sensor means may be installed on the outside of the GN cover. The GN may be used in the treatment of one disease or of several diseases concurrently.

Method of Operation

The processor means may activate several GN devices concurrently, using wireless with a different coding and/or a different frequency for each.

The information from the sensor means in each GN device is transferred through the wireless link to the processor.

Messages regarding the required stimulation are sent from the processor to each GN device.

Chip No. 6 can concurrently communicate with more than one hundred GN devices, to treat one disease or several diseases concurrently.

1.8 Autonomous Power Source

In one embodiment, electric energy is generated from the body's internal organs movement. FIG. 25B illustrates the structure and implantation method for the power source.

The power supply includes a flexible piezoelectric element (1), that may be shaped as a cable or electrode, and coated with a biologically inert material. The element (1) is implanted as illustrated, under the diaphragm's cupola (4), from the right side, endoscopically, and is connected to the chip (3).

Mechanical up/down novements of the diaphragm, occuring during the patient's normal breathing process, will cause periodic deflections in the piezoelectric element (1). An electric voltage is generated in element (1), due to the piezoelectric effect in the transducer. The chip (3) may include a voltage rectifier (2), to transform the AC voltage to DC.

Optionally, unit (2) may also include digitizer means, to allow the system to use the element (1) as a sensor, to measure the breathing characteristics.

The system chip may implement a method (algorithm) to also monitor the patient's breathing and to respond in a preprogrammed manner to changes therein.

Advantages:

a. Reliable, uninterrupted electric power for the system, based on the patient's respiration. The shift of the diaphragm's cupola during breathing may reach about 4 to 8 cm.

b. The above structure and method of operation allows the patient to control the system's operation, by intentionally changing the breathing characteristics such as the rate or depth thereof.

A brief description of the implanted chip's piezoelectric power source charged from the human body kinetic energy

Brief description of the microchip's power source

Its design is outlined in FIG. 25B

Legend:

(1)—A flexible piezoelectric element (electrode-like) coated with a biologically inert material. This element is implanted under the diaphragm's cupola (4), from the right side, endoscopically, and it is connected to the chip (3). The chip comprises an AC/DC transducer (2).

Power Source's Working Mechanism

Mechanical movements of the diaphragm (up and down) occurring at the patient's breathing cause periodic deflections in the piezoelectric element (1), according to the respiration rate. As a result of mechanical motions, the piezoelectric element generates electric pulses which are transmitted to the above-mentioned unit (2), and they are transformed into direct current necessary to power the microchip.

Advantages of the Power Source Described Above:

1—Reliable uninterrupted electric powering of the chip resulting from the patient's respiration (Note: A shift of the diaphragm's cupola at breathing, during an inhalation-exhalation cycle, can reach 4-8 cm).

2—It allows the patient to control the microchip's electric powering and, as a result, to increase or to reduce the microchip's effect by means of a voluntary control of the breathing depth by the patient.

See FIG. 25B—structure and location of implanted power source.

FIG. 25C details the Structure of an external monitoring device

The medical instrument may use an External Non-implanted Display —Programmer—Charging Device. The sensors and biosensors representing micro- and macro-indicating devices of different, located both on the patient's body surface and directly introduced into its tissues, organs, systems (for example, arterial pressure meter introduced into the femoral artery) collect data on functioning of the body's systems.

These data are transmitted to the unit analyzing conditions of the body's systems, organs, tissues (No. 3 on the Diagram). The unit separately analyzes functioning of each of the systems studied enabling both fragmentary and permanent real-time monitoring of the body's systems, organs, tissues.

The medical treatment method, in order to control functioning of the implanted Stimulator and to set its optimal mode, further including the step where the patient is connected (for the period varying between 1 and several days) to a portable external non-implantable monitor collecting and analyzing data on the Electric Stimulator's work, as well as data on a functional condition of the body's systems, organs, and tissues, while this said monitor comprises a unit analyzing functional conditions of the body's systems, organs, and tissues, connected to the sensor means, and this unit is also connected to the monitor's unit of radio-frequency communication with the Electric Stimulator's external radio-frequency communication unit which, in its turn, is connected to a computer via a radio-frequency channel, as well as to the autonomous power supply unit, the latter also being connected to all the above-listed units of the monitor.

The medical treatment method may include the step, performed using the Electric Stimulator's software, of distinguishing between the changes in functional activity of the body's systems, and/or organs, and/or tissues typical of an onset of a disease (symptoms) and the changes in functional activity of the body's systems, and/or organs, and/or tissues, that are not related to symptoms of a disease, but typically occur in the patient's body, while the Electric Stimulator's “learning” of this process is aided by the non-implanted display.

1.9 Contacts, or Indirect Body Measurements Using Adaptive Techniques

Initially, after the implantation of the system, the sensors and biosensors are used to measure the body variables; with time, however, these means are disabled because of the body's inherent characteristics. Other means have been devised to prolong the operation of the system—“Contacts”, together with an adaptive operation of the microcontroller “Chip”:

The contacts measure body variables such as electrical resistance, response to ultrasonic waves and/or response to radio frequency electromagnetic waves. These variables are then compared in the Chip with the readouts from the sensors and biosensors.

Using adaptive algorithms as known in the art, the Chip in time learns the body characteristics as conveyed in the “Contacts” data. That is, a cross-correlation function is compiled, between the sensor and biosensor data on one hand, and the Contacts data on the other hand.

In a subsequent stage, when the sensors and biosensors are disabled, the system can still function using the Contacts, whose data reliably replaces the sensor and biosensor data. Thus, indirect measurements using Contacts replace direct body measurements using the sensors and biosensors.

Location on the chip—Sensitive elements of Contacts, sensors and biosensors can be located both on the chip's coating and under it, as well as at the electrode contacts' endings or any other part of the electrode. Working principle, MEV—Measurement of electric values, pertaining to organs' function Pz—Piezo-effect. Type of signal received: Code signal and/or Analog signal Membrane's structure, receptor, substance,—Silicon membrane or other biologically inert porous material—Special substance or electronic component. Range of values measured, Disease-dependent

Size range of sensitive elements: Micrometers to millimeters.

The medical instrument may further include means for stimulation and/or electric blocking of the body tissues, comprising sensor means for measuring variables in the body, processor means connected to the sensors and biosensors for processing the measured variables and for deciding in real time whether to apply an electric signal to the body tissues, and electrode means implanted at predefined locations and connected to the processor means, for applying the stimulation and/or electric blocking signals to the body tissues.

The medical instrument may further include, in addition to analyzing functional activity of the body's systems, and/or organs, and/or tissues, as well as controlling and analyzing operation and functioning of the implanted Stimulator, the monitor also supports programming or reprogramming of the Electric Stimulator (by means of a computer connected thereto via the radio-frequency communication unit).

The medical instrument according may further include means for running a long-term monitoring of functional activity of the body's systems, organs, tissues, and operation of the Electric Stimulator, while duration of the monitoring may vary between several minutes and several months.

2 Method of Operation of the System, Software, Algorithms

Most commonly, chips operate according to a pre-set program or are manually activated by patients. Chip operation pattern depends of the frequency, duration, and regularity of asphyxial seizures, and availability of reproducible changes of respiration and heart parameters during or before seizures capable of ensuring an efficient operation of biological sensors. In patients having chip 2 implanted, the biological sensor was used approximately in 35-40% of cases. With chips 3 and higher, sensors were applied in 100% of cases.

Three approaches are used concurrently to provide a reliable prevention of seizures: chip programming to automatic activation before seizure; determining the onset of seizures based on the frequency of respiration and systole. Sensors capable of detecting rales may also be used.

3 Method of Treatment Using the New System

The chip's shell is implanted into the subcutaneous fat of the thorax (1-5-generation chips), and the electrodes are connected to the nerves through incisions or punctures.

The biosensor—containing electrodes are implanted in the head tissues (the epilepsy cases) or other parts of the body.

Biosensors and sensors are located in the electrode and in chip casing. The latter is implanted to the right or left of the sternal muscle which allows its biosensors and sensors to detect respiratory murmurs or systole.

Biosensors—equipped electrode may be positioned in various parts of the body depending on location of the nerve it is connected to. Electrode sensors and biosensors make measurements directly in tissues.

Diseases List

Examples of diseases that may be treated using the present invention are listed in Tables 7, 8 and 9, with relevant details pertaining to their treatment.

Examples of diseases are also disclosed with reference to FIGS. 28-48.

Other diseases that may be treated using the present invention may include, among others:
1. Insomnia.
   2. Hypersonmia.
   3. Apnea.
   4. Narcolepsy.
   5. Sudden cardiac arrest at sleep.
   6. Paresis of the vocal cords.
   7. Nervous anorexia.
   8. Obesity.
   9. Bulimia.
   10. Gastric and duodenal ulcer.
   11. Chronic gastroenterocolitis.
   12. Refluxesophagitis.
   13. Gastrointestinal dyskinesia.
   14. Commissural disease.
   15. Crohn's disease.
   16. Hirschsprung's disease-megacolon.
   17. Rectal prolapse.
   18. Chronic duodenal ileus.
   19. Bauhin's valve failure.
   20. Doloichosigmoid.
   21. Chronic intestinal obstruction (commissural disease, megacolon, chronic mes nterial circulation insufficiency, metacolon, doloichosigmoid, cardiac ach lasia.
   22. Schizophrenia with schizophrenic affective disorders and delirium.
   23. Anxiety and depression.
   24. Borderline personality disorder.
   25. Cortical dementia—Alzheimer's disease.
   26: Pick's disease.
   27. Subcortical dementia—supranuclear palsy (paralysis).
   28. Huntington's chorea.
   29. Parkinson's disease.
   30. Multiinfarction dementia.
   31. Involuntary movements.
   32. Stammering.
   33. Epilepsy.
   34. Priapism.
   35. Infantile cerebral paralysis
   36. Paralyses of different etiology.
   37. Syringomyelia.
   38. Progressing myodystrophy and other forms of dystrophy.
   39. Chronic and acute hyperthermia.
   40. Atrophy of the optic nerve.
   41. Chronic periodic pains (angina pectoris, phantom pains, neuritis, nerve root syndromes.
   42. Terminalstage pains.
   43. Migraine.
   44. Cancer.
   45. Hypertension
   46. Hypotension.
   47. Vegetovascular dystony.
   48. Diabetes.
   49. hypoglycemia.
   50. diabetes insipidus.
   51. hypothyrosis . . . hyperthyrosis
   52. adrenal cortex insufficiency.
   53. male and female infertility.
   54. impotence.
   55. adrenal cortex hyperfunction,
   56. dysmenorrhea.
   57. Zollinger-Ellison syndrome.
   58. Dyskinesia of the biliferous tracts.
   59. Chronic hepatitis.
   60. Chronic cholecystopancreatitis.
   61. Cirrhosis.
   62. Osteoporosis.
   63. Periostitis, osteosclerosis of different types,
   64. Hyperostosis.
   65. Chronic osteomyelitis
   66. Flaccidly consolidating fractures.
   67. Rickets.
   68. Perthes disease
   69. anemia.
   70. agranulocytosis.
   71. leucosis.
   72. Immunodeficiency
   73. trauma-related paralyses.
   74. myodystrophy.
   75. myopathy.
   76. Bodybuilding
   77. Hydronephrosis.
   78. Chronic pyelonephritis.
   79. Chronic glomerulonephritis.
   80. Urinary bladder atony.
   81. Chronic cystitis.
   82. Psoriasis.
   83. Neurodermite.
   84. Eczema.
   85. Alopecia.
   86. Hyperkeratosis.
   87. Skin atrophy.
   88. Angiotrophoneurosis.
   89. Drug addiction.
   90. Alcoholism.
   91. Obliterating atherosclerosis and endarteritis.
   92. Ischemic heart disease and angina pectoris.
   93. Cardiac arrhythmia.
   94. Raynaud's disease.
   95. Buerger's disease.
   96. Chronic thromboohlebitis—supranuclear palsy (paralysis).
   97. Postthrombophlebitic syndrome.
   The treatment method for the various diseases is detailed in the present disclosure, with reference to the drawings and the tables herein.
      The Implant Operation Method

The system implantation operations are performed under anesthesia. The microchips are more frequently implanted by means of an endoscopic procedure, i.e., not through incisions, but rather through punctures in the soft tissues.

3.1 Treatment Matrix. For Each Disease a Matrix of: System Structure Method of Operation of the System Locations in the Body for Sensors, Electrodes

Epilepsy Treatment Method

See Table 4—Method of Operation/Algorithm for Epilepsy

1. The chip can detect when an attack begins according to the typical changes in the EEG.

2. The chip detects the attack immediately, at its onset, according to presence of the typical changes in the EEG.

3. The chip can permanently monitor the EEG, both before and during an attack.

4. As per our experience, the chips control the following types of epilepsy:

Grand mal epilepsy,
Petit mal epilepsy
Absence epilepsy
Atonic epilepsy.
   5. If the patient has no “Aura”, and the chip has not detected the first signs of an approaching epileptic attack (which occurs in 10-15% of the cases), the chip will be automatically activated anyway, when the attack has started. In addition to this, the patient him or herself can signal that the chip is to be activated once he or she has felt that the attack is about to begin, because the loss of consciousness does not always develop suddenly.
   6. The chip stimulates the sinocarotid nerve.
   7. The chip's impact on the nerve at severe and mild attacks differs in its duration: the stronger is the attack, the longer is the duration. The impact duration is determined according to the period of presence of the typical changes in the EEG.
   8. The chip remains active until the EEG has become normal, or until other signs of the attack have disappeared completely.
   9. The chips are supplied with power batteries that support their operation during 2-5 years. The batteries can be replaced by means of a minor surgical procedure or, alternatively, they can be recharged via electromagnetic waves from a special device.
   10. The chip's activity never causes a loss of consciousness in the patient, although it is made operative through the reticular formation. The chip's impact is usually not accompanied by negative side effects.

11. The present invention may require further modifications when used in the following cases:

a. If the patient suffers from cancer of any type—additional research may be necessary.

b. If the patient suffers from chronic purulent diseases (due to a risk of the chip's rejection);

c. If the patient works in an area with strong electromagnetic radiation (high-voltage lines service, powerful radio-systems antennas, work with electric arc welding equipment).

Asthma Treatment Method

See Table 5—Method of operation/algorithm for asthma

The periods when an asthma spasm begins and ends are detected, according to the oxygen contents in the patient's tissues.

Time required to stop the spasms The table we sent you earlier contains data on the time required to fully stop the asphyxia attack whose beginning and end are detected with a biosensor.

However, the clinical signs of the attack onset become evident 10-15 minutes later than this is detected with the biosensor. Therefore, the patient feels that the chip's effect is very fast (it takes only a few minutes).

In most cases the attack is stopped at its very onset, that is why the patients do not even suspect that they have suffered one.

Reduction of the daily intake of antiasthmatic preparations:
For Chips 1 and 2—up to a 2-fold reduction, or possibly more
For Chips 3 and 4—up to a 3-fold reduction, or possibly more
For Chip 5—up to a 5-6-fold reduction, or possibly more
For Chips 6—up to a 4-fold reduction, or possibly more
   Note:

There have been recorded cases when the preparations' use could be fully suspended, and a many-year remission of the disease was achieved without any medications, the treatment having involved the chips (3,4,5,6-generation) only.

Angina Pectoris Treatment

In angina pectoris patients the chip stimulates the sinocarotid nerve thus causing a reflex dilatation of the coronary arteries. In obesity patients the chip affects the vagus nerve suppressing the gastric juice secretion, and, as a result, the appetite is reduced.

In diabetes patients, a sugar level drop is achieved by means of stimulating the vagus nerve that innervates the pancreatic gland cells.

See also:

Table 6—Method of sensors/biosensors activation

Table 7—Treatment strategy: System structure and implant locations

Table 8—Electric stimulation parameters (A)

Table 9—Electric stimulation parameters (B)

Notes:

1. Specific values of sensors and biosensors-generated signals, as assigned to each of the codes, are determined separately per each patient.
2. The number of codes is unlimited.
   Legend:
CC—patient develops characteristic changes of the parameter caused by this symptom of ailment (CC1, CC2, etc.).
SI/SA—signal inavailable/signal available.
N—parameter is within the norm, given the state of the specific patient.
SI—signal inavailable

3.2 Inverse Treatment Matrix. For a Specific Structure and Implantation:
List of all the diseases that are concurrently being treated (or can be treated, if diagnosed in the patient)

The affected systems and organs of the body include, for example: the nervous structure of the sympathetic nervous system or the parasympathetic system or the sympathetic nervous system and parasympathetic system and hypoglossal (sinocarotid collector of the Vegetative Nervous System-SCVNS), the central nervous system, as well as neurons of the organ and/or cutaneous nerves and/or depressor nerves.

In a preferred embodiment, a nervous band or group is formed, comprising all, or the majority of, the nerve branches innervating the carotid glome (glomus caroticum).

The carotid glome is found in the area where the common carotid artery splits into the internal and external carotid arteries. See, for example:

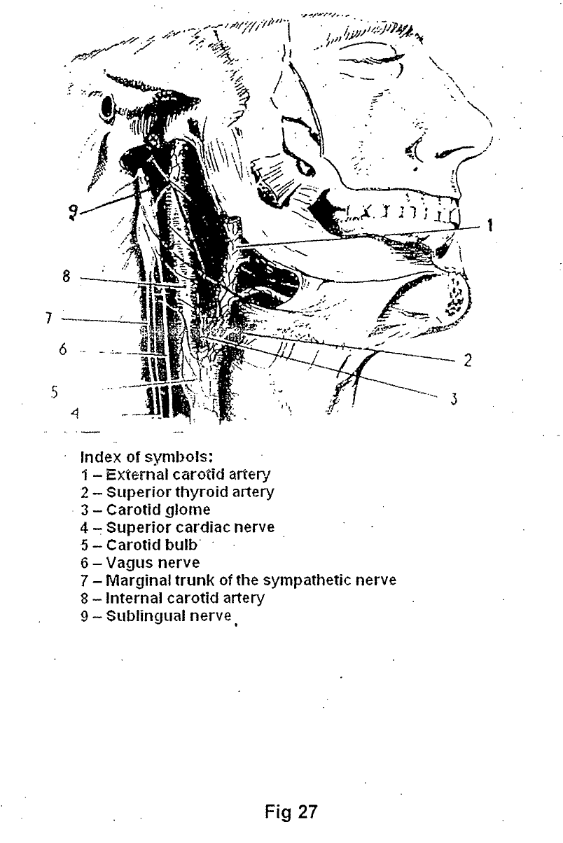

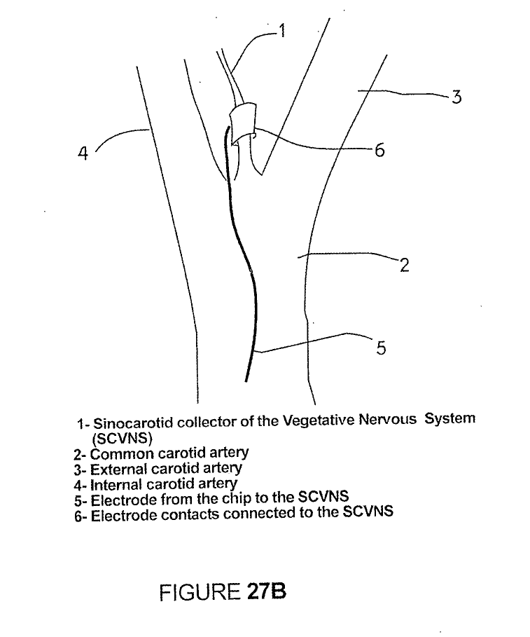

FIG. 27—Preferred Implantation Location in the SCVNS

An active chip electrode is connected to the nerves of sinocarotid reflexogenic zone diverging from carotid glomerulus (glomus caroticum). Chip is normally connected to either one of these nerves (left or right). The technology is equally efficient in left and right nerves. Chip connection to both nerves is slightly more efficient. Chips 4 and 6 are connected to a single nerve of asthmatic patients, since these are equipped with a lot of electrodes. The remaining electrodes are designed to use chips to treat other ailments.

FIG. 27B—Detail of Preferred Implantation Location in the SCVNS.

To activate the above nervous group, an electrode is placed onto the abovedetailed location and is mechanically secured there, for example using a silicone coat. The electrode is connected to the microchip of the system, and may be used to activate the above nerve group when necessary.

The above nervous band or group is formed using surgical tools. Electrode is connected to sinocarotid nerve of asthmatic patients in the area of bifurcation of common carotid artery into internal and external carotid arteries. Actually, this is not sinocarotid nerve itself, but a number of nervous branches which descend to glomus caroticus from simpatico nerves, vagus, and hypoglossal nerve and follow along the internal posterior wall of common carotid artery bifurcation. These nerves are surgically separated from the carotid artery as a cord, in the zone of internal posterior wall of the adventitia area (external tunic of carotid artery).

This formation is called a sinocarotid nerve. Moreover, electrode may be connected to the middle, upper or lower third of sympathetic nerve in the neck, or to the middle, upper or lower third of sympathetic nerve in the thoracic section of sympathetic trunk.

Electrode is connected to the nerve externally: its contacts located on the L-book are slipped over the nerve, with the silicon rubber L-book stitched above the contacts to fix those.

Cholinergic effect is prevented by using special-purpose nerve electric stimulation programs.

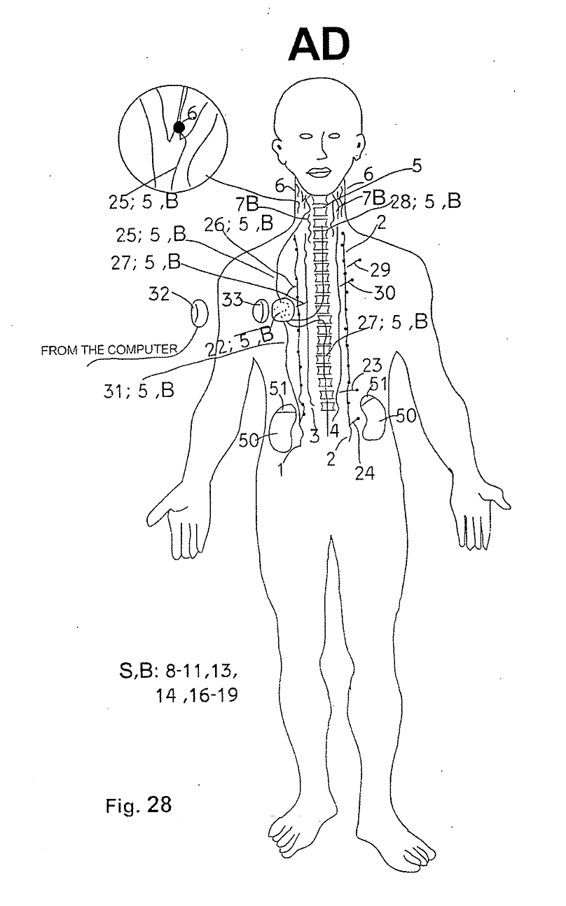

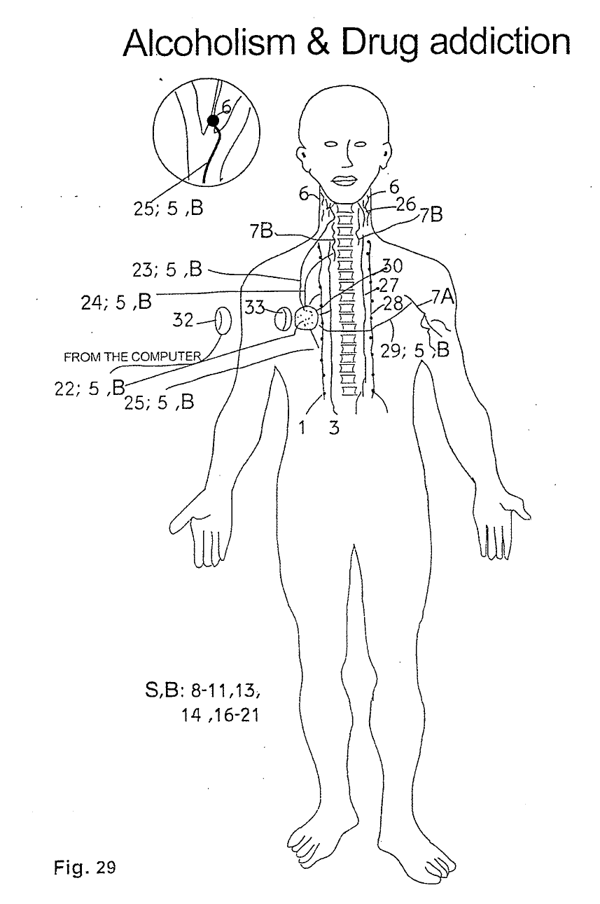

FIG. 29—Preferred Implantation Location—AD

Diseases:

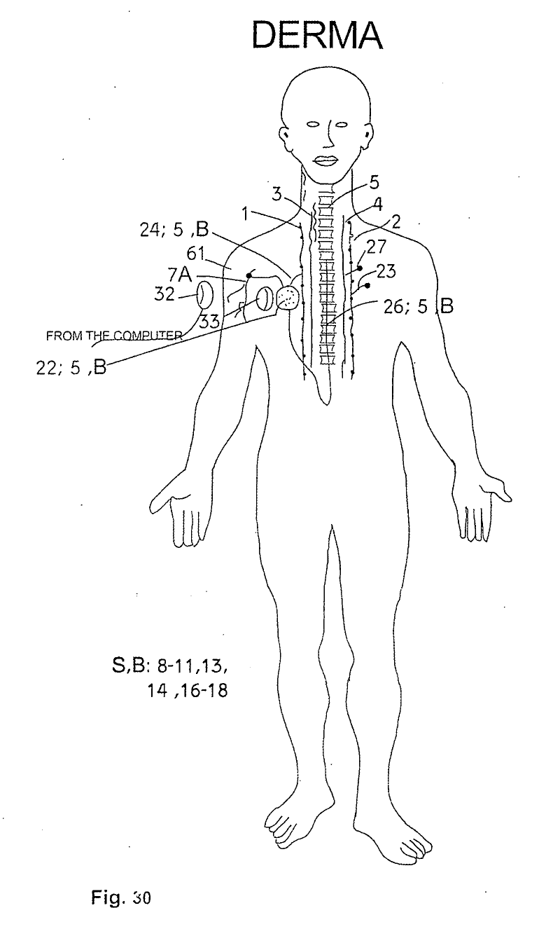

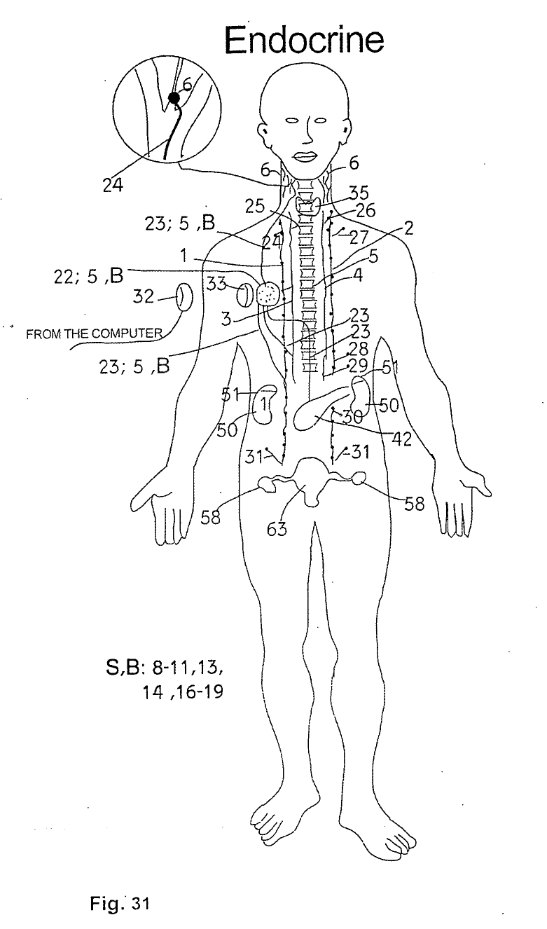

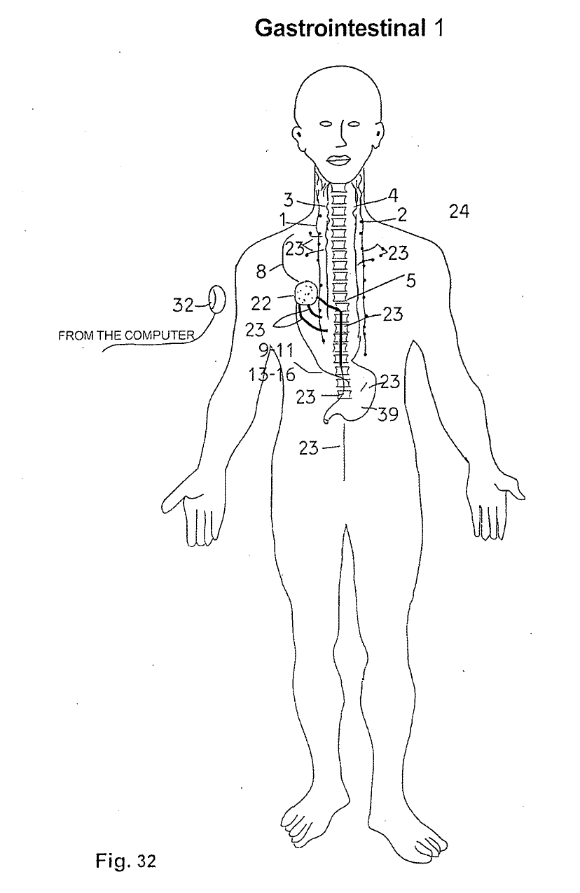

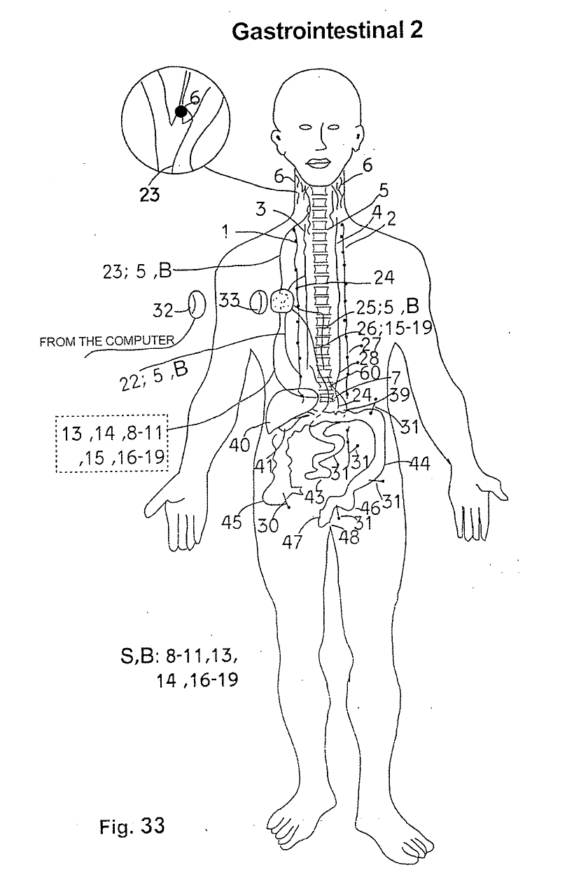

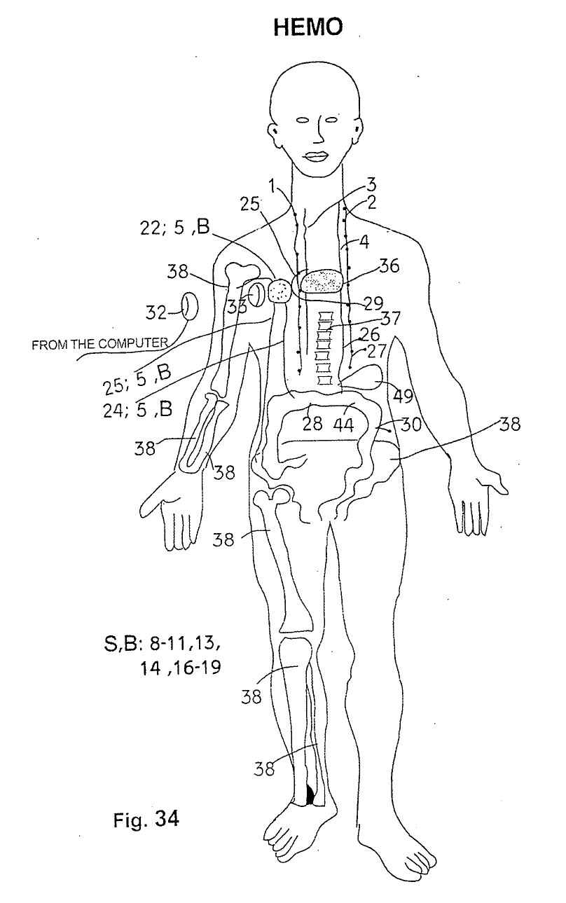

Hypertension of all types (angina pectoris, phantom pains, neuritis, nerve root syndromes [Chip 1, Chip 2, Chip 3, Chip 4, Chip 5, Chip 6]
Hypotension [Chip 1, Chip 2, Chip 3, Chip 4, Chip 5, Chip 6]
Vegetovascular dystony [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
7B—depressor, inhibiting nerves
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
22—microchip with sensors, biosensors and electrodes
23-31—electrodes connected to the nervous structures
32—external radio frequency communications unit
33—external chip controller, additional
   Organs:
50—kidneys
51—adrenal glands
   FIG. 30—Preferred Implantation Location—Alcoholism & Drug Addiction
   Diseases:
Drug addiction [Chip 1, Chip 2, Chip 3, Chip 4, Chip 5, Chip 6]
Alcoholism [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
6—SCVNS: Sinocarotid collector of the Vegetative Nervous System
7A—cutaneous nerve
7B—depressor nerve
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
20—alcohol biosensor
21—narcotic substances biosensor
22—microchip with sensors, biosensors and electrodes
23-30—electrodes connected to the nervous structures
32—external radio frequency communications unit
33—external chip controller, additional
   FIG. 31—Preferred Implantation Location—Derma
   Diseases:
Psoriasis [Chip 1, Chip 2, Chip 3, Chip 4, Chip 5, Chip 6]
Neurodermite [Chip 1, Chip 2, Chip 3, Chip 4, Chip 5, Chip 6]
Eczema [Chip 1, Chip 2, Chip 3, Chip 4, Chip 5, Chip 6]
Alopecia [Chip 1, Chip 2, Chip 3, Chip 4, Chip 5, Chip 6]
Hyperkeratosis [Chip 1, Chip 2, Chip 3, Chip 4, Chip 5, Chip 6]
Skin atrophy [Chip 1, Chip 2, Chip 3, Chip 4, Chip 5, Chip 6]
Angiotrophoneurosis [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
7A—cutaneous nerve
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
22—microchip with sensors, biosensors and electrodes
23-27—electrodes connected to the nervous structures
32—external radio frequency communications unit
33—external chip controller, additional
   Organs:
61—skin
   FIG. 32—Preferred Implantation Location—Endocrine
   Diseases:
Diabetes [Chip 1, Chip 2, Chip 3, Chip 4, Chip 5, Chip 6]
hypoglycemia [Chip 1, Chip 2, Chip 3, Chip 4, Chip 5, Chip 6]
diabetes insipidus [Chip 1, Chip 2, Chip 3, Chip 4, Chip 5, Chip 6]
hypothyrosis, [Chip 1, Chip 2, Chip 3, Chip 4, Chip 5, Chip 6] hyperthyrosis
adrenal cortex insufficiency [Chip 1, Chip 2, Chip 3, Chip 4, Chip 5, Chip 6]
male and female infertility [Chip 1, Chip 2, Chip 3, Chip 4, Chip 5, Chip 6]
impotence [Chip 1, Chip 2, Chip 3, Chip 4, Chip 5, Chip 6]
adrenal cortex hyperfunction, [Chip 1, Chip 2, Chip 3, Chip 4, Chip 5, Chip 6]
dysmenorrhea [Chip 1, Chip 2, Chip 3, Chip 4, Chip 5, Chip 6]
Zollinger-Ellison syndrome [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
22—microchip with sensors, biosensors and electrodes
23-31—electrodes connected to the nervous structures
32—external radio frequency communications unit
33—external chip controller, additional
   Organs:
35—parathyroid gland
42—pancreas
50—kidney
51—adrenal glands
56—prostate
57—seminal vesicles
58—ovaries
59—testicles
63—uterus
   FIG. 33—Preferred Implantation Location—Gastrointestinal 1
   Diseases:
Nervous anorexia [Chip 1, Chip 2, Chip 3, Chip 4, Chip 5, Chip 6]
Obesity [Chip 1, Chip 2, Chip 3, Chip 4, Chip 5, Chip 6]
Bulimia [Chip 1, Chip 2, Chip 3, Chip 4, Chip 5, Chip 6]
Gastric ulcer [Chip 1, Chip 2, Chip 3, Chip 4, Chip 5, Chip 6]
Chronic gastroenterocolitis [Chip 1, Chip 2, Chip 3, Chip 4, Chip 5, Chip 6]
Reflux-esophagitis [Chip 1, Chip 2, Chip 3, Chip 4, Chip 5, Chip 6]
Gastrointestinal dyskinesia [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
   Sensors:
8—arterial pressure sensor
9—heart rate sensor
10—respiration rate sensor
11—body temperature sensor
13—local blood circulation sensor
14—sensor of electric activity of the organs and nervous structures
15—gastric acidity sensor
16—sensor of mechanical activity and murmurs of the organs
   Biosensors:
17—oxygen biosensor
18—glucose biosensor
19—alcohol biosensor
22—microchip with sensors, biosensors and electrodes
23—electrodes connected to the nervous structures
   Organs:
39—stomach
   FIG. 34—Preferred Implantation Location—Gastrointestinal 2
   Diseases:
Nervous anorexia [Chip 1, Chip 2, Chip 3, Chip 4, Chip 5, Chip 6]
Obesity [Chip 1, Chip 2, Chip 3, Chip 4, Chip 5, Chip 6]
Bulimia [Chip 1, Chip 2, Chip 3, Chip 4, Chip 5, Chip 6]
Gastric and duodenal ulcer [Chip 1, Chip 2, Chip 3, Chip 4, Chip 5, Chip 6]
Chronic gastroenterocolitis [Chip 1, Chip 2, Chip 3, Chip 4, Chip 5, Chip 6]
Commissural disease [Chip 1, Chip 2, Chip 3, Chip 4, Chip 5, Chip 6]
Crohn's disease [Chip 1, Chip 2, Chip 3, Chip 4, Chip 5, Chip 6]
Hirschsprung's disease-megacolon [Chip 1, Chip 2, Chip 3, Chip 4, Chip 5, Chip 6]
Rectal prolapse [Chip 1, Chip 2, Chip 3, Chip 4, Chip 5, Chip 6]
Chronic duodenal ileus [Chip 1, Chip 2, Chip 3, Chip 4, Chip 5, Chip 6]
Bauhin's valve failure [Chip 1, Chip 2, Chip 3, Chip 4, Chip 5, Chip 6]
Dolichosigmoid [Chip 2, Chip 3, Chip 4, Chip 5. Chip 6]
Gastrointestinal dyskinesia [Chip 1, Chip 2, Chip 3, Chip 4, Chip 5, Chip 6]
Reflux-esophagitis [Chip 1, Chip 2, Chip 3, Chip 4, Chip 5, Chip 6]

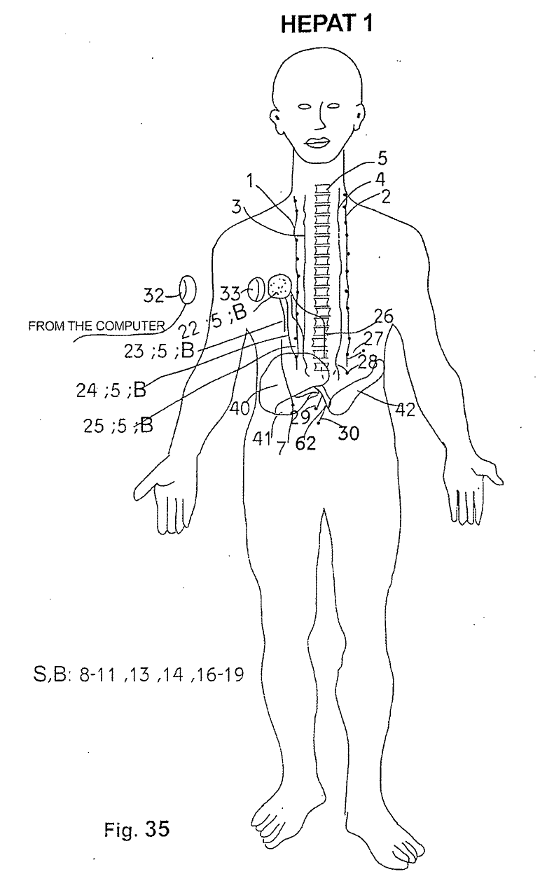

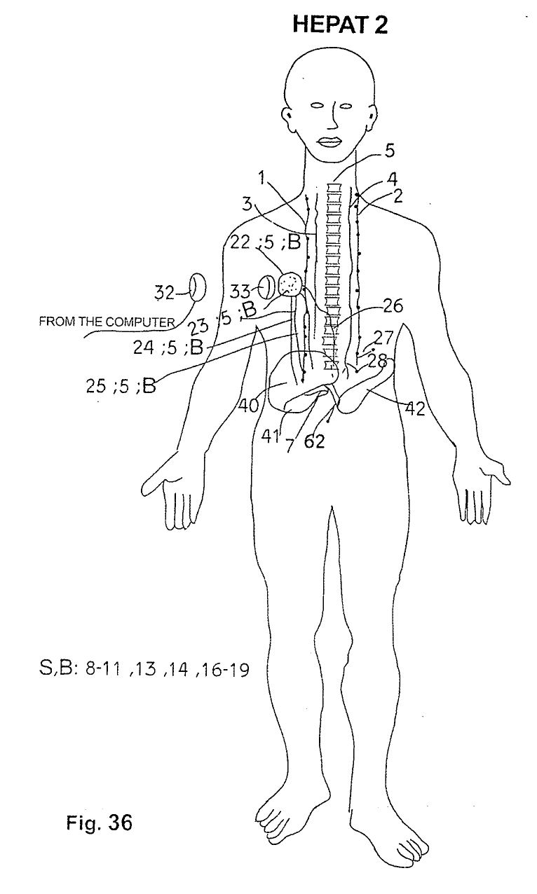

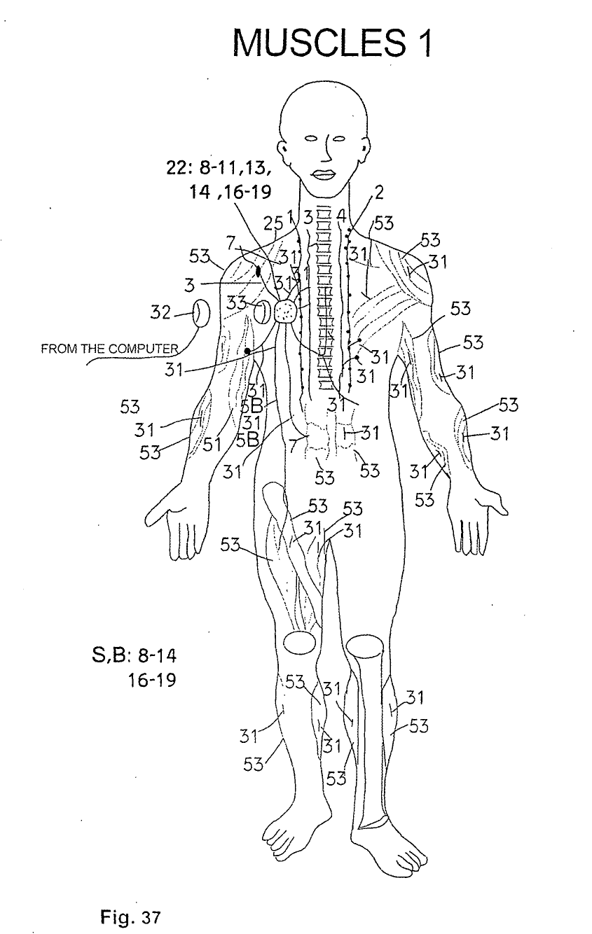

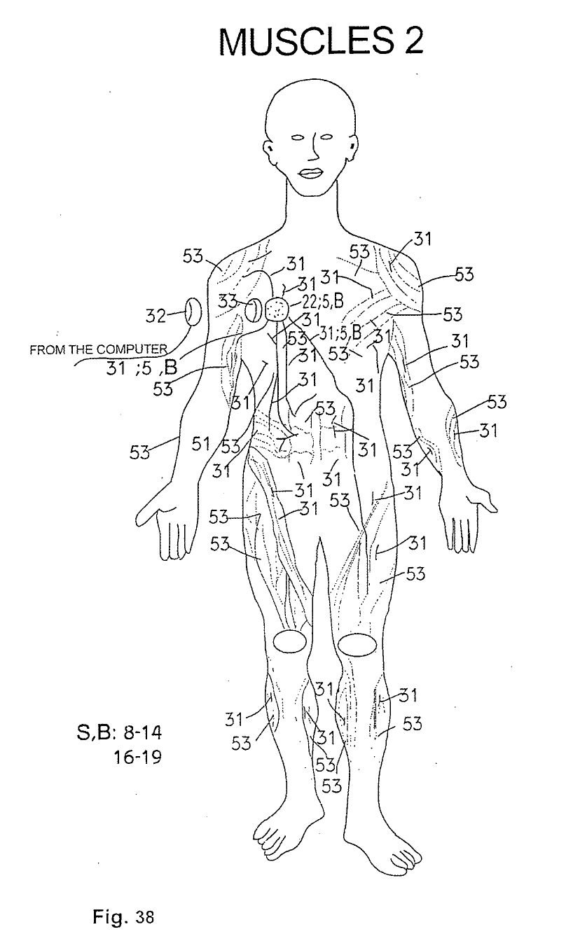

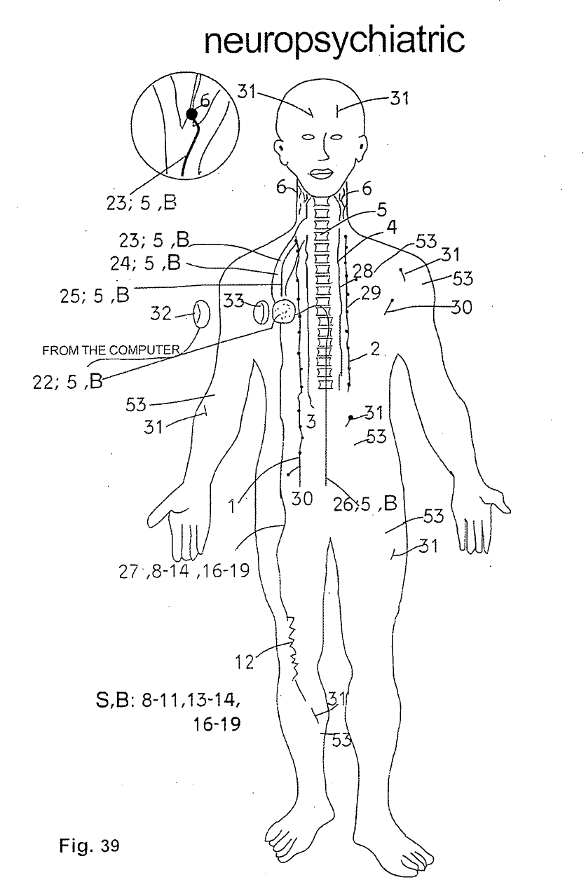

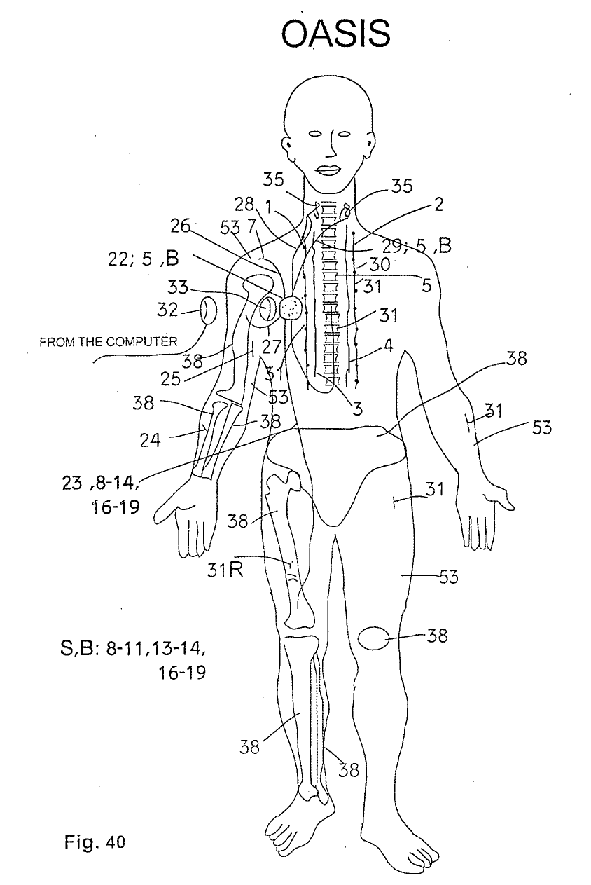

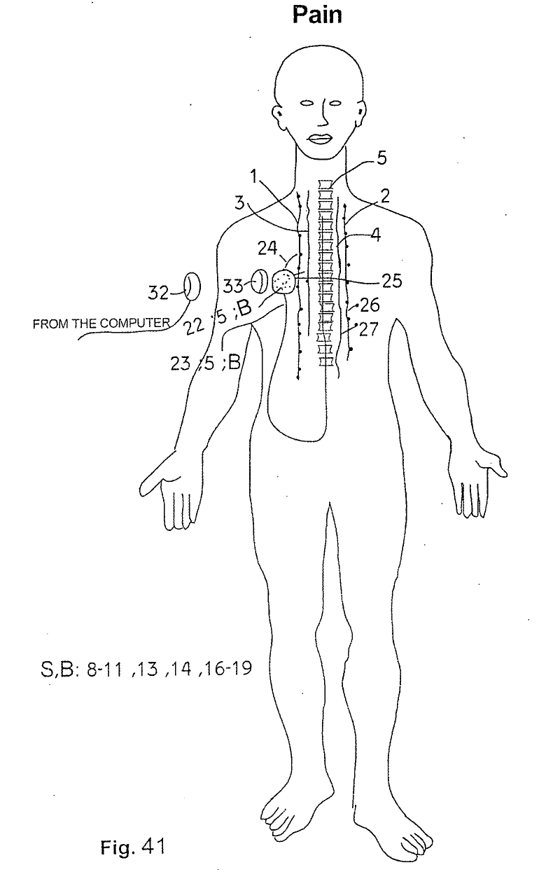

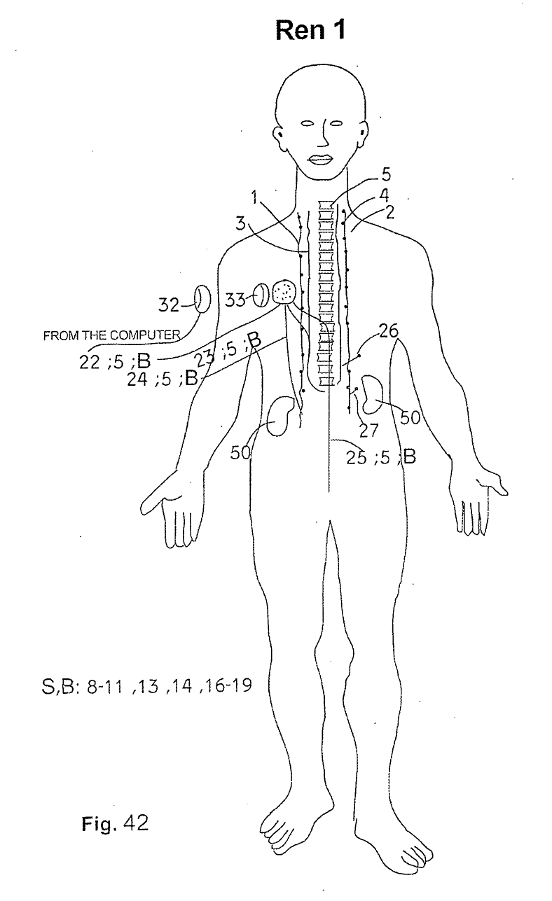

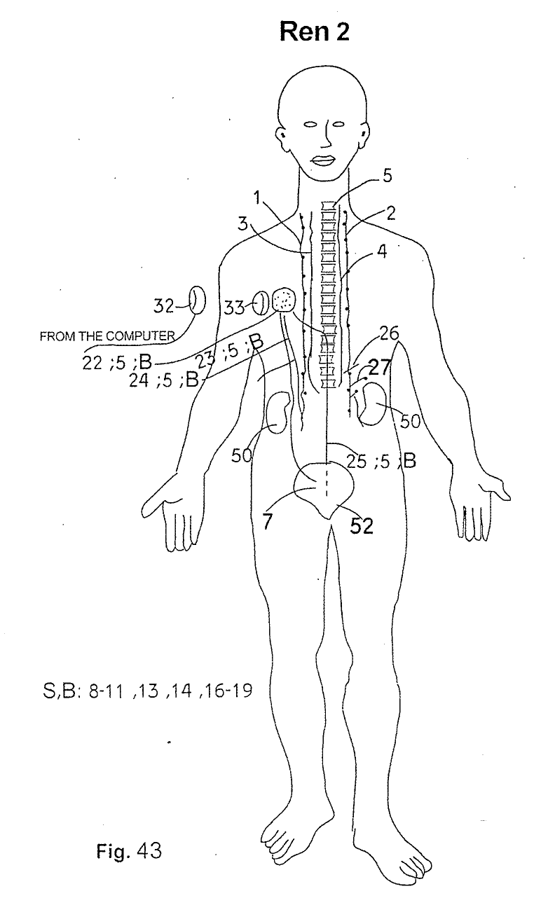

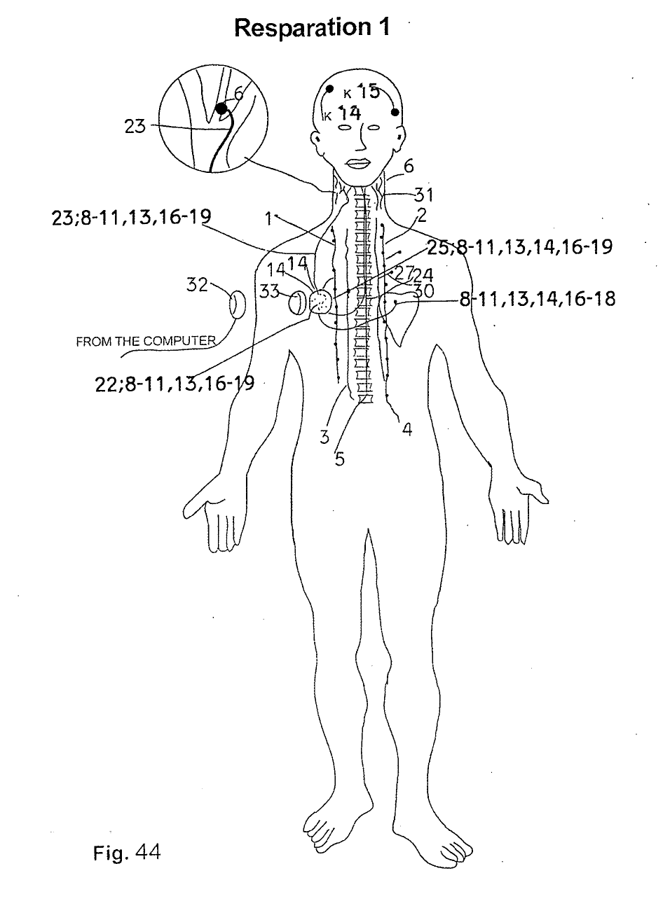

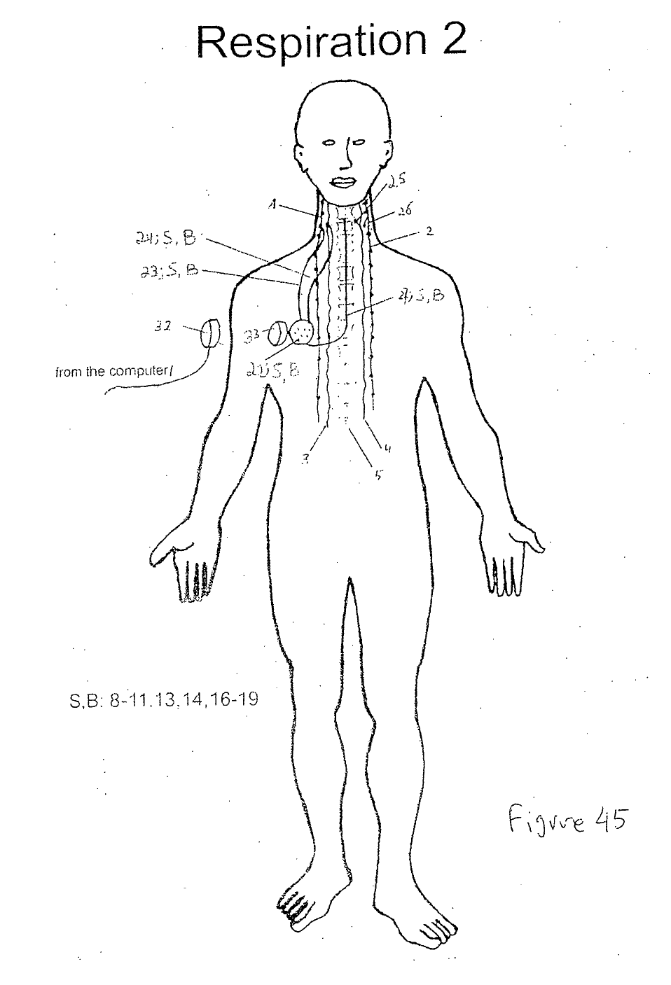

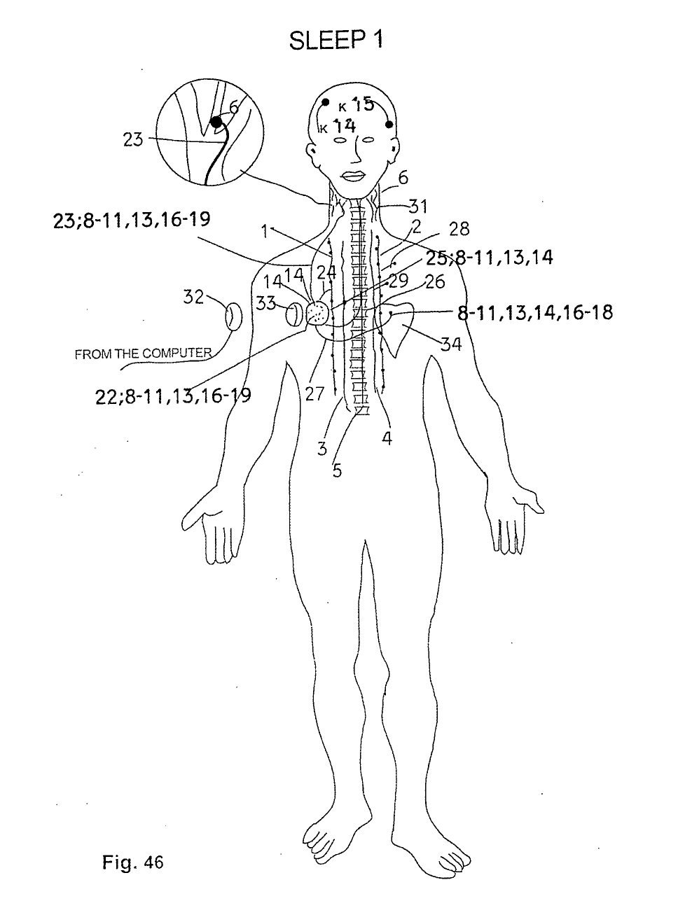

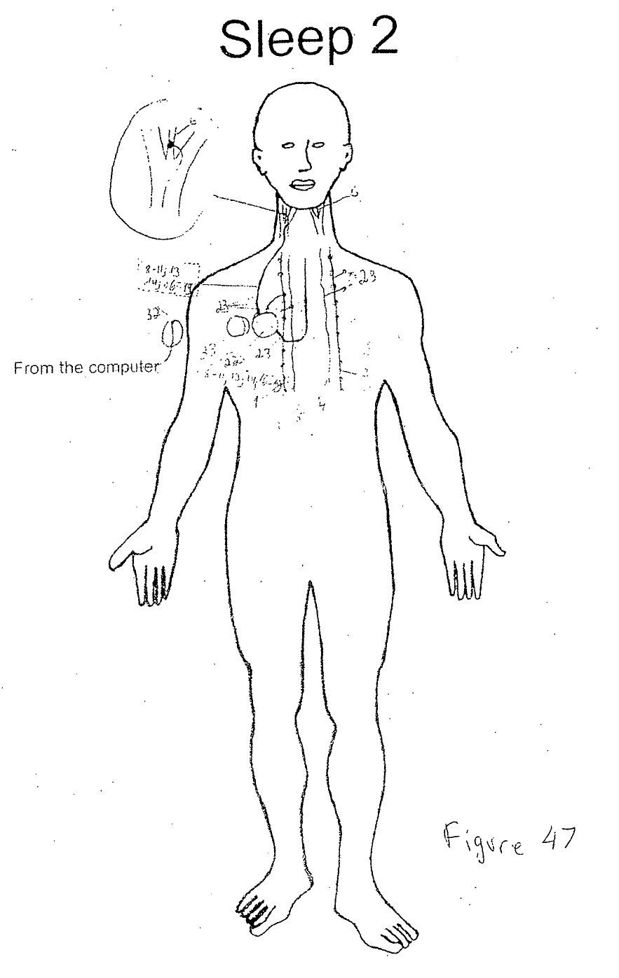

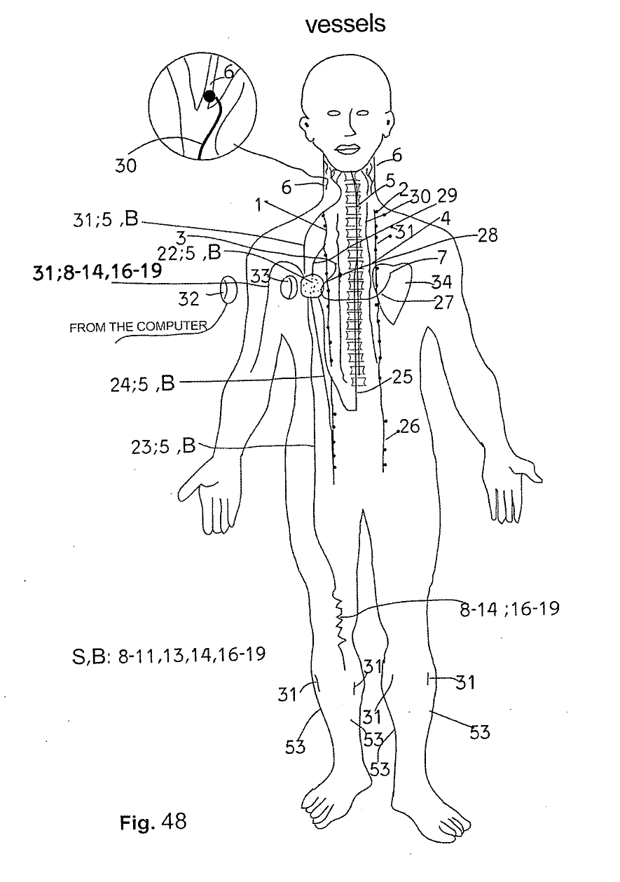

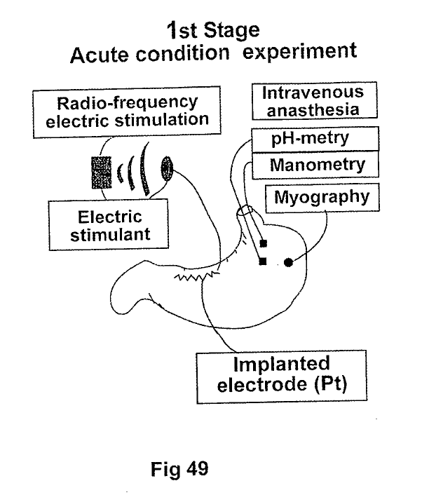

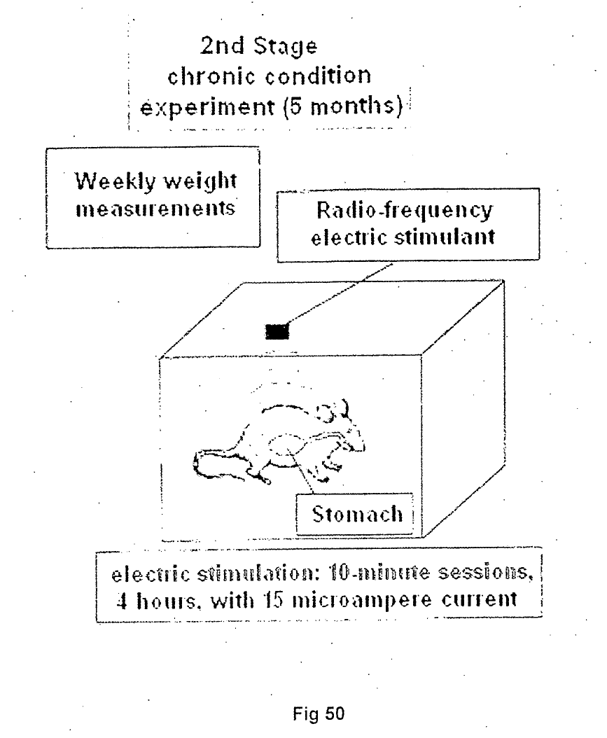

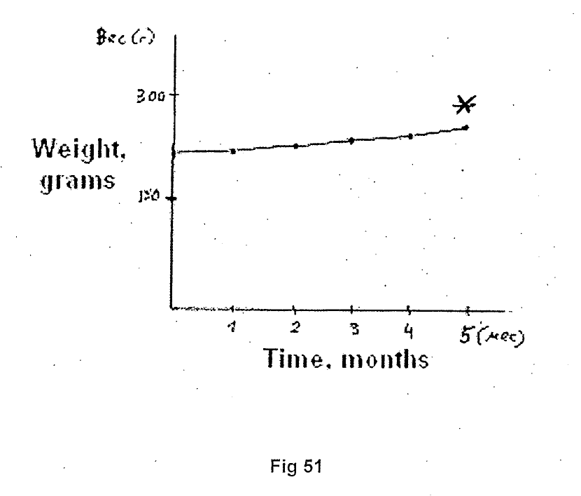

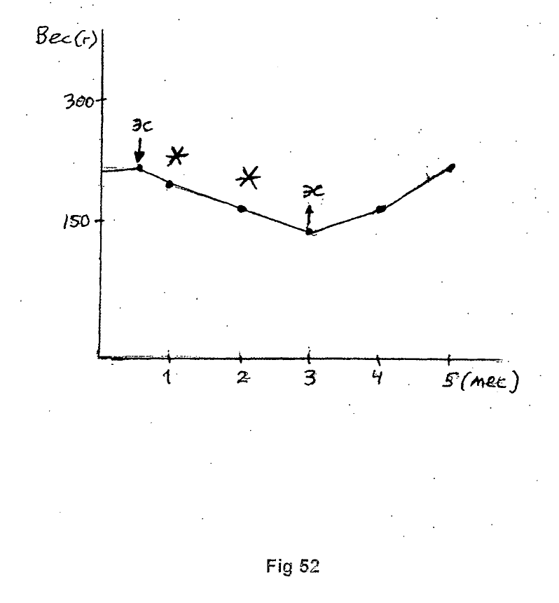

Chronic intestinal obstruction (commissural disease, megacolon, chronic mesenterial circulation insufficiency, metacolon, doloichosigmoid, cardiac
achalasia [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
7—neurons of the organ (stomach)
   Sensors:
8—arterial pressure sensor
9—heart rate sensor
10—respiration rate sensor
11—body temperature sensor
13—local blood circulation sensor
14—sensor of electric activity of the organs and nervous structures
15—gastric acidity sensor
16—sensor of mechanical activity and murmurs of the organs (intestine, heart, lungs, muscles, etc.)
   Biosensors:
17—oxygen biosensor
18—glucose biosensor
19—blood hormones biosensor
22—microchip with sensors, biosensors and electrodes
23-31—electrodes connected to the nervous structures
32—external radio frequency communications unit
33—external chip controller, additional
   Organs:
39—stomach
40—liver
41—gall bladder
43—small intestine
44—large intestine
45—blind gut
46—sigmoid colon
47—rectum
48—rectal sphincter
60—sphincter of the gullet
   FIG. 35—Preferred Implantation Location—Hemo
   Diseases:
anemia [Chip 1, Chip 2, Chip 3, Chip 4, Chip 5, Chip 6]
agranulocytosis [Chip 1, Chip 2, Chip 3, Chip 4, Chip 5, Chip 6]
leucosis [Chip 1, Chip 2, Chip 3, Chip 4, Chip 5, Chip 6]
Immunodeficiency
   Nervous Structures:
1—right sympathetic trunk
2—left sympathetic trunk
3—right vagus nerve
4—left vagus nerve
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
22—microchip with sensors, biosensors and electrodes
23-30—electrodes connected to the nervous structures
32—external radio frequency communications unit
33—external chip controller, additional
   Organs:
36—thymus gland
37—chest bone
38—bones of the limbs, pelvis
44—large intestine
49—spleen
   FIG. 36—Preferred Implantation Location—Hepat 1
   Diseases:
Dyskinesia of the biliferous tracts ( . . . ) [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
7—neurons of the organ (gall bladder)
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles, etc.).
   Biosensors:
17—tissue oxygen biosensor
18—blood glucose biosensor
19—blood hormones biosensor
22—microchip with sensors, biosensors and electrodes
23-30—electrodes connected to the nervous structures
32—external radio frequency communications unit
33—external chip controller, additional
   Organs:
40—liver
41—gall bladder
42—pancreas
62—common bile duct
   FIG. 37—Preferred Implantation Location—Hepat 2
   Diseases:
Chronic hepatitis [Chip 1, Chip 2, Chip 3, Chip 4, Chip 5, Chip 6]
Chronic cholecystopancreatitis [Chip 1, Chip 2, Chip 3, Chip 4, Chip 5, Chip 6]
Cirrhosis [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
7—neurons of the organ (liver)
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
22—microchip with sensors, biosensors and electrodes
23-28—electrodes connected to the nervous structures
32—external radio frequency communications unit
33—external chip controller, additional
   Organs:
40—liver
41—gall blidder
42—pancreas
62—common bile duct
   FIG. 38—Preferred Implantation Location—Muscles 1
   Diseases:
trauma-related paralyses [Chip 1, Chip 2, Chip 3, Chip 4, Chip 5, Chip 6]
myodystrophy [Chip 1, Chip 2, Chip 3, Chip 4, Chip 5, Chip 6]
myopathy [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
7—neurons of the organ-muscles
   Sensors:
8—arterial pressure sensor
9—heart rate sensor
10—respiration rate sensor
11—body temperature sensor
12—Angular shift sensor (for the limbs)
13—local blood circulation sensor
14—sensor of electric activity of the organs and nervous centers
16—sensor of mechanical activity and murmurs of the organs (intestine, heart, lungs, muscles)
   Biosensors:
17—tissue oxygen biosensor
18—blood glucose biosensor
19—blood hormones biosensor
22—microchip with sensors, biosensors and electrodes
31—electrodes connected to the nervous structures
32—external radio frequency communications unit
33—external chip controller, additional
   Organs:
53—muscles
   FIG. 39—Preferred Implantation Location—Muscles 2
   Bodybuilding
   Nervous Structures:
7—neurons of the organ-muscles
   Sensors:
8—arterial pressure sensor
9—heart rate sensor
10—respiration rate sensor
11—body temperature sensor
12—Angular shift sensor (for the limbs)
13—local blood circulation sensor
14—sensor of electric activity of the organs and nervous centers
16—sensor of mechanical activity and murmurs of the organs (intestine, heart, lungs, muscles)
   Biosensors:
17—tissue oxygen biosensor
18—blood glucose biosensor
19—blood hormones biosensor
22—microchip with sensors, biosensors and electrodes
31—electrodes connected to the nervous structures
32—external radio frequency communications unit
33—external chip controller, additional
   Organs:
53—muscles
   FIG. 40—Preferred Implantation Location—Neuropsychiatric
   Diseases:
Schizophrenia with schizophrenic affective disorders and delirium [Chip 1, Chip 2, Chip 3, Chip 4, Chip 5, Chip 6]
Anxiety and depression [Chip 1, Chip 2, Chip 3, Chip 4, Chip 5, Chip 6]
Borderline personality disorder [Chip 1, Chip 2, Chip 3, Chip 4, Chip 5, Chip 6]
Cortical dementia-Alzheimer's disease [Chip 1, Chip 2, Chip 3, Chip 4, Chip 5, Chip 6]
Pick's disease [Chip 1, Chip 2, Chip 3, Chip 4, Chip 5, Chip 6]
Subcortical dementia-supranuclear palsy—(paralysis) [Chip 1, Chip 2, Chip 3, Chip 4, Chip 5, Chip 6]
Huntington's chorea [Chip 1, Chip 2, Chip 3, Chip 4, Chip 5, Chip 6]
Parkinson's disease [Chip 1, Chip 2, Chip 3, Chip 4, Chip 5, Chip 6]
Multi-infarction dementia [Chip 1, Chip 2, Chip 3, Chip 4, Chip 5, Chip 6]
Involuntary movements [Chip 1, Chip 2, Chip 3, Chip 4, Chip 5, Chip 6]
Stammering [Chip 1, Chip 2, Chip 3, Chip 4, Chip 5, Chip 6]
Epilepsy [Chip 1, Chip 2, Chip 3, Chip 4, Chip 5, Chip 6]
Priapism [Chip 1. Chip 2, Chip 3, Chip 4, Chip 5, Chip 6]
Infantile cerebral paralysis [Chip 1, Chip 2, Chip 3, Chip 4, Chip 5, Chip 6]
Paralyses of different etiology [Chip 1, Chip 2, Chip 3, Chip 4, Chip 5, Chip 6]
Syringomyelia [Chip 1, Chip 2, Chip 3, Chip 4, Chip 5, Chip 6]
Progressing myodystrophy and other forms of dystrophy [Chip 1, Chip 2, Chip 3, Chip 4, Chip 5, Chip 6]
Chronic and acute hyperthermia [Chip 1, Chip 2, Chip 3, Chip 4, Chip 5, Chip 6]
Atrophy of the optic nerve [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
   Sensors:
8—arterial pressure sensor
9—heart rate sensor
10—respiration rate sensor
11—body temperature sensor
12—sensor of angular transpositions
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
22—microchip with sensors, biosensors and electrodes
23-31—electrodes connected to the nervous structures
32—external radio frequency communications unit
33—external chip controller, additional
   Organs:
53—muscles
   FIG. 41—Preferred Implantation Location—Osis
   Diseases:
Osteoporosis [Chip 1, Chip 2, Chip 3, Chip 4, Chip 5, Chip 6]
Periostitis, osteosclerosis of different types,
Hyperostosis [Chip 1, Chip 2, Chip 3, Chip 4, Chip 5, Chip 6]
Chronic osteomyelitis
Flaccidly consolidating fractures [Chip 1, Chip 2, Chip 3, Chip 4, Chip 5, Chip 6]
Rickets [Chip 1, Chip 2, Chip 3, Chip 4, Chip 5, Chip 6]
Perthes disease
   Nervous Structures:
1—right sympathetic trunk
2—left sympathetic trunk
3—right vagus nerve
4—left vagus nerve
5—spinal cord
7—neurons of the organ (muscles)
   Sensors:
8—arterial pressure sensor
9—heart rate sensor
10—respiration rate sensor
11—body temperature sensor
12—sensor of angular transpositions
13—local blood circulation sensor
14—sensor of electric activity of the organs and nervous centers
16—sensor of mechanical activity and murmurs of the organs (intestine, heart, lungs, muscles)
   Biosensors:
17—tissue oxygen biosensor
18—blood glucose biosensor
19—blood hormones biosensor
22—microchip with sensors, biosensors and electrodes
23-31—electrodes connected to the nervous structures, bones and muscles
32—external radio frequency communications unit
33—external chip controller, additional
   Organs:
35—parathyroid gland
38—bones of the limbs, pelvis
53—muscles
   FIG. 42—Preferred Implantation Location—Pain
   Diseases:
Chronic periodic pains (angina pectoris, phantom pains, neuritis, nerve root syndromes [Chip 1, Chip 2, Chip 3, Chip 4, Chip 5, Chip 6]
Terminal-stage pains [Chip 1, Chip 2, Chip 3, Chip 4, Chip 5, Chip 6]
Migraine [Chip 1, Chip 2, Chip 3, Chip 4, Chip 5, Chip 6]
Cancer [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
   Sensors:
8—arterial pressure sensor
9—heart rate sensor
10—respiration rate sensor
11—body temperature sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
22—microchip with sensors, biosensors and electrodes
23-27—electrodes connected to the nervous structures
32—external radio frequency communications unit
33—external chip controller, additional
   FIG. 43—Preferred Implantation Location—Ren 1
   Diseases:
Hydronephrosis ( . . . )[Chip 1, Chip 2, Chip 3, Chip 4, Chip 5, Chip 6]
Urinary bladder atony (15) [Chip 1, Chip 2, Chip 3, Chip 4, Chip 5, Chip 6]
Chronic pyelonephritis ( . . . ) [Chip 1, Chip 2, Chip 3, Chip 4, Chip 5, Chip 6]
Chronic glomerulonephritis ( . . . ) [Chip 1, Chip 2, Chip 3, Chip 4, Chip 5, Chip 6]
Chronic cystitis ( . . . ) [Chip 1, Chip 2, Chip 3, Chip 4, Chip 5, Chip 6]
   Note: The framed diseases, structures, sensors, biosensors are to be disregarded as irrelevant.
   Nervous Structures:
1—right sympathetic trunk
2—left sympathetic trunk
3—right vagus nerve
4—left vagus nerve
5—spinal cord
   sinocarotid collector
   neurons of the organ
      Sensors:
6—arterial pressure sensor (1)
7—heart rate sensor (2)
8—respiration rate sensor (3)
9—body temperature sensor (4)
   sensor of angular transpositions (5)
10—local blood circulation sensor (6)
11—sensor of electric activity of the organs and nervous centers (7)
   gastric (juice) acidity sensor (8)
12—sensor of mechanical activity and murmurs of the organs (9)
   Biosensors:
13—tissue oxygen biosensor (1)
14—blood glucose biosensor (2)
15—blood hormones biosensor (3)
   alcohol biosensor (4)
   narcotic substances biosensors (5)
16—microchip with sensors, biosensors and electrodes
17-21—electrodes connected to the nervous structures 1, 2,3,4,5
22—external radio frequency communications unit
23—external chip controller, additional
   Organs:
heart
thyroid gland
thymus gland
chest bone
bones of the
limbs, pelvis
stomach
liver
gall bladder
pancreas
small intestine
large intestine
blind gut
sigmoid colon
rectum
rectal sphincter
spleen
kidneys
adrenal glands
urinary bladder
muscles
arteries
veins
prostate
seminal vesicle
ovaries
testicles
   FIG. 44—Preferred Implantation Location—Ren 2
   Diseases:
Urinary bladder atony [Chip 1, Chip 2, Chip 3, Chip 4, Chip 5, Chip 6]
Chronic cystitis [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
7—neurons of the organ (vesicle)
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs
   Biosensors:
17—tissue oxygen biosensor
18—blood glucose biosensor
19—blood hormones biosensor
22—microchip with sensors, biosensors and electrodes
23-27—electrodes connected to the nervous structures
32—external radio frequency communications unit
33—external chip controller, additional
   Organs:
50—kidneys
52—urinary bladder
   FIG. 45—Preferred Implantation Location—Respiration 1
   Diseases:
Insomnia [Chip 1, Chip 2, Chip 3, Chip 4, Chip 5, Chip 6]
Hypersonmia [Chip 1, Chip 2, Chip 3, Chip 4, Chip 5, Chip 6]
Narcolepsy [Chip 1, Chip 2, Chip 3, Chip 4, Chip 5, Chip 6]
Sudden cardiac arrest at sleep [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles)
   Biosensors:
17—tissue oxygen biosensor
18—blood glucose biosensor
19—blood hormones biosensor
22—microchip with sensors, biosensors and electrodes
23-31—electrodes connected to the nervous structures
32—external radio frequency communications unit
33—external chip controller, additional
   FIG. 46—Preferred Implantation Location—Respiration 2
   Diseases:
Paresis of the vocal cords (27) [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
   Sensors:
8—arterial pressure sensor (1)
9—heart rate sensor (2)
10—respiration rate sensor (3)
11—body temperature sensor (4)
13—local blood circulation sensor (6)
14—sensor of electric activity of the organs and nervous centers (7)
16—sensor of mechanical activity and murmurs of the organs (heart, lungs, muscles) (9)
   Biosensors:
17—tissue oxygen biosensor (1)
18—blood glucose biosensor (2)
19—blood hormones biosensor (3)
22—microchip with sensors, biosensors and electrodes
23-27—electrodes connected to the nervous structures 1, 2,3,4,5,8
32—external radio frequency communications unit
33—external chip controller, additional
   FIG. 47—Preferred Implantation Location—Sleep 1
   Diseases:
Insomnia [Chip 1, Chip 2, Chip 3, Chip 4, Chip 5, Chip 6]
Hypersonmia [Chip 1, Chip 2, Chip 3, Chip 4, Chip 5, Chip 6]
Apnea [Chip 1, Chip 2, Chip 3, Chip 4, Chip 5, Chip 6]
Narcolepsy [Chip 1, Chip 2, Chip 3, Chip 4, Chip 5, Chip 6]
Sudden cardiac arrest at sleep [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
7—neurons of the organ (the heart)
   Sensors:
8—arterial pressure sensor
9—heart rate sensor
10—respiration rate sensor
11—body temperature sensor
13—local blood circulation sensor
14—sensor of electric activity of the organs and nervous centers
16—sensor of mechanical activity and murmurs of the organs (intestine, heart, lungs, muscles)
K14, K15—sensor of electric activity of the brain (EEG)+electrode.
Biosensors:
17—tissue oxygen biosensor
18—blood glucose biosensor
19—blood hormones biosensor
22—microchip with sensors, biosensors and electrodes
23-31—electrodes connected to the nervous structures
32—external radio frequency communications unit
33—external chip controller, additional
   Organs:
34—Heart
   FIG. 48—Preferred Implantation Location—Sleep 2
   Diseases:
Insomnia [Chip 1, Chip 2, Chip 3, Chip 4, Chip 5, Chip 6]
Hypersonmia [Chip 1, Chip 2, Chip 3, Chip 4, Chip 5, Chip 6]
Apnea [Chip 1, Chip 2, Chip 3, Chip 4, Chip 5, Chip 6]
Narcolepsy [Chip 1, Chip 2, Chip 3, Chip 4, Chip 5, Chip 6]
Sudden cardiac arrest at sleep [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
   Sensors:
8—arterial pressure sensor
9—heart rate sensor
10—respiration rate sensor
11—body temperature sensor
13—local blood circulation sensor
14—sensor of electric activity of the organs and nervous structures
16—sensor of mechanical activity and murmurs of the organs (intestine, heart, lungs, muscles)
   Biosensors:
17—tissue oxygen biosensor
18—blood glucose biosensor
19—blood hormones biosensor
22—microchip with sensors, biosensors and electrodes
23—electrodes connected to the nervous structures
32—external radio frequency communications unit
33—external chip controller, additional
   FIG. 49—Preferred Implantation Location—Vessels
   Diseases:
Obliterating atherosclerosis and endarteritis [Chip 1, Chip 2, Chip 3, Chip 4, Chip 5, Chip 6]
Ischemic heart disease and angina pectoris [Chip 1, Chip 2, Chip 3, Chip 4, Chip 5, Chip 6]
Cardiac arrhythmia [Chip 1, Chip 2, Chip 3, Chip 4, Chip 5, Chip 6]
Raynaud's disease [Chip 1, Chip 2, Chip 3, Chip 4, Chip 5, Chip 6]
Buerger's disease [Chip 1, Chip 2, Chip 3, Chip 4, Chip 5, Chip 6]
Chronic thrombophlebitis—supranuclear palsy—(paralysis) [Chip 1, Chip 2, Chip 3, Chip 4, Chip 5, Chip 6]
Postthrombophlebitic syndrome [Chip 1, Chip 2, Chip 3, Chip 4, Chip 5, Chip 6]
   Nervous Structures:
1—right sympathetic trunk
2—left sympathetic trunk
3—right vagus nerve
4—left vagus nerve
5—spinal cord
6—SCVNS: Sinocarotid collector of the Vegetative Nervous System
7—neurons of the organ (heart)
   Sensors:
8—arterial pressure sensor
9—heart rate sensor
10—respiration rate sensor
11—body temperature sensor
12—sensor of angular transpositions
13—local blood circulation sensor
14—sensor of electric activity of the organs and nervous centers
16—sensor of mechanical activity and murmurs of the organs (intestine, heart, lungs, muscles, etc.)
   Biosensors:
17—tissue oxygen biosensor
18—blood glucose biosensor
19—blood hormones biosensor
22—microchip with sensors, biosensors and electrodes
23-31—electrodes connected to the nervous structures
32—external radio frequency communications unit
33—external chip controller, additional
   Organs:
34—Heart
53—muscles
   FIG. 25B—Preferred Implantation Location—Autonomous Power Supply

The structure of the power supply is detailed above. The flexible piezoelectric element (1) is implanted as illustrated, under the diaphragm's cupola (4), from the right side, endoscopically, and is connected to the chip (3).

4 Clinical results, practical experience using the new system and method.

The method has been kept secret, and the system is hidden inside the patient's body

Table 10—Experiments performed for each disease, in animals and humans

Obesity—Experimental Research

Following are the electric stimulation current parameters: frequency: 10-45 hertz, pulse duration: 0.01-0.1 millisecond, amplitude: 15 microampere, voltage: up to 0.5 volt. Programmed electric stimulation sessions were performed on a daily basis, the duration of one session being set at 10 minutes and the frequency of sessions was every four hours. All the animals' weight was monitored on a weekly basis. A half of the animals (13) comprised a control group. After the operation they were placed in a cage without the radio frequency unit.

Results of the Experiments

After the operation, there were 9 surviving rats in the main group, 10 rats remained in the control group.

In the control group a steady growth weight of at least 5% per month was observed among the animals after the operation. (FIG. 53).

FIG. 53. Note: *—Reliable Value Dynamics (?<0.05)

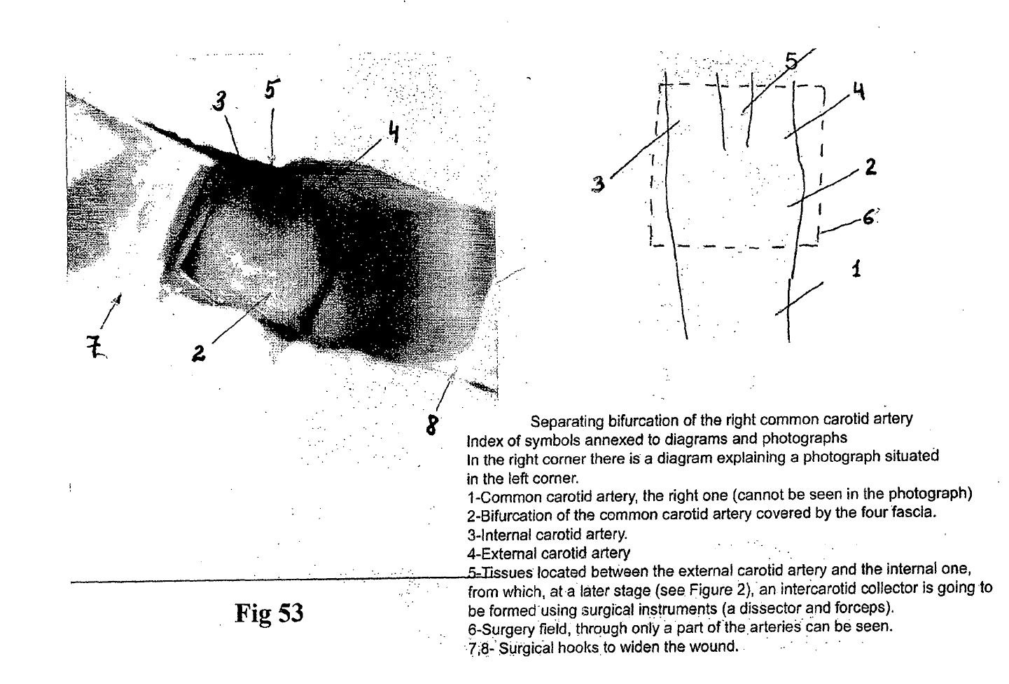

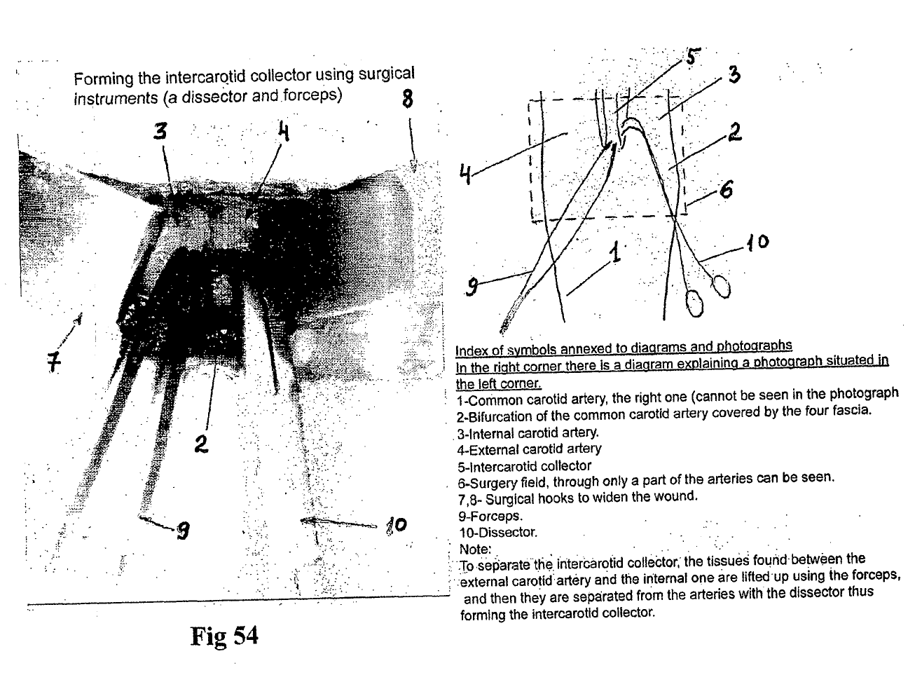

In the main group subjected to periodical programmed electric stimulations of the stomach, no weight increase was recorded in any animal. (FIG. 54).

FIG. 54. Note: *—Reliable Value Dynamics (?<0.05)

On the contrary, 7 rats developed a progressing weight loss of at least 10-15% per month. Two rats died after 4 and 5 weeks respectively after the experiment commencement, and the cause of their death probably was inanition, since the weight loss in them reached 18-25% from the initial value.

After 10 weeks the stimulations were stopped in the main group, that resulted in the surviving rats in a weight growth rehabilitation within 7 to 8 weeks.

Conclusions:

1. The stomach's functional activity can be controlled by means of periodical electric stimulations of the gastric nerves with current of the above-mentioned parameters.

2. Repeated electric stimulations of the nerves of the stomach can cause a stable weight loss in rats.

3. Suspension of electric stimulations of the stomach in the chronic-condition experiment resulted in the weight growth rehabilitation in the rats with stomach-implanted microchips.

4. The method based on periodical electric stimulation of the stomach nerves can be applied to treatment of obese patients being a low-traumatic and more physiological technique, as compared to the conventional surgical procedures.

Analysis of Results of the First Experimental Application of the New Obesity Treatment Technology to Clinical Practice

DESCRIPTION of Uses of the Technology

In a test that has been performed, Microchips have been implanted to 5 obese patients, using 3rd-, 4th- and 5th-generation chips and video-endoscopic surgical techniques to implant them.

The results are presented in Table 11 below.

Conclusions

Periodical electric stimulation of the stomach's nerves by means of the microchips programmed to detect and monitor the stomach's functional activity and to inhibit the latter using the electric stimulation of the above-mentioned nerves, enables a steady predictable weight loss of up to 3 kg per month or more in obese patients.

Chip's Working Algorithms:

PES—programmed periodical electric stimulation, or non-programmed electric stimulation per schedule entered (Chip 1, Chip 2).

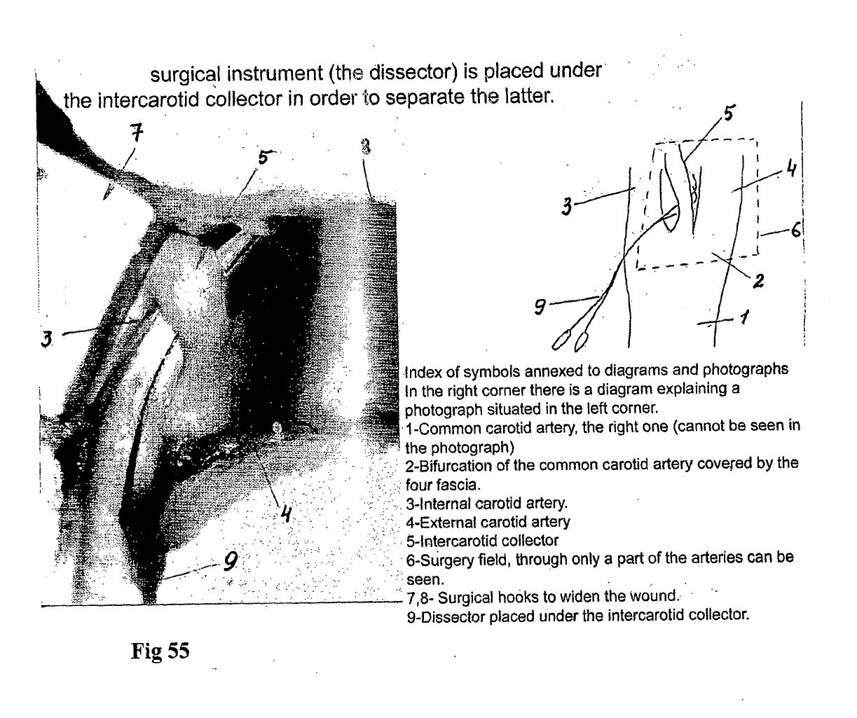

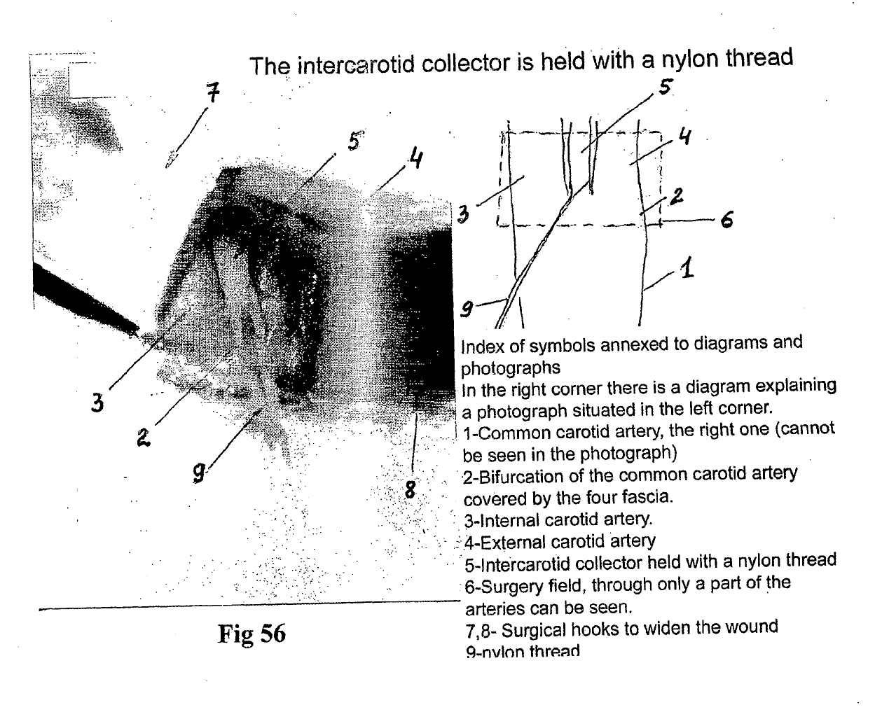

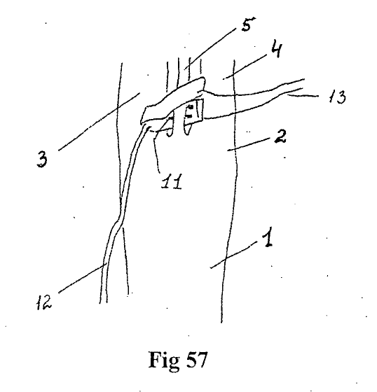

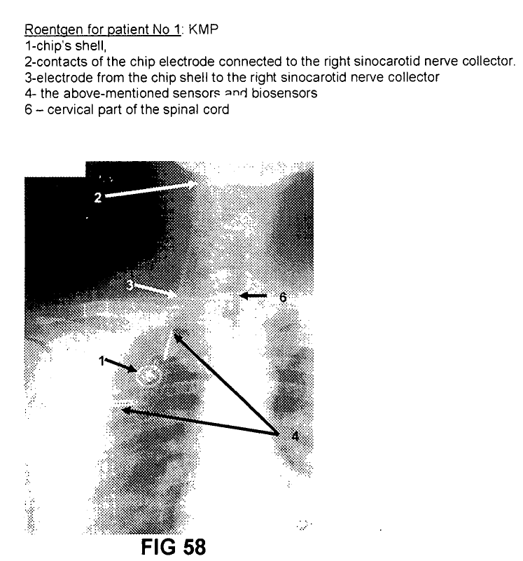

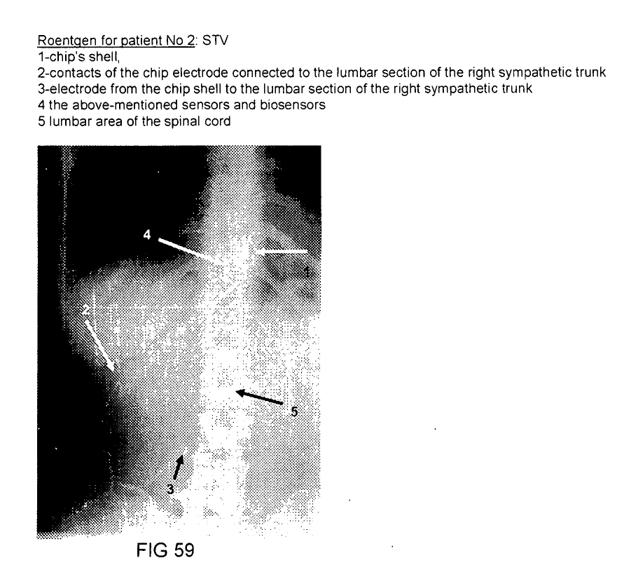

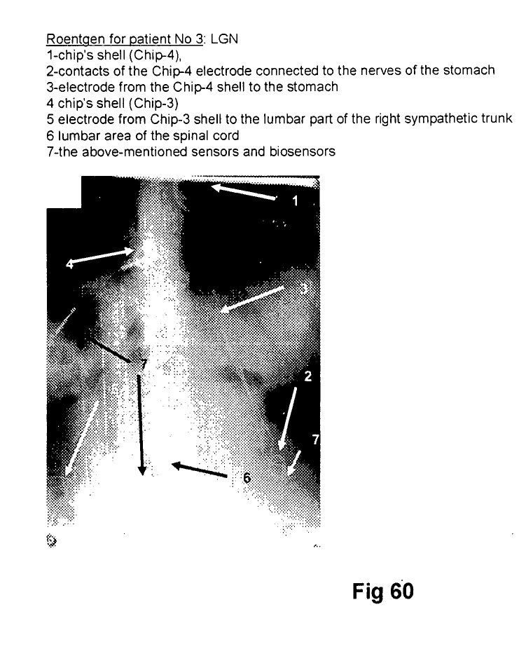

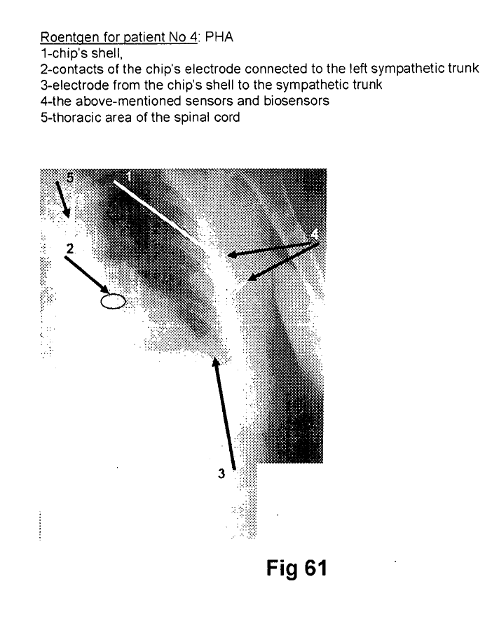

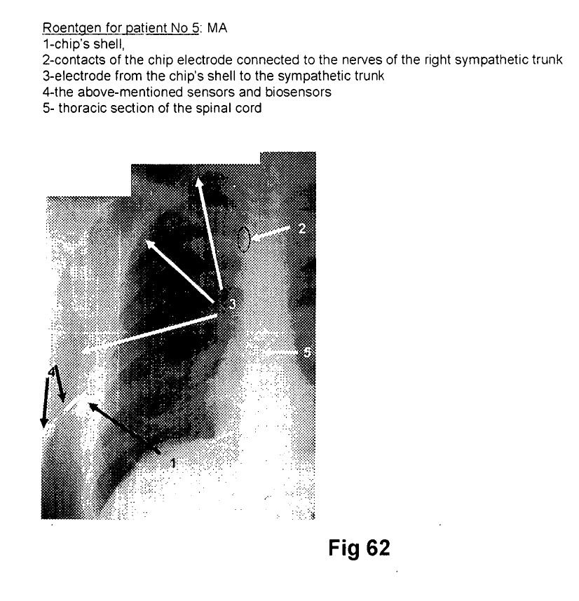

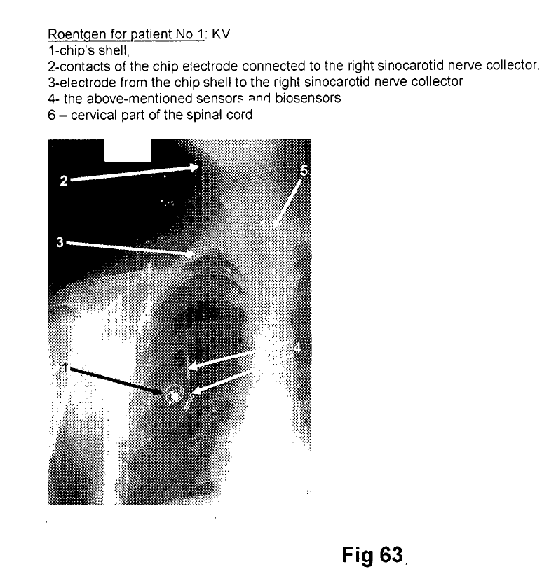

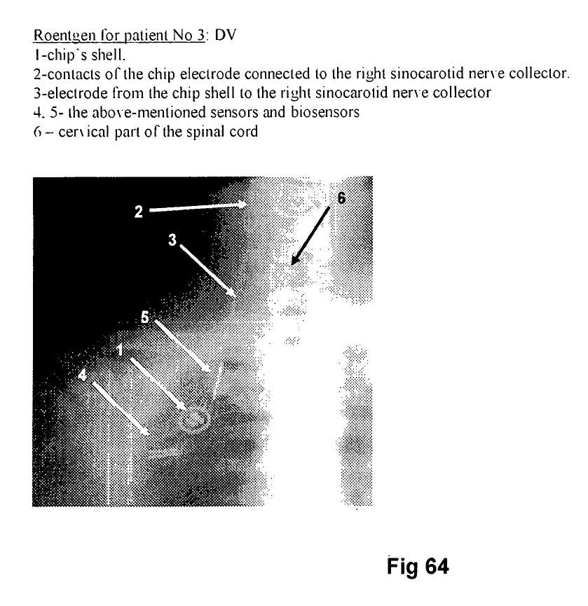

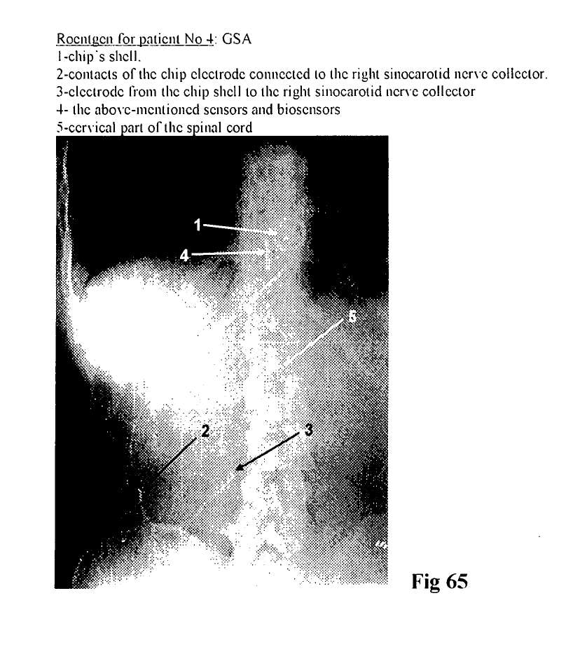

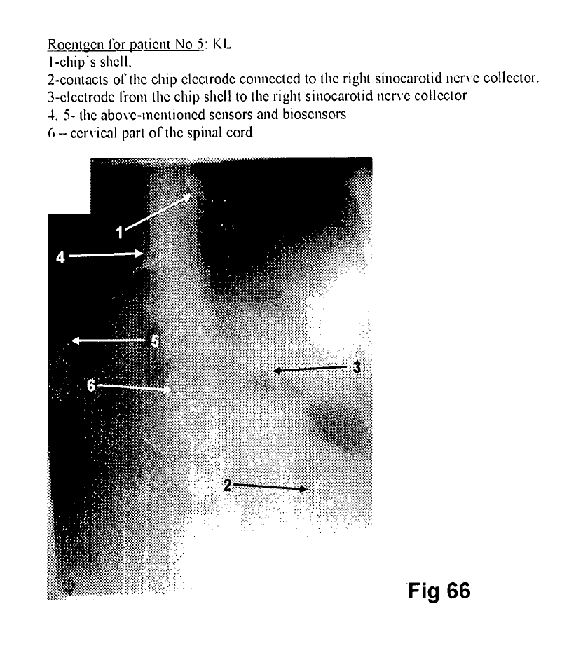

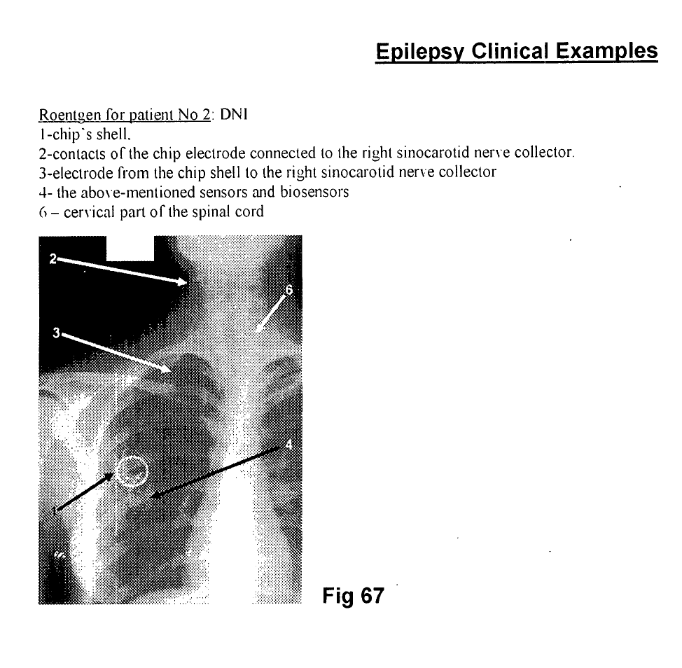

UES—urgent electric stimulation that starts upon detecting (by means of the sensors and biosensors) of a change in the gastric juice acidity level and gastric peristalsis enhancement in order to inhibit stomach functions.
   Table 12—Statistics for obesity treatment, details Mean Data on the 5 Patients from Table 11 above.
   FIGS. 55 to 59—Illustration of surgical procedure
   FIGS. 60 to 69—Roentgen of patient with implanted system
   Table 13—Results of asthma treatment
   Table 14—Asthma surgery data
   Table 15—Epilepsy clinical examples
   Table 16—Epilepsy surgery data
   Table 17—Examples of gastric and duodenal ulcer treatment
   Table 18—Clinical examples of dementia treatment
   Table 19—Clinical examples of treatment of obliterating vascular diseases
   Table 20—List of conditions of the healthy person's body that can be affected with the present invention

### GENERAL NUMBERING FOR DRAWINGS

Nervous Structures:

1—right sympathetic trunk
2—left sympathetic trunk
3—right vagus nerve
4—left vagus nerve
5—spinal cord
6—SCVNS: Sinocarotid collector of the Vegetative Nervous System
7—neurons of the organ (the heart)
7A—cutaneous nerve
7B—depressor nerve
   Sensors:
8—Arterial pressure meter
9—Heart rate meter
10—Respiration rate meter
11—Temperature gage
12—Angular shift sensor (for the limbs)
13—Local blood flow sensor
14—Sensor of electric activity of the organs, nervous centers
15—Gastric juice acidity sensor
16—Murmur sensor (heart, lungs, intestine)
   Biosensors:
17—Biosensor of oxygen contents in the tissues
18—Biosensor of sugar contents in the blood
19—Biosensor of hormone contents in the blood
20—Biosensor of alcohol contents in the blood
21—Biosensor of narcotic substances contents in the blood
22—microchip with sensors, biosensors and electrodes
23-31—electrodes connected to the nervous structures 1, 2,3,4,5,6,7,8
32—external radio frequency communications unit
33—external chip controller, additional
   Organs:
34—heart
35—parathyroid gland
36—thymus gland
37—chest bone
38—bones of the limbs, pelvis
39—stomach
40—liver
41—gall bladder
42—pancreas
43—small intestine
44—large intestine
45—blind gut
46—sigmoid colon
47—rectum
48—rectal sphincter
49—spleen
50—kidneys
51—adrenal glands
52—urinary bladder
53—muscles
54—arteries
55—veins
56—prostate
57—seminal vesicle
58—ovaries
59—testicles
60—sphincter of the gullet
61—skin
62—common bile duct.

### FOLLOWING ARE THE TABLES

The above detailed description includes but several embodiments of the present invention. Various other embodiments are possible and may be devised by a person skilled in the art upon reading the present disclosure.

TABLE 1
System structure and therapeutic applications

| | | | | | | | Chip 6 |
|---|---|---|---|---|---|---|---|
| | | | | | | | “Golden |
| | | | | | | | Needle” |
| | | | | | | | or |
| | | | | | Chip 4 | Chip 5 | “Multi- |
| | | | | | “Multi-Organ | “Visceral | electrode |
| ? | Property | Chip 1 | Chip 2 | Chip 3 | Processor” | Brain” | Wireless Chip” |
| 1 | General View | 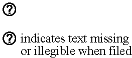 | 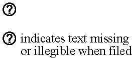 | 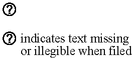 | 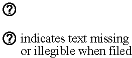 | 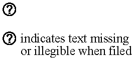 | 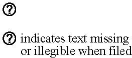 |
| 2 | No. of organs | One | One | One | Up to three | More than three | All the systems |
| | or systems of | | | | | | and organs of |
| | the body that | | | | | | the body can be |
| | can be directly | | | | | | impacted |
| | impacted at a | | | | | | |
| | time | | | | | | |
| 3 | No. Of | One | One | One | Up to three | Up to eight | >100 |
| | channels | | | | | | |
| | through which | | | | | | |
| | the body's | | | | | | |
| | organs and | | | | | | |
| | systems are | | | | | | |
| | impacted | | | | | | |
| 4 | Option to | No | No | Yes | Yes | Yes | Yes |
| | program each | | | | | | |
| | channel of the | | | | | | |
| | microchip | | | | | | |
| | using electro- | | | | | | |
| | magnetic | | | | | | |
| | waves | | | | | | |
| 5 | Power Supply | Radio-frequency | Radio-frequency | Radio-frequency | Radio-frequency | Radio-frequency | Radio-frequency |
| | Type | only, from an | power source | power source | power source | power source | power source |
| | | external power | and a built-in | and a built-in | and a built-in | and a built-in | and a built-in |
| | | unit | autonomous | autonomous | autonomous | autonomous | autonomous |
| | | | power supply | power supply | power supply | power supply | power supply |
| | | | unit | unit | unit | unit, and from | unit, and from |
| | | | | | | the human | the human |
| | | | | | | body's kinetic | body's kinetic |
| | | | | | | energy | energy |
| 6 | Type of the | Sensors | Sensors | Biosensors | Sensors, | Sensors, | Sensors, |
| | chip's sensing | | | | biosensors | biosensors | biosensors |
| | device | | | | | | |
| 7 | Which of the | | | | | | |
| | SENSORS | | | | | | |
| | (gages) listed | | | | | | |
| | below can be | | | | | | |
| | used together | | | | | | |
| | with the chip | | | | | | |
| | for treatment | | | | | | |
| | of the ailments | | | | | | |
| | specified in | | | | | | |
| | Box 15 of this | | | | | | |
| | Table (entry | | | | | | |
| | No. is shown | | | | | | |
| | between the | | | | | | |
| | parenthesis) | | | | | | |
| | LIST OF | | | | | | |
| | SENSORS | | | | | | |
| | 1. Arterial | | | | +(7) | +(7) | +(7) |
| | pressure meter | | | | | | |
| | 2. Heart rate | +(1, 2, 29) | +(1, 2, 29) | +(1, 2, 29) | +(1, 2, 29) | +(1, 2, 29) | +(1, 2, 29) |
| | meter | | | | | | |
| | 3. Respiration | +(1, 2, 29) | +(1, 2, 29) | +(1, 2, 29) | +(1, 2, 29) | +(1, 2, 29) | +(1, 2, 29) |
| | rate meter | | | | | | |
| | 4. Temperature | | | | +(17) | +(17) | +(17) |
| | gage | | | | | | |
| | 5. Angular shift | | | | | | +(9, 10) |
| | sensor (for | | | | | | |
| | the limbs) | | | | | | |
| | 6. Local blood | | | | +(17) | +(17) | +(17) |
| | flow sensor | | | | | | |
| | 7. Sensor of | +(1-29) | +(1-29) | +(1-29) | +(1-29) | +(1-29) | +(1-29) |
| | electric activity | | | | | | |
| | of the organs | | | | | | |
| | [symbol] centers | | | | | | |
| | 8. Gastric [symbol] | | | | +(5, 8) | +(5, 8) | +(5, 8) |
| | sensor | | | | | | |
| | 9. Murmur | +(1, 2, 29) | +(1, 2, 29) | +(1, 2, 29) | +(1, 2, 29) | +(1, 2, 29) | +(1, 2, 29) |
| | sensor (heart, | | | | | | |
| | [symbol] intestine | | | | | | |
| 8 | Which of the | | | | | | |
| | SENSORS | | | | | | |
| | (gages) listed | | | | | | |
| | below can be | | | | | | |
| | used together | | | | | | |
| | with the chip | | | | | | |
| | for treatment | | | | | | |
| | of the ailments | | | | | | |
| | specified in | | | | | | |
| | Box 15 of this | | | | | | |
| | Table (entry | | | | | | |
| | No. is shown | | | | | | |
| | between the | | | | | | |
| | parenthesis) | | | | | | |
| | LIST OF | | | | | | |
| | BIOSENSORS | | | | | | |
| | 1. Biosensor of | | | +(1, 17) | +(1, 17) | +(1, 17) | +(1, 17) |
| | oxygen | | | | | | |
| | contents in the | | | | | | |
| | tissues | | | | | | |
| | 2. Biosensor of | | | | +(6) | +(6) | +(6) |
| | sugar contents | | | | | | |
| | in the blood | | | | | | |
| | 3. Biosensor of | | | | +(13, 14, 26) | +(13, 14, 26) | +(13, 14, 26) |
| | hormone | | | | | | |
| | contents in the | | | | | | |
| | blood | | | | | | |
| | 4. Biosensor of | | | | +(23) | +(23) | +(23) |
| | alcohol | | | | | | |
| | contents in the | | | | | | |
| | blood | | | | | | |
| | 5. Biosensor of | | | | +(24) | +(24) | +(24) |
| | [symbol] substances | | | | | | |
| | contents in the | | | | | | |
| | blood | | | | | | |
| 9 | Parameters of | Electric and | Electric and | Electric activity | Electric activity | Electric activity | Electric activity |
| | the body | mechanical | mechanical | of the organs, | of the organs, | of the organs, | of the organs, |
| | monitored by | activity of the | activity of the | nervous centers, | nervous centers, | nervous centers, | nervous centers, |
| | the chip's | organs, electric | organs, electric | contents of | contents of | contents of | contents of |
| | sensors and | activity of the | activity of the | substances in the | substances in the | substances in the | substances in the |
| | biosensors | nervous centers | nervous centers | tissues | tissues | tissues | tissues |
| | | only | only | (hormones, etc.). | (hormones, etc.), | (hormones, etc.), | (hormones, etc.), |
| | | | | contents of | contents of | contents of | contents of |
| | | | | gases dissolved | gases dissolved | gases dissolved | gases dissolved |
| | | | | in liquid media | in liquid media | in liquid media | in liquid media |
| 10 | No. of organs | One | One | Two | Three | More than three | Monitoring most |
| | or systems of | | | | | | of the systems |
| | the body | | | | | | of the body is |
| | simultaneously | | | | | | possible |
| | monitored by | | | | | | |
| | the chip's | | | | | | |
| | sensors and | | | | | | |
| | biosensors | | | | | | |
| 11 | Chip's | Video | Video | Video | Video | Video | Video |
| | implantation | endosurgical | endosurgical | endosurgical | endosurgical | endosurgical | endosurgical |
| | method. | implantation | implantation | implantation | implantation | implantation | implantation |
| | Average | methods. | methods. | methods. | methods. | methods. | methods, and |
| | duration of the | Hospitalization | Hospitalization | Hospitalization | Hospitalization | Hospitalization | non-invasive |
| | patient's | duration does | duration does | duration does | duration does | duration does | stereoscopic |
| | hospitalization. | not exceed one | not exceed one | not exceed one | not exceed one | not exceed one | techniques, |
| | | day. | day. | day. | day. | day. | controlled by |
| | | | | | | | MRI, etc. |
| | | | | | | | Hospitalization |
| | | | | | | | duration from: |
| | | | | | | | between several |
| | | | | | | | hours up to one |
| | | | | | | | day. |
| 12 | Advantages of | Low cost. High | Low cost. High | Relatively low | Multi-channel | Multi-channel | Unique multi- |
| | the chip in | efficacy. No | efficacy. No | cost, yet varied | feature | feature | channel feature |
| | comparison | replacement is | replacement is | application | | | and large |
| | with other | required. | required. | possibilities | | | number of |
| | existing | | | | | | biosensors, |
| | devices | | | | | | wireless |
| | | | | | | | microelectrodes, |
| | | | | | | | “Golden |
| | | | | | | | Needles”. |
| | Disadvantages | No built-in | No programmed | High cost, | High cost, | High cost, | High cost, |
| | of the chip in | autonomous | modes | complex | complex | complex | complex |
| | comparison | power source | | expensive | expensive | expensive | expensive |
| | with other | | | adjustment | adjustment | adjustment | adjustment |
| | existing | | | equipment | equipment | equipment | equipment |
| | devices | | | | | | |
| 13 | Approximate | 500-1000 | 1500-2000 | 3000-5000 | 7000-10000 | 10000-20000 | >20000 |
| | price, in USD | | | | | | |
| 14 | CHIP'S | | | | | | |
| | MATERIALS | | | | | | |
| | Silicon | + | + | + | + | + | + |
| | Gold | + | + | + | + | | |
| | Platinum | + | + | + | + | + | + |
| | Schungite | + | + | + | + | | |
| | Titanium | + | + | + | + | + | + |
| | (stainless | | | | | | |
| | steel) | | | | | | |
| 15 | Conditions, | | | | | | |
| | ailments that | | | | | | |
| | CANNOT | | | | | | |
| | Be treated | | | | | | |
| | using the chip | | | | | | |
| | 1. Asthma | | | | | | |
| | 2. Epilepsy | | | | | | |
| | 3. Parkinson's | + | + | + | | | |
| | disease | | | | | | |
| | 4. Alzheimer's | + | + | + | | | |
| | disease | | | | | | |
| | 5. Gastric ulcer | | | | | | |
| | 6. Diabetes | + | + | + | | | |
| | 7. Hypertony | | | | | | |
| | 8. Obesity | | | | | | |
| | 9. Infantile | + | + | + | | | |
| | cerebral | | | | | | |
| | paralysis | | | | | | |
| | 10. Post- | + | + | + | | | |
| | traumatic | | | | | | |
| | paralysis | | | | | | |
| | 11. Hirsch- | + | + | + | | | |
| | sprung's | | | | | | |
| | disease | | | | | | |
| | 12. Mental | | | | | | |
| | diseases | | | | | | |
| | 13. Adrenal | | | | | | |
| | cortex failure | | | | | | |
| | 14. Hypo- | | | | | | |
| | thyrosis | | | | | | |
| | 15. Urinary | + | + | | | | |
| | bladder atony | | | | | | |
| | 16. Pain | | | | | | |
| | syndromes | | | | | | |
| | 17. Obliterating | | | | | | |
| | vascular | | | | | | |
| | diseases of the | | | | | | |
| | lower limbs | | | | | | |
| | 18. Consti- | + | + | | | | |
| | pation | | | | | | |
| | 19. Progressing | | | | | | |
| | myodystrophy | | | | | | |
| | 20. Angio- | | | | | | |
| | trophoneurosis | | | | | | |
| | 21. Syringo- | | | | | | |
| | myelia | | | | | | |
| | 22. Neurosis, | | | | | | |
| | depression | | | | | | |
| | 23. Alcoholism | + | + | | | | |
| | 24. Drug | + | + | | | | |
| | addiction | | | | | | |
| | 25. Impotence | + | + | | | | |
| | 27. Paresis of | + | + | | | | |
| | the vocal cords | | | | | | |
| | 28. Osteo- | + | + | | | | |
| | porosis | | | | | | |
| | 29. Angina | | | | | | + |
| | pectoris | | | | | | |
| 16 | Other possible | | | | | | |
| | NON-THERA- | | | | | | |
| | PEUTIC chip | | | | | | |
| | application | | | | | | |
| | options | | | | | | |
| 17 | 1. Programmed | | | | | | + |
| | muscle mass | | | | | | |
| | increase | | | | | | |
| | (bodybuilding) | | | | | | |
| | 2. Automatic | | | | | + | + |
| | sleep control | | | | | | |
| | (chip's mode: | | | | | | |
| | <<alarm | | | | | | |
| | clock>>, | | | | | | |
| | <<sleep>>, | | | | | | |
| | etc.) | | | | | | |
| | 3. Automatic | | | | | + | + |
| | reminder | | | | | | |
| 18 | Electrodes | | | | | | |
| | compatible | | | | | | |
| | with the chip | | | | | | |
| | “Book-type”, | + | + | + | + | + | + |
| | bipolar | | | | | | |
| | Multi-channel, | + | + | + | + | + | + |
| | 2-8-polar and | | | | | | |
| | more | | | | | | |
| | Spiral | + | + | + | + | + | + |
| | “Golden | | | | | | + |
| | needle” | | | | | | |
| | Plate-like | + | + | + | + | + | + |
| | Wire gauze | + | + | + | + | + | + |
| | Coaxial | + | + | + | + | + | + |

TABLE 2
Description of sensors

| | Sensor Name | | |
|---|---|---|---|
| | | | 3 |
| | 1 | 2 | Respiration rate |
| Parameters of Sensors | Arterial pressure sensor | Heart rate sensor | sensor |
| Location on the chip | A - Sensitive elements of sensors and | A - Sensitive elements of sensors and | A - Sensitive elements |
| | biosensors can be located both on the | biosensors can be located both on the | of sensors and |
| | chip's coating and under it, as well as | chip's coating and under it, as well as | biosensors can be |
| | at the electrode contacts' endings or | at the electrode contacts' endings or | located both on the |
| | any other part of the electrode. | any other part of the electrode. | chip's coating and under |
| | B - Sensors, biosensors can be | B - Sensors, biosensors can be | it, as well as at the |
| | implanted into tissues, lumens of | implanted into tissues, lumens of | electrode contacts' |
| | organs and blood vessels. | organs and blood vessels. | endings or any other |
| | | | part of the electrode. |
| | | | B - Sensors, biosensors |
| | | | can be implanted into |
| | | | tissues, lumens of |
| | | | organs and blood |
| | | | vessels. |
| Shape, type | S—Spiral - or crystal-shape element | S—Spiral - or crystal-shape element | S—Spiral - or crystal- |
| | OT—Other-type element | OT—Other-type element | shape element |
| | Bl—“Blot”-shape sensitive element | Bl—“Blot”-shape sensitive element | OT—Other-type |
| | D—“Dot”-shape sensitive element | D—“Dot”-shape sensitive element | element |
| | Int—Intra-organ | Int—Intra-organ | Bl—“Blot”-shape |
| | Ext—External | Ext—External | sensitive element |
| | | | D—“Dot”-shape |
| | | | sensitive element |
| | | | Int—Intra-organ |
| | | | Ext—External |
| Working principle | MEV—Measurement of electric | Pz—Piezo-effect | Pz—Piezo-effect |
| | values, pertaining to organs' function | MEV—Measurement of electric | MEV—Measurement of |
| | Pz—Piezo-effect | values, pertaining to organs' function | electric values, |
| | | | pertaining to organs' |
| | | | function |
| Number of sensitive | Any number | Any number | Any number |
| elements per 1 sm² | | | |
| Type of signal received | Code signal | Code signal | Code signal |
| | Analog signal | Analog signal | Analog signal |
| Membrane's structure, | Silicon membrane or other | Silicon membrane or other | Silicon membrane or |
| receptor, substance | biologically inert porous material | biologically inert porous material | other biologically inert |
| | Special substance or electronic | Special substance or electronic | porous material |
| | component | component | Special substance or |
| | | | electronic component |
| Range of values measured | Disease-dependent | Disease-dependent | Disease-dependent |
| Size range of sensitive | Micrometers | Micrometers | Micrometers |
| elements | millimeters | millimeters | millimeters |
| | Sensor Name | | |
| | | | 6 |
| | 4 | 5 | Local blood flow |
| Parameters of Sensors | Body temperature sensor | Angular transposition sensor | sensor |
| Location on the chip | A - Sensitive elements of sensors and | A - Sensitive elements of sensors and | A - Sensitive elements |
| | biosensors can be located both on the | biosensors can be located both on the | of sensors and |
| | chip's coating and under it, as well as | chip's coating and under it, as well as | biosensors can be |
| | at the electrode contacts' endings or | at the electrode contacts' endings or | located both on the |
| | any other part of the electrode. | any other part of the electrode. | chip's coating and under |
| | B - Sensors, biosensors can be | B - Sensors, biosensors can be | it, as well as at the |
| | implanted into tissues, lumens of | implanted into tissues, lumens of | electrode contacts' |
| | organs and blood vessels. | organs and blood vessels. | endings or any other |
| | | | part of the electrode. |
| | | | B - Sensors, biosensors |
| | | | can be implanted into |
| | | | tissues, lumens of |
| | | | organs and blood |
| | | | vessels. |
| Shape, type | S—Spiral- or crystal-shape element | S—Spiral- or crystal-shape element | S—Spiral- or crystal- |
| | OT—Other-type element | OT—Other-type element | shape element |
| | Bl—“Blot”-shape sensitive element | Bl—“Blot”-shape sensitive element | OT—Other-type |
| | D—“Dot”-shape sensitive element | D—“Dot”-shape sensitive element | element |
| | Int—Intra-organ | Int—Intra-organ | Bl—“Blot”-shape |
| | Ext—External | Ext—External | sensitive element |
| | | | D—“Dot”-shape |
| | | | sensitive element |
| | | | Int—Intra-organ |
| | | | Ext—External |
| Working principle | ThE—Thermoelement | Pz—Piezo-effect | O, R—Optical effect, |
| | | R—resistometry | resistometry |
| | | | ThE—Thermoelement |
| Number of sensitive | Any number | Any number | Any number |
| elements per 1 sm² | | | |
| Type of signal received | Code signal | Code signal | Code signal |
| | Analog signal | Analog signal | Analog signal |
| Membrane's structure, | Silicon membrane or other | Silicon membrane or other | Silicon membrane or |
| receptor, substance | biologically inert porous material | biologically inert porous material | other biologically inert |
| | Special substance or electronic | Special substance or electronic | porous material |
| | component | component | Special substance or |
| | | | electronic component |
| Range of values measured | Disease-dependent | Disease-dependent | Disease-dependent |
| Size range of sensitive | Micrometers | Micrometers | Micrometers |
| elements | millimeters | millimeters | millimeters |
| | Sensor Name | | |
| | | | 9 |
| | | | Sensor of |
| | | | mechanical activity, |
| | | | murmurs and |
| | 7 | | wheezing of organs |
| | Sensor of electric activity of | 8 | (intestine, heart, |
| Parameters of Sensors | organs and nervous centers | Acidity (gastric juice) sensor | lungs, muscles, etc.) |
| Location on the chip | A - Sensitive elements of sensors and | A - Sensitive elements of sensors and | A - Sensitive elements |
| | biosensors can be located both on the | biosensors can be located both on the | of sensors and |
| | chip's coating and under it, as well as | chip's coating and under it, as well as | biosensors can be |
| | at the electrode contacts' endings or | at the electrode contacts' endings or | located both on the |
| | any other part of the electrode. | any other part of the electrode. | chip's coating and under |
| | B - Sensors, biosensors can be | B - Sensors, biosensors can be | it, as well as at the |
| | implanted into tissues, lumens of | implanted into tissues, lumens of | electrode contacts' |
| | organs and blood vessels. | organs and blood vessels. | endings or any other |
| | | | part of the electrode. |
| | | | B - Sensors, biosensors |
| | | | can be implanted into |
| | | | tissues, lumens of |
| | | | organs and blood |
| | | | vessels. |
| Shape, type | S—Spiral- or crystal-shape element | S—Spiral- or crystal-shape element | S—Spiral- or crystal- |
| | OT—Other-type element | OT—Other-type element | shape element |
| | Bl—“Blot”-shape sensitive element | Bl—“Blot”-shape sensitive element | OT—Other-type |
| | D—“Dot”-shape sensitive element | D—“Dot”-shape sensitive element | element |
| | Int—Intra-organ | Int—Intra-organ | Bl—“Blot”-shape |
| | Ext—External | Ext—External | sensitive element |
| | | | D—“Dot”-shape |
| | | | sensitive element |
| | | | Int—Intra-organ |
| | | | Ext—External |
| Working principle | MEV—Measurement of electric | ECh—Electrochemical | Pz—Piezo-effect |
| | values, pertaining to organs' function | | O, R—Optical effect, |
| Number of sensitive | Any number | Any number | Any number |
| elements per 1 sm² | | | |
| Type of signal received | Code signal | Code signal | Code signal |
| | Analog signal | Analog signal | Analog signal |
| Membrane's structure, | Silicon membrane or other | Silicon membrane or other | Silicon membrane or |
| receptor, substance | biologically inert porous material | biologically inert porous material | other biologically inert |
| | Special substance or electronic | Special substance or electronic | porous material |
| | component | component | Special substance or |
| | | | electronic component |
| Range of values measured | Disease-dependent | Disease-dependent | Disease-dependent |
| Size range of sensitive | Micrometers | Micrometers | Micrometers |
| elements | millimeters | millimeters | millimeters |

TABLE 3
Description of biosensors

| | Biosensor Name | | |
|---|---|---|---|
| Parameters | | | 3 |
| of | 1 | 2 | Blood and tissue hormones |
| biosensors | Tissue oxygen biosensor | Blood glucose biosensor | biosensor |
| Location on | A - Sensitive elements of sensors and | A - Sensitive elements of sensors and | A - Sensitive elements of sensors |
| the chip | biosensors can be located both on the | biosensors can be located both on the | and biosensors can be located both |
| | chip's coating and under it, as well as at | chip's coating and under it, as well as at | on the chip's coating and under it, |
| | the electrode contacts' endings or any | the electrode contacts' endings or any | as well as at the electrode contacts' |
| | other part of the electrode. | other part of the electrode. | endings or any other part of the |
| | B - Sensors, biosensors can be | B - Sensors, biosensors can be | electrode. |
| | implanted into tissues, lumens of | implanted into tissues, lumens of | B - Sensors, biosensors can be |
| | organs and blood vessels. | organs and blood vessels. | implanted into tissues, lumens of |
| | | | organs and blood vessels. |
| Shape, type | S—Spiral- or crystal-shape element | S—Spiral- or crystal-shape element | S—Spiral- or crystal-shape element |
| | OT—Other-type element | OT—Other-type element | OT—Other-type element |
| | Bl—“Blot”-shape sensitive element | Bl—“Blot”-shape sensitive element | Bl—“Blot”-shape sensitive element |
| | D—“Dot”-shape sensitive element | D—“Dot”-shape sensitive element | D—“Dot”-shape sensitive element |
| | Int—Intra-organ | Int—Intra-organ | Int—Intra-organ |
| | Ext—External | Ext—External | Ext—External |
| Working | O, R—Optical effect, resistometry | O, R—Optical effect, resistometry | O, R—Optical effect, resistometry |
| principle | Pz—Piezo-effect | MEV—Measurement of electric values, | Pz—Piezo-effect |
| | | pertaining to organs' function | |
| sensitive | Any number | Any number | Any number |
| elements | | | |
| per 1 cm² | | | |
| Type of | Code signal | Code signal | Code signal |
| signal | Analog signal | Analog signal | Analog signal |
| received | | | |
| Membrane's | Silicon membrane or other | Silicon membrane or other | Silicon membrane or other |
| structure, | biologically inert porous material | biologically inert porous material | biologically inert porous material |
| receptor, | Special substance or electronic | Special substance or electronic | Special substance or electronic |
| substance | component | component | component |
| Range of | Disease-dependent | Disease-dependent | Disease-dependent |
| values | | | |
| measured | | | |
| Size range | Micrometers | Micrometers | |
| of sensitive | millimeters | millimeters | |
| elements | | | |

Biosensor Name

| | 4 | |
|---|---|---|
| Parameters of | Blood and tissue alcohol | 5 |
| biosensors | biosensor | Narcotic substances biosensor |
| Location on | A - Sensitive elements of sensors and | A - Sensitive elements of sensors and |
| the chip | biosensors can be located both on the | biosensors can be located both on the |
| | chip's coating and under it, as well as at | chip's coating and under it, as well as at |
| | the electrode contacts' endings or any | the electrode contacts' endings or any |
| | other part of the electrode. | other part of the electrode. |
| | B - Sensors, biosensors can be | B - Sensors, biosensors can be |
| | implanted into tissues, lumens of organs | implanted into tissues, lumens of organs |
| | and blood vessels. | and blood vessels. |
| Shape, type | S—Spiral- or crystal-shape element | S—Spiral- or crystal-shape element |
| | OT—Other-type element | OT—Other-type element |
| | Bl—“Blot”-shape sensitive element | Bl—“Blot”-shape sensitive element |
| | D—“Dot”-shape sensitive element | D—“Dot”-shape sensitive element |
| | Int—Intra-organ | Int—Intra-organ |
| | Ext—External | Ext—External |
| Working | MEV—Measurement of electric values, | ECh—Electrochemical |
| principle | pertaining to organs' function | |
| | ECh—Electrochemical | |
| sensitive | Any number | Any number |
| elements per 1 sm² | | |
| Type of signal | Code signal | Code signal |
| received | Analog signal | Analog signal |
| Membrane's | Silicon membrane or other | Silicon membrane or other |
| structure, | biologically inert porous material | biologically inert porous material |
| receptor, | Special substance or electronic | Special substance or electronic |
| substance | component | component |
| Range of | Disease-dependent | Disease-dependent |
| values | | |
| measured | | |
| Size range of | Micrometers | Micrometers |
| sensitive | millimeters | millimeters |
| elements | | |

TABLE 4
(epilepsy)
Example of Algorithm

| Arbitrary number of | Numbers of sensors | Numbers of sensors |
|---|---|---|
| sensors, biosensors | and biosensors- | and biosensors- |
| (according to the | generated code signals | generated code signals |
| numbering system used | (see Table 3) in charge | (see Table 3) in charge |
| in our summary table) | of ON command | of OFF command |
| Numbers of sensors | | |
| 1 | 2, 4 | 1, 3 |
| 2 | 2, 4 | 1, 3 |
| 3 | 2, 4 | 1, 3 |
| 4 | 2, 4 | 1, 3 |
| 5 | 2, 4 | 1, 3 |
| 6 | 2, 4 | 1, 3 |
| 7 | 2, 4 (EEG) | 1, 3 |
| 8 | — | — |
| 9 | 2, 4 | 1, 3 |
| Numbers of biosensors | | |
| 1 | 2, 4 | 1, 3 |
| 2 | — | — |
| 3 | — | — |
| 4 | — | — |
| 5 | — | — |

TABLE 5
asthma
Example of Algorithm

| Arbitrary number of | Numbers of sensors | Numbers of sensors |
|---|---|---|
| sensors, biosensors | and biosensors- | and biosensors- |
| (according to the | generated code signals | generated code signals |
| numbering system used | (see Table 3) in charge | (see Table 3) in charge |
| in the summary table) | of ON command | of OFF command |
| Numbers of sensors | | |
| 1 | 2, 4 | 1, 3 |
| 2 | 2, 4 | 1, 3 |
| 3 | 2, 4 | 1, 3 |
| 4 | — | — |
| 5 | — | — |
| 6 | — | — |
| 7 | — | — |
| 8 | — | — |
| 9 | 2, 4 | 1, 3 |
| Numbers of biosensors | | |
| 1 | 2, 4 | 1, 3 |
| 2 | — | — |
| 3 | 2, 4 | 1, 3 |
| 4 | — | — |
| 5 | — | — |

TABLE 6
Method of sensors/biosensors activation

| | | Numbers and meaning | | | | |
|---|---|---|---|---|---|---|
| | | of sensors and biosensors-generated code signals | | | | |
| | | (a possible appearance of these signals is shown in FIG. 1) | | | | |
| | | Patient is | Patient is | Patient is | Patient is | |
| | | asleep, no | asleep, onset | awake, no | awake, onset | |
| | | seizure or | of seizure or | seizure or | of seizure or | |
| | | other | other | other | other | “X” state, |
| | Name of sensors, | symptoms of | symptoms of | symptoms of | symptoms of | such as |
| | biosensors, and chip | ailment | ailment | ailment | ailment | walking |
| No | program codes | Code 1 | Code 2 | Code 3 | Code4 | Code . . . |
| | SENSORS | | | | | |
| 1 | Arterial pressure (1) | N | CC1 | N | CC2 | CC3 |
| | (AP) | | | | | |
| 2 | Heart beat rate (2) | N | CC1 | N | CC2 | CC3 |
| | (HBR) | | | | | |
| 3 | Respiratory rate (3) | N | CC1 | N | CC2 | CC3 |
| | (RR) | | | | | |
| 4 | Temperature (4) (t° C.) | N | CC1 | N | CC2 | CC3 |
| 5 | Angular displacement | SI | CC1 | N | CC2 | CC3 |
| | (5) | | | | | |
| 6 | Local circulation (6) | N | CC1 | N | CC2 | CC3 |
| 7 | Electric activity of | N | CC1 | N | CC2 | CC3 |
| | organs (7) | | | | | |
| 8 | pH (8) | N | CC1 | N | CC2 | CC3 |
| 9 | Mechanical activity of | N | CC1 | N | CC2 | CC3 |
| | organs (9) | | | | | |
| | BIOSENSORS | | | | | |
| 10 | Oxygen (1) (pO₂) | N | CC1 | N | CC2 | CC3 |
| 11 | Glucose (2) | N | CC1 | N | CC2 | CC3 |
| 12 | Hormones (3) | N | CC1 | N | CC2 | CC3 |
| 13 | Alcohol (4) | SI/SA | CC1 | N | CC2 | CC3 |
| 14 | Narcotics (5) | SI/SA | CC1 | N | CC2 | CC3 |
| 15 | Chip program codes | Chip OFF | Chip ON | Chip OFF | Chip ON | Chip OFF/ |
| | (built-in) | | | | | Chip ON |

Notes:
1 Specific values of sensors and biosensors-generated signals, as assigned to each of the codes, are determined separately per each patient.
SI—signal inavailable

TABLE 7
Treatment of diseases using the microchips (Table of Diseases)

| | Diseases | | | | | Nerves best mode | Nerves second best (exact |
|---|---|---|---|---|---|---|---|
| | and | | | | Elec- | (exact anatomic | anatomic |
| N | syndromes | Sensors | Biosensors | Chip | trode | definition and location) | definition and location) |
| 1 | Asthma | 8, 9, 10, | 17, 18 | 1-6 | 1-9 | SKD - Sinocarotid collector, right | TSTD - Sympathetic trunk, |
| | | 11, 12, | | | | (over the bifurcation spot of the | thoracic, right (in the superior |
| | | 13, 14, | | | | common carotid artery) | third, at the level T2-T4) |
| | | 16 | | | | SKS - Sinocarotid collector, left | TSTS - Sympathetic trunk, |
| | | | | | | (over the bifurcation spot of the | thoracic, left (in the superior |
| | | | | | | common carotid artery) | third, at the level T7-T-11) |
| 2 | Epilepsy | 8, 9, 10, | 17, 18, 19, | 1-6 | 1-9 | SKD - Sinocarotid collector, right | VS - Vagus nerve, left (in the |
| | | 11, 12, | 20 | | | (over the bifurcation spot of the | superior third of the neck, at the |
| | | 13, 14, | | | | common carotid artery) | level C2-C3) |
| | | 16 | | | | SKS - Sinocarotid collector, left | VD - Vagus nerve, right (in the |
| | | | | | | (over the bifurcation spot of the | superior third of the neck, at the |
| | | | | | | common carotid artery) | level C2-C3) |
| 3 | Parkinson's | 8, 9, 10, | 17, 18 | 1-6 | 1-9 | SKD - Sinocarotid collector, right | VS - Vagus nerve, left (in the |
| | disease | 11, 12, | | | | (over the bifurcation spot of the | superior third of the neck, at the |
| | | 13, 14, | | | | common carotid artery) | level C2-C3) |
| | | 16 | | | | SKS - Sinocarotid collector, left | VD - Vagus nerve, right (in the |
| | | | | | | (over the bifurcation spot of the | superior third of the neck, at the |
| | | | | | | common carotid artery) | level C2-C3) |
| 4 | Alzheimer's | 8, 9, 10, | 17, 18 | 1-6 | 1-9 | SKD - Sinocarotid collector, right | VS - Vagus nerve, left (in the |
| | disease | 11, 12, | | | | (over the bifurcation spot of the | superior third of the neck, at the |
| | | 13, 14, | | | | common carotid artery) | level C2-C3) |
| | | 16 | | | | SKS - Sinocarotid collector, left | VD - Vagus nerve, right (in the |
| | | | | | | (over the bifurcation spot of the | superior third of the neck, at the |
| | | | | | | common carotid artery) | level C2-C3) |
| 5 | Gastric | 8-16, 15 | 17, 18, 19 | 1-6 | 1-9 | VS - Vagus nerve, left (in the | TSTS - Sympathetic trunk, |
| | ulcer | | | | | superior third of the neck, at the | thoracic, left (in the superior |
| | | | | | | level C2-C3) | third, at the level T7-T-11) |
| | | | | | | VD - Vagus nerve, right (in the | TSTD - Sympathetic trunk, |
| | | | | | | superior third of the neck, at the | thoracic, right (in the superior |
| | | | | | | level C2-C3) | third, at the level T2-T4) |
| 6 | Diabetes | 8, 9, 10, | 17, 18, 19 | 1-6 | 1-9 | VS - Vagus nerve, left (in the | TSTS - Sympathetic trunk, |
| | | 11, 12, | | | | superior third of the neck, at the | thoracic, left (in the superior |
| | | 13, 14, | | | | level C2-C3) | third, at the level T7-T-11) |
| | | 16 | | | | VD - Vagus nerve, right (in the | TSTD - Sympathetic trunk, |
| | | | | | | superior third of the neck, at the | thoracic, right (in the superior |
| | | | | | | level C2-C3) | third, at the level T2-T4) |
| 7 | Hypertention | 8, 9, 10, | 17, 18 | 1-6 | 1-9 | DPND - Depressor nerve, right | SKD - Sinocarotid collector, |
| | | 11, 12, | | | | (over the aortic arch or in the | right (over the bifurcation spot |
| | | 13, 14, | | | | middle third of the neck) | of the common carotid artery) |
| | | 16 | | | | DPNS - Depressor nerve, left | SKS - Sinocarotid collector, left |
| | | | | | | (over the aortic arch or in the | (over the bifurcation spot of the |
| | | | | | | middle third of the neck) | common carotid artery) |
| 8 | Obesity | 8-16, 15 | 17, 18 | 1-6 | 1-9 | VS - Vagus nerve, left (in the | TSTS - Sympathetic trunk, |
| | | | | | | superior third of the neck, at the | thoracic, left (in the superior |
| | | | | | | level C2-C3) | third, at the level T7-T-11) |
| | | | | | | VD - Vagus nerve, right (in the | TSTD - Sympathetic trunk, |
| | | | | | | superior third of the neck, at the | thoracic, right (in the superior |
| | | | | | | level C2-C3) | third, at the level T2-T4) |
| 9 | Infantile | 8, 9, 10, | 17 | 1-6 | 1-9 | SP - Spinal cord (at the level T10-T-12) | Nerve collector of the |
| | cerebral | 11, 12, | | | | Nerve collector of the | organ(Muscles) |
| | paralysis | 13, 14, | | | | organ(Muscles) | |
| | | 16 | | | | | |
| 10 | Posttraumatic | 8, 9, 10, | 17 | 1-6 | 1-9 | SP - Spinal cord (at the level T10-T-12) | Nerve collector of the |
| | paralysis | 11, 12, | | | | Nerve collector of the | organ(Muscles) |
| | | 13, 14, | | | | organ(Muscles) | |
| | | 16 | | | | | |
| 11 | Hirschsprung's | 8, 9, 10, | 17 | 1-6 | 1-9 | TSTS - Sympathetic trunk, | Nerve collector of the |
| | disease | 11, 12, | | | | thoracic, left (in the superior | organ(colon) |
| | | 13, 14, | | | | third, at the level T7-T-11) | |
| | | 16 | | | | TSTD - Sympathetic trunk, | |
| | | | | | | thoracic, right (in the superior | |
| | | | | | | third, at the level T2-T4) | |
| 12 | Mental | 8, 9, 10, | 17, 18 | 1-6 | 1-9 | SKD - Sinocarotid collector, right | TSTD - Sympathetic trunk, |
| | diseases | 11, 12, | | | | (over the bifurcation spot of the | thoracic, right (in the superior |
| | | 13, 14, | | | | common carotid artery) | third, at the level T2-T4) |
| | | 16 | | | | SKS - Sinocarotid collector, left | TSTS - Sympathetic trunk, |
| | | | | | | (over the bifurcation spot of the | thoracic, left (in the superior |
| | | | | | | common carotid artery) | third, at the level T7-T-11) |
| 13 | Adrenal | 8, 9, 10, | 17, 18, 19 | 1-6 | 1-9 | TSTD - Sympathetic trunk, | Nerve collector of the organ |
| | cortex | 11, 12, | | | | thoracic, right (in the superior | |
| | failure | 13, 14, | | | | third, at the level T2-T4) | |
| | | 16 | | | | TSTS - Sympathetic trunk, | |
| | | | | | | thoracic, left (in the superior | |
| | | | | | | third, at the level T7-T-11) | |
| 14 | Hypothyrosis | 8, 9, 10, | 17, 19 | 1-6 | 1-9 | TSCS - Left sympathetic trunk | VS - Vagus nerve, left (in the |
| | | 11, 12, | | | | (Left upper cervical third part | superior third of the neck, at the |
| | | 13, 14, | | | | between C2-C3) | level C2-C3) |
| | | 16 | | | | TSTD - Sympathetic trunk, | VD - Vagus nerve, right (in the |
| | | | | | | thoracic, right (in the superior | superior third of the neck, at the |
| | | | | | | third, at the level T2-T4) | level C2-C3) |
| 15 | Urinary | 8, 9, 10, | 17 | 1-6 | 1-9 | Nerve collector of the organ | SP - Spinal cord (at the level |
| | bladder | 11, 12, | | | | (v. bladder) | T10-T-12) |
| | atony | 13, 14, | | | | | |
| | | 16 | | | | | |
| 16 | Pain | 8, 9, 10, | 17 | 1-6 | 1-9 | SP - Spinal cord (at the level T10-T-12) | TSTD - Sympathetic trunk, |
| | syndromes | 11, 12, | | | | | thoracic, right (in the superior |
| | of different | 13, 14, | | | | | third, at the level T2-T4) |
| | genesis | 16 | | | | | TSTS - Sympathetic trunk, |
| | | | | | | | thoracic, left (in the superior |
| | | | | | | | third, at the level T7-T-11) |
| 17 | Obliterating | 8, 9, 10, | 17 | 1-6 | 1-9 | SP - Spinal cord (at the level T10-T-12) | TSTD - Sympathetic trunk, |
| | vascular | 11, 12, | | | | | thoracic, right (in the superior |
| | diseases of | 13, 14, | | | | | third, at the level T2-T4) |
| | the limbs | 16 | | | | | TSTS - Sympathetic trunk, |
| | | | | | | | thoracic, left (in the superior |
| | | | | | | | third, at the level T7-T-11) |
| 18 | Constipation | 8, 9, 10, | 17 | 1-6 | 1-9 | TSCD - Sympathetic trunk, | VD - Vagus nerve, right (in the |
| | | 11, 12, | | | | cervical, right (in the superior | superior third of the neck, at the |
| | | 13, 14, | | | | third of the neck, at the level C2-C3) | level C2-C3) |
| | | 16 | | | | TSCS - Left sympathetic trunk | VS - Vagus nerve, left (in the |
| | | | | | | (Left upper cervical third part | superior third of the neck, at the |
| | | | | | | between C2-C3) | level C2-C3) |
| 19 | Progressing | 8, 9, 10, | 17 | 1-6 | 1-9 | SP - Spinal cord (at the level T10-T-12) | Nerve collector of the |
| | myodystrophy | 11, 12, | | | | Nerve collector of the | organ(Muscles) |
| | | 13, 14, | | | | organ(Muscles) | |
| | | 16 | | | | | |
| 20 | Angiotropho | 8, 9, 10, | 17 | 1-6 | 1-9 | SP - Spinal cord (at the level T10-T-12) | Nerve collector of the |
| | neurosis | 11, 12, | | | | | organ(Muscles) |
| | | 13, 14, | | | | | |
| | | 16 | | | | | |
| 21 | Syringomyelia | 8-14, 16 | 17 | 1-6 | 1-9 | TSTD - Sympathetic trunk, | 3-TSCD - Sympathetic trunk, |
| | | | | | | thoracic, right (in the superior | cervical, right (in the superior |
| | | | | | | third, at the level T2-T4) | third of the neck, at the level |
| | | | | | | TSTS - Sympathetic trunk, | C2-C3) |
| | | | | | | thoracic, left (in the superior | 4-TSCS - Left sympathetic trunk |
| | | | | | | third, at the level T7-T-11) | (Left upper cervical third part |
| | | | | | | | between C2-C3) |
| 22 | Anxiety, | 8-14, 16 | 17 | 1-6 | 1-9 | SKD - Sinocarotid collector, right | 3-TSCD - Sympathetic trunk, |
| | neurosis, | | | | | (over the bifurcation spot of the | cervical, right (in the superior |
| | depression | | | | | common carotid artery) | third of the neck, at the level |
| | | | | | | SKS - Sinocarotid collector, left | C2-C3) |
| | | | | | | (over the bifurcation spot of the | 4-TSCS - Left sympathetic trunk |
| | | | | | | common carotid artery) | (Left upper cervical third part |
| | | | | | | | between C2-C3) |
| 23 | Alcoholism | 8-14, 16, | 17, 18, 20, | 1-6 | 1-9 | DPND - Depressor nerve, right | VS - Vagus nerve, left (in the |
| | | 9, 10, 11 | 21 | | | (over the aortic arch or in the | superior third of the neck, at the |
| | | | | | | middle third of the neck) | level C2-C3) |
| | | | | | | DPNS - Depressor nerve, left | VD - Vagus nerve, right (in the |
| | | | | | | (over the aortic arch or in the | superior third of the neck, at the |
| | | | | | | middle third of the neck) | level C2-C3), |
| | | | | | | | Nerve collector of the |
| | | | | | | | organ (skin) |
| 24 | Drug | 8-14, 16, | 17, 18, 20, | 1-6 | 1-9 | Nerve collector of the | Nerve collector of the |
| | addiction | 9, 10, 11 | 21 | | | organ(skin) | organ(skin) |
| 25 | Impotence | 8-14, 16, | 17, 18 | 1-6 | 1-9 | SP - Spinal cord (at the level T10-T-12) | Nerve collector of the organ |
| | | 13 | | | | | (genitals) |
| 26 | Cardiac | 8-14, 16, | 17 | 1-6 | 1-9 | SKD - Sinocarotid collector, right | Nerve collector of the organ |
| | arrhythmia | 9, 10, 11 | | | | (over the bifurcation spot of the | (cor), |
| | | | | | | common carotid artery) | VS - Vagus nerve, left (in the |
| | | | | | | SKS - Sinocarotid collector, left | superior third of the neck, at the |
| | | | | | | (over the bifurcation spot of the | level C2-C3) |
| | | | | | | common carotid artery) | VD - Vagus nerve, right (in the |
| | | | | | | | superior third of the neck, at the |
| | | | | | | | level C2-C3) |
| 27 | Paresis of | 8-14, 16, | 17 | 1-6 | 1-9 | VS - Vagus nerve, left (in the | 3-TSCD - Sympathetic trunk, |
| | the vocal | 9, 10, 11 | | | | superior third of the neck, at the | cervical, right (in the superior |
| | cords | | | | | level C2-C3) | third of the neck, at the level |
| | | | | | | VD - Vagus nerve, right (in the | C2-C3) |
| | | | | | | superior third of the neck, at the | 4-TSCS - Left sympathetic trunk |
| | | | | | | level C2-C3) | (Left upper cervical third part |
| | | | | | | | between C2-C3) |
| 28 | Osteoporosis | 8-14, 16 | 17 | 1-6 | 1-9 | Nerve collector of the organ | Nerve collector of the |
| | | | | | | (parathyroid gland, muscles) | organ(Muscles) |
| 29 | Angina | 8-14, 16, | 17 | 1-6 | 1-9 | SKD - Sinocarotid collector, right | SP - Spinal cord (at the level |
| | pectoris | 9, 10, 11 | | | | (over the bifurcation spot of the | T10-T-12) |
| | | | | | | common carotid artery) | |
| | | | | | | SKS - Sinocarotid collector, left | |
| | | | | | | (over the bifurcation spot of the | |
| | | | | | | common carotid artery) | |
| 30 | Apnea, | 8-14, 16, | 17 | 1-6 | 1-9 | TSCD - Sympathetic trunk, | SP - Spinal cord (at the level |
| | snoring, | 9, 10, 11 | | | | cervical, right (in the superior | T10-T-12) |
| | hypersomnia | | | | | third of the neck, at the level C2-C3) | |
| | | | | | | TSCS - Left sympathetic trunk | |
| | | | | | | (Left upper cervical third part | |
| | | | | | | between C2-C3) | |
| 31 | Chronic | 8-16, 15 | 17, 18 | 1-6 | 1-9 | VD - Vagus nerve, right (in the | TSTD - Sympathetic trunk, |
| | gastroenterocolitis | | | | | superior third of the neck, at the | thoracic, right (in the superior |
| | | | | | | level C2-C3) | third, at the level T2-T4) |
| | | | | | | VS - Vagus nerve, left (in the | TSTS - Sympathetic trunk, |
| | | | | | | superior third of the neck, at the | thoracic, left (in the superior |
| | | | | | | level C2-C3) | third, at the level T7-T-11) |
| 32 | Ailments, | 8-14, 16 | 17 | 1-6 | 1-9 | Nerve collector of the | Nerve collector of the |
| | associated | | | | | organ(rectum) | organ(rectum) |
| | with a | | | | | | |
| | malfunction | | | | | | |
| | of the | | | | | | |
| | rectum | | | | | | |
| | sphincters | | | | | | |
| 33 | Male and | 8-14, 16 | 17, 19 | 1-6 | 1-9 | Nerve collector of the organ | Nerve collector of the |
| | female | | | | | (genitals) | organ(genitals) |
| | infertility | | | | | | |
| 34 | Stammering | 8-14, 16 | 17 | 1-6 | 1-9 | SKD - Sinocarotid collector, right | 3-TSCD - Sympathetic trunk, |
| | | | | | | (over the bifurcation spot of the | cervical, right (in the superior |
| | | | | | | common carotid artery) | third of the neck, at the level |
| | | | | | | SKS - Sinocarotid collector, left | C2-C3) |
| | | | | | | (over the bifurcation spot of the | 4-TSCS - Left sympathetic trunk |
| | | | | | | common carotid artery) | (Left upper cervical third part |
| | | | | | | | between C2-C3) |
| 35 | Insomnia | 8-14, 16 | 17 | 1-6 | 1-9 | SKD - Sinocarotid collector, right | VD - Vagus nerve, right (in the |
| | | | | | | (over the bifurcation spot of the | superior third of the neck, at the |
| | | | | | | common carotid artery) | level C2-C3) |
| | | | | | | SKS - Sinocarotid collector, left | VS - Vagus nerve, left (in the |
| | | | | | | (over the bifurcation spot of the | superior third of the neck, at the |
| | | | | | | common carotid artery) | level C2-C3) |
| 36 | Bulimia | 8-16, 15 | 17, 18, 19 | 1-6 | 1-9 | VD - Vagus nerve, right (in the | TSTD - Sympathetic trunk, |
| | | | | | | superior third of the neck, at the | thoracic, right (in the superior |
| | | | | | | level C2-C3) | third, at the level T2-T4) |
| | | | | | | VS - Vagus nerve, left (in the | TSTS - Sympathetic trunk, |
| | | | | | | superior third of the neck, at the | thoracic, left (in the superior |
| | | | | | | level C2-C3) | third, at the level T7-T-11) |
| 37 | Reflux- | 8-16, 15 | 17, 18 | 1-6 | 1-9 | VD - Vagus nerve, right (in the | TSTD - Sympathetic trunk, |
| | esophagitis | | | | | superior third of the neck, at the | thoracic, right (in the superior |
| | | | | | | level C2-C3) | third, at the level T2-T4) |
| | | | | | | VS - Vagus nerve, left (in the | TSTS - Sympathetic trunk, |
| | | | | | | superior third of the neck, at the | thoracic, left (in the superior |
| | | | | | | level C2-C3) | third, at the level T7-T-11) |

TABLE 8
Electric stimulation current parameters for different diseases
Electric current parameters for stimulation of nervous structures

| | Names of the | | | | | | | | | |
|---|---|---|---|---|---|---|---|---|---|---|
| | disease from | | | | | | | | | |
| | our patents in | | | | | | | | | |
| | alphabetical | | | | | | | | | |
| | order (in | | | | | | | | | |
| | Russian/ | General range | | | Best-mode value | | | | | |
| No. | Russian) | F | D | A | F | | D | | A | |
| 1 | Agranulocytosis | 1-300 Hz | 0.1-0.8 Ms | 0.2-1.0 V | 0-75 | HZ | 0.1-0.29 | MS | 0.02-0.4 | V |
| 2 | Alcoholism | 1-300 HZ | 0.1-0.8 Ms | 0.2-1.0 V | 150-225 | HZ | 0.7-0.8 | MS | 0.81-1.0 | V |
| 3 | Angiotrophoneurosis | 1-300 HZ | 0.1-0.8 Ms | 0.2-1.0 V | 76-150 | HZ | 0.1-0.29 | MS | 0.4-0.6 | V |
| 4 | Anemia (all types | 1-300 HZ | 0.1-0.8 Ms | 0.2-1.0 V | 0-75 | HZ | 0.1-0.29 | MS | 0.02-0.4 | V |
| | are to be listed)) | | | | | | | | | |
| 5 | Apnea | 1-300 HZ | 0.1-0.8 Ms | 0.2-1.0 V | 225-300 | HZ | 0.7-0.8 | MS | 0.81-1.0 | V |
| 6 | Cardiac | 1-300 HZ | 0.1-0.8 Ms | 0.2-1.0 V | 0-75 | HZ | 0.1-0.29 | MS | 0.02-0.4 | V |
| | arrhythmia, | | | | | | | | | |
| | Ischemic heart | | | | | | | | | |
| | disease | | | | | | | | | |
| 7 | Urinary bladder | 1-300 HZ | 0.1-0.8 Ms | 0.2-1.0 V | 150-225 | HZ | 0.7-0.8 | MS | 0.81-1.0 | V |
| | atony | | | | | | | | | |
| 8 | Atrophy of he | 1-300 HZ | 0.1-0.8 Ms | 0.2-1.0 V | 0-75 | HZ | 0.1-0.29 | MS | 0.02-0.4 | V |
| | optic nerve | | | | | | | | | |
| 9 | Skin atrophy | 1-300 HZ | 0.1-0.8 Ms | 0.2-1.0 V | 0-75 | HZ | 0.1-0.29 | MS | 0.02-0.4 | V |
| 10 | Sardiac achalasia | 1-300 HZ | 0.1-0.8 Ms | 0.2-1.0 V | 0-75 | HZ | 0.1-0.29 | MS | 0.02-0.4 | V |
| 11 | Coagulation | 1-300 HZ | 0.1-0.8 Ms | 0.2-1.0 V | 0-75 | HZ | 0.1-0.29 | MS | 0.02-0.4 | V |
| | disorders | | | | | | | | | |
| 12 | Buerger's disease | 1-300 HZ | 0.1-0.8 Ms | 0.2-1.0 V | 76-150 | HZ | 0.3-0.49 | MS | 0.4-0.6 | V |
| 13 | Hirschsprung's | 1-300 HZ | 0.1-0.8 Ms | 0.2-1.0 V | 76-150 | HZ | 0.1-0.29 | MS | 0.02-0.4 | V |
| | disease- | | | | | | | | | |
| | megacolon | | | | | | | | | |
| 14 | Crohn's disease | 1-300 HZ | 0.1-0.8 Ms | 0.2-1.0 V | 0-75 HZ | | 0.1-0.29 MS | | 0.02-0.4 | V |
| | | | | | 76-150 HZ, | | 0.3-0.49 MS, | | 0.4-0.6 | V |
| | | | | | 150-225 HZ, | | 0.5-0.69 MS | | 0.61-0.8 | V |
| | | | | | 225-300 HZ | | 0.7-0.8 MS | | 0.81-1.0 | V |
| 15 | Parkinson's | 1-300 HZ | 0.1-0.8 Ms | 0.2-1.0 V | 0-75 | HZ | 0.1-0.29 | MS | 0.02-0.4 | V |
| | disease | | | | | | | | | |
| 16 | Pick's disease | 1-300 HZ | 0.1-0.8 Ms | 0.2-1.0 V | 76-150 | HZ | 0.3-0.49 | MS | 0.4-0.6 | V |
| 17 | Raynaud's | 1-300 HZ | 0.1-0.8 Ms | 0.2-1.0 V | 76-150 | HZ | 0.3-0.49 | MS | 0.4-0.6 | V |
| | disease | | | | | | | | | |
| 18 | Alzheimer's | | | | | | | | | |
| | disease | | | | | | | | | |
| 19 | Asthma | 1-300 HZ | 0.1-0.8 Ms | 0.2-1.0 V | 0-75 | HZ | 0.1-0.29 | MS | 0.02-0.4 | V |
| 20 | Bulimia | 1-300 HZ | 0.1-0.8 Ms | 0.2-1.0 V | 0-75 | HZ | 0.1-0.29 | MS | 0.02-0.4 | V |
| 21 | Vegetovascular | 1-300 HZ | 0.1-0.8 Ms | 0.2-1.0 V | 0-75 | HZ | 0.1-0.29 | MS | 0.02-0.4 | V |
| | dystony | | | | | | | | | |
| 22 | Sudden cardiac | 1-300 HZ | 0.1-0.8 Ms | 0.2-1.0 V | 0-75 | HZ | 0.3-0.49 | MS | 0.4-0.6 | V |
| | arrest at sleep | | | | | | | | | |
| 23 | Rectal prolapse | 1-300 HZ | 0.1-0.8 Ms | 0.2-1.0 V | 0-75 | HZ | 0.1-0.29 | MS | 0.02-0.4 | V |
| | | | | | 225-300 | HZ | | | | |
| 24 | Flaccidly | 1-300 HZ | 0.1-0.8 Ms | 0.2-1.0 V | 0-75 | HZ | 0.3-0.49 | MS | 0.02-0.4 | V |
| | consolidating | | | | | | | | | |
| | fractures | | | | | | | | | |
| 25 | Hyperkeratosis | 1-300 HZ | 0.1-0.8 Ms | 0.2-1.0 V | 0-75 | HZ | 0.1-0.29 | MS | 0.02-0.4 | V |
| 26 | Hyperostosis | 1-300 HZ | 0.1-0.8 Ms | 0.2-1.0 V | 76-150 | HZ | 0.5-0.69 | MS | 0.4-0.6 | V |
| 27 | Hypersomnia | 1-300 HZ | 0.1-0.8 Ms | 0.2-1.0 V | 0-75 | HZ | 0.3-0.49 | MS | 0.4-0.6 | V |
| 28 | Hypertension (list | 1-300 HZ | 0.1-0.8 Ms | 0.2-1.0 V | 0-75 | HZ | 0.1-0.29 | MS | 0.02-0.4 | V |
| | all types) | | | | | | | | | |
| 29 | Adrenal cortex | 1-300 HZ | 0.1-0.8 Ms | 0.2-1.0 V | 76-150 | HZ | 0.3-0.49 | MS | 0.02-0.4 | V |
| | hyperfunction | | | | | | | | | |
| 30 | Hypoglycemia | 1-300 HZ | 0.1-0.8 Ms | 0.2-1.0 V | 150-225 | HZ | 0.3-0.49 | MS | 0.4-0.6 | V |
| 31 | Hypothyrosis | 1-300 HZ | 0.1-0.8 Ms | 0.2-1.0 V | 150-225 | HZ | 0.5-0.69 | MS | 0.61-0.8 | V |
| 32 | Hypotension | 1-300 HZ | 0.1-0.8 Ms | 0.2-1.0 V | 150-225 | HZ | 0.5-0.69 | MS | 0.61-0.8 | V |
| 33 | Adrenal cortex | 1-300 HZ | 0.1-0.8 Ms | 0.2-1.0 V | 76-150 | HZ | 0.7-0.8 | MS | 0.61-0.8 | V |
| | hypofunction | | | | | | | | | |
| | (Addison's | | | | | | | | | |
| | disease)) | | | | | | | | | |
| 34 | Subcortical | 1-300 HZ | 0.1-0.8 Ms | 0.2-1.0 V | 0-75 | HZ | 0.1-0.29 | MS | 0.02-0.4 | V |
| | dementia | | | | | | | | | |
| 35 | Multi-infarction | 1-300 HZ | 0.1-0.8 Ms | 0.2-1.0 V | 0-75 | HZ | 0.1-0.29 | MS | 0.02-0.4 | V |
| | dementia | | | | | | | | | |
| | Involuntary | | | | | | | | | |
| | movements | | | | | | | | | |
| 36 | Cortical dementia - | 1-300 HZ | 0.1-0.8 Ms | 0.2-1.0 V | 0-75 | HZ | 0.1-0.29 | MS | 0.02-0.4 | V |
| | Alzheimer's | | | | | | | | | |
| | disease | | | | | | | | | |
| 37 | Depression | | | | | | | | | |
| 38 | Infantile cerebral | 1-300 HZ | 0.1-0.8 Ms | 0.2-1.0 V | 0-75 | HZ | 0.1-0.29 | MS | 0.02-0.4 | V |
| | paralysis | | | | | | | | | |
| 39 | Dyskinesia of the | 1-300 HZ | 0.1-0.8 Ms | 0.2-1.0 V | 0-75 | HZ | 0.1-0.29 | MS | 0.02-0.4 | V |
| | biliferous tracts | | | | | | | | | |
| 40 | Dysmenorrhea | 1-300 HZ | 0.1-0.8 Ms | 0.2-1.0 V | 0-75 | HZ | 0.1-0.29 | MS | 0.02-0.4 | V |
| | (genital diseases) | | | | | | | | | |
| 41 | Dolichosigmoid, | 1-300 HZ | 0.1-0.8 Ms | 0.2-1.0 V | 0-75 | HZ | 0.3-0.49 | MS | 0.4-0.6 | V |
| 42 | Gastrointestinal | 1-300 HZ | 0.1-0.8 Ms | 0.2-1.0 V | 0-75 | HZ | 0.1-0.29 | MS | 0.02-0.4 | V |
| | dyskinesia | | | | | | | | | |
| 43 | Stammering | 1-300 HZ | 0.1-0.8 Ms | 0.2-1.0 V | 76-150 | HZ | 0.1-0.29 | MS | 0.02-0.4 | V |
| 44 | Impotence | 1-300 HZ | 0.1-0.8 Ms | 0.2-1.0 V | 76-150 | HZ | 0.3-0.49 | MS | 0.02-0.4 | V |
| 45 | Insomnia | 1-300 HZ | 0.1-0.8 Ms | 0.2-1.0 V | 0-75 | HZ | 0.1-0.29 | MS | 0.02-0.4 | V |
| 46 | Ischemic heart | 1-300 HZ | 0.1-0.8 Ms | 0.2-1.0 V | 0-75 | HZ | 0.3-0.49 | MS | 0.02-0.4 | V |
| | disease, Angina | | | | | | | | | |
| | pectoris | | | | | | | | | |
| 47 | Coprostasia | | | | | | | | | |
| 48 | Leucosis (list all | 1-300 HZ | 0.1-0.8 Ms | 0.2-1.0 V | 0-75 | HZ | 0.1-0.29 | MS | 0.02-0.4 | V |
| | types) | | | | | | | | | |
| 49 | Migraine | 1-300 HZ | 0.1-0.8 Ms | 0.2-1.0 V | 0-75 | HZ | 0.1-0.29 | MS | 0.02-0.4 | V |
| 50 | Myodystrophy | 1-300 HZ | 0.1-0.8 Ms | 0.2-1.0 V | 76-150 | HZ | 0.3-0.49 | MS | 0.4-0.6 | V |
| 51 | Myopathy | 1-300 HZ | 0.1-0.8 Ms | 0.2-1.0 V | 76-150 | HZ | 0.3-0.49 | MS | 0.4-0.6 | V |
| 52 | Male and female | 1-300 HZ | 0.1-0.8 Ms | 0.2-1.0 V | 0-75 | HZ | 0.1-0.29 | MS | 0.02-0.4 | V |
| | infertility | | | | | | | | | |
| 53 | Narcolepsy | 1-300 HZ | 0.1-0.8 Ms | 0.2-1.0 V | 76-150 | HZ | 0.3-0.49 | MS | 0.02-0.4 | V |
| 54 | Drug addiction | 1-300 HZ | 0.1-0.8 Ms | 0.2-1.0 V | 0-75 | HZ | 0.5-0.69 | MS | 0.4-0.6 | V |
| | (list all types) | | | | | | | | | |
| 55 | Bauhin's valve | 1-300 HZ | 0.1-0.8 Ms | 0.2-1.0 V | 150-225 | HZ | 0.3-0.49 | MS | 0.4-0.6 | V |
| | failure | | | | | | | | | |
| 56 | Adrenal cortex | 1-300 HZ | 0.1-0.8 Ms | 0.2-1.0 V | 0-75 | HZ | 0.5-0.69 | MS | 0.61-0.8 | V |
| | insufficiency | | | | | | | | | |
| 57 | Neurodermite | 1-300 HZ | 0.1-0.8 Ms | 0.2-1.0 V | 0-75 | HZ | 0.1-0.29 | MS | 0.02-0.4 | V |
| 58 | Nervous anorexia | 1-300 HZ | 0.1-0.8 Ms | 0.2-1.0 V | 76-150 | HZ | 0.3-0.49 | MS | 0.4-0.6 | V |
| 59 | Diabetes | 1-300 HZ | 0.1-0.8 Ms | 0.2-1.0 V | 76-150 | HZ | 0.3-0.49 | MS | 0.02-0.4 | V |
| | insipidus | | | | | | | | | |
| 60 | Obliterating | 1-300 HZ | 0.1-0.8 Ms | 0.2-1.0 V | 0-75 | HZ | 0.1-0.29 | MS | 0.02-0.4 | V |
| | atherosclerosis of | | | | | | | | | |
| | the limbs' vessels | | | | | | | | | |
| 61 | Obliterating | 1-300 HZ | 0.1-0.8 Ms | 0.2-1.0 V | 76-150 | HZ | 0.3-0.49 | MS | 0.4-0.6 | V |
| | endarteritis of the | | | | | | | | | |
| | lower limbs' | | | | | | | | | |
| | vessels | | | | | | | | | |
| 62 | Alopecia | 1-300 HZ | 0.1-0.8 Ms | 0.2-1.0 V | 0-75 | HZ | 0.1-0.29 | MS | 0.02-0.4 | V |
| 63 | Obesity | 1-300 HZ | 0.1-0.8 Ms | 0.2-1.0 V | 76-150 | HZ | 0.3-0.49 | MS | 0.4-0.6 | V |
| 64 | Osteoporosis | 1-300 HZ | 0.1-0.8 Ms | 0.2-1.0 V | 0-75 | HZ | 0.1-0.29 | MS | 0.02-0.4 | V |
| 65 | Trauma-related | 1-300 HZ | 0.1-0.8 Ms | 0.2-1.0 V | 0-75 HZ | | 0.1-0.29 MS | | 0.02-0.4 | V |
| | paralyses | | | | 76-150 HZ, | | 0.3-0.49 MS, | | 0.4-0.6 | V |
| | | | | | 150-225 HZ, | | 0.5-0.69 MS | | 0.61-0.8 | V |
| | | | | | 225-300 HZ | | 0.7-0.8 MS | | 0.81-1.0 | V |
| 66 | Paralyses of | 1-300 HZ | 0.1-0.8 Ms | 0.2-1.0 V | 76-150 | HZ | 0.3-0.49 | MS | 0.4-0.6 | V |
| | different etiology | | | | | | | | | |
| 67 | Paresis of the | 1-300 HZ | 0.1-0.8 Ms | 0.2-1.0 V | 76-150 | HZ | 0.1-0.29 | MS | 0.02-0.4 | V |
| | vocal cords | | | | | | | | | |
| 68 | Borderline | 1-300 HZ | 0.1-0.8 Ms | 0.2-1.0 V | 0-75 | HZ | 0.1-0.29 | MS | 0.02-0.4 | V |
| | personality | | | | | | | | | |
| | disorders | | | | | | | | | |
| 69 | Priapism | 1-300 HZ | 0.1-0.8 Ms | 0.2-1.0 V | 76-150 | HZ | 0.3-0.49 | MS | 0.02-0.4 | V |
| 70 | Progressing | 1-300 HZ | 0.1-0.8 Ms | 0.2-1.0 V | 0-75 | HZ | 0.1-0.29 | MS | 0.02-0.4 | V |
| | myodystrophy | | | | | | | | | |
| 71 | Psoriasis | 1-300 HZ | 0.1-0.8 Ms | 0.2-1.0 V | 76-150 | HZ | 0.1-0.29 | MS | 0.4-0.6 | V |
| 72 | Cancer (list | | | | | | | | | |
| | different | | | | | | | | | |
| | localizations and | | | | | | | | | |
| | types) | | | | | | | | | |
| 73 | Rickets | 1-300 HZ | 0.1-0.8 Ms | 0.2-1.0 V | 76-150 | HZ | 0.1-0.29 | MS | 0.02-0.4 | V |
| 74 | Reflux- | 1-300 HZ | 0.1-0.8 Ms | 0.2-1.0 V | 0-75 | HZ | 0.1-0.29 | MS | 0.02-0.4 | V |
| | esophagitis | | | | | | | | | |
| 75 | Sarcoma | | | | | | | | | |
| 76 | Diabetes | 1-300 HZ | 0.1-0.8 Ms | 0.2-1.0 V | 76-150 | HZ | 0.3-0.49 | MS | 0.02-0.4 | V |
| 77 | Syringomyelia | 1-300 HZ | 0.1-0.8 Ms | 0.2-1.0 V | 0-75 | HZ | 0.3-0.49 | MS | 0.4-0.6 | V |
| 78 | Zollinger-Ellison | 1-300 HZ | 0.1-0.8 Ms | 0.2-1.0 V | 0-75 | HZ | 0.1-0.29 | MS | 0.02-0.4 | V |
| | syndrome | | | | | | | | | |
| 79 | Commissural | 1-300 HZ | 0.1-0.8 Ms | 0.2-1.0 V | 0-75 | HZ | 0.3-0.49 | MS | 0.4-0.6 | V |
| | disease | | | | | | | | | |
| 80 | Angina pectoris | | | | | | | | | |
| 81 | Anxiety and | 1-300 HZ | 0.1-0.8 Ms | 0.2-1.0 V | 0-75 | HZ | 0.1-0.29 | MS | 0.02-0.4 | V |
| | depression | | | | | | | | | |
| 82 | Huntington's | 1-300 HZ | 0.1-0.8 Ms | 0.2-1.0 V | 0-75 | HZ | 0.3-0.49 | MS | 0.61-0.8 | V |
| | chorea | | | | | | | | | |
| 83 | Chronic duodenal | 1-300 HZ | 0.1-0.8 Ms | 0.2-1.0 V | 0-75 | HZ | 0.3-0.49 | MS | 0.4-0.6 | V |
| | ileus | | | | | | | | | |
| 84 | Chronic and acute | 1-300 HZ | 0.1-0.8 Ms | 0.2-1.0 V | 76-150 | HZ | 0.1-0.29 | MS | 0.02-0.4 | V |
| | hyperthermia | | | | | | | | | |
| 85 | Chronic intestinal | 1-300 HZ | 0.1-0.8 Ms | 0.2-1.0 V | 76-150 | HZ | 0.3-0.49 | MS | 0.4-0.6 | V |
| | obstruction | | | | | | | | | |
| | (Commissural | | | | | | | | | |
| | disease, | | | | | | | | | |
| | Megacolon, | | | | | | | | | |
| | Chronic | | | | | | | | | |
| | mesenterial | | | | | | | | | |
| | circulation | | | | | | | | | |
| | insufficiency, | | | | | | | | | |
| | Metacolon, | | | | | | | | | |
| | Dolichosigmoid, | | | | | | | | | |
| | Cardiac | | | | | | | | | |
| | achalasia) | | | | | | | | | |
| 86 | Chronic | 1-300 HZ | 0.1-0.8 Ms | 0.2-1.0 V | 76-150 | HZ | 0.3-0.49 | MS | 0.4-0.6 | V |
| | gastroenterocolitis | | | | | | | | | |
| 87 | Chronic hepatitis | 1-300 HZ | 0.1-0.8 Ms | 0.2-1.0 V | 0-75 | HZ | 0.1-0.29 | MS | 0.02-0.4 | V |
| | (list each type in | | | | | | | | | |
| | detail) | | | | | | | | | |
| 88 | Chronic | 1-300 HZ | 0.1-0.8 Ms | 0.2-1.0 V | 0-75 | HZ | 0.1-0.29 | MS | 0.02-0.4 | V |
| | cholecystitis and | | | | | | | | | |
| | cholecystopancreatitis | | | | | | | | | |
| 89 | Chronic cystitis | 1-300 HZ | 0.1-0.8 Ms | 0.2-1.0 V | 0-75 | HZ | 0.5-0.69 | MS | 0.4-0.6 | V |
| 90 | Chronic periodic | 1-300 HZ | 0.1-0.8 Ms | 0.2-1.0 V | 0-75 | HZ | 0.1-0.29 | MS | 0.02-0.4 | V |
| | pains | | | | | | | | | |
| | (Angina pectoris, | | | | | | | | | |
| | Phantom pains, | | | | | | | | | |
| | Neuritis, | | | | | | | | | |
| | Nerve root | | | | | | | | | |
| | syndromes | | | | | | | | | |
| | Terminal-stage | | | | | | | | | |
| | pains | | | | | | | | | |
| | Migraine) | | | | | | | | | |
| 91 | Chronic | 1-300 HZ | 0.1-0.8 Ms | 0.2-1.0 V | 0-75 HZ | | 0.1-0.294 MS | | 0.02-0.4 | V |
| | glomerulonephritis | | | | 76-150 HZ, | | 0.3-0.49 MS, | | 0.4-0.6 | V |
| | | | | | 150-225 HZ, | | 0.5-0.69 MS | | 0.61-0.8 | V |
| | | | | | 225-300 HZ | | 0.7-0.8 MS | | 0.81-1.0 | V |
| 92 | Chronic | 1-300 HZ | 0.1-0.8 Ms | 0.2-1.0 V | 76-150 | HZ | 0.3-0.49 | MS | 0.4-0.6 | V |
| | obstructive | | | | | | | | | |
| | bronchitis | | | | | | | | | |
| 93 | Chronic | 1-300 HZ | 0.1-0.8 Ms | 0.2-1.0 V | 0-75 | HZ | 0.1-0.29 | MS | 0.02-0.4 | V |
| | osteomyelitis | | | | | | | | | |
| 94 | Chronic | 1-300 HZ | 0.1-0.8 Ms | 0.2-1.0 V | 0-75 HZ | | 0.1-0.29 MS | | 0.02-0.4 | V |
| | pyelonephritis | | | | 76-150 HZ, | | 0.3-0.49 MS, | | 0.4-0.6 | V |
| | | | | | 150-225 HZ, | | 0.5-0.69 MS | | 0.61-0.8 | V |
| | | | | | 225-300 HZ | | 0.7-0.8 MS | | 0.81-1.0 | V |
| 95 | Cirrhosis (list all | 1-300 HZ | 0.1-0.8 Ms | 0.2-1.0 V | 0-75 | HZ | 0.1-0.29 | MS | 0.02-0.4 | V |
| | types) | | | | | | | | | |
| 96 | Schizophrenia | 1-300 HZ | 0.1-0.8 Ms | 0.2-1.0 V | 0-75 | HZ | 0.1-0.29 | MS | 0.02-0.4 | V |
| | with | | | | | | | | | |
| | schizophrenic | | | | | | | | | |
| | affective | | | | | | | | | |
| | disorders and | | | | | | | | | |
| | delirium | | | | | | | | | |
| 97 | Eczema | 1-300 HZ | 0.1-0.8 Ms | 0.2-1.0 V | 76-150 | HZ | 0.1-0.29 | MS | 0.02-0.4 | V |

TABLE 9
Electric stimulation current parameters for different diseases
Electric current parameters for stimulation of nervous structures

| | Names of the | | | | | | |
|---|---|---|---|---|---|---|---|
| | disease from our | | | | | | |
| | patents in | | | | | | |
| | alphabetical | | | | | | |
| | order (in Russian/ | Optimal range | | | | | |
| No. | Russian) | F | | D | | A | |
| 1 | Agranulocytosis | 0-75 | HZ | 0-75 | HZ | 0-75 | HZ |
| | | 76-150 | HZ | 76-150 | HZ | 76-150 | HZ |
| 2 | Alcoholism | 76-150 | HZ, | 76-150 | HZ, | 76-150 | HZ, |
| | | 150-225 | HZ | 150-225 | HZ | 150-225 | HZ |
| 3 | Angiotrophoneurosis | 0-75 | HZ | 0-75 | HZ | 0-75 | HZ |
| | | 76-150 | HZ | 76-150 | HZ | 76-150 | HZ |
| 4 | Anemia (all types | 0-75 | HZ | 0-75 | HZ | 0-75 | HZ |
| | are to be listed)) | 76-150 | HZ | 76-150 | HZ | 76-150 | HZ |
| 5 | Apnea | 150-225 | HZ | 150-225 | HZ | 150-225 | HZ |
| | | 225-300 | HZ | 225-300 | HZ | 225-300 | HZ |
| 6 | Cardiac arrhythmia, | 0-75 | HZ | 0-75 | HZ | 0-75 | HZ |
| | Ischemic heart | | | | | | |
| | disease | | | | | | |
| 7 | Urinary bladder | 76-150 | HZ, | 76-150 | HZ, | 76-150 | HZ, |
| | atony | 150-225 | HZ | 150-225 | HZ | 150-225 | HZ |
| 8 | Atrophy of he optic | 0-75 | HZ | 0-75 | HZ | 0-75 | HZ |
| | nerve | | | | | | |
| 9 | Skin atrophy | 0-75 | HZ | 0-75 | HZ | 0-75 | HZ |
| | | 76-150 | HZ | 76-150 | HZ | 76-150 | HZ |
| 10 | Sardiac achalasia | 0-75 | HZ | 0-75 | HZ | 0-75 | HZ |
| | | 76-150 | HZ | 76-150 | HZ | 76-150 | HZ |
| 11 | Coagulation | 0-75 | HZ | 0-75 | HZ | 0-75 | HZ |
| | disorders | | | | | | |
| 12 | Buerger's disease | 0-75 | HZ | 0-75 | HZ | 0-75 | HZ |
| | | 76-150 | HZ | 76-150 | HZ | 76-150 | HZ |
| 13 | Hirschsprung's | 0-75 | HZ | 0-75 | HZ | 0-75 | HZ |
| | disease - megacolon | 76-150 | HZ | 76-150 | HZ | 76-150 | HZ |
| 14 | Crohn's disease | 0-75 | HZ | 0-75 | HZ | 0-75 | HZ |
| | | 76-150 | HZ, | 76-150 | HZ, | 76-150 | HZ, |
| | | 150-225 | HZ, | 150-225 | HZ, | 150-225 | HZ, |
| | | 225-300 | HZ | 225-300 | HZ | 225-300 | HZ |
| 15 | Parkinson's disease | 0-75 | HZ | 0-75 | HZ | 0-75 | HZ |
| | | 76-150 | HZ | 76-150 | HZ | 76-150 | HZ |
| 16 | Pick's disease | 0-75 | HZ | 0-75 | HZ | 0-75 | HZ |
| | | 76-150 | HZ | 76-150 | HZ | 76-150 | HZ |
| 17 | Raynaud's disease | 76-150 | HZ, | 76-150 | HZ, | 76-150 | HZ, |
| | | 150-225 | HZ | 150-225 | HZ | 150-225 | HZ |
| 18 | Alzheimer's disease | | | | | | |
| 19 | Asthma | 0-75 | HZ | 0-75 | HZ | 0-75 | HZ |
| | | 76-150 | HZ | 76-150 | HZ | 76-150 | HZ |
| 20 | Bulimia | 0-75 | HZ | 0-75 | HZ | 0-75 | HZ |
| | | 76-150 | HZ | 76-150 | HZ | 76-150 | HZ |
| 21 | Vegetovascular | 0-75 | HZ | 0-75 | HZ | 0-75 | HZ |
| | dystony | 76-150 | HZ | 76-150 | HZ | 76-150 | HZ |
| 22 | Sudden cardiac | 0-75 | HZ | 0-75 | HZ | 0-75 | HZ |
| | arrest at sleep | 76-150 | HZ | 76-150 | HZ | 76-150 | HZ |
| 23 | Rectal prolapse | 0-75 | HZ | 0-75 | HZ | 0-75 | HZ |
| | | 76-150 | HZ | 76-150 | HZ | 76-150 | HZ |
| 24 | Flaccidly | 0-75 | HZ | 0-75 | HZ | 0-75 | HZ |
| | consolidating | 76-150 | HZ | 76-150 | HZ | 76-150 | HZ |
| | fractures | | | | | | |
| 25 | Hyperkeratosis | 0-75 | HZ | 0-75 | HZ | 0-75 | HZ |
| | | 76-150 | HZ | 76-150 | HZ | 76-150 | HZ |
| 26 | Hyperostosis | 0-75 | HZ | 0-75 | HZ | 0-75 | HZ |
| | | 76-150 | HZ | 76-150 | HZ | 76-150 | HZ |
| 27 | Hypersomnia | 0-75 | HZ | 0-75 | HZ | 0-75 | HZ |
| | | 76-150 | HZ | 76-150 | HZ | 76-150 | HZ |
| 28 | Hypertension (list all | 0-75 | HZ | 0-75 | HZ | 0-75 | HZ |
| | types) | 76-150 | HZ | 76-150 | HZ | 76-150 | HZ |
| 29 | Adrenal cortex | 76-150 | HZ, | 76-150 | HZ, | 76-150 | HZ, |
| | hyperfunction | 150-225 | HZ | 150-225 | HZ | 150-225 | HZ |
| 30 | Hypoglycemia | 76-150 | HZ, | 76-150 | HZ, | 76-150 | HZ, |
| | | 150-225 | HZ | 150-225 | HZ | 150-225 | HZ |
| 31 | Hypothyrosis | 150-225 | HZ, | 150-225 | HZ, | 150-225 | HZ, |
| | | 225-300 | HZ | 225-300 | HZ | 225-300 | HZ |
| 32 | Hypotension | 76-150 | HZ, | 76-150 | HZ, | 76-150 | HZ, |
| | | 150-225 | HZ | 150-225 | HZ | 150-225 | HZ |
| 33 | Adrenal cortex | 0-75 | HZ | 0-75 | HZ | 0-75 | HZ |
| | hypofunction | 76-150 | HZ | 76-150 | HZ | 76-150 | HZ |
| | (Addison's disease)) | | | | | | |
| 34 | Subcortical dementia | 0-75 | HZ | 0-75 | HZ | 0-75 | HZ |
| | | 76-150 | HZ | 76-150 | HZ | 76-150 | HZ |
| 35 | Multi-infarction | 0-75 | HZ | 0-75 | HZ | 0-75 | HZ |
| | dementia | 76-150 | HZ | 76-150 | HZ | 76-150 | HZ |
| | Involuntary | | | | | | |
| | movements | | | | | | |
| 36 | Cortical dementia — | 0-75 | HZ | 0-75 | HZ | 0-75 | HZ |
| | Alzheimer's disease | 76-150 | HZ | 76-150 | HZ | 76-150 | HZ |
| 37 | Depression | | | | | | |
| 38 | Infantile cerebral | 0-75 | HZ | 0-75 | HZ | 0-75 | HZ |
| | paralysis | 76-150 | HZ | 76-150 | HZ | 76-150 | HZ |
| 39 | Dyskinesia of the | 0-75 | HZ | 0-75 | HZ | 0-75 | HZ |
| | biliferous tracts | 76-150 | HZ | 76-150 | HZ | 76-150 | HZ |
| 40 | Dysmenorrhea | 0-75 | HZ | 0-75 | HZ | 0-75 | HZ |
| | (genital diseases) | 76-150 | HZ | 76-150 | HZ | 76-150 | HZ |
| 41 | Dolichosigmoid, | 0-75 | HZ | 0-75 | HZ | 0-75 | HZ |
| | | 76-150 | HZ | 76-150 | HZ | 76-150 | HZ |
| 42 | Gastrointestinal | 0-75 | HZ | 0-75 | HZ | 0-75 | HZ |
| | dyskinesia | 76-150 | HZ | 76-150 | HZ | 76-150 | HZ |
| 43 | Stammering | 76-150 | HZ, | 76-150 | HZ, | 76-150 | HZ, |
| | | 150-225 | HZ | 150-225 | HZ | 150-225 | HZ |
| 44 | Impotence | 0-75 | HZ | 0-75 | HZ | 0-75 | HZ |
| | | 76-150 | HZ | 76-150 | HZ | 76-150 | HZ |
| 45 | Insomnia | 0-75 | HZ | 0-75 | HZ | 0-75 | HZ |
| | | 76-150 | HZ | 76-150 | HZ | 76-150 | HZ |
| 46 | Ischemic heart | 0-75 | HZ | 0-75 | HZ | 0-75 | HZ |
| | disease, Angina | 76-150 | HZ | 76-150 | HZ | 76-150 | HZ |
| | pectoris | | | | | | |
| 47 | Coprostasia | | | | | | |
| 48 | Leucosis (list all | 0-75 | HZ | 0-75 | HZ | 0-75 | HZ |
| | types) | 76-150 | HZ | 76-150 | HZ | 76-150 | HZ |
| 49 | Migraine | 0-75 | HZ | 0-75 | HZ | 0-75 | HZ |
| | | 76-150 | HZ | 76-150 | HZ | 76-150 | HZ |
| 50 | Myodystrophy | 0-75 | HZ | 0-75 | HZ | 0-75 | HZ |
| | | 76-150 | HZ | 76-150 | HZ | 76-150 | HZ |
| 51 | Myopathy | 0-75 | HZ | 0-75 | HZ | 0-75 | HZ |
| | | 76-150 | HZ | 76-150 | HZ | 76-150 | HZ |
| 52 | Male and female | 0-75 | HZ | 0-75 | HZ | 0-75 | HZ |
| | infertility | 76-150 | HZ | 76-150 | HZ | 76-150 | HZ |
| 53 | Narcolepsy | 0-75 | HZ | 0-75 | HZ | 0-75 | HZ |
| | | 76-150 | HZ | 76-150 | HZ | 76-150 | HZ |
| 54 | Drug addiction (list | 0-75 | HZ | 0-75 | HZ | 0-75 | HZ |
| | all types) | 76-150 | HZ | 76-150 | HZ | 76-150 | HZ |
| 55 | Bauhin's valve | 76-150 | HZ, | 76-150 | HZ, | 76-150 | HZ, |
| | failure | 150-225 | HZ | 150-225 | HZ | 150-225 | HZ |
| 56 | Adrenal cortex | 0-75 | HZ | 0-75 | HZ | 0-75 | HZ |
| | insufficiency | 76-150 | HZ | 76-150 | HZ | 76-150 | HZ |
| 57 | Neurodermite | 0-75 | HZ | 0-75 | HZ | 0-75 | HZ |
| | | 76-150 | HZ | 76-150 | HZ | 76-150 | HZ |
| 58 | Nervous anorexia | 0-75 | HZ | 0-75 | HZ | 0-75 | HZ |
| | | 76-150 | HZ | 76-150 | HZ | 76-150 | HZ |
| 59 | Diabetes insipidus | 0-75 | HZ | 0-75 | HZ | 0-75 | HZ |
| | | 76-150 | HZ | 76-150 | HZ | 76-150 | HZ |
| 60 | Obliterating | 0-75 | HZ | 0-75 | HZ | 0-75 | HZ |
| | atherosclerosis of the | 76-150 | HZ | 76-150 | HZ | 76-150 | HZ |
| | limbs' vessels | | | | | | |
| 61 | Obliterating | 0-75 | HZ | 0-75 | HZ | 0-75 | HZ |
| | endarteritis of the | 76-150 | HZ | 76-150 | HZ | 76-150 | HZ |
| | lower limbs' vessels | | | | | | |
| 62 | Alopecia | 0-75 | HZ | 0-75 | HZ | 0-75 | HZ |
| | | 76-150 | HZ | 76-150 | HZ | 76-150 | HZ |
| 63 | Obesity | 0-75 | HZ | 0-75 | HZ | 0-75 | HZ |
| | | 76-150 | HZ | 76-150 | HZ | 76-150 | HZ |
| 64 | Osteoporosis | 0-75 | HZ | 0-75 | HZ | 0-75 | HZ |
| | | 76-150 | HZ | 76-150 | HZ | 76-150 | HZ |
| 65 | Trauma-related | 0-75 | HZ | 0-75 | HZ | 0-75 | HZ |
| | paralyses | 76-150 | HZ, | 76-150 | HZ, | 76-150 | HZ, |
| | | 150-225 | HZ, | 150-225 | HZ, | 150-225 | HZ, |
| | | 225-300 | HZ | 225-300 | HZ | 225-300 | HZ |
| 66 | Paralyses of | 0-75 | HZ | 0-75 | HZ | 0-75 | HZ |
| | different etiology | 76-150 | HZ | 76-150 | HZ | 76-150 | HZ |
| 67 | Paresis of the vocal | 0-75 | HZ | 0-75 | HZ | 0-75 | HZ |
| | cords | 76-150 | HZ | 76-150 | HZ | 76-150 | HZ |
| 68 | Borderline | 0-75 | HZ | 0-75 | HZ | 0-75 | HZ |
| | personality disorders | 76-150 | HZ | 76-150 | HZ | 76-150 | HZ |
| 69 | Priapism | 0-75 | HZ | 0-75 | HZ | 0-75 | HZ |
| | | 76-150 | HZ | 76-150 | HZ | 76-150 | HZ |
| 70 | Progressing | 0-75 | HZ | 0-75 | HZ | 0-75 | HZ |
| | myodystrophy | 76-150 | HZ | 76-150 | HZ | 76-150 | HZ |
| 71 | Psoriasis | 0-75 | HZ | 0-75 | HZ | 0-75 | HZ |
| | | 76-150 | HZ | 76-150 | HZ | 76-150 | HZ |
| 72 | Cancer (list different | | | | | | |
| | localizations and | | | | | | |
| | types) | | | | | | |
| 73 | Rickets | 0-75 | HZ | 0-75 | HZ | 0-75 | HZ |
| | | 76-150 | HZ | 76-150 | HZ | 76-150 | HZ |
| 74 | Reflux-esophagitis | 0-75 | HZ | 0-75 | HZ | 0-75 | HZ |
| | | 76-150 | HZ | 76-150 | HZ | 76-150 | HZ |
| 75 | Sarcoma | | | | | | |
| 76 | Diabetes | 0-75 | HZ | 0-75 | HZ | 0-75 | HZ |
| | | 76-150 | HZ | 76-150 | HZ | 76-150 | HZ |
| 77 | Syringomyelia | 0-75 | HZ | 0-75 | HZ | 0-75 | HZ |
| | | 76-150 | HZ | 76-150 | HZ | 76-150 | HZ |
| 78 | Zollinger-Ellison | 0-75 | HZ | 0-75 | HZ | 0-75 | HZ |
| | syndrome | 76-150 | HZ | 76-150 | HZ | 76-150 | HZ |
| 79 | Commissural disease | 0-75 | HZ | 0-75 | HZ | 0-75 | HZ |
| | | 76-150 | HZ | 76-150 | HZ | 76-150 | HZ |
| 80 | Angina pectoris | | | | | | |
| 81 | Anxiety and | 0-75 | HZ | 0-75 | HZ | 0-75 | HZ |
| | depression | 76-150 | HZ | 76-150 | HZ | 76-150 | HZ |
| 82 | Huntington's chorea | 0-75 | HZ | 0-75 | HZ | 0-75 | HZ |
| | | 76-150 | HZ | 76-150 | HZ | 76-150 | HZ |
| 83 | Chronic duodenal | 0-75 | HZ | 0-75 | HZ | 0-75 | HZ |
| | ileus | 76-150 | HZ | 76-150 | HZ | 76-150 | HZ |
| 84 | Chronic and acute | 0-75 | HZ | 0-75 | HZ | 0-75 | HZ |
| | hyperthermia | 76-150 | HZ | 76-150 | HZ | 76-150 | HZ |
| 85 | Chronic intestinal | 0-75 | HZ | 0-75 | HZ | 0-75 | HZ |
| | obstruction | 76-150 | HZ | 76-150 | HZ | 76-150 | HZ |
| | (Commissural | | | | | | |
| | disease, | | | | | | |
| | Megacolon, | | | | | | |
| | Chronic mesenterial | | | | | | |
| | circulation | | | | | | |
| | insufficiency, | | | | | | |
| | Metacolon, | | | | | | |
| | Dolichosigmoid, | | | | | | |
| | Cardiac achalasia) | | | | | | |
| 86 | Chronic | 76-150 | HZ, | 76-150 | HZ, | 76-150 | HZ, |
| | gastroenterocolitis | 150-225 | HZ | 150-225 | HZ | 150-225 | HZ |
| 87 | Chronic hepatitis | 0-75 | HZ | 0-75 | HZ | 0-75 | HZ |
| | (list each type in | 76-150 | HZ | 76-150 | HZ | 76-150 | HZ |
| | detail) | | | | | | |
| 88 | Chronic cholecystitis | 0-75 | HZ | 0-75 | HZ | 0-75 | HZ |
| | and | 76-150 | HZ | 76-150 | HZ | 76-150 | HZ |
| | cholecystopancreatitis | | | | | | |
| 89 | Chronic cystitis | 0-75 | HZ | 0-75 | HZ | 0-75 | HZ |
| | | 76-150 | HZ | 76-150 | HZ | 76-150 | HZ |
| 90 | Chronic periodic | 0-75 | HZ | 0-75 | HZ | 0-75 | HZ |
| | pains | 76-150 | HZ | 76-150 | HZ | 76-150 | HZ |
| | (Angina pectoris, | | | | | | |
| | Phantom pains, | | | | | | |
| | Neuritis, | | | | | | |
| | Nerve root | | | | | | |
| | syndromes | | | | | | |
| | Terminal-stage pains | | | | | | |
| | Migraine) | | | | | | |
| 91 | Chronic | 0-75 | HZ | 0-75 | HZ | 0-75 | HZ |
| | glomerulonephritis | 76-150 | HZ, | 76-150 | HZ, | 76-150 | HZ, |
| | | 150-225 | HZ, | 150-225 | HZ, | 150-225 | HZ, |
| | | 225-300 | HZ | 225-300 | HZ | 225-300 | HZ |
| 92 | Chronic obstructive | 0-75 | HZ | 0-75 | HZ | 0-75 | HZ |
| | bronchitis | 76-150 | HZ | 76-150 | HZ | 76-150 | HZ |
| 93 | Chronic | 0-75 | HZ | 0-75 | HZ | 0-75 | HZ |
| | osteomyelitis | 76-150 | HZ | 76-150 | HZ | 76-150 | HZ |
| 94 | Chronic | 0-75 | HZ | 0-75 | HZ | 0-75 | HZ |
| | pyelonephritis | 76-150 | HZ, | 76-150 | HZ, | 76-150 | HZ, |
| | | 150-225 | HZ, | 150-225 | HZ, | 150-225 | HZ, |
| | | 225-300 | HZ | 225-300 | HZ | 225-300 | HZ |
| 95 | Cirrhosis (list all | 0-75 | HZ | 0-75 | HZ | 0-75 | HZ |
| | types) | 76-150 | HZ | 76-150 | HZ | 76-150 | HZ |
| 96 | Schizophrenia with | 0-75 | HZ | 0-75 | HZ | 0-75 | HZ |
| | schizophrenic | 76-150 | HZ | 76-150 | HZ | 76-150 | HZ |
| | affective disorders | | | | | | |
| | and delirium | | | | | | |
| 97 | Eczema | 0-75 | HZ | 0-75 | HZ | 0-75 | HZ |
| | | 76-150 | HZ | 76-150 | HZ | 76-150 | HZ |

TABLE 10
Experiments performed for each disease

| | | Experiments |
|---|---|---|
| | | Y/N |
| | | Amount: |
| | Diseases and | A-Animal |
| No | syndromes | H-Human |
| 1 | Asthma | A-126, H-372 |
| 2 | Epilepsy | A-38, H-48 |
| 3 | Parkinson's disease | A-4, H-0 |
| 4 | Alzheimer's disease | A-6, H-0 |
| 5 | Gastric ulcer | A-26, H-12 |
| 6 | Diabetes | A-22, H-9 |
| 7 | Hypertony | A-20, H-9 |
| 8 | Obesity | A-30, H-12 |
| 9 | Infantile cerebral | A-0, H-0 |
| | paralysis | |
| 10 | Posttraumatic | A-0, H-0 |
| | paralysis | |
| 11 | Hirschsprung's | A-0, H-0 |
| | disease | |
| 12 | Mental diseases | A-32, H-9 |
| 13 | Adrenal cortex | A-18, H-2 |
| | failure | |
| 14 | Hypothyrosis | A-32, H-0 |
| 15 | Urinary bladder | A-0, H-0 |
| | atony | |
| 16 | Pain syndromes of | A-6, H-14 |
| | different genesis | |
| 17 | Obliterating vascular | A-0, H-9 |
| | diseases of the limbs | |
| 18 | Constipation | A-0, H-1 |
| 19 | Progressing | A-0, H-0 |
| | myodystrophy | |
| 20 | Angiotrophoneurosis | A-0, H-1 |
| 21 | Syringomyelia | A-0, H-0 |
| 22 | Anxiety, neurosis, | A-32, H-18 |
| | depression | |
| 23 | Alcoholism | A-0, H-0 |
| 24 | Drug addiction | A-0, H-0 |
| 25 | Impotence | A-0, H-1 |
| 26 | Cardiac arrhythmia | A-0, H-3 |
| 27 | Paresis of the vocal | A-0, H-0 |
| | cords | |
| 28 | Osteoporosis | A-0, H-2 |
| 29 | Angina pectoris | A-30, H-13 |
| 30 | Apnea, snoring, | A-0, H-2 |
| | hypersomnia | |
| 31 | Chronic | A-0, H-2 |
| | gastroenterocolitis | |
| 32 | Ailments, associated | A-0, H-0 |
| | with a malfunction | |
| | of the rectum | |
| | sphincters | |
| 33 | Male and female | A-0, H-0 |
| | infertility | |
| 34 | Stammering | A-0, H-1 |
| 35 | Insomnia | A-0, H-2 |
| 36 | Bulimia | A-0, H-2 |
| 37 | Reflux-esophagitis | A-0, H-3 |
| | Total | A-422, H-547 |

TABLE 11
Results of Obesity Treatment

| Clinical History, | | | |
|---|---|---|---|
| Treatment Results | 1 | 2 | 3 |
| Age, sex (m, f) | 56 M | 42 F | 45 M |
| Diagnosis of the Main | Ob-A-2 | Ob-B-2 | Ob-B-2 |
| Disease | | | |
| Complication of the | arthrosis of the lower limbs' | RI - 1 respiratory insufficiency | RI - 2 |
| Main Disease | joints, Myocardial dystrophy, | | Respiratory insufficiency, |
| | hypercholesterolemia, CVI | | Spondylarthrosis |
| Diagnosis of | Asthma intrinsic, Ischemic heart | Ht, Asthma intrinsic, Icenko- | Asthma intrinsic, Icenko-Cushing |
| Associated Ailments | disease | Cushing syndrome | syndrome |
| Duration of the Main | 15 years | 7 years | 9 years |
| Disease | | | |
| Date and Type of | chip implantation, endoscopic | chip implantation, endoscopic | chip implantation, endoscopic |
| Surgery | chip implantation., | chip implantation., 18 Dec. 2001 | chip implantation, 11 Nov. 2001 |
| | 6 Feb. 2002 | | |
| Implanted Chip Type, | Chip 3, 5 cm | Chip 4, 4 cm, | Chip 3, 5 cm |
| Size (cm), | | | |
| Implanted Sensors | Arterial pressure meter | Arterial pressure meter | Arterial pressure meter |
| | Heart rate meter | Heart rate meter | Heart rate meter |
| | Respiration rate meter | Respiration rate meter | Respiration rate meter |
| | Temperature gage | Temperature gage | Temperature gage |
| | Local blood flow sensor - Sensor | Local blood flow sensor - Sensor | Local blood flow sensor - Sensor |
| | of electric activity of the organs, | of electric activity of the organs, | of electric activity of the organs, |
| | nervous centers | nervous centers | nervous centers |
| | Gastric juice acidity sensor | Gastric juice acidity sensor | Gastric juice acidity sensor |
| | Murmur sensor (heart, lungs, | Murmur sensor (heart, lungs, | Murmur sensor (heart, lungs, |
| | intestine) | intestine) | intestine) |
| Implanted Biosensors | Biosensor of oxygen contents in | Biosensor of oxygen contents in | Biosensor of oxygen contents in |
| | the tissues | the tissues | the tissues |
| | Biosensor of sugar contents in | Biosensor of sugar contents in | Biosensor of sugar contents in |
| | the blood | the blood | the blood |
| | Biosensor of hormone contents | Biosensor of hormone contents | Biosensor of hormone contents |
| | in the blood | in the blood | in the blood |
| Power Source | RF power source | RF power source | RF power source |
| Description | | | |
| Name of the nerve the | Vagus nerve | Sympathetic trunk, thoracic part | Sympathetic trunk, thoracic part |
| chip has been | | Vagus nerve | |
| connected to | | | |
| Chip's Working | Programmed periodical electric | Programmed periodical electric | Programmed periodical electric |
| Algorithm | stimulation, or non-programmed | stimulation, or non-programmed | stimulation, or non-programmed |
| | electric stimulation per schedule | electric stimulation per schedule | electric stimulation per schedule |
| | entered (Chip 1, Chip 2). | entered (Chip 1, Chip 2). | entered (Chip 1, Chip 2). |
| | | Urgent electric stimulation that | |
| | | starts upon detecting (by means | |
| | | of the sensors and biosensors) of | |
| | | a change in the gastric juice | |
| | | acidity level and gastric | |
| | | peristalsis enhancement in order | |
| | | to inhibit stomach functions. | |
| Treatment methods | diet therapy (No effect) | diet therapy (No effect) | diet therapy (No effect) |
| used before the | medicinal therapy (No effect) | | |
| operation and their | | | |
| efficacy | | | |
| Clinical History of the | | | |
| main disease and | | | |
| associated ailments | | | |
| before the operation/ . . . | | | |
| months after the | | | |
| operation | | | |
| Body weight: | 82/70.5/72 | 92/89/87 | 88/85/79 |
| Before the operation/ | (after 9 months - 10 kg weight | (after 11 months 5 kg weight Loss) | (after 12 months 9 kg weight loss) |
| one month later/3 | loss) | | |
| months later/ . . . | | | |
| months later (kg) | | | |
| Body Mass Index | 32/28/28.5 | 36/34/32 | 33/32/29 |
| (BMI): Before the | | | |
| operation/one month | | | |
| later/3 months later/ . . . | | | |
| months later | | | |
| (kg/m²) | | | |
| Complications | Anorexia, developed 1 week after | — | — |
| resulting from the | chip activation | | |
| operation, side effects | | | |
| of the method | | | |
| Average | 25/20 | 32/28 | 30/14 |
| hospitalization | | | |
| duration during a 1- | | | |
| year-period (days) | | | |
| before the operation/ | | | |
| during the post- | | | |
| operation period | | | |
| Reduction of daily | 9 months later - 42% reduction | 11 months later - | 12 months later - 42% reduction |
| intake of medicines | | 55% reduction | |
| (X-times) . . . months | | | |
| after the operation | | | |
| (drug therapy of | | | |
| associated ailments) | | | |
| Clinical condition of | I | I | I |
| associated ailments | | | |
| resulting from chip- | | | |
| assisted treatment | | | |

| Clinical History, | | |
|---|---|---|
| Treatment Results | 4 | 5 |
| Age, sex (m, f) | 28 M | 54 M |
| Diagnosis of the Main | Ob-B-2 | Ob-B-2 |
| Disease | | |
| Complication of the | Respiratory insufficiency, | RI-I Respiratory insufficiency, |
| Main Disease | Hyperchlorhydria | Myocardial dystrophy, Apnea |
| Diagnosis of | Asthma intrinsic, Icenko-Cushing | Asthma intrinsic, Icenko-Cushing |
| Associated Ailments | syndrome | syndrome |
| Duration of the Main | 6 years | 11 years |
| Disease | | |
| Date and Type of | chip implantation, endoscopic | chip implantation, |
| Surgery | chip implantation., 3 Feb. 2002 | 8 May 2002 |
| Implanted Chip Type, | Chip 5, 5 cm | Chip 4, 5 cm |
| Size (cm), | | |
| Implanted Sensors | Arterial pressure meter | Arterial pressure meter |
| | Heart rate meter | Heart rate meter |
| | Respiration rate meter | Respiration rate meter |
| | Temperature gage | Temperature gage |
| | Local blood flow sensor - Sensor | Local blood flow sensor - Sensor |
| | of electric activity of the organs, | of electric activity of the organs, |
| | nervous centers | nervous centers |
| | Gastric juice acidity sensor | Gastric juice acidity sensor |
| | Murmur sensor (heart, lungs, | Murmur sensor (heart, lungs, |
| | intestine) | intestine) |
| Implanted Biosensors | Biosensor of oxygen contents in | Biosensor of oxygen contents in |
| | the tissues | the tissues |
| | Biosensor of sugar contents in | Biosensor of sugar contents in |
| | the blood | the blood |
| | Biosensor of hormone contents | Biosensor of hormone contents |
| | in the blood | in the blood |
| Power Source | Built-in power source of the chip | Built-in power source of the chip + RF |
| Description | | power source |
| Name of the nerve the | Vagus nerve | Vagus nerve |
| chip has been | Sympathetic trunk, thoracic part | |
| connected to | | |
| Chip's Working | Programmed periodical electric | Programmed periodical electric |
| Algorithm | stimulation, or non-programmed | stimulation, or non-programmed |
| | electric stimulation per schedule | electric stimulation per schedule |
| | entered (Chip 1, Chip 2). | entered (Chip 1, Chip 2). |
| | Urgent electric stimulation that | Urgent electric stimulation that |
| | starts upon detecting (by means | starts upon detecting (by means |
| | of the sensors and biosensors) of | of the sensors and biosensors) of |
| | a change in the gastric juice | a change in the gastric juice |
| | acidity level and gastric | acidity level and gastric |
| | peristalsis enhancement in order | peristalsis enhancement in order |
| | to inhibit stomach functions. | to inhibit stomach functions. |
| Treatment methods | diet therapy (No effect) | diet therapy (No effect) |
| used before the | | |
| operation and their | | |
| efficacy | | |
| Clinical History of the | | |
| main disease and | | |
| associated ailments | | |
| before the operation/ . . . | | |
| months after the | | |
| operation | | |
| Body weight: | 90/86/83 | 102/95/94 |
| Before the operation/ | (after 9 months - 7 kg weight | (after 6 months - 8 kg weight |
| one month later/3 | loss) | loss) |
| months later/ . . . | | |
| months later (kg) | | |
| Body Mass Index | 35/33/32 | 37/35/33 |
| (BMI): Before the | | |
| operation/one month | | |
| later/3 months later/ . . . | | |
| months later | | |
| (kg/m²) | | |
| Complications | — | — |
| resulting from the | | |
| operation, side effects | | |
| of the method | | |
| Average | 41/20 | 26/19 |
| hospitalization | | |
| duration during a 1- | | |
| year-period (days) | | |
| before the operation/ | | |
| during the post- | | |
| operation period | | |
| Reduction of daily | 9 months later - | 6 months later - |
| intake of medicines | 43% reduction | 38% reduction |
| (X-times) . . . months | | |
| after the operation | | |
| (drug therapy of | | |
| associated ailments) | | |
| Clinical condition of | I | I |
| associated ailments | | |
| resulting from chip- | | |
| assisted treatment | | |

Conclusion:
Fast weight loss in 1 patient (20%);
Slow weight loss in 5 patients (80%)

TABLE 12
Statistics for obesity treatment

| Clinical History and Treatment Results | Mean Value |
|---|---|
| Age | 45 |
| Sex | 3 men, 2 women |
| Duration of the Main Disease (years) | 9.6 |
| Body weight: | 90.8/85.1/83 |
| Before the operation/one month later/3 months | |
| later/6-12 months later (kg) | |
| Body Mass Index (BMI): Before the operation/one | 34.6/32.4/30.9 |
| month later/3 months later/ | |
| 6-12 months later (kg/m²) | |
| Average hospitalization duration during a 1-year- | 30.8/20.2 |
| period before the operation/during the post- | |
| operation period (days) | |

TABLE 13
Asthma Clinical Examples

| Patient No 5 | Patient No 4 | Patient No 3 | Patient No 2 | Patient No 1 | |
|---|---|---|---|---|---|
| (MA) | (PHA) | (LGN) | (STV) | (KMP) | Age |
| 34 M | 68 F | 42 F | 46 F | 55 M | & Sex |
| 1. Euphylline (tablets) | 1. Euphylline (tablets) | 1. Euphylline (tablets) - | 1. Euphylline (tablets) | 1. Euphylline (tablets) | [symbol] |
| 2. Berotek (inhalation) | 2. Berotek (inhalation) | 2. Berotek (inhalation) | 2. Berotek (inhalation) | 2. Berotek (inhalation) | [symbol] |
| | 3. theophedrine (tablets) | 3. theophedrine (tablets) | | | [symbol] |
| | | | | | [symbol] |
| 1. Becotide (inhalation) | 1. Prednisolone (tablets) | 1. Prednisolone (tablets) | 1. Polcartolon (tablets) | 1. Polcartolon (tablets) | [symbol] |
| | 2. Becotide (inhalation) | | | 2. Becotide (inhalation) | [symbol] |
| | | | | | [symbol] |
| | | | | | [symbol] |
| 1. Euphylline (tablets) - | 1. Euphylline (tablets) - | 1. Euphylline (tablets) - | 1. Euphylline (tablets) - | 1. Euphylline (tablets) - | [symbol] |
| suspended | reduction50% | suspended | 50% reduction | 67% reductin | [symbol] |
| 2. Berotek (inhalation) - | 2. Berotek (inhalation) - | 2. Berotek (inhalation) - | 2. Berotek (inhalation) - | 2. Berotek (inhalation) - | [symbol] |
| 86% reduction (the | reduction67% | 80% reduction | 75% reduction | 84% reduction | [symbol] |
| patient uses the | 3. theophedrine (tablets) | 3. theophedrine (tablets) - | | | |
| preparation not more than | reduction50% - | 50% reduction | | | |
| once a month) | | | | | |
| 1. Becotide (inhalation) - | 1. Prednisolone (tablets) - | 1. Prednisolone (tablets) - | 1. Polcartolon (tablets) - | 1. Polcartolon (tablets) - | [symbol] |
| suspended after the | 67% reduction | 84% reduction | 50% reduction | 55% reduction | [symbol] |
| operation | 2. Becotide (inhalation) - | | | 2. Becotide (inhalation) - | [symbol] |
| | 75% reduction | | | 75% reduction | [symbol] |
| Intrinsic asthma, severe | Intrinsic asthma, severe | Intrinsic asthma, severe | intrinsic asthma, severe | Intrinsic asthma, severe | [symbol] |
| condition, hormone- | condition, hormone- | condition, hormone- | condition, hormone- | condition, hormone- | [symbol] |
| dependent form. | dependent form. | dependent form. | dependent form. | dependent form. | |
| | Complications: 1^{st}-2^{nd}- | | | Complications: 1^{st}-degree | |
| | degree respiratory | | | respiratory insufficiency. | |
| | insufficiency 1-2 CT. | | | | |
| Chronic obstructive | Chronic obstructive | Pulmonary emphyzema. | Chronic obstructive | 2^{nd}-stage hypertension. | [symbol] |
| bronchitis. | bronchitis. | 3-rd degree obesity. | bronchitis, uterine fibroid | chronic obstructive | [symbol] |
| | | | tumor. | bronchitis. | |
| | | | | | [symbol] |
| | | | | | [symbol] |
| 15 years | 26 years | 30 years | 18 years | 15 years | [symbol] |
| | | | | | [symbol] |
| before the operation/3 | before the operation/2.5 | before the operation/6 | before the operation/3 | before the operation/4 | [symbol] |
| years after the operation | years after the operation | years after the operation | years after the operation | years after the operation | [symbol] |
| a) mean frequency of | a) mean frequency of | a) mean frequency of | a) mean frequency of | a) mean frequency of | |
| attacks: 7/1-0. Asphyxia | attacks: 9/3 | attacks: 10/2 | attacks: 8/2 | attacks: 6/1 | |
| attacks occur very | b) mean duration of | b) mean duration of | b) mean duration of | b) mean duration of | |
| seldom (once a month). | attacks (minutes): 20/7 | attacks (minutes): 20/5 | attacks (minutes): 18/5 | attacks (minutes): 15/5 | |
| and the patient only | c) Mean remission | c) Mean remission | c) Mean remission | c) Mean remission | |
| suffers from a mild | duration (days): 29/65 | duration (days): 21/69 | duration (days): 31/106 | duration (days): 28/94 | |
| dyspnea, disappearing on | | | | | |
| its own without | | | | | |
| medications. | | | | | |
| b) mean duration of | | | | | |
| attacks (minutes): 19/6 | | | | | |
| c) Mean remission | | | | | |
| duration (days): 42/300 | | | | | |
| | | | | | [symbol] |
| | | | | | [symbol] |
| | | | | | [symbol] |
| | | | | | [symbol] |
| | | | | | [symbol] |
| | | | | | FVC |
| | | | | | FEV1 |
| | | | | | FVC/ |
| | | | | | FEV1 |
| | | | | | PEFR |
| | | | | | FEF 50 |
| | | | | | [symbol] |
| | | | | | [symbol] |
| FVC: 56/62 | FVC: 47/50 | FVC: 58/62 | FVC: 57/61 | FVC: 49/57 | FVC |
| FEVI: 45/59 | FEVI: 45/48 | FEVI: 44/44 | FEVI: 41/42 | FEVI: 35/41 | FEV1 |
| FVC/FEVI | FVC/FEVI | FVC/FEVI | FVC/FEVI | FVC/FEVI | FVC/ |
| BEF: 43/50 | BEF: 41/43 | BEF: 45/46 | BEF: 44/45 | BEF: 38/41 | FEV1 |
| FEF50: 44/56 | FEF50: 44/46 | FEF50: 42/45 | FEE50: 49/50 | FEF50: 32/44 | PEFR |
| | | | | | FEF 50 |
| a) bronchi dilatation test | a) bronchi dilatation test | a) bronchi dilatation test | a) bronchi dilatation test | a) bronchi dilatation test | [symbol] |
| results (0.2 mg Berotek | results (0.2 mg Berotek | results (0.2 mg Berotek | results (0.2 mg Berotek | results (0.2 mg Berotek | [symbol] |
| inhalation) | inhalation): | inhalation): | inhalation): | inhalation): | [symbol] |
| before the operation: | before the operation: | before the operation: | before the operation: | before the operation: | [symbol] |
| FEVI increase by 15% | FEVI increase by 8% | FEVI increase by 12% | FEVI increase by 10% | FEVI increase by 8% | [symbol] |
| 6years after the operation | 2 after the operation | 6years after the operation | 6years after the operation | 6years after the operation | [symbol] |
| FEVI increase by 18% | FEVI increase by 13% | FEVI increase by 16% | FEVI increase by 16% | FEVI increase by 14% | [symbol] |
| b) bronchi constriction | b) bronchi constriction | b) bronchi constriction | b) bronchi constriction | b) bronchi constriction | |
| test results (0.22 mg/ml | test results (0.22 mg/ml | test results (0.22 mg/ml | test results (0.22 mg/ml | test results (0.2 mg/m | |
| histamine inhalation): | histamine inhalation): | histamine inhalation): | histamine inhalation): | histamine inhalation): | |
| before the operation: | before the operation: | before the operation: | before the operation: | before the operation: | |
| FEVI decrease by 12% | FEVI decrease by 14% | FEVI decrease by 11% | FEVI decrease by 14% | FEVI decrease by 18% | |
| 3 years after the | 2 years after the | 6 years after the | 3 years after the | 4 years after the | |
| operation: FEVI | operation: FEVI decrease | operation: FEVI | operation: FEVI | operation: FEVI decrease | |
| decrease by 8% | by 8% | decrease by 9% | decrease by 8% | by 10% | |
| Jun. 15, 1999 | Jun. 16, 2000 | Mar. 18, 1996 | Apr. 16, 1999 | Jun. 15, 1998 | [symbol] |
| | | Oct. 5, 1999 | | | |
| To the right sympathetic | thoracic section of the | Chip-3: into the lumbar | the lumbar section of the | The right sinocarotid | [symbol] |
| trunk, in its thoracic part.. | left sympathetic trunk | section of the right | right sympathetic trunk. | nerve collector. | [symbol] |
| | | sympathetic trunk | | | |
| | | Chip-4: nerves of the | | | |
| | | stomach | | | |
| Chip-5, up to 5 cm in | Chip-3, up to 4 cm in | Chip-3, up to 4 cm in | Chip-3, up to 4 cm in | Chip-3, up to 4 cm in | [symbol] |
| diameter, external Radio- | diameter, external Radio- | diameter, external Radio- | diameter, external Radio- | diameter, external Radio- | [symbol] |
| frequency power source. | frequency power source. | frequency power source; | frequency power source. | frequency power source. | [symbol] |
| | | Chip-4, up to 7 cm in | | | |
| | | diameter, external Radio- | | | |
| | | frequency power source. | | | |
| Sensors: Respiratory rate | Sensors: Respiratory rate | Sensors: Respiratory rate | Sensors: Respiratory rate | Sensors: Respiratory rate | [symbol] |
| sensor, heart rate sensor, | sensor, heart rate sensor, | sensor, heart rate sensor, | sensor, heart rate sensor, | sensor, heart rate sensor, | [symbol] |
| body temperature sensor, | body temperature sensor, | body temperature sensor, | body temperature sensor, | body temperature sensor, | [symbol] |
| wheezing sensor, tissue | wheezing sensor, tissue | wheezing sensor, | wheezing sensor, tissue | wheezing sensor, arterial | [symbol] |
| oxygen biosensor. | oxygen biosensor | stomach murmur sensor, | oxygen biosensor | pressure sensor, tissue | [symbol] |
| | | gastric juice acidity | | oxygen biosensor | [symbol] |
| | | sensor, arterial pressure | | | [symbol] |
| | | sensor, blood oxygen | | | [symbol] |
| | | biosensor | | | |
| Start stimulation upon | start stimulation upon | Start stimulation upon | Start stimulation upon | Start stimulation upon | Chip's |
| detection of wheezing | detection of wheezing | detection of wheezing | detection of wheezing | detection of wheezing | oper- |
| appearance, respiratory | appearance, respiratory | appearance, respiratory | appearance, respiratory | appearance, respiratory | ational |
| rate acceleration, as well | rate acceleration, as well | rate acceleration, as well | rate acceleration, as well | rate acceleration, arterial | al- |
| as in an automatic mode, | as in an automatic mode, | as in an automatic mode, | as in an automatic mode, | pressure increase, as well | gorithm |
| with 5-6-hour intervals | with 3-4-hour intervals | with 3-4-hour intervals, | with 4-5-hour intervals, | as in an automatic mode, | |
| for prevention purposes. | | in accordance with attack | in accordance with attack | with 3-4-hour intervals, | |
| | | frequency in this specific | frequency in this specific | in accordance with attack | |
| | | patient, before the attack | patient, before the attack | “schedule” and frequency | |
| | | onset, for prevention | onset, for prevention | in this specific patient. | |
| | | purposes. Start | purposes. | | |
| | | stimulation of the nerves | | | |
| | | of the stomach (nervous | | | |
| | | collectors) from Chip-4 | | | |
| | | upon detection of an | | | |
| | | increase in the gastric | | | |
| | | peristalsis, gastric juice | | | |
| | | acidity, as well as before | | | |
| | | meals, in an automatic | | | |
| | | mode. | | | |
| | Mean duration of | obesity | Duration of remissions of | hypertension | Clinical |
| | remission of the chronic | 11 kg weight loss during | the chronic bronchitis has | Mean remission duration: | history |
| | bronchitis: | 3 years. | grown by 2.1 times. | Before the operation - 38 | of the |
| | Before the operation - 36 | | | days, | asso- |
| | days | | | After the operation - 122 | ciated |
| | 2 years after the | | | days. | ailment, |
| | operation - 65 days. | | | Before the operation the | tracking |
| | | | | patient was suffering 2-3 | the |
| | | | | hypertension crises per | changes |
| | | | | year; after the operation | result- |
| | | | | no such crises have been | ing |
| | | | | observed. | from |
| | | | | | using |
| | | | | | the chip |
| A considerable | A) Regarding the main | A) Regarding the main | A) Regarding the main | A) Regarding the main | Con- |
| improvement has been | disease, a considerable | disease, a considerable | disease, a considerable | disease, a considerable | clusion |
| observed: almost no | improvement has been | improvement has been | improvement has been | improvement has been | |
| asphyxia attacks occur, | observed: frequency of | observed: frequency of | observed: frequency of | observed: frequency of | |
| daily intake of | attacks dropped by 3 | attacks dropped by 5 | attacks dropped by 4 | attacks dropped by 6 | |
| medication reduced by | times at least, daily | times at least, daily | times at least, daily | times at least, daily | |
| more than 7 times, a | intake of medication | intake of medication | intake of medication | intake of medication | |
| stable (3-year)remission | reduced by more than 2 | reduced by more than 2 | reduced by more than 3 | reduced by more than 2.3 | |
| has been achieved after | times, a 2-fold increase | times, a minimal 3-fold | times, a minimal 3.1-fold | times, a minimal 3-fold | |
| the operation. | of the remission duration | increase of the remission | increase of the remission | increase of the remission | |
| | has been achieved. | duration has been | duration has been | duration has been | |
| | B) Refgarding the | achieved. | achieved. | achieved. | |
| | associated ailment the | B) Refgarding the | B) Regarding the | B) Regarding the | |
| | remission duration has | associated ailment | associated ailment - | associated ailment | |
| | increased by more than | (obesity), the patient lost | chronic bronchitis | (hypertension), the | |
| | 1.5 times. | 11 kg during 3 years. | stabilization, its acute | patient has not been | |
| | | | periods reduced by more | suffering from | |
| | | | than 2 times. | hypertension crises. | |

TABLE 14
Asthma surgery data

| | | | |
|---|---|---|---|
| 2002 | 54 | Chip2 - 42 patients | Asthma forms: |
| | | Chip3 - 6 patients | Extrinsic - 1 |
| | | Chip4 - 2 patients | Intrinsic - 79 |
| | | Chip5 - 2 patients | Chip's implantation |
| | | Chip6 - 2 patients | method: |
| | | | Via conventional surgical |
| | | | accesses - 46 patients; |
| | | | Video-endothoracoscopy - |
| | | | 6 patients; |
| | | | Stereotaxic accesses - 2 |
| | | | patients |
| 1993-2002 | | Total: 324 patients | |
| | | Chip1 - 25 patients | |
| | | Chip2 - 275 patients | |
| | | Chip3 - 10 patients | |
| | | Chip4 - 6 patients | |
| | | Chip5 - 4 patients | |
| | | Chip6 - 4 patients | |

TABLE 15
Epilepsy clinical examples
Epilepsy Clinical Examples

| Patient No 5 | Patient No 4 | Patient No 3 | Patient No 2 | Patient No 1 | |
|---|---|---|---|---|---|
| (KL) | (GSA) | (DV) | (DNI) | (KV) | Age & |
| 19 F | 43 M | 31 M | 38 F | 28 M | Sex |
| Generalized idiopathic | Generalized idiopathic | Localized focal | Generalized idiopathic | Localized partial | [symbol] |
| epilepsy with infrequent | epilepsy with frequent | idiopathic epilepsy with | epilepsy with frequent | idiopathic epilepsy | [symbol] |
| tonoclonic seizures. | tonoclonic seizures. | infrequent tonoclonic | tonoclonic seizures | | Partial/ |
| | | seizures; Atonic | | | general- |
| | | seizures, absences, petit | | | ized |
| | | mal, grand mal. | | | Simple/ |
| | | | | | complex |
| 10 years | 35 years | 20 years | 30 years | 16 years | [symbol] |
| | | | | | [symbol] |
| | | | | | [symbol] |
| aura/prodrom before | aura/prodrom before | aura/prodrom before | aura/prodrom before | aura/prodrom before | [symbol] |
| seizures: spasm of the | seizures: nausea. | seizures: spasm of the | seizures: none | seizures: spasm of the | Aura/ |
| left Musculus | | right upper eyelid. | | right nodding muscle | prodrom |
| Gastrocnemius | | | | | |
| | | | | | [symbol] |
| | | | | | [symbol] |
| EEG results: before the | EEG results: before the | results: before the | results: before | EEG results: before | EEG |
| operation - paroxysmal | operation - paroxysmal | operation - paroxysmal | operation - paroxysmal | the operation - | [symbol] |
| activity in the temporal | activity in the | activity in the left | activity in the | paroxysmal activity | |
| lobes occurring at rest; | parietotemporal lobes | temporal lobe occurring | parietotemporal lobes | in the left temporal | |
| after the operation - no | occurring at rest; after | both at hyperventilation; | occurring both at | lobe occurring both at | |
| paroxysmal activity | the operation - | after the operation - no | hyperventilation and | hyperventilation and | |
| detected. | paroxysmal activity in | paroxysmal activity | without it; after the | without it; after the | |
| | the parietotemporal | detected. | operation - single | operation - single | |
| | lobes at hyperventilation | | “peak-wave”-type | “peak-wave”-type | |
| | only; phtostimulations in | | complexes in the | complexes in the left | |
| | the parietotemporal | | temporal lobes | temporal lobe | |
| | lobes. | | occurring at | occurring at | |
| | | | hyperventilation only. | hyperventilation only. | |
| CT results: no pathology | CT results: | CT results: Chiari's | CT results: no pathology | CT results: no | [symbol] |
| detected. | hydrocephalus, | syndrome symptoms. | detected. | pathology | CT/MRI/ |
| | arachnoiditis symptoms. | | | detected. | PET/ |
| | | | | | SPECT |
| | | | | | [symbol] |
| before the operation/1 | | before the operation/x | before the operation/1 | before the operation/2 | Status |
| years after the operation | | years after the operation | years after the | years after the | epilepticus |
| Annual frequency of | Annual frequency of | Annual frequency of | Annual frequency of | operation | [symbol] |
| Status Epilepticus: 1/0 | Status Epilepticus: 2/0 | Status Epilepticus: 1/0 | Status Epilepticus: 2/0 | Annual frequency of | [symbol] |
| | | | | Status Epilepticus: 3/0 | |
| None | 2^{nd}-stage hypertension | Intrinsic moderate | None | None | [symbol] |
| | | asthma | | | |
| before the operation/1 | before the operation/1 | before the operation/x | before the operation/1 | before the operation/2 | [symbol] |
| year after the operation | year after the operation | years after the operation | years after the | years after the | [symbol] |
| a. mean monthly | a. mean monthly | a. mean monthly | a. mean monthly | operation | |
| frequency of seizures: | frequency of seizures: | frequency of seizures: | frequency of seizures: | a. mean monthly | |
| grand mal - 2/0, petit | grand mal - 7/1, petit | grand mal - 3/0, atonic - | grand mal - 6/1, petit | frequency of seizures: | |
| mal - 3/0, absences - | mal - 10/2 | 8/2 | mal - 8/1 | 10/2 - absences, grand | |
| 4/0 | b. mean duration of | b. mean duration of | b mean duration of | mal - up to 2/0. | |
| b. mean duration of | seizures (minutes): 19/5 | seizures (minutes): | seizures (minutes): 15/0 | b. mean duration of | |
| seizures (minutes): 20/0 | (grand mal), 9/1 (petit | c. Mean remission | (grand mal), 8/3 (petit | seizures (minutes): | |
| (grand mal), 7/0 (petit | mal) | duration (days): 51/126 | mal) | 2/0.5 - absences, | |
| mal), 2/0 (absences) | c. Mean remission | | c. Mean remission | 15/0 - grand mal | |
| c. Mean remission | duration (days): 14/58 | | duration (days): 20/92 | c. Mean remission | |
| duration (days): 42/152 | | | | duration (days): | |
| | | | | 44/118 | |
| | | | | | [symbol] |
| | | | | | [symbol] |
| before the operation/1 | before the operation/1 | before the operation/x | before the operation/1 | before the operation/2 | [symbol] |
| years after the operation | years after the operation | years after the operation | years after the operation | years after the | [symbol] |
| 1. Diphenylhydantion | 1. Diphenylhydantion | 1. Diphenylhydantion | 1. Carbamazepine - | operation | |
| Sodium - suspended | Sodium - 34% | Sodium - 34% reduction | suspended | 1. Phenobarbital | |
| Carbamazepine - | reduction | 2. Phenobarbital Sodium - | 2. Phenobarbital Sodium - | Sodium - suspended | |
| suspended | 2. Carbamazepine - | no changes | 50% reduction | 2. Decapine - 50% | |
| 2. Phenobarbital Sodium - | 52% reduction | | 3. Decapine - 50% | reduction | |
| 50% reduction | 3. Phenobarbital Sodium - | | reduction | | |
| 3. Decapine - suspended | 50% reduction | | 4. Lamotrigine - 33% | | |
| | 4. Decapine - suspended | | reduction | | |
| | 5. Lamotrigine - 34% | | | | |
| | reduction | | | | |
| May 4, 2002 | Jun. 11, 2001 | Mar. 11, 1998 | Oct. 6, 2001 | May 8, 2000 | [symbol] |
| To the right sinocarotid | To the right sinocarotid | To the right sinocarotid | To the right sinocarotid | To the right | [symbol] |
| nerve collector. | nerve collector. | nerve collector. | nerve collector. | sinocarotid | [symbol] |
| | | | | nerve collector. | |
| Chip-5, up to 5 cm in | Chip-3, up to 5 cm in | Chip-3, tip to 5 cm in | Chip-3, up to 5 cm in | Chip-3, up to 4 cm in | [symbol] |
| diameter, external | diameter, external | diameter, external | diameter, external | diameter, external | [symbol] |
| Radio-frequency power | Radio-frequency power | Radio-frequency power | Radio-frequency | Radio-frequency | [symbol] |
| source. | source. | source. | source. | power source. | |
| Sensors: Respiratory | Sensors: Respiratory | Sensors: Respiratory | Sensors: Respiratory | Sensors: Respiratory | [symbol] |
| rate sensor, heart rate | rate sensor, heart rate | rate sensor, heart rate | rate sensor, heart rate | rate sensor, heart rate | [symbol] |
| sensor, body | sensor, body | sensor, body | sensor, body | sensor, body | [symbol] |
| temperature sensor, | temperature sensor, | temperature sensor, | temperature sensor, | temperature sensor, | [symbol] |
| electric muscular | electric muscular | wheezing sensor, | electric muscular | electric muscular | [symbol] |
| activity sensor, arterial | activity sensor, arterial | electric muscular | activity sensor, arterial | activity sensor, oxygen | [symbol] |
| pressure sensor, tissue | pressure sensor, tissue | activity sensor, arterial | pressure sensor, tissue | biosensor | [symbol] |
| oxygen biosensors. | oxygen biosensors. | pressure sensor, tissue | oxygen biosensor | | |
| | | oxygen biosensor | | | |
| Start stimulation upon | Start stimulation upon | Start stimulation upon | Start stimulation upon | Start stimulation upon | Chip's op- |
| detection of respiratory | detection of respiratory | detection of respiratory | detection of abrupt | detection of epileptic | erational |
| rate and pulse | rate and pulse | rate and pulse | respiratory rate and | muscular spasms, | algorithm |
| acceleration, muscular | acceleration, as well as | acceleration, muscular | pulse acceleration, as | respiratory rate and | |
| spasms, as well as in an | in an automatic mode, | spasms. as well as in an | well as in an automatic | pulse acceleration, as | |
| automatic mode, with 3-4- | with 3-5-hour intervals, | automatic mode, with | mode, with 5-8-hour | well as in an automatic | |
| hour intervals, for | for seizure prevention | 10-12-hour intervals, for | intervals, for seizure | mode, with 5-8-hour | |
| seizure prevention | purposes. | seizure prevention | prevention purposes. | intervals, for seizure | |
| purposes. | | purpose | | prevention purposes. | |
| | before the operation/1 | before the operation/x | | | Clinical |
| | year after hile operation | year after the operation | | | history |
| | mean remission duration | mean remission | | | of the |
| | (of the hypertension): | duration | | | associated |
| | Before the operation - | Before the operation - | | | ailment. |
| | 26 days, | 43 days, | | | tracking |
| | During the year after the | 4.5 years after the | | | the |
| | operation - 70 days, | operation - 106 days. | | | changes |
| | | The hormonal drug | | | resulting |
| | | therapy (Prednisolone) | | | from using |
| | | has been suspended. | | | the chip |
| a. considerable | a. Regarding the main | a. Regarding the main | A considerable | A considerable | Con- |
| improvement has been | disease, a considerable | disease, a considerable | improvement has been | improvement has been | clusion |
| observed: no seizures, | improvement has been | improvement has been | observed: frequency of | observed: frequency of | |
| daily intake of | observed: frequency of | observed: frequency of | seizures dropped by 6 | seizures dropped by 5 | |
| medications reduced by | seizures dropped by | seizures dropped by 3 | times at least, Status | times at least, daily | |
| more than 2 times, a | 86%, daily intake of | times at least, daily | Epilepticus has | intake of medications | |
| minimal 3-fold increase | medications reduced by | intake of medications | disappeared, daily | reduced by more | |
| of the remission | more than 35%, a | reduced by more than | intake of medications | than 2 times. a | |
| duration has been | minimal 3-fold increase | 34% a minimal 2-fold | reduced by more than | minimal 2-fold | |
| achieved. | of the remission duration | increase of the remission | 1.5 times. a minimal 4- | increase of the | |
| | has been achieved: | duration has been | fold increase of the | remission duration | |
| | Status Epilepticus has | achieved. | remission duration has | has been achieved. | |
| | disappeared. | b. Regarding the | been achieved. | | |
| | b. Regarding the | associated ailment, a | | | |
| | associated ailment | minimal 2-fold increase | | | |
| | (hypertension), a | of the remission | | | |
| | minimal 50% increase of | duration has taken place, | | | |
| | the remission duration | hormonal drug therapy | | | |
| | has taken place. | has been suspended. | | | |

TABLE 16
Epilepsy surgery data

| | Total | | |
|---|---|---|---|
| | no. of | | |
| Surgery | operated | Name of the chip | |
| Year | patients | used | Notes |
| 1998 | 3 | Chip1 - 1 patient | Types of epileptic seizure: |
| | | Chip2 - 2 patients | Generalized tonicoclonic - |
| | | | 2 patients; |
| | | | Absence - 1 patient |
| 1999 | 3 | Chip2 - 1 patient | Types of epileptic seizure: |
| | | Chip4 - 2 patients | Generalized tonicoclonic - |
| | | | 2 patients; |
| | | | Absence - 1 patient |
| 2000-2002 | 8 | Chip2 - 4 patients | Types of epileptic seizure: |
| | | Chip3 - 1 patient | Generalized tonicoclonic - |
| | | Chip4 - 1 patient | 4 patients; |
| | | Chip5 - 1 patient | Atonic - 1 patient; |
| | | Chip6 - 1 patient | Absence - 2 patients; |
| | | | Mal petit - 1 patient |
| | | Total: 14 patients | |
| | | Chip1 - 1 patient | |
| | | Chip2 - 7 patients | |
| | | Chip3 - 1 patient | |
| | | Chip4 - 3 patients | |
| | | Chip5 - 1 patient | |
| | | Chip6 - 1 patient | |

TABLE 17
Examples of gastric and duodenal ulcer treatment
Clinical examples of gastric and duodenal ulcer treatment using implanted microchips

| Medical history, | Clinical | | | |
|---|---|---|---|---|
| treatment methods, and | Examples | | | |
| long-term results | 1 | 2 | 3 | 4 |
| Age, sex (m/f) | 44(M) | 53(M) | 35(M) | 38(M) |
| Duration of the disease | 3 years | 7 years | 10 years | 8 years |
| (years) | | | | |
| Diagnosis | Gastric ulcer | Duodenal ulcer | Gastric ulcer | Gastric ulcer |
| Complications: | | PANCR | | PANCR |
| Penetration into pancreas | | STENOS | | |
| Duodenal stenosis | | | | |
| Associated diseases | Obesity | Asthma | | Chronic hepatitis |
| Type of the chip | Chip 4 | chip 5 | Chip 3 | Chip 6 |
| implanted, | | | | |
| Name of the nerve the | 1. Vagus nerve | 1. Vagus nerve | 1. Sympathetic | 1. Sympathetic |
| chip has been connected | 2. Sympathetic | | trunk, thoracic | trunk, thoracic |
| to | trunk, thoracic | | section | section |
| | section | | | 2. Vagus nerve |
| Chip's program (programs) | GU-28 | GU-15 | GU-6 | GU-28A |
| Duration of the surgery | 95 | 86 | 26 | 38 |
| (minutes) | | | | |
| Complication during and | — | — | — | — |
| after the operation, | | | | |
| including technology- | | | | |
| related complications | | | | |
| Reduction of daily intake | Cymetidine-1,5 | Cymetidine - 60% | Cymetidine - 38% | Cymetidine - 50% |
| of medicines (X-times) | Gastrocelline-2,0 | Gastrocelline - | Gastrocelline - | Gastrocelline - |
| after the operation: | Alphogel - SUSP. | SUSP. | SUSP. | SUSP. Alphogel - |
| (before operation/one | Omeprasol - | Alphogel - 17% | Alphogel - SUSP. | 29% |
| year later) | SUSP. | Omeprasol - 57% | Omeprasol - SUSP | Omeprasol - |
| | | | | SUSP |
| Average annual duration | 29/14 | 30/25 | 24/10 | 26/2 |
| of hospitalization (sick | | | | |
| leave), days, | | | | |
| One year before the | | | | |
| operation/last year | | | | |

TABLE 18
Examples of dementia treatment
Clinical Examples of Dementia Treatment Using Implanted Microchips.

| Clinical history, treatment | | | | |
|---|---|---|---|---|
| methods and long-term | Clinical Examples | | | |
| results | 1 | 2 | 3 | 4 |
| Age, sex (m/f) | 45(F) | 68(F) | 54(M) | 50(M) |
| Duration of the disease (years) | 5 years | 26 years | 21 years | 12 years |
| Diagnosis | Depression Syndrome | Dementia | Dementia | Depression Syndrome |
| Associated diseases | Asthma | 1. Hypertension | Cirrhosis of the Liver | 1. Chronic |
| | | 2. Epilepsy | | Hepatitis |
| | | 3. Chronic | | 2. Schizophrenia |
| | | Hepatitis | | |
| Type of the chip implanted, | CHIP 2 | CHIP 4 | CHIP 2 | CHIP 3 |
| Name of the nerve the chip | Sinocarotid Collector | Sinocarotid Collector | Sympathetic Trunk, | Vagus Nerve |
| has been connected to | | | Cervical section | |
| Chip's program (programs) | DEPR-A | DEM-16 | DEM-4 | DEPR-12 |
| Duration of the surgery | 96 | 118 | 47 | 28 |
| (minutes) | | | | |
| Complication during and after | — | — | — | — |
| the operation, including | | | | |
| technology-related | | | | |
| complications | | | | |
| Reduction of daily intake of | A-Susp. | A-2,5 | B-1,4 | A-3,6 |
| medicines (X-times) after the | C-2,2 | B-2,1 | | D-Susp. |
| operation: | | | | |
| (before operation/one year | | | | |
| later) | | | | |
| A) Medicine name - | | | | |
| Amytriptiline | | | | |
| B) Medicine name - | | | | |
| Haloperidol | | | | |
| C) Medicine name - Sertraline | | | | |
| D) Medicine name - | | | | |
| Fluoxetine | | | | |
| Average annual duration of | 56/32 | 38/25 | 25/18 | 29/0 |
| hospitalization (sick leave), | | | | |
| days, | | | | |
| One year before the operation/ | | | | |
| last year | | | | |

| Clinical history, treatment | | | |
|---|---|---|---|
| methods and long-term | Clinical Examples | | |
| results | 5 | 6 | 7 |
| Age, sex (m/f) | 42(M) | 57(F) | 47(F) |
| Duration of the disease (years) | 7 years | 18 years | 9 years |
| Diagnosis | Depression Syndrome | Depression Syndrome | Depression Syndrome |
| Associated diseases | Asthma | 1. Asthma | Cirrhosis of the Liver |
| | | 2. Schizophrenia | |
| Type of the chip implanted, | CHIP 2 | CHIP 5 | CHIP 6 |
| Name of the nerve the chip | Sinocarotid Collector | 1. Sinocarotid | Vagus Nerve |
| has been connected to | | Collector | |
| | | 2. Vagus Nerve | |
| Chip's program (programs) | DEPR-15 | DEPR-16 | DEPR-120 |
| Duration of the surgery | 57 | 132 | 18 |
| (minutes) | | | |
| Complication during and after | — | — | — |
| the operation, including | | | |
| technology-related | | | |
| complications | | | |
| Reduction of daily intake of | | A-Susp. | A-1,5 |
| medicines (X-times) after the | | D-1,6 | C-Susp. |
| operation: | | | |
| (before operation/one year | | | |
| later) | | | |
| A) Medicine name - | | | |
| Amytriptiline | | | |
| B) Medicine name - | | | |
| Haloperidol | | | |
| C) Medicine name - Sertraline | | | |
| D) Medicine name - | | | |
| Fluoxetine | | | |
| Average annual duration of | 34/24 | 41/0 | 18/0 |
| hospitalization (sick leave), | | | |
| days, | | | |
| One year before the operation/ | | | |
| last year | | | |

| Clinical Examples | | |
|---|---|---|
| 5 | 6 | 7 |
| 42(M) | 57(F) | 47(F) |
| 7 years | 18 years | 9 years |
| Depression Syndrome | Depression Syndrome | Depression Syndrome |
| Asthma | 1. Asthma | Cirrhosis of the Liver |
| | 2. Schizophrenia | |
| CHIP 2 | CHIP 5 | CHIP 6 |
| Sinocarotid Collector | 1. Sinocarotid Collector | Vagus Nerve |
| | 2. Vagus Nerve | |
| DEPR-15 | DEPR-16 | DEPR-120 |
| 57 | 132 | 18 |
| — | — | — |
| | A-Susp. | A-1,5 |
| | D-1,6 | C-Susp. |
| 34/24 | 41/0 | 18/0 |

TABLE 19
examples of treatment of obliterating vascular diseases
Clinical examples of treatment of obliterating vascular diseases
of the lower limbs and pain syndromes with background angina pectoris using implanted microchips

| Medical | | | | | | |
|---|---|---|---|---|---|---|
| history, | | | | | | |
| treatment | Clinical Examples | | | | | |
| methods, | 1 | 2 | 3 | 4 | 5 | 6 |
| and long-term | Age, sex (m/f) | | | | | |
| results | 67-M | 59-M | 48-M | 46-M | 37-M | 61-M |
| Duration of the | 15 | 5 | 8 | 3 | 2 | 21 |
| disease (years) | | | | | | |
| Diagnosis, type | Obliterating vascular | Obliterating vascular | END (2B) | Obliterating vascular | END(3) | Obliterating |
| of seizures (in | endarteritis of the | endarteritis of the | | endarteritis of the lower | | vascular |
| the parenthesis) | lower limbs (degree 3) | lower limbs (degree | | limbs (degree 2)) | | endarteritis of the |
| | | 2) | | | | lower limbs (degree |
| | | | | | | 3) |
| Associated | Angina pectoris | Asthma | Asthma | Chronicobstructive | Chronicobstructive | Angina pectoris |
| diseases | | Hypertension | Angina pectoris | bronchitis | bronchitis | |
| Type of the | Chip 4, | Chip 5, | Chip 6, | Chip 4 | Chip 4 | Chip3 |
| chip Implanted | | | | | | |
| where and | 1. Spinal cord | 1. Spinal cord | 1. Spinal cord | 1. Spinal cord | 1. Spinal cord | 1. Spinal cord |
| how connected | 2. Sympathetic trunk, | 2. Sympathetic trunk, | | 2. Vagus nerve | | |
| | thoracic section | thoracic section | | 3. Sympathetic trunk, | | |
| | | | | thoracic section | | |
| Chip's program | AP-SP-STT-2 | OBL-SP-STT-18 | AP-10 | OBL-SP, VN, STT-2 | OBL-4 | AP-SP-17 |
| (programs) | | | | | | |
| Duration of the | 93 | 98 | 116 | 105 | 121 | 45 |
| surgery | | | | | | |
| (minutes) | | | | | | |
| After how | 25/60 | 180/500 | 200/350 | 160/300 | 25/100 | 20/25 |
| many meters of | | | | | | |
| walking did | | | | | | |
| pain in the | | | | | | |
| musculus | | | | | | |
| gastrocnemius | | | | | | |
| appear? Before | | | | | | |
| operation/3 | | | | | | |
| months after | | | | | | |
| operation | | | | | | |
| (Charcot's | | | | | | |
| syndrome) | | | | | | |
| Does the chip | + | + | + | + | + | + |
| dull the pain? | | | | | | |
| (+− Y, | | | | | | |
| −− N) | | | | | | |
| Average | 2.3 | 1.9 | 1.4 | 1.5 | 2.5 | 1.3 |
| reduction in | | | | | | |
| medications | | | | | | |
| doses 3 months | | | | | | |
| after operation | | | | | | |
| (X-times): | | | | | | |
| Value of | 15% | 18% | 20% | 10% | 22% | 9% |
| increase of | 2° C. | 1.8° C. | 2° C. | 1.2° C. | 1.6° C. | 0.9° C. |
| the capillary | | | | | | |
| circulation in | | | | | | |
| the shins | | | | | | |
| (%) (and their | | | | | | |
| temperature | | | | | | |
| in ° C.) at the | | | | | | |
| chip's | | | | | | |
| activation | | | | | | |
| Average annual | 30/7 | 32/0 | 28/10 | 20/0 | 35/20 | 31/30 |
| duration of | | | | | | |
| hospitalization | | | | | | |
| (sick leave), | | | | | | |
| days, One year | | | | | | |
| before the | | | | | | |
| operation/last | | | | | | |
| year | | | | | | |

TABLE 20
Conditions in a healthy patient that may be treated
LIST OF CONDITIONS OF THE HEALTHY PERSON'S BODY
THAT CAN BE CORRECTED WITH THE CHIPS

| | | Expected effect from the |
|---|---|---|
| | | microchip's application (a |
| | Name of Condition (a reference | reference to the technology |
| | to a relevant code in ISD-10 is | No. in the Patent is |
| No. | provided in the parentheses) | provided in the parentheses) |
| 1 | Skin. subcutaneous fat | Shaping, weight loss |
| | Excessive development of the | (liposuction) |
| | subcutaneous fat in different parts | (Technology No. . . . ) |
| | of the body (ISD-10 | |
| | Code: . . . ) | |
| 2 | Turgor decrease. skin elasticity | Skin anti-aging effect |
| | loss, wrinkles | (Technology No. . . . ) |
| | (ISD-10 Code: . . . ) | |
| 3 | Excessive skin dryness or | [symbol] |
| | excessive sweating (ISD-10) | (Technology No. . . . ) |
| | Code: . . . ) | |
| 4 | Muscular System | Shaping. increase of the |
| | Flabbiness of the muscles, their | muscle mass and strength |
| | tonicity degradation due to | (Technology No. . . . ) |
| | hypodynamia. Fitness and body | |
| | shape deterioration | |
| | (ISD-10 Code: . . . ) | |
| 5 | Mammal Glands | Improvement of the shape |
| | Small size. underdeveloped | and function |
| | glandular tissue | (Technology No. . . . ) |
| | [symbol] | |
| | [symbol] | |
| | (ISD-10 Code: . . . ) | |

1. A medical instrument implanted in a patient's body, which uses electrical and/or electromagnetic means of stimulation and/or inhibition (blocking) for the treatment of various diseases or specific states in a healthy person.

2. The medical instrument according to claim 1, wherein the diseases include asthma, Agranulocytosis, Anemia, Atrophy of he optic nerve, Skin atrophy, Sardiac achalasia, Coagulation disorders, Buerger's disease, Crohn's disease, Pick's disease, Raynaud's disease, Vegetovascular dystony, Sudden cardiac arrest at sleep, Rectal prolapse, Flaccidly consolidating fractures, Hyperkeratosis, Hyperostosis, Adrenal cortex hyperfunction, Hypoglycemia, Subcortical dementia, Multi-infarction dementia Involuntary movements, Dyskinesia of the biliferous tracts, Dysmenorrhea (genital diseases), Dolichosigmoid, Stammering, Impotence, Leucosis, Migraine, Myodystrophy, Myopathy, Male and female infertility, Narcolepsy, Bauhin's valve failure, Neurodermite, Obliterating atherosclerosis of the limbs' vessels, Obliterating endarteritis of the lower limbs' vessels, Alopecia, Paralyses of different etiology, Paresis of the vocal cords, Progressing myodystrophy, Psoriasis, Rickets, Zollinger-Ellison syndrome, Commissural disease, Huntington's chorea, Chronic duodenal ileus, Chronic and acute hyperthermia, Chronic gastroenterocolitis, Chronic cholecystitis and cholecystopancreatitis, Chronic cystitis, Chronic glomerulonephritis, Chronic obstructive bronchitis, Chronic osteomyelitis, Chronic pyelonephritis, Cirrhosis, Eczema, Pulmonary emphysema, Enuresis and/or Duodenal ulcer,

3. The medical instrument according to claim 1, wherein the specific states in a healthy person include dieting, losing wrinkles, restoring skin flexibility, dry skin, excessive perspiration, increasing the volume and power of muscles, enlarging or reducing breasts size.

4. The medical instrument according to claim 1, comprising sensor means for measuring variables inside the body, processor means for processing the signals and issuing therapeutical signals, and electrodes for applying these signals, all implanted in the patient's body, and wherein the sensor means include one or more of sensors and/or biosensors means.

5. The medical instrument according to claim 1, wherein the processor means comprises digital computer means for processing information received from sensor means and for generating stimulation and/or inhibition therapeutical signals responsive to the received information, the therapeutical signals are transferred to electrodes by wire and/or wireless means, wireless means include RF/electromagnetic waves, the number of electrodes may vary between one to several hundred, and an electrode is used to apply a therapeutical signal to a target body tissue or nerve or organ.

6. The medical instrument according to claim 1, wherein the characteristics of the applied signal are adjusted to effective values according to the disease or body state to be treated, preset parameters and signals received from the sensor means, these characteristics include the amplitude of the signal, its duration and/or frequency.

7. The medical instrument according to claim 1, wherein the electrodes operate concurrently to apply signals to the patient's body, each electrode may convey a separate signal from the processor means, and each signal is devised using a different algorithm, as suitable for a specific disease and a specific target organ.

8. The medical instrument according to claim 1, wherein each electrode is be implanted in a different location in the body, to allow a plurality of electrodes to be implanted in various places, according to a patient's disease or physiological situation to be treated.

9. The medical instrument according to claim 1, wherein, for purposes of treatment of various diseases in one patient, the electric stimulator's electrodes are simultaneously connected to different tissues, nervous structures, organs directly relating to each specific disease, while a separate channel or channels of the Electric Stimulator, its separate program or programs, separate non-interrelated algorithm or algorithms are used for treatment of each respective disease in the patient.

10. The medical instrument according to claim 1, wherein the algorithms in the processor means are programmed in advance, and/or are updated in real time, the system may learn and adapt itself according to signals from the body, or may receive external programming signals.

11. The medical instrument according to claim 1, wherein the activation oft the electrodes, under the processor's control, occur in several modes, alternately or as set up:
A. Automatic mode, using a feedback circuit comprising the sensor means, the processor and the electrodes;
B. On demand mode from a preset program stored in the processor
C. Manual mode, initiated by patient or the physician.

12. The medical instrument according to claim 1, wherein the information received by the sensor means is transferred to the processor, where it is processed according to the patient's disease or physical state, and the processor selects only the sensors which are relevant in each case.

13. The medical instrument according to claim 1, further including means for providing electrical power to the instrument, including an implanted primary or secondary battery, wherein the secondary battery may be recharged from an external source, connected for example through wires or wireless such as RF waves, and the system may include means for converting the body kinetic energy to electrical energy that may charge the battery.

14. The medical instrument according to claim 1, further including one or more additional processors, each implanted in a different place in the body; an optional central processor collects and processes data from the various processors in the body.

15. The medical instrument according to claim 1, wherein the electrode is implanted to affect systems and organs of the body including the nervous structure of the sympathetic nervous system or the parasympathetic system or the sympathetic nervous system and parasympathetic system and hypoglossal (sinocarotid collector of the Vegetative Nervous System-SCVNS), the central nervous system, as well as neurons of the organ and/or cutaneous nerves and/or depressor nerves.

16. The medical instrument according to claim 15, wherein the electrode is implanted to affect a nervous band or group comprising all, or the majority of, the nerve branches innervating the carotid glome (glomus caroticum).

17. A medical treatment method using an instrument implanted in a patient's body, which uses electrical and/or electromagnetic means of stimulation and/or inhibition for the treatment of various diseases or specific states in a healthy person.

18. The medical treatment method according to claim 17 which, for stimulating and/or inhibiting the functional activity of the body's systems and separate organs and tissues, uses pulse currents of parameters as detailed in the present disclosure.

19. The medical treatment method according to claim 17 which, in order to control functioning of the implanted stimulator and to set its optimal mode, further including the step where the patient is connected (for the period varying between 1 and several days) to a portable external non-implantable monitor collecting and analyzing data on the Electric Stimulator's work, as well as data on a functional condition of the body's systems, organs, and tissues, while this said monitor comprises a unit analyzing functional conditions of the body's systems, organs, and tissues, connected to the sensor means, and this unit is also connected to the monitor's unit of radio-frequency communication with the Electric Stimulator's external radio-frequency communication unit which, in its turn, is connected to a computer via a radio-frequency channel, as well as to the autonomous power supply unit, the latter also being connected to all the above-detailed units of the monitor.

20. The medical treatment method according to claim 17 further including the step, performed using the Electric Stimulator's software, of distinguishing between the changes in functional activity of the body's systems, and/or organs, and/or tissues typical of an onset of a disease (symptoms) and the changes in functional activity of the body's systems, and/or organs, and/or tissues, that are not related to symptoms of a disease, but typically occur in the patient's body, while the Electric Stimulator's “learning” of this process is aided by a non-implanted display.

21. The medical instrument according to claim 1, further including means for electric stimulation and/or blocking of the body tissues, comprising sensor means for measuring variables in the body, processor means connected to the sensors and biosensors for processing the measured variables and for deciding in real time whether to apply an electric signal to the body tissues, and electrode means implanted at predefined locations and connected to the processor means, for applying the stimulation and/or electric blocking signals to the body tissues.

22. The medical instrument according to claim 1, wherein its sensors and biosensors located on the biologically inert coating thereof, contain, as an ingredient of their receptors, the porous conducting mineral Schungite with self-cleaning pores, while the sensors and biosensors are connected to the electric stimulator's units.

23. The medical instrument according to claim 1, wherein the Electric Stimulator has the surfaces of its shell and of the electrodes made to also host contacts, in addition to sensors and biosensors, which, when implementing algorithms of the Electric Stimulator's program, enable to selectively connect either a certain number of the sensors, biosensors, contacts or all of them together to the Electric Stimulator's units, thus creating a required configuration of the electrode(s), with a specific location and number of sensors, biosensors, this configuration being best adapted to accomplish the preliminarily set specific goals and objectives.

24. The medical instrument according to claim 1, further including tiny metallic plates which send electrical signals, preferably at a high rate, towards a body tissue; This enables to measure the electrical resistance of the tissue and its changes responsive to activating signals from the instrument's processor, before, during and after the activation of a therapeutic sequence; this gives an indirect measurement of the patient's body behavior.

25. The medical treatment method according to claim 17 wherein, using electrical measurements, use changes in resistance, which are compared with the readings from the sensor means; The system thus learns the correlation between the sensor means readings and the indirect measurements; when the original sensor means eventually are put out of use because of organic matter deposits thereon, the indirect measurements can still be used to evaluate the body's conditions; the system thus includes a structure with means for learning the patient body's characteristics; a method for learning these characteristics and replacing the original sensor means with indirect sensors has been disclosed.

26. The medical instrument according to claim 1, wherein the contacts of the Electric Stimulator can have different shapes which allows to connect them to both surfaces of the tissues, organs, and their internal structures.

27. The medical instrument according to claim 1, wherein the the external coating of the Electric Stimulator may host from a few to thousands contacts, while each of the latter can be connected to either one, several or all channels of the Electric Stimulator.

28. The medical instrument according to claim 1, wherein the contacts of the Electric Stimulator can have different shapes, sizes, locations, while each of the contacts may be connected to both one or a specific group of channels and all the channels together, and wherein The connection can be either algorithm-based or supported by the relevant design solution.

29. The medical instrument according to claim 1, wherein the processor means contain all or part of the elements No. 1,2,3,5,6,8,9,10,11,14,15,16,17,18,19 as indicated in Table 1.

30. The medical instrument according to claim 1, wherein the processor includes means for activating from one electrode up to several hundred electrodes, and wherein Each electrode may receive a different activating signal from the processor, according to a different algorithm, and/or for a different disease; The processor includes means for receiving signals from one sensor means, and up to several hundreds sensor means units, of one type or of various types.

31. The medical instrument according to claim 1, wherein the processor may compute activating signals for one disease or for a plurality of diseases, up to several hundred diseases concurrently; the required signals are then applied to the patient's organs through the electrodes; the processor also decides, in each case, whether to apply stimulating or inhibiting signals, according to information from the sensor means and/or the clinical findings.

32. The medical instrument according to claim 1, wherein the Electric Stimulator has the feature that it has channels (units generating output pulses or units generating address codes, connected to the relevant electrodes) in the amount of 1 up to thousands, while each of these channels can be controlled with different algorithms of the programs.

33. The medical instrument according to claim 1, wherein the Electric Stimulator's shell contains the unit generating address codes and output pulses for the electrodes, connected to the electromagnetic communication unit of the implanted, enabling a wireless connection between the Electric Stimulator and with the implanted or non-implanted electrodes, while in order to support connection with the electrodes it is possible to use both individual address codes for each electrode and codeless non-address communication.

34. The medical instrument according to claim 1, wherein the Electric Stimulator contains an option (a software option) to enable the Electric Stimulator's programming and control functions by means of connecting telecommunications channels, including the Internet, radio channels, satellite communication channels, fiber optics communication channels, cellular telephone channels, etc, to the external radio-frequency communication unit or the internal radio-frequency communication unit of the Electric Stimulator.

35. The medical instrument according to claim 1, wherein the Electric Stimulator contains means for controlling the duration and frequency of electric stimulation sessions can be carried our using the Electric Stimulator's programmable microprocessor (unit(s) generating output pulses and/or the unit generating address codes and output pulses for the electrodes), or using the Electric Stimulator's timer based on the signals sent by the sensors and/or biosensors of different types, while setting up the duration and frequency of electric stimulation sessions is effected separately, aided by different sensors and biosensors (for example, session duration is controlled with the respiration rate sensors, while the frequency is set up with the heart rate sensor).

36. The medical instrument according to claim 1, wherein the Electric Stimulator contains means for activation of the channel(s) of its unit(s) generating output pulses and/or unit generating address codes and output pulses for the electrodes, as well as selection of the program(s) and algorithm(s) for stimulation and/or inhibition of functional activity of the body's organs and tissues can be carried out in the three following modes (using each other separately, or in different combinations between them, the three together simultaneously): first mode,—automatic mode set up in accordance with the type of signals transmitted by the sensors, biosensors to the Electric Stimulator's units, as well as per the input algorithm(s) of the program(s); second mode,—set up regardless of the signals transmitted by the sensors, biosensors, depending on the input algorithm(s) of the program(s) only; third mode,—direct (voluntary) control of the above-mentioned functions and programs of the Electric Stimulator by the patient or doctor using the external radio-frequency communication unit of the Electric Stimulator or the internal radio-frequency communication unit thereof.

37. The medical treatment method according to claim 17, wherein, using means in the Electric Stimulator that makes it resemble the natural functioning of separate nervous centers and the entire human nervous system, further include the step, in addition to the existing involuntary algorithms and programs, controlling functions of the systems and organs, there is also an option to voluntarily control a number of the body's organs and systems (for example, voluntary regulation of respiration rate, breath depth, etc.)

38. The medical treatment method according to claim 81, wherein the Electric Stimulator and Therapy Method has two following modes of operation, an they can be activated both simultaneously and separately: First Mode—supports regular (preventive) electric stimulation sessions for the prevention of acute symptoms of the disease; Second Mode—supports therapeutic electric stimulation sessions, and it is activated when acute symptoms of the disease (attacks, seizures, etc.) occur; As a result, as it also in the case of the First Mode, functional activity of the body's systems, and/or organs, and/or tissues is stimulated and/or inhibited.

39. The medical treatment method according to claim 17, wherein, in order to assess efficiency of the implanted Stimulator, functional activity of the body's systems, and/or organs, and/or tissues is studied before conducting electric stimulation sessions and thereafter using the sensors, biosensors as well as the methods widely used in the common clinical practice, while a detected decrease in the functional activity of the body's systems, and/or organs, and/or tissues is deemed an inhibition symptom, while an enhancement thereof is considered to be a symptom of stimulation of the functional activity of the body's systems, and/or organs, and/or tissues.

40. The medical instrument according to claim 1, wherein the Electric Stimulator or electric stimulators include means that allow the device to be controlled with one or several similar implanted electric stimulators, while all of the these “affiliated” implanted electric stimulators are controlled with one main electric stimulator, that has the same units, but different algorithms.

41. The medical instrument according to claim 1, further including a noncontact communication channel with a receiver outside a patient's body, and wherein the processor further includes means for sending the measured variables to the external receiver.

42. The medical treatment method according to claim 17, wherein a change of the stimulation mode (stimulation session frequency and session duration) depend on the sensor's signal value (that, in its turn, depends on a particular time period of the day, the patient's activity, and severity of symptoms of the disease).

43. The medical treatment method according to claim 17, further including the step of Accurate adaptation of the stimulation mode to individual physical activity of the patient's body or to a symptoms severity degree of any specific ailment.

44. The medical treatment method according to claim 17, further including an increase or reduction of current, voltage, output pulses duration, and stimulation sessions frequency and duration by increasing or reducing signals of biological sensors, wherein:
1) Biological sensors are located directly on the chip and electrode housing;
2) The majority of chip units are connected with its radiofrequency communication component, which enables a direct control and adaptability thereof by means of the external programming unit;
3) Stimulation sessions and intervals of determining the nerve electric activity proceed in succession and not concurrently;
4) The chip operation modes are programmed by external programming unit (setup of simulation thresholds, working algorithms, etc.);
5) Power battery of the stand-alone power unit is charged by the following two sources: external programming unit, and bioelectric activity of nerve; power supply to the chip can be provided by electromagnetic waves transmitted from the external programming unit;
6) To add reliability, onset of seizure or deterioration of other ailment symptoms are identified according to a set of parameters;
7) Indicator informs the patients, when the stimulation device starts and finishes working.

45. The medical treatment method according to claim 17, further including an increase or reduction of current, voltage, output pulses' duration, and stimulation sessions frequency and duration by increasing or reducing signals of biological sensors, wherein:
1) Increase or reduction of current, voltage, output pulses duration, and stimulation sessions frequency and duration via each channel depending on an increase or reduction of signals of sensors and biological sensors;
2) Changing of sequence and combination of activated chip channels depending on the biosensors' signals values;
3) A parallel control of several organs using various stimulation programs to resume their functions;
4) Biosensors design, concurrent monitoring of the state of several body organs and systems;
5) Use of at least two isolated channels to control each organ or system in order to provide a concurrent effect on several nerve centers and optimize the results of treatment.

46. The medical treatment method according to claim 17, further including an increase or reduction of current, voltage, output pulses duration, and stimulation sessions frequency and duration by increasing or reducing signals of biological sensors, wherein:
1) Increase or reduction of current, voltage, output pulses duration, and stimulation sessions frequency and duration via each channel depending on an increase or reduction of signals of sensors and biological sensors;
2) Changing of sequence and combination of activated chip channels depending on the current state of stimulated organs and tissues, as determined by biosensors;
3) Wireless implantable families of secondary microelectrodes with individual programming of operation modes and parent chip connection by means of electromagnetic waves;
4) Biosensors design;
5) Monitoring of body systems and functions by means of various biological sensors,
6) Concurrent stimulation of over 100 nerves, organs, and muscles using various programs with a parallel monitoring of the results.

47. The medical instrument according to claim 1, further including means for its being powered from electromagnetic waves transmitted by the external radio-frequency communication unit to the internal radio-frequency communication unit, and it also can be powered by the autonomous power supply unit capable of automatic recharging from the patient's body kinetic energy.

48. The medical instrument according to claim 1, further including autonomous power supply unit which is charged from the patient's body kinetic energy, for which purpose this said power supply unit is connected with an electrode, via a transducer, to an adaptor converting mechanical motions of the diaphragmatic muscle occurring during the process of breathing into electric current—A flexible piezoelectric element (electrode-like) coated with a biologically inert material; This element is implanted under the diaphragm's cupola, from the right side, endoscopically, and it is connected to the chip; The chip comprises an AC/DC transducer.

49. The medical instrument according to claim 1, wherein its unit or units for generating output pulses, as well as its unit generating address codes are programmable via the external radio frequency communication unit and via the internal radio frequency communication unit (or they are non-programmable while being operated via the above-mentioned communication units).

50. The medical instrument according to claim 1, wherein, to increase the device's safety level, it can be powered from the three following sources: autonomous power unit (battery), and/or kinetic energy of the patient's body and/or by means of electromagnetic waves from the external non-implanted display, and/or remote control unit, while selecting of a source or sources is enabled by the algorithms of the software input into the Electric Stimulator, and/or remote control unit, and/or display.

51. The medical instrument according to claim 1, wherein the processor is multifunctional, including means for its operation in one of several predefined modes.

52. The medical instrument according to claim 1, wherein it includes means for a therapeutic effect applicable to treatment of diseases, and to correcting functional activity of the body's systems, and/or organs and/or tissues in healthy persons, using the self-adjusting algorithms that adapts the device to current needs of the body, while these said algorithms can be selected with the remote control unit and/or display.

53. The medical instrument according to claim 1, wherein it includes means for enabling the algorithm(s) of its program(s) to be changed in real-time depending on type of the signals sent by the sensors and/or biosensors, and due to this feature the optimal and most efficient algorithm of the program is selected, this being best adapted to the needs of the patient's body in a specific period of time, to allow Dynamic, self-adjusting algorithms.

54. The medical treatment method according to claim 17, wherein its external radio-frequency communication unit and the internal radio-frequency communication unit enable and support a reciprocal connection between external non-implanted devices and each of the Electric Stimulator's units and/or channels separately, while address codes may be used for this purpose.

55. The medical treatment method according to claim 17, wherein a Therapy Method and Method for Correction of Functional Activity of the Body's Systems, and or Organs; and/or Tissues are used to identify, to detect certain conditions of the body using the sensors and biosensors, one or several indirect parameters may be considered, these parameters being typical of each specific patient; in order to identify these typical indirect properties, functional activity of the body's systems, organs, tissues is repeatedly (twice and up to several times) examined and analyzed when the above-mentioned conditions become pronounced using the display; or applying the methods and equipment widely used in the clinical practice, thus establishing regular changes in the selected homeostatic parameters, which are to be taken into account for purposes of identification of the above-mentioned conditions at a later stage.

56. The medical treatment method according to claim 109, wherein upon development of the hypoglycemic coma in one of the patients, the following repeated regular changes are observed: a drop in the arterial pressure of at least 20 mm of the mercury column, a minimal pulse acceleration of 30 beats per minutes a 30% acceleration of the respiration rate, while these changes typically coincide with a drop in the blood glucose level amounting to 50% deviation from the norm.

57. The medical treatment method according to claim 17, wherein to obtain the maximal therapeutic effect, prior to using the Electric Stimulator, the patient is examined in order to detect typical disease-related phenomena, this being followed by the development of individual programs and algorithms of the Electric Stimulator aimed at correcting main adverse effects of the disease(s).

58. The medical treatment method according to claim 17, wherein general coordination of interrelated operation of all the channels, and control of functional activity of the body's systems, and/or organs, and/or tissues with stimulation and/or inhibition via the above-mentioned channels, is supported with a separate (main) algorithm (a “visceral conductor”) of the program, while this said algorithm can flexibly perform real-time changes thus optimally adapting the Electric Stimulator's channels to current needs of the patient body.

59. The medical treatment method according to claim 17, wherein the function of algorithms in the software of the unit(s) generating output pulses, or of the unit generating address codes and output pulses for the electrodes is based on identification of code signals sent by sensors and biosensors of various types, while each of these signals reflects a singular specific condition of the patient and or his/her body's systems, and/or organs, and/or tissues (for example, Code 1 sent by Sensor No. 1 identifies the patient's condition during night time, when a spasm of the bronchi is at an onset, and, as a result wheezing in the lung appears, while Code 2 sent by Sensor 1 reflects the patient's condition during night time when no asphyxia attack and spasm of the bronchi occur), and by means of analyzing such codes Electric Stimulator's CPU generates commands to activate and/or deactivate electric stimulation through one or several channels, commands to set the optimal current parameters, stimulation modes, aimed at obtaining physiological effects of the stimulation and/or inhibition of functional activity of the body's systems, and/or organs, and/or tissues (object-oriented functional programming).

60. The medical instrument according to claim 1, wherein the electrode means include a plurality of electrodes implanted in several locations in the patient's body.

61. The medical instrument according to claim 1, wherein, for purposes of stimulation and/or inhibition of the functional activity of the body's systems, organs and tissues, electrically non-interconnected implanted electrodes of 0.13 microns up to 15 mm in size in the amount varying between 1 up to thousands are simultaneously used, while these said electrodes are connected to the electromagnetic communication unit in a wireless fashion, via electromagnetic and/or other waves or they are connected with wires to the unit generating output pulses and/or to the unit generating address codes and output pulses.

62. The medical instrument according to claim 1, wherein the implanted or non-implanted electrodes connected to the electromagnetic communication unit of the Electric Stimulator in a wireless fashion, via electromagnetic and/or other waves contain, in their shell, the electromagnetic communication and power supply unit, the latter being connected, either directly or via the electrode's decoder, to the unit generating output pulses, which in its turn is connected to a contact or contacts of the wireless electrode, while the electrode's memory unit can also be connected to the unit generating output pulses, and the electrode's power supply unit can be connected to all the above-mentioned units of the electrode.

63. The medical instrument according to claim 1 wherein its shell can also simultaneously perform the function of an electrode or electrodes in addition to its main function; a “Tablet-shape” electric stimulator can be achieved.

64. The medical instrument according to claim 1, wherein for purposes of electric stimulation or inhibition of functional activity of the body's systems, organs and tissues; electrodes of different shapes varying in number of current conducting cores (wires) and contacts (channels) are used, while these electrodes are connected, either in a wireless fashion or with wires, to the unit generating output pulses or to the electromagnetic communication unit of the Electric Stimulator.

65. The medical instrument according to claim 1 further including an audio and/or light, and/or vibration, and/or temperature, and/or cutaneoelectric indicating device providing the patient with data on conditions of the Electric Stimulator, his/her own body, as well as with other information transmitted by the external and/or internal radio-frequency communications units to the Electric Stimulator, is connected to the unit generating output pulses, and/or to the unit generating address codes and output pulses for the electrodes, and/or to the internal radio-frequency communication unit, and/or to the external radio-frequency communication unit, and/or to an electrode, and/or to electrodes.

66. The medical instrument according to claim 1, further including in addition to analyzing functional activity of the body's systems, and/or organs, and/or tissues, as well as controlling and analyzing operation and functioning of the implanted Stimulator, the monitor also supports programming or reprogramming of the Electric Stimulator (by means of a computer connected thereto via the radio-frequency communication unit).

67. The medical instrument according to claim 1, further including means for running a long-term monitoring of functional activity of the body's systems, organs, tissues, and operation of the Electric Stimulator; while duration of the monitoring may vary between several minutes and several months.

68. The medical instrument according to claim 1, further including External Non-Implanted Display-Programmer—Charging Device and wherein the sensors and biosensors representing micro- and macro-indicating devices of different, located both on the patient's body surface and directly introduced into its tissues, organs, systems (for example, arterial pressure meter introduced into the femoral artery) collect data on functioning of the body's systems, and wherein these data are transmitted to the unit analyzing conditions of the body's systems, organs, tissues (No. 3 on the Diagram). The unit separately analyzes functioning of each of the systems studied enabling both fragmentary and permanent real-time monitoring of the body's systems, organs, tissues.

69. The medical instrument according to claim 1, further including means to recharge the battery of the Electric Stimulator, and/or to program the latter via the Unit No. 4.

70. The medical instrument according to claim 1, wherein the means for covering the processor (chip) from the outside include a Silicon coating.

71. The medical instrument according to claim 1, wherein the means for covering the processor (chip) from the outside include a Silicon mixed with Shungite.

72. The medical instrument according to claim 1, wherein the means for covering the processor (chip) from the outside include Silicon completely covered on the outside with Shungite.

73. The medical instrument according to claim 1, wherein the electric stimulator include coating means that are external biologically inert coating is made of a silicon compound whose molecular mass varies between a few and a thousand units, while this coating can host sensors, biosensors, contacts connected to the Electric Stimulator's units.

74. The medical instrument according to claim 1, wherein the means for covering the processor (chip) are external biologically inert coating is made of a porous material, while the pores-size varies between a few nanometers and a few micrometers, and the pores themselves are evenly spread all over the entire coating surface with their concentration on one square millimeter.

75. The medical treatment method according to claim 17, wherein, in order to obtain a therapeutic effect, the electrode or electrodes of the Electric Stimulator are implanted into the subcutaneous fat, and/or into nerves (vagus nerve and or sinocarotid), and/or into various organs and/or into blood vessels and/or into the spinal cord, and/or into bones or into the medullar canal, are connected to different areas of the skin with the Electric Stimulator being implanted, in accordance with specific treatment techniques for each of the diseases.

76. The medical treatment method according to claim 17, wherein the electrode is implanted to affect systems and organs of the body including the nervous structure of the sympathetic nervous system or the parasympathetic system or the sympathetic nervous system and parasympathetic system and hypoglossal (sinocarotid collector of the Vegetative Nervous System-SCVNS), the central nervous system, as well as neurons of the organ and/or cutaneous nerves and/or depressor nerves.

77. The medical treatment method according to claim 17, wherein the electrode is implanted to affect a nervous band or group comprising all, or the majority of, the nerve branches innervating the carotid glome (glomus caroticum).

78. The medical treatment method according to claim 17, wherein the electrode is implanted to affect systems and/or organs of the body as indicated in Tables 1 to 19 of the present disclosure.

79. The medical treatment method according to claim 17, wherein the method of treatment by electro-stimulator as applied for treating Asthma, using one or more electrodes as detailed in claim X, wherein the electrodes are implanted in the following locations inside a patient's body:
Best mode—right sinocarotid collector (over the bifurcation spot of the common carotid artery) and/or left sinocarotid collector (over the bifurcation spot of the common carotid artery) or Second best mode—right sympathetic trunk, thoracic (in the superior third, at the level T2-T4) and/or left sympathetic trunk, thoracic (in the superior third, at the level T7-T-11); while using the following sensors: Arterial pressure meter, Heart rate meter, Respiration rate meter, Temperature gage, Angular shift sensor (for the limbs), Local blood flow sensor. Sensor of electric activity of the organs nervous centers: Murmur sensor (heart, lungs: intestine), while using the following bio-sensors:
Biosensor of oxygen contents in the tissues
Biosensor of sugar contents in the blood
and while the electro-stimulation parameters are as follows:
Current frequency: 1-300 HZ, Duration of current pulses: 0.1-0.8 Ms,
Amplitude of current pulses: 0.02-1 V.

80. The medical treatment method according to claim 17, wherein treatment by electro-stimulator for treating Epilepsy, using one or more electrodes wherein the electrodes are implanted in the following locations inside a patient's body:
Best mode—right sinocarotid collector (over the bifurcation spot of the common carotid artery) and/or left sinocarotid collector (over the bifurcation spot of the common carotid artery) or Second best mode—left Vagus nerve (in the superior third of the neck, at the level C2-C3) and/or right Vagus nerve (in the superior third of the neck, at the level C2-C3) while using the following sensors: Arterial pressure meter, Heart rate meter, Respiration rate meter. Temperature gage, Angular shift sensor (for the limbs), Local blood flow sensor, Sensor of electric activity of the organs: nervous centers, Murmur sensor (heart, lungs, intestine), while using the following bio-sensors:
Biosensor of oxygen contents in the tissues. Biosensor of sugar contents in the blood, Biosensor of hormone contents in the blood, Biosensor of alcohol contents in the blood
and while the electro-stimulation parameters are as follows:
Current frequency: 1-300 HZ, Duration of current pulses: 0.1-0.8 Ms,
Amplitude of current pulses: 0.02-1 V.

81. The medical treatment method according to claim 17, wherein for treating Obesity, wherein the electrodes are implanted in the following locations inside a patient's body:
Best mode—Left vagus nerve (in the superior third of the neck, at the level C2-C3) and/or right vagus nerve (in the superior third of the neck, at the level C2-C3) or Second best mode—left sympathetic trunk, thoracic (in the superior third, at the level T7-T-11) and/or right sympathetic trunk, thoracic (in the superior third, at the level T2-T4) while using the following sensors:
Arterial pressure meter. Heart rate meter. Respiration rate meter, Temperature gage, Angular shift sensor (for the limbs), Local blood flow sensor, Sensor of electric activity of the organs, nervous centers, Gastric juice acidity sensor, while using the following bio-sensors:
Biosensor of oxygen contents in the tissues, Biosensor of sugar contents in the blood, and while the electro-stimulation parameters are as follows:
Current frequency: 1-300 HZ, Duration of current pulses: 0.1-0.8 Ms,
Amplitude of current pulses: 0.02-1 V.

82. The medical treatment method according to claim 17, wherein for treating Hypertension, wherein the electrodes are implanted in the following locations inside a patient's body:
Best mode—Right depressor nerve (over the aortic arch or in the middle third of the neck) and/or left depressor nerve (over the aortic arch or in the middle third of the neck) or Second best mode—Right sinocarotid collector (over the bifurcation spot of the common carotid artery) and/or left sinocarotid collector (over the bifurcation spot of the common carotid artery) while using the following sensors: Arterial pressure meter, Heart rate meter, Respiration rate meter, Temperature gage, Angular shift sensor (for the limbs), Local blood flow sensor, Sensor of electric activity of the organs, nervous centers Murmur sensor (heart, lungs, intestine), while using the following bio-sensors: Biosensor of oxygen contents in the tissues, Biosensor of sugar contents in the blood, and while the electro-stimulation parameters are as follows: Current frequency: 1-300 HZ, Duration of current pulses: 0.1-0.8 Ms. Amplitude of current pulses: 0.02-1 V.

83. The medical treatment method according to claim 17, wherein for treating Diabetes, wherein the electrodes are implanted in the following locations inside a patient's body: Best mode—left vagus nerve (in the superior third of the neck, at the level C2-C3) and/or right vagus nerve (in the superior third of the neck, at the level C2-C3) or Second best mode—Left sympathetic trunk, thoracic (in the superior third, at the level T7-T-11) and/or right sympathetic trunk, thoracic (in the superior third, at the level T2-T4) while using the following sensors: Arterial pressure meter, Heart rate meter; Respiration rate meter, Temperature gage, Angular shift sensor (for the limbs), Local blood flow sensor. Sensor of electric activity of the organs, nervous centers Murmur sensor (heart, lungs: intestine), while using the following bio-sensors: Biosensor of oxygen contents in the tissues, Biosensor of sugar contents in the blood. Biosensor of hormone contents in the blood and while the electro-stimulation parameters are as follows:
Current frequency: 1-300 HZ, Duration of current pulses: 0.1-0.8 Ms,
Amplitude of current pulses: 0.02-1 V.

84. The medical treatment method according to claim 17, wherein for treating Angina Pectoris, wherein the electrodes are implanted in the following locations inside a patient's body: Best mode—right sinocarotid collector (over the bifurcation spot of the common carotid artery) and/or left sinocarotid collector (over the bifurcation spot of the common carotid artery) or Second best mode—Spinal cord (at the level T10-T-12) while using the following sensors: Arterial pressure meter, Heart rate meter, Respiration rate meter Temperature gage, Angular shift sensor (for the limbs), Local blood flow sensor. Sensor of electric activity of the organs, nervous centers, Murmur sensor (heart, lungs, intestine): while using the following bio-sensors:
Biosensor of oxygen contents in the tissues and while the electro-stimulation parameters are as follows:
Current frequency: 1-300 HZ, Duration of current pulses: 0.1-0.8 Ms,
Amplitude of current pulses: 0.02-1 V.

85. The medical treatment method according to claim 17, wherein for treating Alcoholism, wherein the electrodes are implanted in the following locations inside a patient's body: Best mode—Right depressor nerve, (over the aortic arch or in the middle third of the neck) and/or left depressor nerve (over the aortic arch or in the middle third of the neck) or Second best mode—Left vagus nerve (in the superior third of the neck, at the level C2-C3) and/or right vagus nerve (in the superior third of the neck, at the level C2-C3) and/or Nerve collector of the organ (skin) while using the following sensors:
Arterial pressure meter. Heart rate meter, Respiration rate meter. Temperature gage, Angular shift sensor (for the limbs), Local blood flow sensor. Sensor of electric activity of the organs, nervous centers. Murmur sensor (heart, lungs, intestine), while using the following bio-sensors:
Biosensor of oxygen contents in the tissues. Biosensor of sugar contents in the blood. Biosensor of alcohol contents in the blood
Biosensor of narcotic substances contents in the blood and while the electro-stimulation parameters are as follows:
Current frequency: 1-300 HZ, Duration of current pulses: 0.1-0.8 Ms.
Amplitude of current pulses: 0.02-1 V.

86. The method of treatment by electro-stimulator as detailed in claim 17 applied for treating Drug addiction, using one or more electrodes as detailed in claim X, wherein the electrodes are implanted in the following locations inside a patient's body:
Left vagus nerve (in the superior third of the neck, at the level C2-C3) and/or right vagus nerve (in the superior third of the neck, at the level C2-C3) and/or Left sympathetic trunk, thoracic (in the superior third, at the level T7-T-11) and/or right sympathetic trunk, thoracic (in the superior third, at the level T2-T4) and/or Right sympathetic trunk; cervical (in the superior third of the neck, at the level C2-C3) or Left sympathetic trunk (Left upper cervical third part between C2-C3) and/or right sinocarotid collector (over the bifurcation spot of the common carotid artery) and/or left sinocarotid collector (over the bifurcation spot of the common carotid artery) and/or Spinal cord and/or right depressor nerve (over the aortic arch or in the middle third of the neck) and/or left depressor nerve (over the aortic arch or in the middle third of the neck) and/or Nerve collector of the organ while using the following sensors:
Arterial pressure meter, Heart rate meter, Respiration rate meter. Temperature gage, Angular shift sensor (for the limbs), Local blood flow sensor, Sensor of electric activity of the organs, nervous centers. Murmur sensor (heart, lungs, intestine), while using the following bio-sensors:
Biosensor of oxygen contents in the tissues, Biosensor of sugar contents in the blood, Biosensor of alcohol contents in the blood. Biosensor of narcotic substances contents in the blood and while the electro-stimulation parameters are as follows:
Current frequency: 1-300 HZ. Duration of current pulses: 0.1-0.8 Ms,
Amplitude of current pulses: 0.02-1 V.

87. The method of treatment by electro-stimulator as detailed in claim 17 applied for treating Parkinson's disease, using one or more electrodes as detailed in claim X, wherein the electrodes are implanted in the following locations inside a patient's body: Best mode—Left sinocarotid collector (over the bifurcation spot of the common carotid artery) and/or right sinocarotid collector (over the bifurcation spot of the common carotid artery) or Second best mode—Left vagus nerve (in the superior third of the neck, at the level C2-C3) and/or right vagus nerve (in the superior third of the neck, at the level C2-C3) while using the following sensors: Arterial pressure meter, Heart rate meter, Respiration rate meter, Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues Biosensor of sugar contents in the blood, and while the electro-stimulation parameters are as follows:
Current frequency: 1-300 HZ, Duration of current pulses: 0.1-0.8 Ms.
Amplitude of current pulses: 0.02-1 V.

88. The method of treatment by electro-stimulator as detailed in claim 17 applied for treating Alzheimer's disease, using one or more electrodes as detailed in claim X, wherein the electrodes are implanted in the following locations inside a patient's body: Best mode—Left sinocarotid collector (over the bifurcation spot of the common carotid artery) and/or right sinocarotid collector (over the bifurcation spot of the common carotid artery) or Second best mode—Left vagus nerve (in the superior third of the neck, at the level C2-C3) and/or right vagus nerve (in the superior third of the neck, at the level C2-C3) while using the following sensors: Arterial pressure meter, Heart rate meter, Respiration rate meter, Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues
Biosensor of sugar contents in the blood and while the electro-stimulation parameters are as follows:
Current frequency: 1-300 HZ, Duration of current pulses: 0.1-0.8 Ms,
Amplitude of current pulses: 0.02-1 V.

89. The method of treatment by electro-stimulator as detailed in claim 17 applied for treating Gastric ulcer, using one or more electrodes as detailed in claim X, wherein the electrodes are implanted in the following locations inside a patient's body, Best mode—Left vagus nerve (in the superior third of the neck, at the level C2-C3) and/or right vagus nerve (in the superior third of the neck, at the level C2-C3), or Second best mode—Left Sympathetic trunk, thoracic (in the superior third, at the level, T7-T-11) and/or right sympathetic trunk, thoracic (in the superior third, at the level T2-T4) while using the following sensors: Arterial pressure meter. Heart rate meter, Respiration rate meter, Temperature gage, Angular shift sensor (for the limbs), Local blood flow sensor, Sensor of electric activity of the organs, nervous centers, Gastric juice acidity sensor, Murmur sensor (heart, lungs, intestine), while using the following bio-sensors: Biosensor of oxygen contents in the tissues. Biosensor of sugar contents in the blood. Biosensor of hormone contents in the blood and while the electro-stimulation parameters are as follows:
Current frequency: 1-300 HZ. Duration of current pulses: 0.1-0.8 Ms,
Amplitude of current pulses: 0.02-1 V.

90. The method of treatment by electro-stimulator as detailed in claim 17 applied for treating Pain syndromes of different genesis, using one or more electrodes, wherein the electrodes are implanted in the following locations inside a patient's body: Best mode—Spinal cord (at the level T10-T-12) or Second best mode—Right sympathetic trunk, thoracic (in the superior third, at the level T2-T4) and/or left sympathetic trunk, thoracic (in the superior third, at the level T7-T-11) while using the following sensors: Arterial pressure meter Heart rate meter. Respiration rate meter. Temperature gage, Angular shift sensor (for the limbs), Local blood flow sensor. Sensor of electric activity of the organs, nervous centers, Murmur sensor (heart, lungs, intestine)
while using the following bio-sensors: Biosensor of oxygen contents in the tissues, and while the electro-stimulation parameters are as follows:
Current frequency: 1-300 HZ, Duration of current pulses: 0.1-0.8 Ms.
Amplitude of current pulses: 0.02-1 V.

91. The method of treatment by electro-stimulator as detailed in claim 17 applied for treating Osteoporosis, using one or more electrodes, wherein the electrodes are implanted in the following locations inside a patient's body: Best mode—Nerve collector of the organ (parathyroid gland, muscles) or Second best mode—Nerve collector of the organ (muscles) while using the following sensors: Arterial pressure meter, Heart rate meter, Respiration rate meter, Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues, and while the electro-stimulation parameters are as follows: Current frequency: 1-300 HZ, Duration of current pulses: 0.1-0.8 Ms, Amplitude of current pulses: 0.02-1 V.

92. The method of treatment by electro-stimulator as detailed in claim 17 applied for treating Constipation, using one or more electrodes, wherein the electrodes are implanted in the following locations inside a patient's body: Best mode—Right sympathetic trunk: cervical (in the superior third of the neck, at the level C2-C3) and/or Left sympathetic trunk (Left upper cervical third part between C2-C3) or Second best mode—Right vagus nerve (in the superior third of the neck, at the level C2-C3) and/or Left vagus nerve (in the superior third of the neck, at the level C2-C3) while using the following sensors: Arterial pressure meter, Heart rate meter, Respiration rate meter Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues, and while the electro-stimulation parameters are as follows: Current frequency: 1-300 HZ. Duration of current pulses: 0.1-0.8 Ms, Amplitude of current pulses: 0.02-1 V.

93. The method of treatment by electro-stimulator as detailed in claim 17 applied for treating Apnea, using one or more electrodes as detailed, wherein the electrodes are implanted in the following locations inside a patient's body: Best mode—Right sympathetic trunk, cervical (in the superior third of the neck, at the level C2-C3) and/or Left sympathetic trunk (Left upper cervical third part between C2-C3) or Second best mode—Spinal cord (at the level T10-T-12) while using the following sensors: Arterial pressure meter, Heart rate meter, Respiration rate meter, Temperature gage, Angular shift sensor (for the limbs), Local blood flow sensor. Sensor of electric activity of the organs, nervous centers, Murmur sensor (heart, lungs, intestine), while using the following bio-sensors: Biosensor of oxygen contents in the tissues and while the electro-stimulation parameters are as follows:
Current frequency: 1-300 HZ, Duration of current pulses: 0.1-0.8 Ms,
Amplitude of current pulses: 0.02-1 V.

94. The method of treatment by electro-stimulator as detailed in claim 17 applied for treating Insomnia, using one or more electrodes as detailed, wherein the electrodes are implanted in the following locations inside a patient's body: Best mode—Right sinocarotid collector (over the bifurcation spot of the common carotid artery) and/or left sinocarotid collector (over the bifurcation spot of the common carotid artery) or Second best mode—Right vagus nerve (in the superior third of the neck, at the level C2-C3) and/or left vagus nerve (in the superior third of the neck, at the level C2-C3) while using the following sensors: Arterial pressure meter, Heart rate meter, Respiration rate meter. Temperature gage, Angular shift sensor (for the limbs), Local blood flow sensor, Sensor of electric activity of the organs, nervous centers Murmur sensor (heart: lungs, intestine), while using the following bio-sensors: Biosensor of oxygen contents in the tissues
and while the electro-stimulation parameters are as follows:
Current frequency: 1-300 HZ, Duration of current pulses: 0.1-0.8 Ms, Amplitude of current pulses: 0.02-1 V.

95. The method of treatment by electro-stimulator as detailed in claim 17 applied for treating Hypersomnia, using one or more electrodes as detailed, wherein the electrodes are implanted in the following locations inside a patient's body: Best mode—Right sympathetic trunk, cervical (in the superior third of the neck, at the level C2-C3) and/or Left sympathetic trunk (Left upper cervical third part between C2-C3) or Second best mode—Spinal cord (at the level T10-T-12) while using the following sensors: Arterial pressure meter, Angular shift sensor (for the limbs), Local blood flow sensor, Sensor of electric activity of the organs, nervous centers, Murmur sensor (heart, lungs, intestine) while using the following bio-sensors: Biosensor of oxygen contents in the tissues, and while the electro-stimulation parameters are as follows:
Current frequency: 1-300 HZ, Duration of current pulses: 0.1-0.8 Ms,
Amplitude of current pulses: 0.02-1 V.

96. The method of treatment by electro-stimulator as detailed in claim 17 applied for treating Obliterating vascular diseases of the limbs, using one or more electrodes as detailed, wherein the electrodes are implanted in the following locations inside a patient's body: Best mode—Spinal cord (at the level T10-T-12) or Second best mode—right sympathetic trunk, thoracic (in the superior third; at the level T2-T4) and/or left sympathetic trunk, thoracic (in the superior third at the level T7-T-11) while using the following sensors: Arterial pressure meter, Heart rate meter Respiration rate meter
Angular shift sensor (for the limbs), Local blood flow sensor. Sensor of electric activity of the organs, nervous centers, Murmur sensor (heart, lungs, intestine), while using the following bio-sensors: Biosensor of oxygen contents in the tissues, and while the electro-stimulation parameters are as follows: Current frequency: 1-300 HZ. Duration of current pulses: 0.1-0.8 Ms, Amplitude of current pulses: 0.02-1 V.

97. The method of treatment by electro-stimulator as detailed in claim 17 applied for treating Urinary bladder atony using one or more electrodes as detailed, wherein the electrodes are implanted in the following locations inside a patient's body: Best mode—Nerve collector of the organ (v. bladder) or Second best mode—Spinal cord (at the level T10-T-12) while using the following sensors: Arterial pressure meter. Heart rate meter, Respiration rate meter; Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues, and while the electro-stimulation parameters are as follows: Current frequency: 1-300 HZ, Duration of current pulses: 0.1-0.8 Ms, Amplitude of current pulses: 0.02-1 V.

98. The method of treatment by electro-stimulator as detailed in claim 17 applied for treating Ailments associated with a malfunction of the rectum sphincters, using one or more electrodes as detailed wherein the electrodes are implanted in the following locations inside a patient's body: Best mode—Nerve collector of the organ (rectum) while using the following sensors, Arterial pressure meter, Heart rate meter, Respiration rate meter, Temperature gage, Angular shift sensor (for the limbs), Local blood flow sensor, Sensor of electric activity of the organs, nervous centers, Murmur sensor (heart, lungs, intestine), while using the following bio-sensors:
Biosensor of oxygen contents in the tissues, and while the electro-stimulation parameters are as follows: Current frequency: 1-300 HZ, Duration of current pulses: 0.1-0.8 Ms. Amplitude of current pulses: 0.02-1 V.

99. The method of treatment by electro-stimulator as detailed in claim 17 applied for treating Bulimia, using one or more electrodes as detailed wherein the electrodes are implanted in the following locations inside a patient's body: Best mode—Right vagus nerve (in the superior third of the neck, at the level C2-C3) and/or left vagus nerve (in the superior third of the neck, at the level C2-C3) or Second best mode—Right sympathetic trunk, thoracic (in the superior third, at the level T2-T4) and/or left sympathetic trunk thoracic (in the superior third, at the level T7-T-11) while using the following sensors: Arterial pressure meter. Heart rate meter, Respiration rate meter Temperature gage Angular shift sensor (for the limbs), Local blood flow sensor, Sensor of electric activity of the organs, nervous centers. Gastric juice acidity sensor, Murmur sensor (heart, lungs, intestine) while using the following bio-sensors-Biosensor of oxygen contents in the tissues Biosensor of sugar contents in the blood, Biosensor of hormone contents in the blood, and while the electro-stimulation parameters are as follows-Current frequency: 1-300 HZ. Duration of current pulses-0.1-0.8 Ms. Amplitude of current pulses: 0.02-1 V.

100. The method of treatment by electro-stimulator as detailed in claim 17 applied for treating Reflux-esophagitis: using one or more electrodes as detailed wherein the electrodes are implanted in the following locations inside a patient's body: Best mode—Right vagus nerve (in the superior third of the neck, at the level C2-C3) and/or left vagus nerve (in the superior third of the neck, at the level C2-C3) or Second best mode—Right sympathetic trunk, thoracic (in the superior third, at the level T2-T4) and/or left sympathetic trunk, thoracic (in the superior third, at the level T7-T-11) while using the following sensors: Arterial pressure meter, Heart rate meter, Respiration rate meter Temperature gage, Angular shift sensor (for the limbs), Local blood flow sensor. Sensor of electric activity of the organs, nervous centers, Gastric juice acidity sensor: Murmur sensor (heart, lungs, intestine), while using the following bio-sensors:Biosensor of oxygen contents in the tissues, Biosensor of sugar contents in the blood, and while the electro-stimulation parameters are as follows: Current frequency: 1-300 HZ. Duration of current pulses: 0.1-0.8, Ms, Amplitude of current pulses: 0.02-1 V.

101. The method of treatment by electro-stimulator as detailed in claim 17 applied for treating Infantile cerebral paralysis using one or more electrodes as detailed wherein the electrodes are implanted in the following locations inside a patient's body: Best mode—Spinal cord (at the level T10-T-12) and Nerve collector of the organ (Muscles) or Second best mode—Nerve collector of the organ (Muscles) while using the following sensors: Arterial pressure meter. Heart rate meter; Respiration rate meter, Temperature gage, Angular shift sensor (for the limbs), Local blood flow sensor, Sensor of electric activity of the organs, nervous centers, Murmur sensor (heart, lungs, intestine) while using the following bio-sensors: Biosensor of oxygen contents in the tissues, and while the electro-stimulation parameters are as follows:
Current frequency: 1-300 HZ, Duration of current pulses: 0.1-0.8 Ms.
Amplitude of current pulses: 0.02-1 V.

102. The method of treatment by electro-stimulator as detailed in claim 17 applied for treating Posttraumatic paralysis using one or more electrodes as detailed, wherein the electrodes are implanted in the following locations inside a patient's body: Best mode—Spinal cord (at the level T10-T-12) and Nerve collector of the organ (Muscles) or Second best mode—Nerve collector of the organ (Muscles) while using the following sensors, Arterial pressure meter. Heart rate meter, Respiration rate meter, Temperature gage, Angular shift sensor (for the limbs), Local blood flow sensor, Sensor of electric activity of the organs, nervous centers. Murmur sensor (heart, lungs, intestine, while using the following bio-sensors: Biosensor of oxygen contents in the tissues, and while the electro-stimulation parameters are as follows:
Current frequency: 1-300 HZ. Duration of current pulses: 0.1-0.8 Ms,
Amplitude of current pulses: 0.02-1 V.

103. The method of treatment by electro-stimulator as detailed in claim 17 applied for treating Hirschsprung's disease using one or more electrodes as detailed wherein the electrodes are implanted in the following locations inside a patient's body: Best mode—Left sympathetic trunk, thoracic (in the superior third, at the level T7-T-11) and/or right sympathetic trunk, thoracic (in the superior third, at the level T2-T4) or Second best mode—Nerve collector of the organ (colon) while using the following sensors: Arterial pressure meter, Heart rate meter, Respiration rate meter, Temperature gage, Angular shift sensor (for the limbs), Local blood flow sensor, Sensor of electric activity of the organs, nervous centers. Murmur sensor (heart: lungs: intestine) while using the following bio-sensors: Biosensor of oxygen contents in the tissues, and while the electro-stimulation parameters are as follows:
Current frequency: 1-300 HZ. Duration of current pulses: 0.1-0.8 Ms,
Amplitude of current pulses: 0.02-1 V.

104. The method of treatment by electro-stimulator as detailed in claim 17 applied for treating Depression using one or more electrodes as detailed wherein the electrodes are implanted in the following locations inside a patient's body: Best mode—Right sinocarotid collector (over the bifurcation spot of the common carotid artery) and/or left sinocarotid collector (over the bifurcation spot of the common carotid artery) or Second best mode—right sympathetic trunk, cervical (in the superior third of the neck, at the level C2-C3) and/or left sympathetic trunk (Left upper cervical third part between C2-C3) while using the following sensors: Arterial pressure meter, Heart rate meter, Respiration rate meter, Temperature gage, Angular shift sensor (for the limbs), Local blood flow sensor, Sensor of electric activity of the organs, nervous centers. Murmur sensor (heart, lungs, intestine), while using the following bio-sensors: Biosensor of oxygen contents in the tissues
Biosensor of sugar contents in the blood
and while the electro-stimulation parameters are as follows:
Current frequency: 1-300 HZ, Duration of current pulses: 0.1-0.8 Ms,
Amplitude of current pulses: 0.02-1 V.

105. The method of treatment by electro-stimulator as detailed in claim 17 applied for treating Schizophrenia, using one or more electrodes as detailed wherein the electrodes are implanted in the following locations inside a patient's body: Best mode—Right sinocarotid collector (over the bifurcation spot of the common carotid artery) and/or left sinocarotid collector (over the bifurcation spot of the common carotid artery) or Second best mode—right sympathetic trunk, thoracic (in the superior third, at the level T2-T4) and/or left sympathetic trunk, thoracic (in the superior third, at the level T7-T-11) while using the following sensors: Arterial pressure meter, Heart rate meter, Respiration rate meter, Temperature gage, Angular shift sensor (for the limbs). Local blood flow sensor, Sensor of electric activity of the organs, nervous centers, Murmur sensor (heart, lungs, intestine), while using the following bio-sensors: Biosensor of oxygen contents in the tissues, Biosensor of sugar contents in the blood
and while the electro-stimulation parameters are as follows:
Current frequency: 1-300 HZ, Duration of current pulses: 0.1-0.8 Ms.
Amplitude of current pulses: 0.02-1 V.

106. The method of treatment by electro-stimulator as detailed in claim 17 applied for treating Borderline personality disorders using one or more electrodes as detailed wherein the electrodes are implanted in the following locations inside a patient's body: Best mode—Right sinocarotid collector (over the bifurcation spot of the common carotid artery) and/or left sinocarotid collector (over the bifurcation spot of the common carotid artery) or Second best mode—right sympathetic trunk, thoracic (in the superior third, at the level T2-T4) and/or left sympathetic trunk, thoracic (in the superior third, at the level T7-T-11) while using the following sensors: Arterial pressure meter, Heart rate meter, Respiration rate meter, Temperature gage, Angular shift sensor (for the limbs), Local blood flow sensor, Sensor of electric activity of the organs, nervous centers, Murmur sensor (heart, lungs, intestine, while using the following bio-sensors: Biosensor of oxygen contents in the tissues. Biosensor of sugar contents in the blood
and while the electro-stimulation parameters are as follows:
Current frequency: 1-300 HZ, Duration of current pulses: 0.1-0.8 Ms.
Amplitude of current pulses-0.02-1 V.

107. The method of treatment by electro-stimulator as detailed in claim 17 applied for treating Nervous anorexia using one or more electrodes as detailed wherein the electrodes are implanted in the following locations inside a patient's body: Best mode—Right sinocarotid collector (over the bifurcation spot of the common carotid artery) and/or left sinocarotid collector (over the bifurcation spot of the common carotid artery) or Second best mode—right sympathetic trunk, thoracic (in the superior third, at the level T2-T4) and/or left sympathetic trunk, thoracic (in the superior third, at the level T7-T-11) while using the following sensors: Arterial pressure meter, Heart rate meter. Respiration rate meter, Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues, Biosensor of sugar contents in the blood, and while the electro-stimulation parameters are as follows: Current frequency: 1-300 HZ, Duration of current pulses: 0.1-0.8. Ms, Amplitude of current pulses: 0.02-1 V.

108. The method of treatment by electro-stimulator as detailed in claim 17 applied for treating Adrenal cortex hypofunction (Addison's disease), using one or more electrodes as detailed, wherein the electrodes are implanted in the following locations inside a patient's body: Best mode—Right sympathetic trunk, thoracic (in the superior third, at the level T2-T4) and/or left sympathetic trunk, thoracic (in the superior third, at the level T7-T-11) or Second best mode—Nerve collector of the organ while using the following sensors: Arterial pressure meter: Heart rate meter, Respiration rate meter Temperature gage, Angular shift sensor (for the limbs), Local blood flow sensor, Sensor of electric activity of the organs, nervous centers. Murmur sensor (heart, lungs, intestine), while using the following bio-sensors: Biosensor of oxygen contents in the tissues, Biosensor of sugar contents in the blood. Biosensor of hormone contents in the blood
and while the electro-stimulation parameters are as follows:
Current frequency: 1-300 HZ, Duration of current pulses: 0.1-0.8 Ms,
Amplitude of current pulses: 0.02-1 V.

109. The method of treatment by electro-stimulator as detailed in claim 17 applied for treating Adrenal cortex insufficiency using one or more electrodes as detailed wherein the electrodes are implanted in the following locations inside a patient's body: Best mode—Right sympathetic trunk, thoracic (in the superior third, at the level T2-T4) and/or left sympathetic trunk, thoracic (in the superior third, at the level T7-T-11) or Second best mode—Nerve collector of the organ while using the following sensors: Arterial pressure meter. Heart rate meter, Respiration rate meter. Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues. Biosensor of sugar contents in the blood, Biosensor of hormone contents in the blood, and while the electro-stimulation parameters are as follows:
Current frequency: 1-300 HZ, Duration of current pulses: 0.1-0.8 Ms,
Amplitude of current pulses: 0.02-1 V.

110. The method of treatment by electro-stimulator as detailed in claim 17 applied for treating Hypothyrosis using one or more electrodes as detailed wherein the electrodes are implanted in the following locations inside a patient's body: Best mode—Left sympathetic trunk (Left upper cervical third part between C2-C3) and/or right sympathetic trunk, thoracic (in the superior third, at the level T2-T4) or Second best mode—Left vagus nerve (in the superior third of the neck, at the level C2-C3) and/or right vagus nerve (in the superior third of the neck, at the level C2-C3) while using the following sensors: Arterial pressure meter, Heart rate meter, Respiration rate meter Temperature gage, Angular shift sensor (for the limbs), Local blood flow sensor, Sensor of electric activity of the organs, nervous centers, Murmur sensor (heart, lungs, intestine), while using the following bio-sensors: Biosensor of oxygen contents in the tissues, Biosensor of hormone contents in the blood, and while the electro-stimulation parameters are as follows: Current frequency: 1-300 HZ. Duration of current pulses: 0.1-0.8 Ms, Amplitude of current pulses: 0.02-1 V.

111. The method of treatment by electro-stimulator as detailed in claim 17 applied for treating Progressing myodystrophy, using one or more electrodes as detailed wherein the electrodes are implanted in the following locations inside a patient's body: Best mode—Spinal cord (at the level T10-T-12) and Nerve collector of the organ (Muscles) or Second best mode—Nerve collector of the organ (Muscles) while using the following sensors: Arterial pressure meter. Heart rate meter. Respiration rate meter, Temperature gage Angular shift sensor (for the limbs), Local blood flow sensor, Sensor of electric activity of the organs, nervous centers, Murmur sensor (heart: lungs, intestine) while using the following bio-sensors: Biosensor of oxygen contents in the tissues, and while the electro-stimulation parameters are as follows:
Current frequency: 1-300 HZ. Duration of current pulses: 0.1-0.8 Ms,
Amplitude of current pulses: 0.02-1 V.

112. The method of treatment by electro-stimulator as detailed in claim 17 applied for treating Angiotrophone neurosis, using one or more electrodes as detailed wherein the electrodes are implanted in the following locations inside a patient's body: Best mode—Spinal cord (at the level T10-T-12) or Second best mode—Nerve collector of the organ (Muscles) while using the following sensors: Arterial pressure meter. Heart rate meter, Respiration rate meter, Temperature gage, Angular shift sensor (for the limbs), Local blood flow sensor. Sensor of electric activity of the organs, nervous centers Murmur sensor (heart, lungs, intestine), while using the following bio-sensors: Biosensor of oxygen contents in the tissues
and while the electro-stimulation parameters are as follows:
Current frequency: 1-300 HZ, Duration of current pulses: 0.1-0.8 Ms.
Amplitude of current pulses: 0.02-1 V.

113. The method of treatment by electro-stimulator as detailed in claim 17 applied for treating Syringomyelia, using one or more electrodes as detailed, wherein the electrodes are implanted in the following locations inside a patient's body: Best mode—Right sympathetic trunk, thoracic (in the superior third, at the level T2-T4) and/or left sympathetic trunk, thoracic (in the superior third; at the level T7-T-11) or Second best mode—Right sympathetic trunk, cervical (in the superior third of the neck, at the level C2-C3) and/or left sympathetic trunk (Left upper cervical third part between C2-C3) while using the following sensors: Arterial pressure meter. Heart rate meter, Respiration rate meter. Temperature gage, Angular shift sensor (for the limbs), Local blood flow sensor, Sensor of electric activity of the organs, nervous centers Murmur sensor (heart, lungs, intestine), while using the following bio-sensors: Biosensor of oxygen contents in the tissues
and while the electro-stimulation parameters are as follows:
Current frequency: 1-300 HZ, Duration of current pulses: 0.1-0.8 Ms,
Amplitude of current pulses: 0.02-1 V.

114. The method of treatment by electro-stimulator as detailed in claim 17 applied for treating Impotence, using one or more electrodes as detailed wherein the electrodes are implanted in the following locations inside a patient's body: Best mode—Nerve collector of the organ (skin) while using the following sensors: Arterial pressure meter, Heart rate meter Respiration rate meter, Temperature gage, Angular shift sensor (for the limbs) Local blood flow sensor, Sensor of electric activity of the organs, nervous centers, Murmur sensor (heart, lungs, intestine), while using the following bio-sensors: Biosensor of oxygen contents in the tissues, Biosensor of sugar contents in the blood. Biosensor of alcohol contents in the blood, Biosensor of narcotic substances contents in the blood
and while the electro-stimulation parameters are as follows:
Current frequency: 1-300 HZ. Duration of current pulses: 0.1-0.8 Ms,
Amplitude of current pulses: 0.02-1 V.

115. The method of treatment by electro-stimulator as detailed in claim 17 applied for treating Paresis of the vocal cords, using one or more electrodes as detailed, wherein the electrodes are implanted in the following locations inside a patient's body: Best mode—Left vagus nerve (in the superior third of the neck, at the level C2-C3) and/or right vagus nerve (in the superior third of the neck, at the level C2-C3) or Second best mode—Right sympathetic trunk, cervical (in the superior third of the neck, at the level C2-C3) and/or Left sympathetic trunk (Left upper cervical third part between C2-C3) while using the following sensors: Arterial pressure meter, Heart rate meter, Respiration rate meter, Temperature gage, Angular shift sensor (for the limbs), Local blood flow sensor, Sensor of electric activity of the organs, nervous centers Murmur sensor (heart, lungs, intestine), while using the following bio-sensors: Biosensor of oxygen contents in the tissues
and while the electro-stimulation-parameters are as follows:
Current frequency: 1-300 HZ, Duration of current pulses: 0.1-0.8 Ms:
Amplitude of current pulses: 0.02-1 V.

116. The method of treatment by electro-stimulator as detailed in claim 17 applied for treating Snoring, using one or more electrodes as detailed wherein the electrodes are implanted in the following locations inside a patient's body: Best mode—Right sympathetic trunk, cervical (in the superior third of the neck, at the level C2-C3) and/or Left sympathetic trunk (Left upper cervical third part between C2-C3) or Second best mode—Spinal cord (at the level T10-T-12) while using the following sensors:
Arterial pressure meter, Heart rate meter. Respiration rate meter, Temperature gage, Angular shift sensor (for the limbs), Local blood flow sensor, Sensor of electric activity of the organs, nervous centers. Murmur sensor (heart, lungs, intestine), while using the following bio-sensors:
Biosensor of oxygen contents in the tissues
and while the electro-stimulation 1 parameters are as follows:
Current frequency: 1-300 HZ, Duration of current pulses: 0.1-0.8 Ms,
Amplitude of current pulses: 0.02-1 V.

117. The method of treatment by electro-stimulator as detailed in claim 17 applied for treating Chronic gastroenterocolitis, using one or more electrodes as detailed wherein the electrodes are implanted in the following locations inside a patient's body: Best mode—Right vagus nerve (in the superior third of the neck, at the level C2-C3) and/or left vagus nerve (in the superior third of the neck, at the level C2-C3) or Second best mode—Right sympathetic trunk, thoracic (in the superior third, at the level T2-T4) and/or left sympathetic trunk, thoracic (in the superior third, at the level T7-T-11) while using the following sensors: Arterial pressure meter. Heart rate meter, Respiration rate meter Temperature gage, Angular shift sensor (for the limbs): Local blood flow sensor. Sensor of electric activity of the organs, nervous centers. Murmur sensor (heart, lungs, intestine), while using the following bio-sensors:
Biosensor of oxygen contents in the tissues, Biosensor of sugar contents in the blood, and while the electro-stimulation parameters are as follows:
Current frequency: 1-300 HZ, Duration of current pulses: 0.1-0.8 Ms,
Amplitude of current pulses: 0.02-1 V.

118. The method of treatment by electro-stimulator as detailed in claim 17 applied for treating Chronic gastroenterocolitis, using one or more electrodes as detailed wherein the electrodes are implanted in the following locations inside a patient's body: Best mode—Right vagus nerve (in the superior third of the neck, at the level C2-C3) and/or left vagus nerve, left (in the superior third of the neck, at the level C2-C3) or Second best mode—Right sympathetic trunk, thoracic (in the superior third, at the level T2-T4) and/or left sympathetic trunk, thoracic, left (in the superior third, at the level T7-T-11) while using the following sensors: Arterial pressure meter, Heart rate meter, Respiration rate meter, Temperature gage, Angular shift sensor (for the limbs), Local blood flow sensor. Sensor of electric activity of the organs, nervous centers Murmur sensor (heart, lungs, intestine), while using the following bio-sensors: Biosensor of oxygen contents in the tissues, Biosensor of sugar contents in the blood, and while the electro-stimulation parameters are as follows: Current frequency: 1-300 HZ, Duration of current pulses: 0.1-0.8 Ms, Amplitude of current pulses: 0.02-1 V.

119. The method of treatment by electro-stimulator as detailed in claim 17 applied for treating Stammering, using one or more electrodes as detailed, wherein the electrodes are implanted in the following locations inside a patient's body: Best mode—Sinocarotid collector, right (over the bifurcation spot of the common carotid artery) and/or left sinocarotid collector, left (over the bifurcation spot of the common carotid artery) or Second best mode—Right sympathetic trunk, cervical (in the superior third of the neck, at the level C2-C3) and/or Left sympathetic trunk (Left upper cervical third part between C2-C3) while using the following sensors:
Arterial pressure meter. Heart rate meter, Respiration rate meter, Temperature gage, Angular shift sensor (for the limbs), Local blood flow sensor, Sensor of electric activity of the organs, nervous centers, Murmur sensor (heart, lungs, intestine), while using the following bio-sensors:
Biosensor of oxygen contents in the tissues
and while the electro-stimulation parameters are as follows:
Current frequency: 1-300 HZ. Duration of current pulses: 0.1-0.8 Ms,
Amplitude of current pulses: 0.02-1 V.

120. The method of treatment by electro-stimulator as detailed in claim 17 applied for treating Agranulocytosis, Anemia, Atrophy of he optic nerve: Skin atrophy, Sardiac achalasia, Coagulation disorders; Buerger's disease, Crohn's disease, Pick's disease, Raynaud's disease, Vegetovascular dystony, Sudden cardiac arrest at sleep, Rectal prolapse, Flaccidly consolidating fractures, Hyperkeratosis; Hyperostosis, Adrenal cortex hyperfunction, Hypoglycemia, Subcortical dementia, Multi-infarction dementia Involuntary movements, Dyskinesia of the biliferous tracts, Dysmenorrhea (genital diseases), Dolichosigmoid, Stammering, Impotence, Leucosis: Migraine, Myodystrophy, Myopathy; Male and female infertility, Narcolepsy, Bauhin's valve failure, Neurodermite, Obliterating atherosclerosis of the limbs' vessels, Obliterating endarteritis of the lower limbs' vessels, Alopecia, Paralyses of different etiology, Paresis of the vocal cords, Progressing myodystrophy, Psoriasis. Rickets, Zollinger-Ellison syndrome, Commissural disease, Huntington's chorea, Chronic duodenal ileus. Chronic and acute hyperthermia, Chronic gastroenterocolitis, Chronic cholecystitis and cholecystopancreatitis. Chronic cystitis. Chronic glomerulonephritis, Chronic obstructive bronchitis, Chronic osteomyelitis, Chronic pyelonephritis. Cirrhosis. Eczema. Pulmonary emphysema, Enuresis and/or Duodenal ulcer, using one or more electrode as detailed wherein the electrodes are implanted in the following locations inside a patient's body:
Sinocarotid collector, right (over the bifurcation spot of the common carotid artery)
And/or Sinocarotid collector, left (over the bifurcation spot of the common carotid artery) And/or Sympathetic trunk, thoracic, right (in the superior third, at the level T2-T4)
And/or Sympathetic trunk, thoracic, left (in the superior third, at the level T7-T-11) And/or Vagus nerve, left (in the superior third of the neck, at the level C2-C3)
And/or Vagus nerve, right (in the superior third of the neck, at the level C2-C3) And/or Sympathetic trunk, cervical, right (in the superior third of the neck, at the level C2-C3) And/or Left sympathetic trunk (Left upper cervical third part between C2-C3)
And/or Spinal cord (at the level T10-T-12) And/or Nerve collector of the organ (Muscles) And/or Depressor nerve, right (over the aortic arch or in the middle third of the neck) And/or Depressor nerve, left (over the aortic arch or in the middle third of the neck) while using the following sensors.
Arterial pressure meter, Heart rate meter. Respiration rate meter, Temperature gage, Angular shift sensor (for the limbs), Local blood flow sensor, Sensor of electric activity of the organs, nervous centers, Gastric juice acidity sensor, Murmur sensor (heart, lungs, intestine), while using the following bio-sensors: Biosensor of oxygen contents in the tissues. Biosensor of sugar contents in the blood, Biosensor of hormone contents in the blood Biosensor of alcohol contents in the blood. Biosensor of narcotic substances contents in the blood, and while the electro-stimulation parameters are as follows:Current: frequency: 1-300 HZ Duration of current pulses-0.1-0.8 Ms. Amplitude of current pulses: 0.02-1 V.

121. The medical treatment method according to claim 17 wherein, in order to increase the safety level of the implanted Stimulator (to prevent one patient from accidental using of non-implanted parts of the Electric Stimulator that belongs to another patient), each Electric Stimulator is assigned a data code (PIN code, similarly to cellular phones), to make sure the Electric Stimulator is used on an individual basis only, while due to this feature it also becomes possible to remotely (for example, via the Internet) replace the Electric Stimulator's software, to monitor and to examine the patient's condition from large distances.

122. The medical treatment method according to claim 17 wherein a PIN-card (like in cellular phones) is either inserted into the Electric Stimulator, or a relevant data code can be saved in the memory unit thereof.

123. The medical treatment method according to claim 17 wherein the codes will also be used for manufacturing accountability purposes which is important in order to prevent other companies from faking the device.

124. The medical treatment method according to claim 17 wherein upon occurrence of abnormal functioning of the body's systems, organs, tissues, either as a result of the Electric Stimulator's operation or regardless thereof, endangering the patient's health, the Electric Stimulator's indicating device informs both the patient and medical personnel of such occurrence via the internal and/or external radio-frequency communication units, being specific about a certain side effect that has developed, after which the Electric Stimulator is automatically deactivated, in case this said side effect has developed as a response to any program of the Electric Stimulator; while such Electric Stimulator's programs identified during the Stimulator's adjustment using the external display, are either removed from the device, or they are replaced with the programs that do not cause side effects.

125. The medical treatment method according to claim 17 wherein to increase the Electric Stimulator's reliability and safety for the patient, the device has an option, in case of its breakdown, allowing the Electric Stimulator to directly transmit stimulating pulses to the electrode (electrodes) from the external radio-frequency communication unit, via the internal radio-frequency communication unit, for which purpose the latter is connected to the electrode(s) through the backup communication unit.

126. The medical treatment method according to claim 17 wherein its internal radio-frequency communication unit is made selectively sensitive to only those signals sent by the external units and addressed thereto, that have an identification information code belonging to a specific Electric Stimulator (PIN-type code), while the signals which do not contain the above-mentioned code do not affect the Electric Stimulator's operation and cannot control it.

127. The medical treatment method according to claim 17 wherein its internal radio-frequency communication unit is made selectively sensitive to only those signals sent by the external units and addressed thereto, to enable the information code enables security preventing any unauthorized access to the Electric Stimulator.

128. The medical treatment method according to claim 17 applied for Correction of Functional Activity of the Body's Systems, and or Organs, and/or Tissues in a healthy person that has the following distinct feature: the optimal mode of impacting the body with the Electric Stimulator (electric current parameters, duration of electric stimulation sessions, etc.) are selected and controlled automatically, and/or by the doctor, and/or by the patient in a real time frame in accordance with the program algorithms input the Electric Stimulator, and subject to an analysis conducted by means of implanted or non-implanted sensor(s) and/or biosensor(s) of one or a set of homeostatic properties of the body: contents and concentration of substances, gases in the body's tissues and liquid media, and values of electric, mechanical and/or acoustic activity of the organs and tissues; in order to obtain the maximal correcting effect (of stimulation or inhibition), one or several systems, and/or tissues, and/or organs of the body are simultaneously subjected to the electric stimulation through individual channels, electrodes, program algorithms or through one, common program and algorithms.

129. The medical treatment method according to claim 17 applied for a healthy person wherein a principle of long-term (“calendar”) programming is used in the Electric Stimulator, and this includes application of the following three types of programs:
First-Type Programs—programming of the Electric Stimulator for the period of 1-30 days, Second-Type Programs—programming of the Electric Stimulator for the period of 1 up to 12 months, in accordance with the signals sent by the sensors and biosensors, or irregardless thereof, Third-Type Programs—programming of the Electric Stimulator for the period of more than 12 months (years).

130. The medical treatment method according to claim 17 applied for a healthy person that have the following distinct features. Electric Stimulator and the electrode(s) thereof can be implanted into different parts of the body, including tissues, organs and/or vessels, while the Electric Stimulator's shell is implanted either into the subcutaneous fat of the right or left subclavian areas, or into the subcutaneous fat of the anterior abdominal wall, in the left hypochondriumn or into the lumbar area, either form the right or left.

131. The medical treatment method according to claim 17 applied for settings of the implanted Stimulator, control and analysis thereof, as well as control and analysis of functional activity of the body's systems, organs and tissues impacted with the Electric Stimulator, may be carried out without the external non-implanted display and it can be based or the existing methods and equipment widely used and radio electronics.

132. The medical instrument according to claim 1, wherein its external radio-frequency communication unit can be connected to the external non-implanted display monitoring conditions of the body's systems, organs; tissues, and the non-implanted or temporarily implanted into the patient's body sensors and biosensors are connected thereto; while the data on the body's systems are transmitted from the display, via the internal radio-frequency communication unit, to the implanted Electric Stimulator's units, being additional to and/or replacing the data, received by these said units from the implanted sensors and biosensors.

133. The medical instrument according to claim 1, further including sensors usable as catheter means without electrode: the received data can be displayed using any display means, A Non-Implanted External Display may be used that, according to Clause B above, has the following distinct feature: it allows to monitor functional activity of the respiratory; digestive, cardiovascular, nervous, immune, urinary and/or temperature control systems, while both the implanted or non-implanted sensors and biosensors of the Display or Electric Stimulator can be used for this purposes.

134. The medical instrument according to claim 1, further including an option that allows additional, both implanted and non-implanted units to be connected thereto.

135. The medical instrument according to claim 1, further including additional non-implanted units which are connected to the Electric Stimulator via the external radio-frequency communication unit and internal radio-frequency communication unit, while the additional implanted units, which are connected to the Electric Stimulator's shell, units or electrodes, such connection being made with electrode(s) or without them; using plug-and-socket units.

136. The medical instrument according to claim 1, wherein each of its main units can be both implanted and non-implanted, while being connected to the remaining units of the Electric Stimulator, found in the shell or electrode(s) thereof via the external radio-frequency communication unit, internal radio-frequency communication unit (for the non-implanted units), or with an electrode or plug-and socket unit(s) (for the implanted ones).

137. The implanted system according to claim 1, further including a noncontact communication channel with a transmitter outside a patient's body, and wherein the processor is programmable, including means for loading a working program therein from the external transmitter.

138. The medical instrument according to claim 1, wherein the diseases include Asthma, Insomnia, Hypersonmia, Apnea, Narcolepsy, Sudden cardiac arrest at sleep, Paresis of the vocal cords, Nervous anorexia, Obesity, Bulimia, Gastric and duodenal ulcer, Chronic gastroenterocolitis, Refluxesophagitis, Gastrointestinal dyskinesia. Commissural disease, Crohn's disease, Hirschsprung's disease—megacolon, Rectal prolapse, Chronic duodenal ileus, Bauhin's valve failure, Doloichosigmoid. Chronic intestinal obstruction (commissural disease, megacolon, chronic mesenterial circulation insufficiency, metacolon, doloichosigmoid, cardiac achalasia, Schizophrenia with schizophrenic affective disorders and delirium, Anxiety and depression, Borderline personality disorder, Emphysema Cortical dementia-Alzheimer's disease. Pick's disease, Subcortical dementia-supranuclear palsy (paralysis), Huntington's chorea Parkinson's disease, Multiinfarction dementia. Involuntary movements, Stammering and/or Epilepsy.

139. The medical instrument according to claim 1, wherein the diseases include Priapism, Infantile cerebral paralysis Paralyses of different etiology Syringomyelia, Progressing myodystrophy and other forms of dystrophy, Chronic and acute hyperthermia Atrophy of the optic nerve, Chronic periodic pains (angina pectoris, phantom pains, neuritis, nerve root syndromes Terminalstage pains Migraine, Cancer pain, Hypertension, Hypotension Vegetovascular dystony, Diabetes, hypoglycemia, diabetes insipidus hypothyrosis, hyperthyrosis adrenal cortex insufficiency, male and female infertility, impotence, adrenal cortex hyperfunction, dysmenorrhea, Zollinger-Ellison syndrome. Dyskinesia of the biliferous tracts. Chronic hepatitis. Chronic cholecystopancreatitis, Cirrhosis, Osteoporosis, Periostitis; osteosclerosis of different types, Hyperostosis, Chronic osteomyelitis Flaccidly consolidating fractures and/or Rickets.

140. The medical instrument according to claim 1, wherein the diseases include Perthes disease, anemia, agranulocytosis, leucosis. Immunodeficiency trauma-related paralyses, myodystrophy; myopathy, Bodybuilding Hydronephrosis. Chronic pyelonephritis, Chronic glomerulonephritis, Urinary bladder atony, Chronic cystitis, Psoriasis, Neurodermite, Eczema, Alopecia, Hyperkeratosis, Skin atrophy, Angiotrophoneurosis, Drug addiction. Alcoholism, Obliterating atherosclerosis and endarteritis, Ischemic heart disease and angina pectoris, Cardiac arrhythmia, Raynaud's disease, Buerger's disease, Chronic thrombophlebitis-supranuclear palsy (paralysis) and/or Postthrombophlebitic syndrome.

141. A medical instrument implanted in a patient's body, comprising sensor means for measuring variables inside the body, processor means for processing the signals and monitoring the patient's health, and transmitter means for transmitting results of the monitoring to a receiver external to the patient's body.

142. The medical instrument according to claim 202, further including recording means for storing results of the patient monitoring, and wherein the processor compares the monitoring results with stored previous results and issues a warning or alarm if a undesirable change is detected.

143. The medical instrument according to claim 202, further including receiver means for receiving an external request for information and means in the processor for checking the validity of the request, and means for transmitting the monitoring results to an external receiver upon receiving a legitimate request.

144. The medical instrument according to claim 202, wherein the external receiver further including means for connecting to electronic equipment and/or Internet, for the processing and/or transfer of the monitoring results.

145. The medical instrument according to claim 205, wherein the processor means is programmed to perform a continuous monitoring of the patient and for issuing an alarm in real time if an undesirable change in the sensor's reading is detected.

146. The medical instrument according to claim 205, wherein the processor means is programmed to issue a report of the internal health state of the patient upon receiving a predefined request.

147. The medical instrument according to claim 205, wherein the processor means is programmed to perform a test at predefined time periods such as once per year or once per month, and to transmit the results of the test to an external receiver.

148. The medical treatment method according to claim 17, wherein stimulating and/or inhibiting the functional activity of the nervous structure of the sympathetic nervous system, the parasympathetic system or the sympathetic nervous system and parasympathetic system and hypoglossal (sinocarotid collector of the Vegetative Nervous System-SCVNS), the central nervous system, and/or neurons of the organ and/or cutaneous nerves and/or depressor nerves.

149. The medical treatment method according to claim 17, wherein a nervous band or group is formed, comprising all, or the majority of, the nerve branches innervating the carotid glome (glomus caroticum).

150. The medical treatment method according to claim 17, using Contacts, or indirect body measurements and using adaptive techniques for measuring body variables.

151. The medical treatment method according to claim 211 wherein during an initial stage after the implantation of the system; the sensors and biosensors are used to measure the body variables, together with the contacts, and wherein the variables from contacts are then compared in the Chip with the readouts from the sensors and biosensors.

152. The medical instrument according to claim 4, wherein the electrode is implanted to affect systems and organs of the body including the nervous structure of the sympathetic nervous system or the parasympathetic system or the sympathetic nervous system and parasympathetic system and hypoglossal (sinocarotid collector of the Vegetative Nervous System-SCVNS), the central nervous system, as well as neurons of the organ and/or cutaneous nerves and/or depressor nerves.
